(19) United States
(12) Patent Application Publication
(10) Pub. No.: US 2026/0207773 A1
(43) Pub. Date: Jul. 23, 2026

(54) RECOMBINANT AAV MUTANT VECTORS WITH CARDIAC AND SKELETAL MUSCLE-SPECIFIC TARGETING MOTIFS AND COMPOSITIONS CONTAINING SAME

(71) Applicant: The Trustees of the University of Pennsylvania, Philadelphia, PA (US)

(72) Inventors: Jenny A. Sidrane, Phoenixville, PA (US); Rosemary Meggersee, Philadelphia, PA (US); Joshua Joyner Sims, Villanova, PA (US); Jacob Hoffman, Philadelphia, PA (US); James M. Wilson, Philadelphia, PA (US)

(73) Assignee: The Trustees of the University of Pennsylvania, Philadelphia, PA (US)

(21) Appl. No.: 19/139,488

(22) PCT Filed: Dec. 15, 2023
(86) PCT No.: PCT/US2023/084192

§ 371 Date: Jun. 16, 2025

Related U.S. Application Data
(60) Provisional application No. 63/387,941, filed on Dec. 17, 2022.
(60) Provisional application No. 63/514,404, filed on Jul. 19, 2023.

(51) Int. Cl. A61K 48/00; A61K 47/64; C07K 7/06; C07K 14/005; C12N 15/86

(52) U.S. Cl. CPC A61K 48/0041; A61K 47/64; C07K 7/06; C07K 14/005; C12N 15/86; C07K 2319/01; C12N 2750/14122; C12N 2750/14143; C12N 2750/14145

(57) ABSTRACT

Provided herein are compositions including muscle (cardiac and/or skeletal) cell-targeting peptides linked thereto or inserted in a targeting protein of a recombinant vector having at least one exogenous peptide comprising Xn-RGD-n-mer-Xm. Compositions providing such conjugates, targeting peptides, or recombinant vectors having an engineered capsid or envelope protein are provided as are uses thereof.

### REFERENCE TO AN ELECTRONIC SEQUENCE LISTING

The contents of the electronic sequence listing (“UPN-22-10068PCT.xml”; Size: 183,452 bytes; and Date of Creation: Dec. 5, 2023) are herein incorporated by reference in their entirety.

### BACKGROUND OF THE INVENTION

The adeno-associated virus (AAV) is currently the gene therapy vector of choice. AAVs can deliver a transgene that is stably expressed long-term from a non-integrating genome are not associated with any human diseases. However, AAV-mediated gene therapy is currently limited to treatment of a small number of diseases due to challenges in delivery and tropism.

Treatment approaches based on AAV vectors have been approved by the US Food and Drug Administration and other worldwide regulatory authorities for the treatment of Leber congenital amaurosis, lipoprotein lipase deficiency, and spinal muscular atrophy. A central challenge for gene therapy is the difficulty of modulating and targeting expression of a therapeutic transgene in vivo, more specifically targeting a particular tissue, e.g., heart and skeletal muscle.

There is a high and unmet need in the adult- and early-onset forms of muscle cell-related pathologies (cardiac and skeletal). In such instance, lack of effective treatment can result in high rates of transplant and/or death. Various cardio- and skeletal-muscle cell-based diseases are amenable to AAV gene replacement therapy, but there is a need for specific and effective targeting to muscles tissue.

There remains a need for vectors that can specifically target selected tissues and cell types.

### SUMMARY OF THE INVENTION

In one aspect, provided herein is a recombinant adeno-associated particle (rAAV) comprising: (a) an adeno-associated virus (AAV) capsid comprising VP1 proteins, VP2 proteins and VP3 proteins, wherein the capsid proteins have an amino acid sequence comprising a hypervariable region comprising an exogenous targeting peptide, wherein the exogenous targeting peptide comprises “Xn-n-mer-Xm”, wherein: (i) Xn is 0, 1, 2 or 3 amino acid residues independently selected from any amino acid; (ii) the n-mer is IIRGDPA (SEQ ID NO: 1), AVIRGDV (SEQ ID NO: 2), IVRGDPA (SEQ ID NO: 8), MIRGDVK (SEQ ID NO: 9), AQHRGDV (SEQ ID NO: 10), VSRGDPN (SEQ ID NO: 11), VSRGDPA (SEQ ID NO: 12), PLVRGDI (SEQ ID NO: 13), PYVRGDP (SEQ ID NO: 14), VVRGDPQ (SEQ ID NO: 15), VVQRGDV (SEQ ID NO: 17), QHRGDTQ (SEQ ID NO: 18), QIRGDLR (SEQ ID NO: 19), RGDYAQV (SEQ ID NO: 20), IGRGDPN (SEQ ID NO: 21), RGDLHGY (SEQ ID NO: 22), RGDYSTM (SEQ ID NO: 23), or PYQRGDH (SEQ ID NO: 24), or an n-mer sequence of at least 6 consecutive amino acids of any one of the n-mers; and (iii) Xm is 0, 1, 2, or 3 amino acid residues independently selected from any amino acid; and (b) a vector genome packaged in the AAV capsid, wherein the vector genome comprises a nucleic acid sequence encoding a gene product under control of sequences which direct expression thereof. In certain embodiments, the rAAV comprises exogenous targeting peptide comprising an n-mer that is (a) IIRGDPA (SEQ ID NO: 1); or (b) AVIRGDV (SEQ ID NO: 2). In certain embodiments, the exogenous targeting peptide is inserted between any two contiguous amino acids in the hypervariable region VIII (HVRVIII) or the hypervariable region IV (HVRIV) at a suitable location of a parental AAV capsid. In certain embodiments, the parental AAV capsid is an AAV9, AAV8, AAV7, AAV6, AAV5, AAV4, AAV3, AAV1, AAVhu68, AAVhu95, AAVhu96, or AAVrh91 capsid. In certain embodiments, the exogenous targeting peptide is inserted in the hypervariable region between amino acids 588 and 589 in an AAV9 parental capsid as determined based on the numbering of VP1 amino acid sequence of SEQ ID NO: 25, or an analogous position in an AAV8, AAV7, AAV6, AAV5, AAV4, AAV3, AAV1, AAVhu68, AAVhu95, AAVhu96, or AAVrh91 parental AAV capsid. In certain embodiments, the exogenous targeting peptide is immediately preceded by the native AAV residues, e.g., “AQ”. In certain embodiments, the rAAV capsid comprises AAV VP1, AAV VP2 and AAV VP3 proteins having a mutant AAV VP3 region of: amino acids 204 to 743 of any one of SEQ ID NO: 73, 75, 77, 79, 81, 83, 85, 87, 89, 91, 93, 95, 97, 99, 101, 103, 105, or 107, wherein each of the VP1 proteins, VP2 proteins, and VP3 proteins have heterogenous populations which further comprise highly deamidated residues in positions N57, N329, N452 and N512 (e.g., independently about 50% to about 100% deamidated), wherein the deamidated position numbers are based on the residue positions of SEQ ID NO: 25 or SEQ ID NO: 26. In certain embodiments, the rAAV capsid comprises AAV VP1 proteins, AAV VP2 proteins and AAV VP3 proteins, wherein the VP1 has a mutant sequence of: amino acids 1 to 743 of any one of SEQ ID NO: 73, 75, 77, 79, 81, 83, 85, 87, 89, 91, 93, 95, 97, 99, 101, 103, 105, or 107, wherein the AAV VP1 proteins, AAV VP2 proteins and AAV VP3 proteins further comprise highly deamidated residues in positions N57, N329, N452 and N512, wherein the deamidated position numbers are based on the residue positions of SEQ ID NO: 25 or SEQ ID NO: 26 (e.g., independently about 50% to about 100% deamidated). In certain embodiments, the n-mer is encoded by the nucleic acid sequence of any one of SEQ ID NO: 108-125, or a sequence at least 95% identical to any one of SEQ ID NO: 108-125.

In another aspect, provided herein is a recombinant muscle cell-targeting peptide, wherein the recombinant muscle cell-targeting peptide comprises “Xn-n-mer-Xm”, wherein: (a) Xn is 0, 1, 2, or 3 amino acid residues independently selected from any amino acid; (b) the n-mer is: IIRGDPA (SEQ ID NO: 1), AVIRGDV (SEQ ID NO: 2), IVRGDPA (SEQ ID NO: 8), MIRGDVK (SEQ ID NO: 9), AQHRGDV (SEQ ID NO: 10), VSRGDPN (SEQ ID NO: 11), VSRGDPA (SEQ ID NO: 12), PLVRGDI (SEQ ID NO:13), PYVRGDP (SEQ ID NO: 14), VVRGDPQ (SEQ ID NO: 15), VVQRGDV (SEQ ID NO: 17), QHRGDTQ (SEQ ID NO: 18), QIRGDLR (SEQ ID NO: 19), RGDYAQV (SEQ ID NO: 20), IGRGDPN (SEQ ID NO: 21), RGDLHGY (SEQ ID NO: 22), RGDYSTM (SEQ ID NO: 23), or PYQRGDH (SEQ ID NO: 24), or an n-mer sequence of at least 6 consecutive amino acids of any one of the n-mers; and (iii) Xm is 0, 1, 2, or 3 amino acid/s independently selected from any amino acid; and optionally further conjugated to a nanoparticle, a second molecule, or a viral capsid protein. In certain embodiments, the recombinant muscle cell-targeting peptide targets a cardiac muscle cell and/or a skeletal muscle cell, optionally a gastrocnemius muscle cell.

In another aspect, provided herein is a nucleic acid molecule comprising a mutant AAV capsid VP1 gene comprising a nucleic acid sequence encoding an n-mer of: IIRGDPA (SEQ ID NO: 1), AVIRGDV (SEQ ID NO: 2), RGDYAQV (SEQ ID NO: 20), RGDLHGY (SEQ ID NO: 22), PYQRGDH (SEQ ID NO: 24), IVRGDPA (SEQ ID NO: 8), MIRGDVK (SEQ ID NO: 9), AQHRGDV (SEQ ID NO: 10), VSRGDPN (SEQ ID NO: 11), VSRGDPA (SEQ ID NO: 12), PLVRGDI (SEQ ID NO: 13), PYVRGDP (SEQ ID NO: 14), VVRGDPQ (SEQ ID NO: 15), VVQRGDV (SEQ ID NO: 17), QHRGDTQ (SEQ ID NO: 18), QIRGDLR (SEQ ID NO: 19), IGRGDPN (SEQ ID NO: 21), or RGDYSTM (SEQ ID NO: 23). In certain embodiments, the nucleic acid sequence encoding the n-mer comprises: (a) SEQ ID NO: 108 or a sequence at least 99% identical thereto (encoding IIRGDPA); (b) SEQ ID NO: 109 or a sequence at least 99% identical thereto (encoding AVIRGDV); (c) SEQ ID NO: 110 or a sequence at least 99% identical thereto (encoding IVRGDPA); (d) SEQ ID NO: 111 or a sequence at least 99% identical thereto (encoding MIRGDVK); (e) SEQ ID NO: 112 or a sequence at least 99% identical thereto (encoding AQHRGDV); (f) SEQ ID NO: 113 or a sequence at least 99% identical thereto (encoding VSRGDPN); (g) SEQ ID NO: 114 or a sequence at least 99% identical thereto (encoding VSRGDPA); (h) SEQ ID NO: 115 or a sequence at least 99% identical thereto (encoding PLVRGDI); (i) SEQ ID NO: 116 or a sequence at least 99% identical thereto (encoding PYVRGDP); (j) SEQ ID NO: 117 or a sequence at least 99% identical thereto (encoding VVRGDPQ); (k) SEQ ID NO: 118 or a sequence at least 99% identical thereto (encoding VVQRGDV); (1) SEQ ID NO: 119 or a sequence at least 99% identical thereto (encoding QHRGDTQ); (m) SEQ ID NO: 120 or a sequence at least 99% identical thereto (encoding QIRGDLR); (n) SEQ ID NO: 121 or a sequence at least 99% identical thereto (encoding RGDYAQV); (o) SEQ ID NO: 122 or a sequence at least 99% identical thereto (encoding IGRGDPN); (p) SEQ ID NO: 123 or a sequence at least 99% identical thereto (encoding RGDLHGY); (q) SEQ ID NO: 124 or a sequence at least 99% identical thereto (encoding RGDYSTM); or (r) SEQ ID NO: 125 or a sequence at least 99% identical thereto (encoding PYQRGDH). In certain embodiments, the nucleic acid sequence encoding the AAV capsid protein comprises SEQ ID NO: 72, 74, 76, 78, 80, 82, 84, 86, 88, 90, 92, 94, 96, 98, 100, 102, 104, or 106.

In certain embodiments, a fusion polypeptide or protein comprising a muscle cell-targeting peptide and a fusion partner that comprises at least one polypeptide or protein is provided herein. In certain embodiments, a composition comprising a fusion polypeptide or protein as provided herein and one or more of a physiologically compatible carrier, excipient, and/or aqueous suspension base.

Provided herein are also compositions and methods for using an rAAV, a muscle cell targeting peptide, a fusion polypeptide or protein, and/or a composition as described herein of for delivering a therapeutic to a patient in need thereof. In certain embodiments, the therapeutic is targeted to a muscle cell, optionally wherein the muscle cell is a cardiac muscle cell or a skeletal muscle cell, optionally a gastrocnemius muscle cell, deltoid muscle cell, a soleus muscle cell, a biceps brachii muscle cell or a diaphragm muscle cell.

In certain embodiments, a method is provided for targeting a therapy to a muscle cell in a patient in need thereof, the method comprising administering the patient an rAAV as described herein. In certain embodiments, a method is provided for treating a cardiac muscle and/or a skeletal muscle disorder and/or a disease in a subject in need thereof, the method comprising delivering to the subject a stock of an rAAV as described herein. In certain embodiments, a method is provided for treating of one or more of cardiac and/or skeletal (e.g., gastrocnemius, deltoid, a soleus, a biceps brachii) muscle-based disorders, and/or a disease in a subject in need thereof, the method comprising delivering to the subject a stock of an rAAV described herein, wherein the encoded gene product is a protein, optionally an antibody.

These and other embodiments and advantages of the invention will be apparent from the specification, including, without limitation, the detailed description of the invention.

### BRIEF DESCRIPTION OF THE FIGURES

FIG. 1A shows plotted heart enrichment scores from RGD screen round 2 for top performing heart candidates in comparison to the top literature capsids. FIG. 1B shows muscle enrichment scores from RGD screen round 2 for top performing heart and muscle candidates in comparison to the top heart candidates. FIG. 1C shows gastrocnemius enrichment scores from RGD screen round 2 for top performing muscle candidates in comparison to the top literature heart capsids.

FIGS. 2A to 2C show RNA, DNA, and protein expression levels in the tissue samples collected from heart left ventricle following administration of AAV9, AAV9-IIRGDPA, and AAV9-AVIRGDV vectors. FIG. 2A shows DNA levels, plotted as GC/diploid cell. FIG. 2B shows RNA levels, plotted as copy number (#)/100 ng RNA. FIG. 2C shows protein expression levels, plotted as picograms (pg) GFP/microgram (μg) protein.

FIGS. 3A to 3C show RNA, DNA, and protein expression levels in the tissue samples collected from heart right ventricle following administration of AAV9, AAV9-IIRGDPA, and AAV9-AVIRGDV vectors. FIG. 3A shows DNA levels, plotted as GC/diploid cell. FIG. 3B shows RNA levels, plotted as copy number (#)/100 ng RNA. FIG. 3C shows protein expression levels, plotted as pg GFP/μg protein.

FIGS. 4A to 4C show RNA, DNA, and protein expression levels in the tissue samples collected from gastrocnemius following administration of AAV9, AAV9-IIRGDPA, and AAV9-AVIRGDV vectors. FIG. 4A shows DNA levels, plotted as GC/diploid cell. FIG. 4B shows RNA levels, plotted as copy number (#)/100 ng RNA. FIG. 4C shows protein expression levels, plotted as pg GFP/μg protein.

FIGS. 5A to 5C show RNA, DNA, and protein expression levels in the tissue samples collected from liver following administration of AAV9, AAV9-IIRGDPA, and AAV9-AVIRGDV vectors. FIG. 5A shows DNA levels, plotted as GC/diploid cell. FIG. 5B shows RNA levels, plotted as copy number (#)/100 ng RNA. FIG. 5C shows protein expression levels, plotted as pg GFP/μg protein.

FIG. 6 shows RNA/DNA ratios (normalized to AAV9) for AAV9, AAV9-IIRGDPA, and AAV9-AVIRGDV vector biodistribution.

FIG. 7 shows an alignment of the specified region of the amino acid sequences of the various AAV capsid proteins: AAV9 (amino acids 566 to 615 of AAV9 capsid; SEQ ID NO: 27), AAV8 (amino acids 565 to 614 of AAV8 capsid; SEQ ID NO: 28), AAV7 (amino acids 567 to 616 of AAV7; SEQ ID NO: 29), AAV6 (amino acids 550 to 599 of AAV6 capsid; SEQ ID NO: 30), AAV5 (amino acids 556 to 605 of AAV5; SEQ ID NO: 31), AAV4 (amino acids 558 to 607 of AAV4 capsid; SEQ ID NO: 32), AAV3B (amino acids 564 to 613 of AAV3B capsid; SEQ ID NO: 33), AAV2 (amino acids 566 to 615 of AAV2 capsid; SEQ ID NO: 34), and AAV1 (amino acids 566 to 615 of AAV1 capsid; SEQ ID NO: 35). The HVRVIII region in which the targeting peptide may be inserted (based on structural analysis) is shown.

FIG. 8 shows percent GFP-positive area as quantified from IHC analysis of heart (longitudinal) tissue samples.

FIG. 9 shows percent GFP-positive area as quantified from IHC analysis of heart (transverse) tissue samples.

FIG. 10 shows RNA/DNA ratios (normalized to AAV9) for AAV9, AAV9-IIRGDPA, and AAV9-X various vectors as analyzed in heart tissue.

FIG. 11 shows RNA/DNA ratios (normalized to AAV9) for AAV9, AAV9-IIRGDPA, and AAV9-X various vectors as analyzed in liver tissues.

FIG. 12A shows RNA levels in heart tissue post-rAAV transduction, plotted as RNA transcript/100 ng. FIG. 12B shows DNA levels in heart tissue post-rAAV transduction, plotted as GC per diploid genome. FIG. 12C shows RNA levels in liver tissue post-rAAV transduction, plotted as RNA transcript/100 ng. FIG. 12D shows DNA levels in liver tissue post-rAAV transduction, plotted as GC per diploid genome.

FIG. 13A shows RNA/DNA ratios (normalized to AAV9) for AAV9, AAV9-AVIRGDV, and AAV9-X various vectors as analyzed in heart tissue. FIG. 13B shows RNA/DNA ratios (normalized to AAV9) for AAV9, AAV9-AVIRGDV, and AAV9-X various vectors as analyzed in liver tissues. FIG. 13C shows RNA levels in heart tissue post-rAAV transduction, plotted as RNA transcript/100 ng. FIG. 13D shows DNA levels in heart tissue post-rAAV transduction, plotted as GC per diploid genome. FIG. 13E shows RNA levels in liver tissue post-rAAV transduction, plotted as RNA transcript/100 ng. FIG. 13F shows DNA levels in liver tissue post-rAAV transduction, plotted as GC per diploid genome.

FIG. 14A shows DNA levels in heart tissue post-rAAV transduction, plotted as GC/diploid cell. FIG. 14B shows RNA levels in heart tissue post-rAAV transduction, plotted as copy number (#)/100 ng RNA. FIG. 14C shows protein expression levels in heart tissue post-rAAV transduction, plotted as pg GFP/μg protein.

FIG. 15A shows DNA levels in liver tissue post-rAAV transduction, plotted as GC/diploid cell. FIG. 15B shows RNA levels in liver tissue post-rAAV transduction, plotted as copy number (#)/100 ng RNA. FIG. 15C shows protein expression levels in liver tissue post-rAAV transduction, plotted as pg GFP/μg protein.

FIG. 16A shows DNA levels in gastrocnemius tissue post-rAAV transduction, plotted as GC/diploid cell. FIG. 16B shows RNA levels in gastrocnemius tissue post-rAAV transduction, plotted as copy number (#)/100 ng RNA. FIG. 16C shows protein expression levels in gastrocnemius tissue post rAAV transduction, plotted as pg GFP/μg protein.

FIG. 17 shows GFP-expression levels in iPSCM cells and C2C12 cells post-rAAV transduction, plotted as Relative Light Unit (RLU, from microplate reading).

FIG. 18A shows titers of rAAV (AAV9, AAV9-IIRGDPA, and AAV9-AVIRGDV vectors) plotted as GC/mL as measured in PBS or PBS with 0.001% Pluronic formulations, and as obtained from Mega and Small scale of rAAV preparations. FIG. 18B shows pooled titers of rAAV (AAV9, AAV9-IIRGDPA, and AAV9-AVIRGDV vectors) plotted as GC/mL as measured in PBS or PBS with 0.001% Pluronic formulations, and as obtained from Mega and Small scale of rAAV preparations.

FIG. 19A shows results of an ongoing survival study, plotted as probability of survival. These results show similar and/or better survival observed in mice when administered AAV9-IIRGDPA or AAV9-AVIRGDV in comparison to AAVhu68 vectors, all comprising TT1. FIG. 19B shows body weights, plotted as grams (g).

FIG. 20A shows representative ISH microscopy image of heart tissue. FIG. 20B show quantified percent ISH-positive cardiomyocytes in various treatment groups.

FIG. 21A shows RNA levels, plotted as normalized RNA transcript copy number (#)/100 ng RNA in heart tissue. FIG. 21B shows RNA levels in liver tissue, plotted as normalized RNA transcript copy number (#)/100 ng RNA. FIG. 21C shows DNA levels, plotted as normalized GC per diploid cell. FIG. 21D shows DNA levels in heart tissue, plotted as normalized GC per diploid cell in liver tissue. FIG. 21E shows protein (GFP) expression levels in heat tissue, plotted as normalized pg GFP per μg total protein. FIG. 21F shows protein (GFP) expression levels ine liver tissue, plotted as normalized pg GFP per μg total protein.

FIG. 22 shows RNA biodistribution results in various muscle tissue subtypes.

FIG. 23 shows DNA biodistribution results in various muscle tissue subtypes.

FIG. 24 shows protein (GFP) biodistribution results in various muscle tissue subtypes.

FIG. 25A shows results of in situ hybridization (ISH) analysis in gastrocnemius tissue, plotted as percent GFP positive as normalized to an AAV9 control. FIG. 25B shows results of ISH analysis in diaphragm tissue, plotted as percent GFP positive as normalized to an AAV9 control. FIG. 25C shows results of ISH analysis in biceps femoris tissue, plotted as percent GFP positive as normalized to an AAV9 control. FIG. 25D shows results of ISH analysis in gluteus maximus tissue, plotted as percent GFP positive as normalized to an AAV9 control. FIG. 25E shows results of ISH analysis in deltoid tissue, plotted as percent GFP positive as normalized to an AAV9 control. FIG. 25F shows results of ISH analysis in soleus tissue, plotted as percent GFP positive as normalized to an AAV9 control. FIG. 25G shows results of ISH analysis in vastus lateralis tissue, plotted as percent GFP positive as normalized to an AAV9 control.

FIG. 26A shows TT2 DNA levels in left ventricle, plotted as GC/diploid cell, following AAVhu68. TT2, AAV9-IIRGDPA. TT2, or AAV9-AVIRGDV.TT2 administration. FIG. 26B shows TT2 RNA levels in left ventricle, plotted as vector GC/100 ng total RNA (as normalized to U6), following AAVhu68. TT2, or AAV9-IIRGDPA. TT2, AAV9-AVIRGDV.TT2 administration. FIG. 26C shows TT2 DNA levels in septum, plotted as GC/diploid cell, following AAVhu68.TT2, AAV9-IIRGDPA.TT2, or AAV9-AVIRGDV.hTT2 administration. FIG. 26D shows TT2 RNA levels in septum, plotted as vector GC/100 ng total RNA (as normalized to U6), following AAVhu68.TT2, AAV9-IIRGDPA. TT2, or AAV9-AVIRGDV.TT2 administration. FIG. 26E shows TT2 DNA levels in left atrium, plotted as GC/diploid cell, following AAVhu68. TT2, AAV9-IIRGDPA.TT2, or AAV9-AVIRGDV.TT2 administration. FIG. 26F shows TT2 RNA levels in left atrium, plotted as vector GC/100 ng total RNA (as normalized to U6), following AAVhu68.TT2, AAV9-IIRGDPA. TT2, or AAV9-AVIRGDV.TT2 administration. FIG. 26G shows TT2 DNA levels in liver, plotted as GC/diploid cell, following AAVhu68.TT2, AAV9-IIRGDPA. TT2, or AAV9-AVIRGDV.TT2 administration. FIG. 26H shows TT2 RNA levels in liver, plotted as vector GC/100 ng total RNA (as normalized to U6), following AAVhu68.TT2, AAV9-IIRGDPA. TT2, or AAV9-AVIRGDV.TT2 administration. FIG. 26I shows TT2 DNA levels in diaphragm, plotted as GC/diploid cell, following AAVhu68.TT2, AAV9-IIRGDPA. TT2, or AAV9-AVIRGDV.TT2 administration. FIG. 26J shows TT2 RNA levels in diaphragm, plotted as vector GC/100 ng total RNA (as normalized to U6), following AAVhu68.TT2, AAV9-IIRGDPA.TT2, or AAV9-AVIRGDV.TT2 administration. FIG. 26K shows TT2 DNA levels in quadriceps, plotted as GC/diploid cell, following AAVhu68.TT2, AAV9-IIRGDPA. TT2, or AAV9-AVIRGDV.TT2 administration. FIG. 26L shows TT2 RNA levels in quadriceps, plotted as vector GC/100 ng total RNA (as normalized to U6), following AAVhu68.TT2, AAV9-IIRGDPA. TT2, or AAV9-AVIRGDV.TT2 administration.

FIG. 27A shows results of ISH analysis, plotted as percent ISH-positive cells in tissue from left heart ventricle. FIG. 27B shows results of ISH analysis, plotted as percent ISH-positive cells in tissue from intraventricular septum of heart. FIG. 27C shows results of ISH analysis, plotted as percent ISH-positive cells in tissue of right heart ventricle. FIG. 27D shows results of ISH analysis, plotted as percent ISH-positive cells in diaphragm tissue. FIG. 27E shows results of ISH analysis, plotted as percent ISH-positive cells in quadricep tissue.

FIG. 28A shows measured levels of aspartate aminotransferase (AST) in blood samples on D0 to D90 following AAVhu68.TT2, AAV9-IIRGDPA. TT2, or AAV9-AVIRGDV.TT2 administration. FIG. 28B shows measured levels of alanine aminotransferase (ALT) in blood samples on DO to D90 following AAVhu68.TT2, AAV9-IIRGDPA.TT2, or AAV9-AVIRGDV.TT2 administration. FIG. 28C shows measured levels of platelet count in blood samples on DO to D90 following AAVhu68.TT2, AAV9-IIRGDPA.TT2, or AAV9-AVIRGDV.TT2 administration. FIG. 28D shows measured levels of d dimer in blood samples on DO to D90 following AAVhu68.TT2, AAV9-IIRGDPA.TT2, or AAV9-AVIRGDV.TT2 administration. FIG. 28E shows measured levels of troponin I in blood samples on DO to D90 following AAVhu68.TT2, AAV9-IIRGDPA.TT2, or AAV9-AVIRGDV.TT2 administration.

### DETAILED DESCRIPTION OF THE INVENTION

In certain embodiments, provided herein are recombinant muscle cell-targeting peptides and nucleic acid sequences encoding the same. Also provided herein are fusion proteins, modified proteins, engineered viral capsids (e.g., recombinant adeno-associated virus (rAAV) capsid) and other moieties comprising a targeting peptide, wherein the targeting peptide is “Xn-n-mer-Xm”, wherein the Xn is 0, 1, 2 or 3 amino acid residues independently selected from any amino acid, wherein the n-mer is IIRGDPA (SEQ ID NO: 1), AVIRGDV (SEQ ID NO: 2), IVRGDPA (SEQ ID NO: 8), MIRGDVK (SEQ ID NO: 9), AQHRGDV (SEQ ID NO: 10), VSRGDPN (SEQ ID NO: 11), VSRGDPA (SEQ ID NO: 12), PLVRGDI (SEQ ID NO:13), PYVRGDP (SEQ ID NO: 14), VVRGDPQ (SEQ ID NO: 15), PTRGDVK (SEQ ID NO: 16), VVQRGDV (SEQ ID NO: 17), QHRGDTQ (SEQ ID NO: 18), QIRGDLR (SEQ ID NO: 19), RGDYAQV (SEQ ID NO: 20), IGRGDPN (SEQ ID NO: 21), RGDLHGY (SEQ ID NO: 22), RGDYSTM (SEQ ID NO: 23), or PYQRGDH (SEQ ID NO: 24), or an n-mer sequence of at least 6, at least 7, or the full-length consecutive amino acids of any one of the n-mers, and wherein the Xm is 0, 1, 2, or 3 amino acid residues independently selected from any amino acid. Also provided herein are nucleic acid sequences encoding the fusion proteins, modified proteins, and engineered viral capsids. In certain embodiments, this exogenous targeting motif modifies the native tissue specificity of the source (parental) protein, viral vector, viral capsid, or other moiety. In certain embodiments, compositions having one or more of the exogenous targeting peptides have enhanced or altered muscle cell-targeting. In certain embodiments, compositions having one or more of the targeting peptides have enhanced or altered cardiac and/or skeletal muscle cell-targeting (e.g., targeting of gastrocnemius muscle cells). In certain embodiments, viral vectors having modified capsids containing the targeting motifs exhibit increased transduction of AAV production cells in vitro.

Advantageously, in certain embodiments provided herein is a recombinant muscle cell-targeting peptide (also referred to as “targeting peptide”, “exogenous targeting peptide”), wherein the recombinant muscle cell-targeting peptide comprises “Xn-n-mer-Xm”, wherein the Xn is 0, 1, 2 or 3 amino acid residues independently selected from any amino acid, wherein the n-mer is IIRGDPA (SEQ ID NO: 1), AVIRGDV (SEQ ID NO: 2), IVRGDPA (SEQ ID NO: 8), MIRGDVK (SEQ ID NO: 9), AQHRGDV (SEQ ID NO: 10), VSRGDPN (SEQ ID NO: 11), VSRGDPA (SEQ ID NO: 12), PLVRGDI (SEQ ID NO:13), PYVRGDP (SEQ ID NO: 14), VVRGDPQ (SEQ ID NO: 15), PTRGDVK (SEQ ID NO: 16), VVQRGDV (SEQ ID NO: 17), QHRGDTQ (SEQ ID NO: 18), QIRGDLR (SEQ ID NO: 19), RGDYAQV (SEQ ID NO: 20), IGRGDPN (SEQ ID NO: 21), RGDLHGY (SEQ ID NO: 22), RGDYSTM (SEQ ID NO: 23), or PYQRGDH (SEQ ID NO: 24), or an n-mer sequence of at least 6, at least 7, or full-length consecutive amino acids of any one of the n-mers, and wherein the Xm is 0, 1, 2, or 3 amino acid residues independently selected from any amino acid.

In certain embodiments, provided herein is an engineered rAAV capsid comprising an exogenous targeting peptide, wherein the exogenous targeting peptide is “Xn-n-mer-Xm”, wherein the Xn is 0, 1, 2 or 3 amino acid residues independently selected from any amino acid, wherein the n-mer is: IIRGDPA (SEQ ID NO: 1), AVIRGDV (SEQ ID NO: 2), IVRGDPA (SEQ ID NO: 8), MIRGDVK (SEQ ID NO: 9), AQHRGDV (SEQ ID NO: 10), VSRGDPN (SEQ ID NO: 11), VSRGDPA (SEQ ID NO: 12), PLVRGDI (SEQ ID NO: 13), PYVRGDP (SEQ ID NO: 14), VVRGDPQ (SEQ ID NO: 15), PTRGDVK (SEQ ID NO: 16), VVQRGDV (SEQ ID NO: 17), QHRGDTQ (SEQ ID NO: 18), QIRGDLR (SEQ ID NO: 19), RGDYAQV (SEQ ID NO: 20), IGRGDPN (SEQ ID NO: 21), RGDLHGY (SEQ ID NO: 22), RGDYSTM (SEQ ID NO: 23), or PYQRGDH (SEQ ID NO: 24), or an n-mer sequence of at least 6, at least 7, or full-length consecutive amino acids of any one of the n-mers, and wherein Xm is 0, 1, 2, or 3 amino acid residues independently selected from any amino acid. In certain embodiments, the exogenous targeting peptide provided herein provides significant transduction advantages in muscle cells, including cardiac muscle cells and/or skeletal muscle cells, optionally gastrocnemius muscle cells, deltoid muscle cells, soleus muscle cells, biceps brachii muscle cells, or diaphragm muscle cells, as compared to a parental capsid (e.g., AAV9, or another clade F capsid, or another clade capsid).

In certain embodiments, provided herein is an engineered rAAV capsid comprising an exogenous targeting peptide comprising “Xn-IIRGDPA (SEQ ID NO: 1)-Xm”, wherein the Xn is 0, 1, 2 or 3 amino acid residues independently selected from any amino acid, wherein the Xm is 0, 1, 2, or 3 amino acid residues independently selected from any amino acid, wherein the exogenous targeting peptide provides significant transduction advantages in muscle cells, including cardiac muscle cells and/or skeletal muscle cells, optionally gastrocnemius muscle cells, as compared to a parental capsid. In certain embodiments, provided herein is an engineered rAAV capsids comprising an exogenous targeting peptide comprising “Xn-AVIRGDV (SEQ ID NO: 2)-Xm”, wherein Xn is 0, 1, 2 or 3 amino acid residues independently selected from any amino acid, wherein Xm is 0, 1, 2, or 3 amino acid residues independently selected from any amino acid, wherein the exogenous targeting peptide provides significant transduction advantages in muscle cells, including cardiac muscle cells and/or skeletal muscle cells, optionally gastrocnemius muscle cells, as compared to a parental capsid. In certain embodiments, provided herein is an engineered rAAV capsid comprising an exogenous targeting peptide comprising “Xn-IVRGDPA (SEQ ID NO: 8)-Xm”, wherein Xn is 0, 1, 2 or 3 amino acid residues independently selected from any amino acid, wherein Xm is 0, 1, 2, or 3 amino acid residues independently selected from any amino acid, wherein the exogenous targeting peptide provides significant transduction advantages in muscle cells, including the cardiac muscle cells and/or skeletal muscle cells, optionally gastrocnemius muscle cells, as compared to a parental capsid. In certain embodiments, provided herein is an engineered rAAV capsid comprising an exogenous targeting peptide comprising “Xn-MIRGDVK (SEQ ID NO: 9)-Xm”, wherein the Xn is 0, 1, 2 or 3 amino acid residues independently selected from any amino acid, wherein the optional Xm is 0, 1, 2, or 3 amino acid residues independently selected from any amino acid, wherein the exogenous targeting peptide provides significant transduction advantages in muscle cells, including cardiac muscle cells and/or skeletal muscle cells, optionally gastrocnemius muscle cells, as compared to a parental capsid. In certain embodiments, provided herein is an engineered rAAV capsid comprising an exogenous targeting peptide comprising “Xn-AQHRGDV (SEQ ID NO: 10)-Xm”, wherein the Xn is 0, 1, 2 or 3 amino acid residues independently selected from any amino acid, wherein the Xm is 0, 1, 2, or 3 amino acid residues independently selected from any amino acid, wherein the exogenous targeting peptide provides significant transduction advantages in muscle cells, including cardiac muscle cells and/or skeletal muscle cells, optionally gastrocnemius muscle cells, as compared to a parental capsid. In certain embodiments, provided herein is an engineered rAAV capsid comprising an exogenous targeting peptide comprising “Xn-VSRGDPN (SEQ ID NO: 11)-Xm”, wherein Xn is 0, 1, 2 or 3 amino acid residues independently selected from any amino acid, wherein Xm is 0, 1, 2, or 3 amino acid residues independently selected from any amino acid, wherein the exogenous targeting peptide provides significant transduction advantages in muscle cells, including cardiac muscle cells and/or skeletal muscle cells, optionally gastrocnemius muscle cells, as compared to a parental capsid. In certain embodiments, provided herein is an engineered rAAV capsid comprising an exogenous targeting peptide comprises “Xn-VSRGDPA (SEQ ID NO: 12)-Xm”, wherein the Xn is 0, 1, 2 or 3 amino acid residues independently selected from any amino acid, wherein Xm is 0, 1, 2, or 3 amino acid residues independently selected from any amino acid, wherein the exogenous targeting peptide provides significant transduction advantages in muscle cells, including cardiac muscle cells and/or skeletal muscle cells, optionally gastrocnemius muscle cells, as compared to a parental capsid. In certain embodiments, provided herein is an engineered rAAV capsid comprising an exogenous targeting peptide comprising “Xn-PLVRGDI (SEQ ID NO: 13)-Xm”, wherein Xn is 0, 1, 2 or 3 amino acid residues independently selected from any amino acid, wherein Xm is 0, 1, 2, or 3 amino acid residues independently selected from any amino acid, wherein the exogenous targeting peptide provides significant transduction advantages in muscle cells, including cardiac muscle cells and/or skeletal muscle cells, optionally gastrocnemius muscle cells, as compared to a parental capsid. In certain embodiments, provided herein is an engineered rAAV capsid comprising an exogenous targeting peptide comprising “Xn-PYVRGDP (SEQ ID NO: 14)-Xm”, wherein Xn is 0, 1, 2 or 3 amino acid residues independently selected from any amino acid, wherein Xm is 0, 1, 2, or 3 amino acid residues independently selected from any amino acid, wherein the exogenous targeting peptide provides significant transduction advantages in muscle cells, including the cardiac muscle cells and/or skeletal muscle cells, optionally gastrocnemius muscle cells, as compared to a parental capsid. In certain embodiments, provided herein is an engineered rAAV capsid comprising an exogenous targeting peptide comprising “Xn-VVRGDPQ (SEQ ID NO: 15)-Xm”, wherein Xn is 0, 1, 2 or 3 amino acid residues independently selected from any amino acid, wherein Xm is 0, 1, 2, or 3 amino acid residues independently selected from any amino acid, wherein the exogenous targeting peptide provides significant transduction advantages in muscle cells, including cardiac muscle cells and/or skeletal muscle cells, optionally gastrocnemius muscle cells, as compared to a parental capsid. In certain embodiments, provided herein is an engineered rAAV capsid comprising an exogenous targeting peptide comprising “Xn-PTRGDVK (SEQ ID NO: 16)-Xm”, wherein Xn is 0, 1, 2 or 3 amino acid residues independently selected from any amino acid, wherein Xm is 0, 1, 2, or 3 amino acid residues independently selected from any amino acid, wherein the exogenous targeting peptide provides significant transduction advantages in muscle cell, including cardiac muscle cells and/or skeletal muscle cells, optionally gastrocnemius muscle cells, as compared to a parental capsid. In certain embodiments, provided herein is an engineered rAAV capsid comprising an exogenous comprising “Xn-VVQRGDV (SEQ ID NO: 17)-Xm”, wherein Xn is 0, 1, 2 or 3 amino acid residues independently selected from any amino acid, wherein Xm is 0, 1, 2, or 3 amino acid residues independently selected from any amino acid, wherein the exogenous targeting peptide provides significant transduction advantages in muscle cells, including cardiac muscle cells and/or skeletal muscle cells, optionally gastrocnemius muscle cells, as compared to a parental capsid. In certain embodiments, provided herein is an engineered rAAV capsid comprising an exogenous targeting peptide comprising “Xn-QHRGDTQ (SEQ ID NO: 18)-Xm”, wherein Xn is 0, 1, 2 or 3 amino acid residues independently selected from any amino acid, wherein Xm is 0, 1, 2, or 3 amino acid residues independently selected from any amino acid, wherein the exogenous targeting peptide provides significant transduction advantages in muscle cells, including the cardiac muscle cells and/or skeletal muscle cells, optionally gastrocnemius muscle cells, as compared to a parental capsid. In certain embodiments, provided herein is an engineered rAAV capsid comprising an exogenous targeting peptide comprising “Xn-QIRGDLR (SEQ ID NO: 19)-Xm”, wherein the Xn is 0, 1, 2 or 3 amino acid residues independently selected from any amino acid, wherein the Xm is 0, 1, 2, or 3 amino acid residues independently selected from any amino acid, wherein the exogenous targeting peptide provides significant transduction advantages in muscle cells, including the cardiac muscle cells and/or skeletal muscle cells, optionally gastrocnemius muscle cells, as compared to a parental capsid. In certain embodiments, provided herein is an engineered rAAV capsid comprising an exogenous targeting peptide comprising “Xn-RGDYAQV (SEQ ID NO: 20)-Xm”, wherein Xn is 0, 1, 2 or 3 amino acid residues independently selected from any amino acid, wherein Xm is 0, 1, 2, or 3 amino acid residues independently selected from any amino acid, wherein the exogenous targeting peptide provides significant transduction advantages in muscle cells, including cardiac muscle cells and/or skeletal muscle cells, optionally gastrocnemius muscle cells, as compared to a parental capsid. In certain embodiments, provided herein is an engineered rAAV capsid comprising an exogenous targeting peptide comprising “Xn-IGRGDPN (SEQ ID NO: 21) Xm”, wherein Xn is 0, 1, 2 or 3 amino acid residues independently selected from any amino acid, wherein Xm is 0, 1, 2, or 3 amino acid residues independently selected from any amino acid, wherein the exogenous targeting peptide provides significant transduction advantages in muscle cells, including cardiac muscle cells and/or skeletal muscle cells, optionally gastrocnemius muscle cells, as compared to a parental capsid. In certain embodiments, provided herein is an engineered rAAV capsid comprising an exogenous targeting peptide comprising “Xn-RGDLHGY (SEQ ID NO: 22)-Xm”, wherein Xn is 0, 1, 2 or 3 amino acid residues independently selected from any amino acid, wherein Xm is 0, 1, 2, or 3 amino acid residues independently selected from any amino acid, wherein the exogenous targeting peptide provides significant transduction advantages in muscle cells, including cardiac muscle cells and/or skeletal muscle cells, optionally gastrocnemius muscle cells, as compared to a parental capsid. In certain embodiments, provided herein is an engineered rAAV capsid comprising an exogenous targeting peptide comprising Xn-RGDYSTM (SEQ ID NO: 23)-Xm”, wherein Xn is 0, 1, 2 or 3 amino acid residues independently selected from any amino acid, wherein Xm is 0, 1, 2, or 3 amino acid residues independently selected from any amino acid, wherein the exogenous targeting peptide provides significant transduction advantages in muscle cell, including cardiac muscle cells and/or skeletal muscle cells, optionally gastrocnemius muscle cells, as compared to a parental capsid. In certain embodiments, provided herein is an engineered rAAV capsid comprising am exogenous targeting peptide comprising “Xn-PYQRGDH (SEQ ID NO: 24)-Xm”, wherein Xn is 0, 1, 2 or 3 amino acid residues independently selected from any amino acid, wherein Xm is 0, 1, 2, or 3 amino acid residues independently selected from any amino acid, wherein the exogenous targeting peptide provides significant transduction advantages in muscle cells, including cardiac muscle cells and/or skeletal muscle cells, optionally gastrocnemius muscle cells, as compared to a parental capsid.

In certain embodiments, provided herein is an rAAV comprising a mutant AAV capsid having an exogenous targeting peptide as identified herein. In certain embodiments, the mutant AAV capsid comprises an exogenous targeting peptide which is immediately preceded by flanking amino acids that are mutated, as compared to parental AAV capsid. In certain embodiments, the mutated flanking amino acids, together with 1, 2, 3, 4, 5, or 6 inserted amino acids comprise the exogenous targeting peptide. In certain embodiments, the entirety of the exogenous targeting peptide is inserted into the parental AAV capsid. In still other embodiments, the amino acid sequence inserted into a capsid protein may comprise all or a fragment of the exogenous targeting peptide at the carboxy (COO—) or amino terminus (N—) (i.e., via insertion of the 5′ or 3′ coding sequences therefor) and further comprises 0-3 flanking amino acid residues as provided in the above formulae. In certain embodiments, the engineered rAAV capsids comprising the targeting peptides, as provided herein, demonstrate reduced transduction (i.e., de-targeted/ing) to liver as compared to their parental capsid (e.g., AAV9 or another clade F capsid (e.g., AAVhu68, AAVhu31, AAVhu32, AAVhu95, AAVhu96) or other modification thereof). In certain embodiments, engineered rAAV capsids comprising the targeting peptides, as provided herein, demonstrate reduced transduction (i.e., de-targeted/ing) to spleen as compared to their parental capsid (e.g., AAV9 or another clade F capsid or other modification thereof. The targeting peptide may be linked to a recombinant protein (e.g., for enzyme replacement therapy) or polypeptide (e.g., an immunoglobulin) to form a fusion protein or a conjugate to target a desired tissue (e.g., muscle cell, cardiac muscle cell, skeletal muscle cell, gastrocnemius muscle cell). Additionally, the targeting peptide may be linked to a liposome and/or a nanoparticle (a lipid nanoparticle, LNP) forming a peptide-coated liposome and/or LNP to target the desired tissue. Sequences encoding at least one copy of a targeting peptide and optional linking sequences may be fused in frame with the coding sequence for a recombinant protein and co-expressed with the protein or polypeptide to provide fusion proteins or conjugates. Alternatively, other synthetic methods may be used to form a conjugate with a protein, polypeptide or another moiety (e.g., DNA, RNA, or a small molecule). In certain embodiments, multiple copies of a targeting peptide are in the fusion protein/conjugate. Suitable methods for conjugating a targeting peptide to a recombinant protein include modifying the amino (N)-terminus and one or more residues on a recombinant human protein (e.g., an enzyme) using a first crosslinking agent to give rise to a first crosslinking agent modified recombinant human protein, modifying the amino (N)-terminus of a short extension linker region preceding a targeting peptide using a second crosslinking agent to give rise to a second crosslinking agent modified variant target peptide, and then conjugating the first crosslinking agent modified recombinant human protein to the second crosslinking agent modified variant targeting peptide containing a short extension linker. Other suitable methods for conjugating a targeting peptide to a recombinant protein include conjugating a first crosslinking agent modified recombinant human protein to one or more second crosslinking agent modified variant targeting peptides, wherein the first crosslinking agent modified recombinant protein comprises a recombinant protein characterized as having a chemically modified N-terminus and one or more modified lysine residues and the one or more second crosslinking agent modified variant targeting peptides comprise one or more variant targeting peptides comprising a modified N-terminal amino acid of a short extension linker preceding the targeting peptide. Still other suitable methods for conjugating a targeting peptide to a protein, polypeptide, nanoparticle, or another biologically useful chemical moiety may be selected. See, e.g., U.S. Pat. No. 9,545,450 B2 (NHS-phosphine cross-linking agents; NHS-Azide cross-linking agents); US Published Patent Application No. US 2018/0185503 A1 (aldehyde-hydrazide crosslinking).

In certain embodiments, an exogenous targeting peptide is engineered (i.e., inserted) at a suitable site within a protein or polypeptide (e.g., a viral capsid protein). In certain embodiments, a targeting peptide is flanked at its amino (N—) (e.g., optional Xn) and/or carboxy (COO—) (e.g., optional Xm) terminus by a short extension linker. Such a linker may be about 1 to about 20 amino acid residues in length, or about 2 to about 20 amino acids residues, or about 1 to about 15 amino acid residues, or about 2 to about 12 amino acid residues, or about 2 to about 7 amino acid residues in length. The short extension linker can also be about 10 amino acids in length. The presence and length of a linker at the N-terminus is independently selected from a linker at the carboxy-terminus, and the presence and length of a linker at the carboxy terminus is independently selected from a linker at the N-terminus. Suitable short extension linkers can be provided using an about 5-amino acid flexible GS extension linker (glycine-glycine-glycine-glycine-serine), an about 10-amino acid extension linker comprising 2 flexible GS linkers, an about 15-amino acid extension linker comprising 3 flexible GS linkers, an about 20-amino acid extension linker comprising 4 flexible GS linkers, or any combination thereof.

In certain embodiment, a composition is provided which is useful for targeting a muscle cell. In certain embodiments, the composition comprises an engineered capsid (e.g., rAAV capsid), fusion protein or another conjugate comprising at least one exogenous targeting peptide comprising: a core amino acid sequence (e.g., “n-mer”) of IIRGDPA (SEQ ID NO: 1) flanked at its amino terminus and/or carboxy terminus of the core sequence by 0, 1, 2, or 3 amino acid residues (e.g., Xn and Xm, respectively), and optionally further conjugated to a nanoparticle, a second molecule, or a viral capsid protein. In certain embodiments, the composition comprises an engineered capsid, fusion protein or another conjugate comprising at least one exogenous targeting peptide comprising: a core amino acid sequence (e.g., “n-mer”) of AVIRGDV (SEQ ID NO: 2) flanked at its amino terminus and/or carboxy terminus of the core sequence by 0, 1, 2, or 3 amino acid residues (e.g., Xn and Xm, respectively), and optionally further conjugated to a nanoparticle, a second molecule, or a viral capsid protein. In certain embodiments, the composition comprises an engineered capsid, fusion protein or another conjugate comprising at least one exogenous targeting peptide comprising: a core amino acid sequence that is IVRGDPA (SEQ ID NO: 8), MIRGDVK (SEQ ID NO: 9), AQHRGDV (SEQ ID NO: 10), VSRGDPN (SEQ ID NO: 11), VSRGDPA (SEQ ID NO: 12), PLVRGDI (SEQ ID NO:13), PYVRGDP (SEQ ID NO: 14), VVRGDPQ (SEQ ID NO: 15), PTRGDVK (SEQ ID NO: 16), VVQRGDV (SEQ ID NO: 17), QHRGDTQ (SEQ ID NO: 18), QIRGDLR (SEQ ID NO: 19), RGDYAQV (SEQ ID NO: 20), IGRGDPN (SEQ ID NO: 21), RGDLHGY (SEQ ID NO: 22), RGDYSTM (SEQ ID NO: 23), or PYQRGDH (SEQ ID NO: 24), or an n-mer sequence of at least 6 consecutive amino acids of any one of the n-mers, flanked at its amino terminus and/or carboxy terminus of the core sequence by 0, 1, 2, or 3 amino acid residues (e.g., Xn and Xm, respectively), and optionally further conjugated to a nanoparticle, a second molecule, or a viral capsid protein.

In certain embodiments, the targeting peptide core amino acid sequence is encoded by a nucleic acid sequence.

In certain embodiments, the targeting peptide core (e.g., “n-mer”) is IIRGDPA (SEQ ID NO: 1). In certain embodiments, the targeting peptide core is AVIRGDV (SEQ ID NO: 2). In certain embodiments, the targeting peptide core is IVRGDPA (SEQ ID NO: 8). In certain embodiments, the targeting peptide core is MIRGDVK (SEQ ID NO: 9). In certain embodiments, the targeting peptide core is AQHRGDV (SEQ ID NO: 10). In certain embodiments, the targeting peptide core is VSRGDPN (SEQ ID NO: 11). In certain embodiments, the targeting peptide core is VSRGDPA (SEQ ID NO: 12). In certain embodiments, the targeting peptide core is PLVRGDI (SEQ ID NO: 13). In certain embodiments, the targeting peptide core is PYVRGDP (SEQ ID NO: 14). In certain embodiments, the targeting peptide core is VVRGDPQ (SEQ ID NO: 15). In certain embodiments, the targeting peptide core is PTRGDVK (SEQ ID NO: 16). In certain embodiments, the targeting peptide core is VVQRGDV (SEQ ID NO: 17). In certain embodiments, the targeting peptide core is QHRGDTQ (SEQ ID NO: 18). In certain embodiments, the targeting peptide core is QIRGDLR (SEQ ID NO: 19). In certain embodiments, the targeting peptide core is RGDYAQV (SEQ ID NO: 20). In certain embodiments, the targeting peptide core is IGRGDPN (SEQ ID NO: 21). In certain embodiments, the targeting peptide core is RGDLHGY (SEQ ID NO: 22). In certain embodiments, the targeting peptide core is RGDYSTM (SEQ ID NO: 23). In certain embodiments, the targeting peptide core is PYQRGDH (SEQ ID NO: 24). In certain embodiments, more than one copy of a targeting peptide is present in a conjugate or modified protein (e.g., a parvovirus capsid, rAAV capsid). In certain embodiments, two or more different targeting peptide cores are present.

In certain embodiments, a composition is provided which is useful for targeting a muscle cell. In certain embodiments, a composition is provided which is useful for targeting a cardiac muscle cell (i.e., heart tissue). In certain embodiments, a composition is provided which is useful for targeting a skeletal muscle cell. In certain embodiments, a composition is provided which is useful for targeting a gastrocnemius muscle cell. In certain embodiments, a composition is provided which is useful for targeting a muscle cell, while also de-targeting cells in liver. In certain embodiments, a composition is provided which is useful for targeting a muscle cell, while also de-targeting cells in the spleen.

Provided herein also the composition comprising an engineered rAAV capsid, fusion protein or another conjugate comprising at least one exogenous targeting peptide comprising: the amino acid core sequence IIRGDPA (SEQ ID NO: 1), AVIRGDV (SEQ ID NO: 2), IVRGDPA (SEQ ID NO: 8), MIRGDVK (SEQ ID NO: 9), AQHRGDV (SEQ ID NO: 10), VSRGDPN (SEQ ID NO: 11), VSRGDPA (SEQ ID NO: 12), PLVRGDI (SEQ ID NO:13), PYVRGDP (SEQ ID NO: 14), VVRGDPQ (SEQ ID NO: 15), PTRGDVK (SEQ ID NO: 16), VVQRGDV (SEQ ID NO: 17), QHRGDTQ (SEQ ID NO: 18), QIRGDLR (SEQ ID NO: 19), RGDYAQV (SEQ ID NO: 20), IGRGDPN (SEQ ID NO: 21), RGDLHGY (SEQ ID NO: 22), RGDYSTM (SEQ ID NO: 23), and/or PYQRGDH (SEQ ID NO: 24), wherein the inserted targeting peptide is flanked at the amino terminus and/or the carboxy terminus of the motif by 0, 1, 2, 3 amino acids, and optionally further conjugated to a nanoparticle, a second molecule, or a viral capsid protein.

Examples of suitable proteins, including enzymes, immunoglobulins, therapeutic proteins, immunogenic polypeptides, nanoparticles, DNA, RNA, and other moieties (e.g., small molecules, etc.) for targeting are described in more detail below. These and other biologic and chemical moieties are suitable for use with the targeting peptide(s) provided herein.

In certain embodiments, provided herein is a composition comprising a nucleic acid molecule, wherein the nucleic acid molecule is a DNA molecule or RNA molecule, e.g., naked DNA, naked plasmid DNA, messenger RNA (mRNA), linked to a targeting peptide sequence motif described herein. In some embodiments, the nucleic acid molecule is further coupled with various compositions and nano particles, including, e.g., micelles, liposomes, cationic lipid-nucleic acid compositions, poly-glycan compositions and other polymers, lipid and/or cholesterol-based-nucleic acid conjugates, and other constructs such as are described herein. See, e.g., WO2014/089486, US 2018/0353616A1, US2013/0037977A1, WO2015/074085A1, U.S. Pat. No. 9,670,152B2, and U.S. Pat. No. 8,853,377B2, X. Su, et al., Mol. Pharmaceutics, 2011, 8 (3), pp 774-787; web publication: Mar. 21, 2011; WO2013/182683, WO 2010/053572 and WO 2012/170930, all of which are incorporated herein by reference. In certain embodiments, the targeting peptide motif is chemically linked to a nanoparticle surface, wherein the nanoparticle encapsulates a nucleic acid molecule. In some embodiments, the nanoparticle comprising the targeting peptide linked to the surface is designed for targeted tissue-specific delivery. In some embodiments, two or more different targeting peptides are linked to the surface of the nanoparticle. Suitable chemical linking or cross-linking include those known to one skilled in the art.

### Capsids

In certain embodiments, a recombinant parvovirus is provided which has a modified parvovirus capsid, wherein the capsid includes an amino acid sequence comprising a hypervariable region comprising an exogenous targeting peptide, wherein the exogenous targeting peptide comprises “Xn-n-mer-Xm”, wherein: (i) Xn is 0, 1, 2 or 3 amino acid residues independently selected from any amino acid; (ii) the n-mer is IIRGDPA (SEQ ID NO: 1), AVIRGDV (SEQ ID NO: 2), IVRGDPA (SEQ ID NO: 8), MIRGDVK (SEQ ID NO: 9), AQHRGDV (SEQ ID NO: 10), VSRGDPN (SEQ ID NO: 11), VSRGDPA (SEQ ID NO: 12), PLVRGDI (SEQ ID NO: 13), PYVRGDP (SEQ ID NO: 14), VVRGDPQ (SEQ ID NO: 15), PTRGDVK (SEQ ID NO: 16), VVQRGDV (SEQ ID NO: 17), QHRGDTQ (SEQ ID NO: 18), QIRGDLR (SEQ ID NO: 19), RGDYAQV (SEQ ID NO: 20), IGRGDPN (SEQ ID NO: 21), RGDLHGY (SEQ ID NO: 22), RGDYSTM (SEQ ID NO: 23), or PYQRGDH (SEQ ID NO: 24), or an n-mer sequence of at least 6, at least 7, or full-length consecutive amino acids of any one of the n-mers; and (iii) Xm is 0, 1, 2, or 3 amino acid residues independently selected from any amino acid. Such a recombinant parvovirus may be a hybrid bocavirus/AAV or a recombinant AAV vector (rAAV). In other embodiments, other viral vectors may be generated having one or more exogenous targeting peptides in an exposed capsid protein to modulate and/or alter the targeting specificity of the viral vector as compared to a parental vector, wherein the one or more exogenous targeting peptide comprises “Xn-n-mer-Xm”, wherein: (i) Xn is 0, 1, 2 or 3 amino acid residues independently selected from any amino acid; (ii) the n-mer is IIRGDPA (SEQ ID NO: 1), AVIRGDV (SEQ ID NO: 2), IVRGDPA (SEQ ID NO: 8), MIRGDVK (SEQ ID NO: 9), AQHRGDV (SEQ ID NO: 10), VSRGDPN (SEQ ID NO: 11), VSRGDPA (SEQ ID NO: 12), PLVRGDI (SEQ ID NO:13), PYVRGDP (SEQ ID NO: 14), VVRGDPQ (SEQ ID NO: 15), PTRGDVK (SEQ ID NO: 16), VVQRGDV (SEQ ID NO: 17), QHRGDTQ (SEQ ID NO: 18), QIRGDLR (SEQ ID NO: 19), RGDYAQV (SEQ ID NO: 20), IGRGDPN (SEQ ID NO: 21), RGDLHGY (SEQ ID NO: 22), RGDYSTM (SEQ ID NO: 23), or PYQRGDH (SEQ ID NO: 24), or an n-mer sequence of at least 6, at least 7, or full-length consecutive amino acids of any one of the n-mers; and (iii) Xm is 0, 1, 2, or 3 amino acid residues independently selected from any amino acid.

The exogenous targeting peptide may be inserted (and/or engineered via mutation of sequences encoding flanking amino acid residue(s)) into a hypervariable loop (HVR) VIII (also referenced as HVR8) at any suitable location. For example, based on the numbering of the AAV9 capsid, the peptide is inserted with linkers of various lengths between amino acids 588 and 589 (Q-A) of the AAV9 capsid protein, based on the numbering of the AAV9 VP1 (also referenced as Vp1 or vp1) amino acid sequence: SEQ ID NO: 25. See, also, WO 2019/168961, published Sep. 6, 2019, including Table G providing the deamidation pattern for AAV9 and WO 2020/160582, filed Sep. 7, 2018. The amino acid residue locations (i.e., amino acid numbering reference) are identical in AAVhu68 (SEQ ID NO: 26). However, another site may be selected within HVRVIII. Alternatively, another exposed loop HVR (e.g., HVRIV) may be selected for the insertion. Comparable HVR regions may be selected in other capsids. In certain embodiments, the location for the HVRVIII and HVRIV is determined using an algorithm and/or alignment technique as described in U.S. Pat. No. 9,737,618 B2 (column 15, lines 3-23), and U.S. Pat. No. 10,308,958 B2 (column 15, line 46-column 16, line 6), which are incorporated herein by reference in its entirety. In certain embodiments, the targeting peptide may be inserted (and/or engineered) into a hypervariable loop HVRVIII as described in U.S. Provisional Patent Application No. 63/119,863, filed Dec. 1, 2020, and International Patent Application No. PCT/US2021/061312, filed Dec. 1, 2022, which are incorporated herein by reference in their entireties. In certain embodiments, AAV1 capsid protein is selected as the parental capsid, wherein the targeting peptide with linkers of various lengths is inserted in a suitable location of the HVRVIII region of amino acid 582 to 585, or HVRIV region of amino acid 456 to 459 based on vp1 numbering (Gurda, B L., et al., Capsid Antibodies to Different Adeno-Associated Virus Serotypes Bind Common Regions, 2012, Journal of Virology, Jun. 12, 2013, 87 (16): 9111-91114). In certain embodiments, AAV8 is selected as the parental capsid, wherein the targeting peptide with linkers of various length is inserted in a suitable location of HVRVIII region of amino acid 586 to 591, or HVRIV region of amino acid 456 to 460, based on VP1 numbering (Gurda, B L., et al., Mapping a Neutralizing epitope onto the Capsid of Adeno-Associated Virus Serotype 8, 2012, Journal of Virology, May 16, 2012, 86 (15): 7739-7751).

In certain embodiments, the parental AAV capsid is an AAV9, AAVhu68, AAVhu31, AAVhu32, AAV8, AAV7, AAV6, AAV5, AAV4, AAV3, AAV1, AAVhu95, AAVhu96, or AAVrh91 capsid.

In certain embodiments, the exogenous targeting peptide is engineered and/or inserted in the hypervariable region between amino acids 588 and 589 in an AAV9 parental capsid as determined based on the numbering of VP1 amino acid sequence of SEQ ID NO: 25, or an analogous position in an AAVhu68, AAVhu31, AAVhu32, AAVhu95, AAVhu96, AAV8, AAV7, AAV6, AAV5, AAV4, AAV3, AAV1, or AAVrh91 parental AAV capsid.

In certain embodiments, the exogenous targeting peptide has an amino acid sequence at its carboxy terminus and its amino terminus which is immediately preceded by “AQ” at position 588 of the parental capsid, which is a Clade F capsid, for example, an AAV9, AAVhu68, AAVhu31, AAVhu32, AAVhu95, or AAVhu96 capsid.

In certain embodiments, the residues of the parental AAV capsid sequence is preserved (i.e., there are no substitutions and/or deletions in the 1, 2, and/or 3 amino acid residues at the N-terminus and/or C-terminus immediately preceding the target peptide insert, as compared to that of the parental AAV capsid amino acid sequence). In certain embodiments, there are no deletions in the 1, 2 and/or 3 amino acid residues as compared to that of the parental AAV capsid amino acid sequence at the N-terminus and/or C-terminus immediately preceding the target peptide insert. In certain embodiments, one or more amino acid residues, as compared to that of the parental AAV capsid amino acid sequence, are modified at the N-terminus and/or C-terminus immediately preceding the n-mer, and/or to provide the mutant AAV capsid with one or more residues of the n-mer sequence. In certain embodiments, one or more amino acid residues, as compared to that of the parental AAV capsid amino acid sequence, are modified at the positions at the N-terminus and/or C-terminus immediately flanking the n-mer, and/or to provide the mutant AAV with one or more residues of the n-mer sequence.

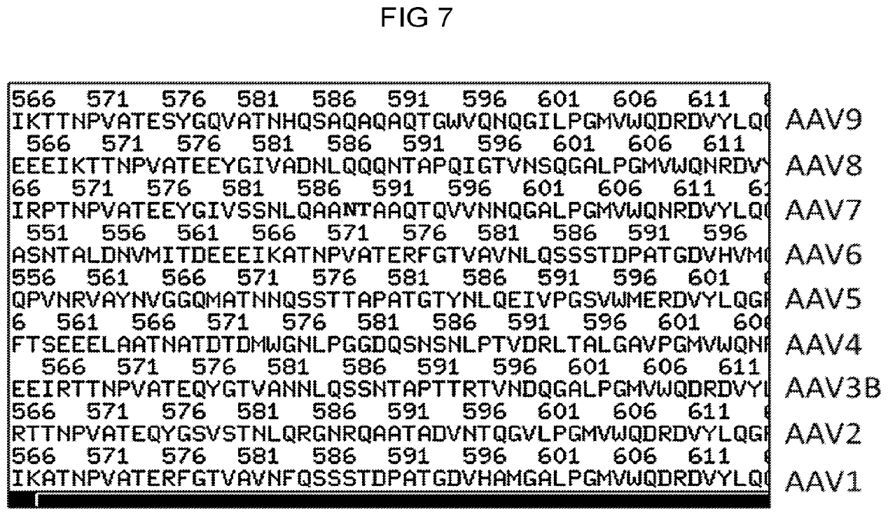

In certain embodiments, AAV9 is selected as the parental capsid, wherein the targeting peptide with linkers of various length(s) is inserted (and/or engineered) in a suitable location of HVRVIII region of amino acid 588 and 589 (Q-A), based on VP1 numbering. In other embodiments, AAVhu68 or another clade F capsid is selected as the parental capsid. In certain embodiments, AAV8 is selected as the parental capsid, wherein the targeting peptide with linkers of various length(s) is inserted (and/or engineered) in a suitable location of HVRVIII region of amino acid 590 and 591 (N-T), based on VP1 numbering. In certain embodiments, AAV7 is selected as the parental capsid, wherein the targeting peptide with linkers of various length(s) is inserted (and/or engineered) in a suitable location of HVRVIII region of amino acid 589 and 590 (N-T), based on VP1 numbering. In certain embodiments, AAV6 is selected as the parental capsid, wherein the targeting peptide with linkers of various length(s) is inserted (and/or engineered) in a suitable location of HVRVIII region of amino acid 588 and 589 (S-T), based on VP1 numbering. In certain embodiments, AAV5 is selected as the parental capsid, wherein the targeting peptide with linkers of various length(s) is inserted (and/or engineered) in a suitable location of HVRVIII region of amino acid 577 and 578 (T-T), based on VP1 numbering. In certain embodiments, AAV4 is selected as the parental capsid, wherein the targeting peptide with linkers of various length(s) is inserted (and/or engineered) in a suitable location of HVRVIII region of amino acid 586 and 587 (S-N), based on VP1 numbering. In certain embodiments, AAV3/3B is selected as the parental capsid, wherein the targeting peptide with linkers of various length(s) is inserted (and/or engineered) in a suitable location of HVRVIII region of amino acid 588 and 589 (N-T), based on VP1 numbering. In certain embodiments, AAV2 is selected as the parental capsid, wherein the targeting peptide with linkers of various length(s) is inserted (and/or engineered) in a suitable location of HVRVIII region of amino acid 587 and 589 (N-R), based on VP1 numbering. In certain embodiments, AAV1 is selected as the parental capsid, wherein the targeting peptide with linkers of various length(s) is inserted (and/or engineered) in a suitable location of HVRVIII region of amino acid 588 and 589 (S-T), based on VP1 numbering. See also, FIG. 7 which shows an alignment of the specified region of the amino acid sequences of the various AAV capsid proteins of AAV9 (amino acids 566 to 615 of AAV9 capsid; SEQ ID NO: 27), AAV8 (amino acids 565 to 614 of AAV8 capsid; SEQ ID NO: 28), AAV7 (amino acids 567 to 616 of AAV7; SEQ ID NO: 29), AAV6 (amino acids 550 to 599 of AAV6 capsid; SEQ ID NO: 30), AAV5 (amino acids 556 to 605 of AAV5; SEQ ID NO: 31), AAV4 (amino acids 558 to 607 of AAV4 capsid; SEQ ID NO: 32), AAV3B (amino acids 564 to 613 of AAV3B capsid; SEQ ID NO: 33), AAV2 (amino acids 566 to 615 of AAV2 capsid; SEQ ID NO: 34), and AAV1 (amino acids 566 to 615 of AAV1 capsid; SEQ ID NO: 35), which is focused on the region HVRVIII in which the targeting peptide may be inserted (based on structural analysis).

In certain embodiments, the parental capsid is modified to comprise “Xn-n-mer-Xm”, wherein: (i) Xn is 0, 1, 2 or 3 amino acid residues independently selected from any amino acid; (ii) the n-mer is IIRGDPA (SEQ ID NO: 1), AVIRGDV (SEQ ID NO: 2), IVRGDPA (SEQ ID NO: 8), MIRGDVK (SEQ ID NO: 9), AQHRGDV (SEQ ID NO: 10), VSRGDPN (SEQ ID NO: 11), VSRGDPA (SEQ ID NO: 12), PLVRGDI (SEQ ID NO:13), PYVRGDP (SEQ ID NO: 14), VVRGDPQ (SEQ ID NO: 15), PTRGDVK (SEQ ID NO: 16), VVQRGDV (SEQ ID NO: 17), QHRGDTQ (SEQ ID NO: 18), QIRGDLR (SEQ ID NO: 19), RGDYAQV (SEQ ID NO: 20), IGRGDPN (SEQ ID NO: 21), RGDLHGY (SEQ ID NO: 22), RGDYSTM (SEQ ID NO: 23), or PYQRGDH (SEQ ID NO: 24), or an n-mer sequence of at least 6, at least 7, or full-length consecutive amino acids of any one of the n-mers; and (iii) Xm is 0, 1, 2, or 3 amino acid residues independently selected from any amino acid, wherein the parental capsid is a Clade F AAV (e.g., AAVhu68, AAV9, AAVhu31, AAVhu32, AAVhu95, AAVhu96), Clade E (e.g., AAV8), or Clade A AAV (e.g., AAV1, AAVrh91)) capsid, or a non-parvovirus capsid (e.g., herpes simplex virus, etc.) in order enhance expression and/or otherwise modulate targeting to muscle cell (e.g., heart (cardiac cell), or skeletal (gastrocnemius) cell). See, e.g., WO 2020/223231, published Nov. 5, 2020 (rh91, including table with deamidation pattern), U.S. Provisional Patent Application No. 63/065,616, filed Aug. 14, 2020, U.S. Provisional Patent Application No. 63/109,734, filed Nov. 4, 2020, and International Patent Application No. PCT/US21/45945, filed Aug. 13, 2021, which is now published as WO 2022/036220, all of which are incorporated herein by reference in their entireties. In certain embodiments, AAV capsids having reduced capsid deamidation may be selected. See, e.g., PCT/US19/19804 and PCT/US18/19861, both filed Feb. 27, 2019, and incorporated by reference in their entireties.

In certain embodiments, the mutant capsids described herein are characterized by having a deamidation pattern similar to their parental AAV, e.g., such as described in US 2020/0056159, published Feb. 20, 2020 (AAVhu68; highly deamidated in N57, N329, N452 and N512), with minor optional amounts of deamidation); US 2020/0407750, published Dec. 31, 2020 (AAV9, highly deamidated in N57, N329, N452 and N512), each of which is incorporated herein by reference in its entirety.

In certain embodiments, a recombinant adeno-associated virus particle (rAAV) is provided comprising: (a) an adeno-associated virus (AAV) capsid comprising VP1 proteins, VP2 proteins and VP3 proteins, wherein the capsid proteins have an amino acid sequence comprising a hypervariable region comprising an exogenous targeting peptide, wherein the exogenous targeting peptide comprises “Xn-n-mer-Xm”, wherein: (i) Xn is 0, 1, 2 or 3 amino acid residues independently selected from any amino acid; (ii) the n-mer is IIRGDPA (SEQ ID NO: 1), AVIRGDV (SEQ ID NO: 2), IVRGDPA (SEQ ID NO: 8), MIRGDVK (SEQ ID NO: 9), AQHRGDV (SEQ ID NO: 10), VSRGDPN (SEQ ID NO: 11), VSRGDPA (SEQ ID NO: 12), PLVRGDI (SEQ ID NO: 13), PYVRGDP (SEQ ID NO: 14), VVRGDPQ (SEQ ID NO: 15), PTRGDVK (SEQ ID NO: 16), VVQRGDV (SEQ ID NO: 17), QHRGDTQ (SEQ ID NO: 18), QIRGDLR (SEQ ID NO: 19), RGDYAQV (SEQ ID NO: 20), IGRGDPN (SEQ ID NO: 21), RGDLHGY (SEQ ID NO: 22), RGDYSTM (SEQ ID NO: 23), or PYQRGDH (SEQ ID NO: 24), or an n-mer sequence of at least 6, at least 7, or full-length consecutive amino acids of any one of the n-mers; and (iii) Xm is 0, 1, 2, or 3 amino acid residues independently selected from any amino acid; and (b) a vector genome packaged in the AAV capsid, wherein the vector genome comprises a nucleic acid sequence encoding a gene product under control of sequences which direct expression thereof. In certain embodiments, a rAAV comprises capsid proteins having an amino acid sequence comprising a hypervariable region comprising an exogenous targeting peptide, wherein the exogenous targeting peptide comprises: IIRGDPA (SEQ ID NO: 1), AVIRGDV (SEQ ID NO: 2), IVRGDPA (SEQ ID NO: 8), MIRGDVK (SEQ ID NO: 9), AQHRGDV (SEQ ID NO: 10), VSRGDPN (SEQ ID NO: 11), VSRGDPA (SEQ ID NO: 12), PLVRGDI (SEQ ID NO:13), PYVRGDP (SEQ ID NO: 14), VVRGDPQ (SEQ ID NO: 15), PTRGDVK (SEQ ID NO: 16), VVQRGDV (SEQ ID NO: 17), QHRGDTQ (SEQ ID NO: 18), QIRGDLR (SEQ ID NO: 19), RGDYAQV (SEQ ID NO: 20), IGRGDPN (SEQ ID NO: 21), RGDLHGY (SEQ ID NO: 22), RGDYSTM (SEQ ID NO: 23), or PYQRGDH (SEQ ID NO: 24), and/or combination of any thereof. In certain embodiments, the rAAV comprising capsid proteins comprising exogenous targeting peptide as described herein (i.e., engineered rAAV capsid) has a greater muscle specificity, targeting, and/or efficacy as compared to a parental rAAV capsid.

In certain embodiments, provided herein is a recombinant adeno-associated viral particle (rAAV) comprising an AAV capsid, wherein the AAV capsid is not an AAV2 capsid. In certain embodiments, the rAAV comprises an AAV capsid, wherein the AAV capsid is not a mutant AAV2 capsid comprising NDVRAVS (SEQ ID NO: 36) sequence. In certain embodiments, the rAAV comprises an AAV2 capsid wherein the AAV2 capsid protein comprises at least one or more of the exogenous targeting peptides comprising “Xn n-mer-Xm”, wherein: (i) Xn is 0, 1, 2 or 3 amino acid residues independently selected from any amino acid; (ii) the n-mer is IIRGDPA (SEQ ID NO: 1), AVIRGDV (SEQ ID NO: 2), IVRGDPA (SEQ ID NO: 8), MIRGDVK (SEQ ID NO: 9), AQHRGDV (SEQ ID NO: 10), VSRGDPN (SEQ ID NO: 11), VSRGDPA (SEQ ID NO: 12), PLVRGDI (SEQ ID NO: 13), PYVRGDP (SEQ ID NO: 14), VVRGDPQ (SEQ ID NO: 15), PTRGDVK (SEQ ID NO: 16), VVQRGDV (SEQ ID NO: 17), QHRGDTQ (SEQ ID NO: 18), QIRGDLR (SEQ ID NO: 19), RGDYAQV (SEQ ID NO: 20), IGRGDPN (SEQ ID NO: 21), RGDLHGY (SEQ ID NO: 22), RGDYSTM (SEQ ID NO: 23), or PYQRGDH (SEQ ID NO: 24), or an n-mer sequence of at least 6, at least 7, or full-length consecutive amino acids of any one of the n-mers; and (iii) Xm is 0, 1, 2, or 3 amino acid residues independently selected from any amino acid.

In certain embodiments, the rAAV comprises an AAV9 capsid having AAV9 capsid proteins comprising one or more the exogenous targeting peptides comprising “Xn-n-mer-Xm”, wherein: (i) Xn is 0, 1, 2 or 3 amino acid residues independently selected from any amino acid; (ii) the n-mer is IIRGDPA (SEQ ID NO: 1), AVIRGDV (SEQ ID NO: 2), IVRGDPA (SEQ ID NO: 8), MIRGDVK (SEQ ID NO: 9), AQHRGDV (SEQ ID NO: 10), VSRGDPN (SEQ ID NO: 11), VSRGDPA (SEQ ID NO: 12), PLVRGDI (SEQ ID NO: 13), PYVRGDP (SEQ ID NO: 14), VVRGDPQ (SEQ ID NO: 15), PTRGDVK (SEQ ID NO: 16), VVQRGDV (SEQ ID NO: 17), QHRGDTQ (SEQ ID NO: 18), QIRGDLR (SEQ ID NO: 19), RGDYAQV (SEQ ID NO: 20), IGRGDPN (SEQ ID NO: 21), RGDLHGY (SEQ ID NO: 22), RGDYSTM (SEQ ID NO: 23), or PYQRGDH (SEQ ID NO: 24), or an n-mer sequence of at least 6, at least 7, or the full-length consecutive amino acids of any one of the n-mers; and (iii) Xm is 0, 1, 2, or 3 amino acid residues independently selected from any amino acid. In certain embodiments, the rAAV comprises an AAV9 capsid wherein the AAV9 capsid protein comprises a hypervariable region comprising an exogenous targeting peptide, wherein the exogenous targeting peptide comprises: IIRGDPA (SEQ ID NO: 1), AVIRGDV (SEQ ID NO: 2), IVRGDPA (SEQ ID NO: 8), MIRGDVK (SEQ ID NO: 9), AQHRGDV (SEQ ID NO: 10), VSRGDPN (SEQ ID NO: 11), VSRGDPA (SEQ ID NO: 12), PLVRGDI (SEQ ID NO:13), PYVRGDP (SEQ ID NO: 14), VVRGDPQ (SEQ ID NO: 15), PTRGDVK (SEQ ID NO: 16), VVQRGDV (SEQ ID NO: 17), QHRGDTQ (SEQ ID NO: 18), QIRGDLR (SEQ ID NO: 19), RGDYAQV (SEQ ID NO: 20), IGRGDPN (SEQ ID NO: 21), RGDLHGY (SEQ ID NO: 22), RGDYSTM (SEQ ID NO: 23), PYQRGDH (SEQ ID NO: 24), and/or combination of any thereof. In certain embodiments, the rAAV comprises an AAV9 capsid wherein the AAV9 capsid proteins comprise a hypervariable region comprising an exogenous targeting peptide, wherein the exogenous targeting peptide comprises IIRGDPA (SEQ ID NO: 1). In certain embodiments, the rAAV comprises an AAV9 capsid wherein the AAV9 capsid proteins comprise a hypervariable region comprising an exogenous targeting peptide, wherein the exogenous targeting peptide comprises AVIRGDV (SEQ ID NO: 2).

In certain embodiments, the rAAV comprises an AAVhu68 capsid having AAVhu68 capsid proteins comprising one or more of exogenous targeting peptides comprising “Xn-n-mer-Xm”, wherein: (i) Xn is 0, 1, 2 or 3 amino acid residues independently selected from any amino acid; (ii) the n-mer is IIRGDPA (SEQ ID NO: 1), AVIRGDV (SEQ ID NO: 2), IVRGDPA (SEQ ID NO: 8), MIRGDVK (SEQ ID NO: 9), AQHRGDV (SEQ ID NO: 10), VSRGDPN (SEQ ID NO: 11), VSRGDPA (SEQ ID NO: 12), PLVRGDI (SEQ ID NO: 13), PYVRGDP (SEQ ID NO: 14), VVRGDPQ (SEQ ID NO: 15), PTRGDVK (SEQ ID NO: 16), VVQRGDV (SEQ ID NO: 17), QHRGDTQ (SEQ ID NO: 18), QIRGDLR (SEQ ID NO: 19), RGDYAQV (SEQ ID NO: 20), IGRGDPN (SEQ ID NO: 21), RGDLHGY (SEQ ID NO: 22), RGDYSTM (SEQ ID NO: 23), or PYQRGDH (SEQ ID NO: 24), or an n-mer sequence of at least 6, at least 7, or the full-length consecutive amino acids of any one of the n-mers; and (iii) Xm is 0, 1, 2, or 3 amino acid residues independently selected from any amino acid. In certain embodiments, the rAAV comprises AA Vhu68 capsid proteins comprising a hypervariable region comprising an exogenous targeting peptide, wherein the exogenous targeting peptide comprises: IIRGDPA (SEQ ID NO: 1), AVIRGDV (SEQ ID NO: 2), IVRGDPA (SEQ ID NO: 8), MIRGDVK (SEQ ID NO: 9), AQHRGDV (SEQ ID NO: 10), VSRGDPN (SEQ ID NO: 11), VSRGDPA (SEQ ID NO: 12), PLVRGDI (SEQ ID NO:13), PYVRGDP (SEQ ID NO: 14), VVRGDPQ (SEQ ID NO: 15), PTRGDVK (SEQ ID NO: 16), VVQRGDV (SEQ ID NO: 17), QHRGDTQ (SEQ ID NO: 18), QIRGDLR (SEQ ID NO: 19), RGDYAQV (SEQ ID NO: 20), IGRGDPN (SEQ ID NO: 21), RGDLHGY (SEQ ID NO: 22), RGDYSTM (SEQ ID NO: 23), PYQRGDH (SEQ ID NO: 24), and/or combination of any thereof.

In certain embodiments, the rAAV comprises an AAV9 capsid having AAV9 capsid proteins comprising one or more exogenous peptides comprising “Xn-IIRGDPA (SEQ ID NO: 1)-Xm”, wherein Xn is 0, 1, 2 or 3 amino acid residues independently selected from any amino acid, and wherein Xm is 0, 1, 2, or 3 amino acid residues independently selected from any amino acid. In certain embodiments, the rAAV comprises an AAV9 capsid having AAV9 capsid proteins comprising one or more of the exogenous peptides comprising IIRGDPA (SEQ ID NO: 1).

In certain embodiments, the rAAV comprises an AAV9 capsid having AAV9 capsid proteins comprising one or more exogenous peptides comprising “Xn-AVIRGDV (SEQ ID NO: 2)-Xm”, wherein Xn is 0, 1, 2 or 3 amino acid residues independently selected from any amino acid, and wherein Xm is 0, 1, 2, or 3 amino acid residues independently selected from any amino acid. In certain embodiments, the rAAV comprises an AAV9 capsid having AAV9 capsid proteins comprising one or more exogenous peptides comprising AVIRGDV (SEQ ID NO: 2).

In certain embodiments, the rAAV comprises an AAV9 capsid having AAV9 capsid proteins comprising one or more of the exogenous peptides comprising “Xn-IVRGDPA (SEQ ID NO: 8)-Xm”, wherein Xn is 0, 1, 2 or 3 amino acid residues independently selected from any amino acid, and wherein Xm is 0, 1, 2, or 3 amino acid residues independently selected from any amino acid. In certain embodiments, the rAAV comprises an AAV9 capsid having AAV9 capsid proteins comprising one or more exogenous peptides comprising IVRGDPA (SEQ ID NO: 8).

In certain embodiments, the rAAV comprises an AAV9 capsid having AAV9 capsid proteins comprising one or more exogenous peptides comprising “Xn-MIRGDVK (SEQ ID NO: 9)-Xm”, wherein Xn is 0, 1, 2 or 3 amino acid residues independently selected from any amino acid, and wherein Xm is 0, 1, 2, or 3 amino acid residues independently selected from any amino acid. In certain embodiments, the rAAV comprises an AAV9 capsid having AAV9 capsid proteins comprising one or more exogenous peptides comprising MIRGDVK (SEQ ID NO: 9).

In certain embodiments, the rAAV comprises an AAV9 capsid having AAV9 capsid protein comprises at least one or more of the exogenous peptides comprising “Xn-AQHRGDV (SEQ ID NO: 10)-Xm”, wherein Xn is 0, 1, 2 or 3 amino acid residues independently selected from any amino acid, and wherein Xm is 0, 1, 2, or 3 amino acid residues independently selected from any amino acid. In certain embodiments, the rAAV comprises an AAV9 capsid having AAV9 capsid protein comprises at least one or more of the exogenous peptides comprising AQHRGDV (SEQ ID NO: 10).

In certain embodiments, the rAAV comprises an AAV9 capsid having AAV9 capsid proteins comprising one or more exogenous peptides comprising “Xn-VSRGDPN (SEQ ID NO: 11)-Xm”, wherein Xn is 0, 1, 2 or 3 amino acid residues independently selected from any amino acid, and wherein Xm is 0, 1, 2, or 3 amino acid residues independently selected from any amino acid. In certain embodiments, the rAAV comprises an AAV9 capsid having AAV9 capsid proteins comprising one or more of exogenous peptides comprising VSRGDPN (SEQ ID NO: 11).

In certain embodiments, the rAAV comprises an AAV9 capsid having AAV9 capsid proteins comprising one or more exogenous peptides comprising “Xn-VSRGDPA (SEQ ID NO: 12)-Xm”, wherein Xn is 0, 1, 2 or 3 amino acid residues independently selected from any amino acid, and wherein Xm is 0, 1, 2, or 3 amino acid residues independently selected from any amino acid. In certain embodiments, the rAAV comprises an AAV9 capsid having AAV9 capsid proteins comprising one or more exogenous peptides comprising VSRGDPA (SEQ ID NO: 12).

In certain embodiments, the rAAV comprises an AAV9 capsid having AAV9 capsid proteins comprising one or more exogenous peptides comprising “Xn-PLVRGDI (SEQ ID NO: 13)-Xm”, wherein Xn is 0, 1, 2 or 3 amino acid residues independently selected from any amino acid, and wherein Xm is 0, 1, 2, or 3 amino acid residues independently selected from any amino acid. In certain embodiments, the rAAV comprises an AAV9 capsid having AAV9 capsid proteins comprising one or more exogenous peptides comprising PLVRGDI (SEQ ID NO: 13).

In certain embodiments, the rAAV comprises an AAV9 capsid having AAV9 capsid proteins comprising one or more exogenous peptides comprising “Xn-PYVRGDP (SEQ ID NO: 14)-Xm”, wherein Xn is 0, 1, 2 or 3 amino acid residues independently selected from any amino acid, and wherein Xm is 0, 1, 2, or 3 amino acid residues independently selected from any amino acid. In certain embodiments, the rAAV comprises an AAV9 capsid having AAV9 capsid proteins comprising one or more exogenous peptides comprising PYVRGDP (SEQ ID NO: 14).

In certain embodiments, the rAAV comprises an AAV9 capsid having AAV9 capsid proteins comprising one or more of the exogenous peptides comprising “Xn-VVRGDPQ (SEQ ID NO: 15)-Xm”, wherein Xn is 0, 1, 2 or 3 amino acid residues independently selected from any amino acid, and wherein Xm is 0, 1, 2, or 3 amino acid residues independently selected from any amino acid. In certain embodiments, the rAAV comprises an AAV9 capsid having AAV9 capsid proteins comprising one or more exogenous peptides comprising VVRGDPQ (SEQ ID NO: 15).

In certain embodiments, the rAAV comprises an AAV9 capsid having AAV9 capsid protein comprises one or more of the exogenous peptides comprising “Xn-PTRGDVK (SEQ ID NO: 16)-Xm”, wherein Xn is 0, 1, 2 or 3 amino acid residues independently selected from any amino acid, and wherein Xm is 0, 1, 2, or 3 amino acid residues independently selected from any amino acid. In certain embodiments, the rAAV comprises an AAV9 capsid having AAV9 capsid proteins comprising one or more exogenous peptides comprising PTRGDVK (SEQ ID NO: 16).

In certain embodiments, the rAAV comprises an AAV9 capsid having AAV9 capsid proteins comprising one or more exogenous peptides comprising “Xn-VVQRGDV (SEQ ID NO: 17)-Xm”, wherein Xn is 0, 1, 2 or 3 amino acid residues independently selected from any amino acid, and wherein Xm is 0, 1, 2, or 3 amino acid residues independently selected from any amino acid. In certain embodiments, the rAAV comprises an AAV9 capsid having AAV9 capsid proteins comprising one or more exogenous peptides comprising VVQRGDV (SEQ ID NO: 17).

In certain embodiments, the rAAV comprises an AAV9 capsid having AAV9 capsid proteins comprising one or more exogenous peptides comprising “Xn-QHRGDTQ (SEQ ID NO: 18)-Xm”, wherein Xn is 0, 1, 2 or 3 amino acid residues independently selected from any amino acid, and wherein Xm is 0, 1, 2, or 3 amino acid residues independently selected from any amino acid. In certain embodiments, the rAAV comprises an AAV9 capsid having AAV9 capsid proteins comprising one or more exogenous peptides comprising QHRGDTQ (SEQ ID NO: 18).

In certain embodiments, the rAAV comprises an AAV9 capsid having AAV9 capsid proteins comprising one or more exogenous peptides comprising “Xn-QIRGDLR (SEQ ID NO: 19)-Xm”, wherein Xn is 0, 1, 2 or 3 amino acid residues independently selected from any amino acid, and wherein Xm is 0, 1, 2, or 3 amino acid residues independently selected from any amino acid. In certain embodiments, the rAAV comprises an AAV9 capsid having AAV9 capsid proteins comprising one or more exogenous peptides comprising QIRGDLR (SEQ ID NO: 19).

In certain embodiments, the rAAV comprises an AAV9 capsid having AAV9 capsid proteins comprising one or more exogenous peptides comprising “Xn-RGDYAQV (SEQ ID NO: 20)-Xm”, wherein Xn is 0, 1, 2 or 3 amino acid residues independently selected from any amino acid, and wherein Xm is 0, 1, 2, or 3 amino acid residues independently selected from any amino acid. In certain embodiments, the rAAV comprises an AAV9 capsid having AAV9 capsid proteins comprising one or more exogenous peptides comprising RGDYAQV (SEQ ID NO: 20).

In certain embodiments, the rAAV comprises an AAV9 capsid having AAV9 capsid proteins comprising one or more exogenous peptides comprising “Xn-IGRGDPN (SEQ ID NO: 21)-Xm”, wherein Xn is 0, 1, 2 or 3 amino acid residues independently selected from any amino acid, and wherein Xm is 0, 1, 2, or 3 amino acid residues independently selected from any amino acid. In certain embodiments, the rAAV comprises an AAV9 capsid having AAV9 capsid proteins comprising one or more exogenous peptides comprising IGRGDPN (SEQ ID NO: 21).

In certain embodiments, the rAAV comprises an AAV9 capsid having AAV9 capsid proteins comprising one or more exogenous peptides comprising “Xn-RGDLHGY (SEQ ID NO: 22)-Xm”, wherein Xn is 0, 1, 2 or 3 amino acid residues independently selected from any amino acid, and wherein Xm is 0, 1, 2, or 3 amino acid residues independently selected from any amino acid. In certain embodiments, the rAAV comprises an AAV9 capsid having AAV9 capsid protein comprising one or more exogenous peptides comprising RGDLHGY (SEQ ID NO: 22).

In certain embodiments, the rAAV comprises an AAV9 capsid having AAV9 capsid proteins comprising one or more exogenous peptides comprising “Xn-RGDYSTM (SEQ ID NO: 23)-Xm”, wherein Xn is 0, 1, 2 or 3 amino acid residues independently selected from any amino acid, and wherein Xm is 0, 1, 2, or 3 amino acid residues independently selected from any amino acid. In certain embodiments, the rAAV comprises an AAV9 capsid having AAV9 capsid proteins comprising one or more exogenous peptides comprising RGDYSTM (SEQ ID NO: 23).

In certain embodiments, the rAAV comprises an AAV9 capsid having AAV9 capsid proteins comprising one or more exogenous peptides comprising “Xn-PYQRGDH (SEQ ID NO: 24)-Xm”, wherein Xn is 0, 1, 2 or 3 amino acid residues independently selected from any amino acid, and wherein Xm is 0, 1, 2, or 3 amino acid residues independently selected from any amino acid. In certain embodiments, the rAAV comprises an AAV9 capsid having AAV9 capsid proteins comprising one or more exogenous peptides comprising PYQRGDH (SEQ ID NO: 24).

In certain embodiments, the rAAV comprises a mutant AAV9-IIRGDPA capsid or a mutant AAVhu68-IIRGDPA capsid, which comprises mutant VP1, mutant VP2, and mutant VP3 proteins, each having a heterogenous population of proteins comprising the IIRGDPA (SEQ ID NO: 1) peptide insert. In certain embodiments, the proteins are further characterized by having three or four highly deamidated asparagines in positions: N57, N329, N452 and N512, and optional deamidation in other positions within the parental capsid sequence. In certain embodiments, the rAAV has a capsid comprising AAV9-IIRGDPA (or AAVhu68-IIRGDPA)-in which each the AAV VP1, AAV VP2, AAV VP3 proteins are a heterogenous population and comprise the AAV VP3 region of SEQ ID NO: 73 (about amino acid 203 to about amino acid 736 based on the residue positions in SEQ ID NO: 25 (AAV9)). The proteins further comprising a heterogenous population of each of the capsid proteins having deamidation in about 50% to about 100% of positions N57 (VP1 only), N329, N452, and/or N512, based on the parental capsid residue positions. In certain embodiments, the percentage of deamidation in one or more of these positions is over 60%, over 70%, over 80%, over 90%, over 95%, or about 70% to about 100%, or values therebetween.

The percentage of deamidation in each of the highly deamidated positions may differ from each other within a rAAV capsid. For example, the percent of deamidation at position N57 in an AAV capsid, may differ from the percent of deamidation at each of the other highly deamidated residues, which may have higher (or lower) deamidation percentages. Similarly, the percent of deamidation at position N329, N452, and/or N512, may each vary from one another. In certain embodiments, the percentage of deamidation for each of the highly deamidated positions is determined for the total VP proteins in a single rAAV capsid or a stock of rAAV capsids. In certain embodiments, a parental clade F capsid comprises VP proteins which are highly deamidated in all four of these positions. In certain embodiments, the percentage of deamidation in one or more of these highly deamidated positions is over 50%, over 55%, over 60%, over 65%, over 70%, over 75%, over 80%, over 85%, over 90%, over 95%, or about 70% to about 100%, or values therebetween. In certain embodiments, there may be minor deamidation in other positions within the capsid. In certain embodiments, there are no post-translational modifications within the peptide insert (e.g., AVIRGDV) of the mutant capsid. In certain embodiments, the rAAV comprises a mutant AAV9-AVIRGDV capsid or a mutant AAVhu68-AVIRGDV capsid, which comprises mutant VP1, mutant VP2, and mutant VP3 proteins, each having a heterogenous population of proteins comprising the AVIRGDV (SEQ ID NO: 2) peptide insert. In certain embodiments, the proteins are further characterized by having three or four highly deamidated asparagines in positions: N57, N329, N452 and/or N512, and optional deamidation in other positions within the parental capsid sequence. In certain embodiments, a rAAV has a capsid comprising AAV9-AVIRGDV (or AAVhu68-AVIRGDV)-VP proteins in which each the VP1, VP2 VP3 proteins are a heterogenous population and comprise the VP3 region of SEQ ID NO: 75 (about amino acid 203 to about amino acid 736 based on the residue positions in SEQ ID NO: 25 (AAV9)). The proteins further comprising deamidation in about 50% to about 100% of positions N57 (VP1 only), N329, N452, and/or N512, based on the parental capsid residue positions. In certain embodiments, the capsid comprises VP proteins which are highly deamidated in all of these positions. In certain embodiments, the percentage of deamidation in one or more of these highly deamidated positions is over 50%, over 55%, over 60%, over 65%, over 70%, over 75%, over 80%, over 85%, over 90%, over 95%, or about 70% to about 100%, or values therebetween.

In certain embodiments, the rAAV comprises a mutant AAV9-IVRGDPA capsid or a mutant AAVhu68-IVRGDPA capsid, which comprises mutant VP1, mutant VP2, and mutant VP3 proteins, each having a heterogenous population of proteins comprising the IVRGDPA (SEQ ID NO: 8) peptide insert. In certain embodiments, the proteins are further characterized by having three or four highly deamidated asparagines in positions: N57, N329, N452 and/or N512, and optional deamidation in other positions within the parental capsid sequence. In certain embodiments, a rAAV has a capsid comprising AAV9-IVRGDPA (or AAVhu68-IVRGDPA)-VP proteins in which each the VP1, VP2 VP3 proteins are a heterogenous population and comprise the VP3 region of SEQ ID NO: 77 (about amino acid 203 to about amino acid 736 based on the residue positions in SEQ ID NO: 25 (AAV9)). The proteins further comprising deamidation in about 50% to about 100% of positions N57 (VP1 only), N329, N452, and/or N512, based on the parental capsid residue positions. In certain embodiments, the capsid comprises VP proteins which are highly deamidated in all of these positions. In certain embodiments, the percentage of deamidation in one or more of these highly deamidated positions is over 50%, over 55%, over 60%, over 65%, over 70%, over 75%, over 80%, over 85%, over 90%, over 95%, or about 70% to about 100%, or values therebetween. In certain embodiments, the rAAV comprises a mutant AAV9-MIRGDVK capsid or a mutant AAVhu68-MIRGDVK capsid, which comprises mutant VP1, mutant VP2, and mutant VP3 proteins, each having a heterogenous population of proteins comprising the MIRGDVK (SEQ ID NO: 9) peptide insert. In certain embodiments, the proteins are further characterized by having three or four highly deamidated asparagines in positions: N57, N329, N452 and/or N512, and optional deamidation in other positions within the parental capsid sequence. In certain embodiments, a rAAV has a capsid comprising AAV9-MIRGDVK (or AAVhu68-MIRGDVK)-VP proteins in which each the VP1, VP2 VP3 proteins are a heterogenous population and comprise the VP3 region of SEQ ID NO: 79 (about amino acid 203 to about amino acid 736 based on the residue positions in SEQ ID NO: 25 (AAV9)). The proteins further comprising deamidation in about 50% to about 100% of positions N57 (VP1 only), N329, N452, and/or N512, based on the parental capsid residue positions. In certain embodiments, the capsid comprises VP proteins which are highly deamidated in all of these positions. In certain embodiments, the percentage of deamidation in one or more of these highly deamidated positions is over 50%, over 55%, over 60%, over 65%, over 70%, over 75%, over 80%, over 85%, over 90%, over 95%, or about 70% to about 100%, or values therebetween.

In certain embodiments, the rAAV comprises a mutant AAV9-AQHRGDV capsid or a mutant AAVhu68-AQHRGDV capsid, which comprises mutant VP1, mutant VP2, and mutant VP3 proteins, each having a heterogenous population of proteins comprising the AQHRGDV (SEQ ID NO: 10) peptide insert. In certain embodiments, the proteins are further characterized by having three or four highly deamidated asparagines in positions: N57, N329, N452 and/or N512, and optional deamidation in other positions within the parental capsid sequence. In certain embodiments, a rAAV has a capsid comprising AAV9-AQHRGDV (or AAVhu68-AQHRGDV)-VP proteins in which each the VP1, VP2 VP3 proteins are a heterogenous population and comprise the VP3 region of SEQ ID NO: 81 (about amino acid 203 to about amino acid 736 based on the residue positions in SEQ ID NO: 25 (AAV9)). The proteins further comprising deamidation in about 50% to about 100% of positions N57 (VP1 only), N329, N452, and/or N512, based on the parental capsid residue positions. In certain embodiments, the capsid comprises VP proteins which are highly deamidated in all of these positions. In certain embodiments, the percentage of deamidation in one or more of these highly deamidated positions is over 50%, over 55%, over 60%, over 65%, over 70%, over 75%, over 80%, over 85%, over 90%, over 95%, or about 70% to about 100%, or values therebetween.

In certain embodiments, the rAAV comprises a mutant AAV9-VSRGDPN capsid or a mutant AAVhu68-VSRGDPN capsid, which comprises mutant VP1, mutant VP2, and mutant VP3 proteins, each having a heterogenous population of proteins comprising the VSRGDPN (SEQ ID NO: 11) peptide insert. In certain embodiments, the proteins are further characterized by having three or four highly deamidated asparagines in positions: N57, N329, N452 and/or N512, and optional deamidation in other positions within the parental capsid sequence. In certain embodiments, a rAAV has a capsid comprising AAV9-VSRGDPN (or AAVhu68-VSRGDPN)-VP proteins in which each the VP1, VP2 VP3 proteins are a heterogenous population and comprise the VP3 region of SEQ ID NO: 83 (about amino acid 203 to about amino acid 736 based on the residue positions in SEQ ID NO: 25 (AAV9)). The proteins further comprising deamidation in about 50% to about 100% of positions N57 (VP1 only), N329, N452, and/or N512, based on the parental capsid residue positions. In certain embodiments, the capsid comprises VP proteins which are highly deamidated in all of these positions. In certain embodiments, the percentage of deamidation in one or more of these highly deamidated positions is over 50%, over 55%, over 60%, over 65%, over 70%, over 75%, over 80%, over 85%, over 90%, over 95%, or about 70% to about 100%, or values therebetween.

In certain embodiments, the rAAV comprises a mutant AAV9-VSRGDPA capsid or a mutant AAVhu68-VSRGDPA capsid, which comprises mutant VP1, mutant VP2, and mutant VP3 proteins, each having a heterogenous population of proteins comprising the VSRGDPA (SEQ ID NO: 12) peptide insert. In certain embodiments, the proteins are further characterized by having three or four highly deamidated asparagines in positions: N57, N329, N452 and/or N512, and optional deamidation in other positions within the parental capsid sequence. In certain embodiments, a rAAV has a capsid comprising AAV9-VSRGDPA (or AAVhu68-VSRGDPA)-VP proteins in which each the VP1, VP2 VP3 proteins are a heterogenous population and comprise the VP3 region of SEQ ID NO: 85 (about amino acid 203 to about amino acid 736 based on the residue positions in SEQ ID NO: 25 (AAV9)). The proteins further comprising deamidation in about 50% to about 100% of positions N57 (VP1 only), N329, N452, and/or N512, based on the parental capsid residue positions. In certain embodiments, the capsid comprises VP proteins which are highly deamidated in all of these positions. In certain embodiments, the percentage of deamidation in one or more of these highly deamidated positions is over 50%, over 55%, over 60%, over 65%, over 70%, over 75%, over 80%, over 85%, over 90%, over 95%, or about 70% to about 100%, or values therebetween.

In certain embodiments, the rAAV comprises a mutant AAV9-PLVRGDI capsid or a mutant AAVhu68-PLVRGDI capsid, which comprises mutant VP1, mutant VP2, and mutant VP3 proteins, each having a heterogenous population of proteins comprising the PLVRGDI (SEQ ID NO: 13) peptide insert. In certain embodiments, the proteins are further characterized by having three or four highly deamidated asparagines in positions: N57, N329, N452 and/or N512, and optional deamidation in other positions within the parental capsid sequence. In certain embodiments, a rAAV has a capsid comprising AAV9-PLVRGDI (or AAVhu68-PLVRGDI)-VP proteins in which each the VP1, VP2 VP3 proteins are a heterogenous population and comprise the VP3 region of SEQ ID NO: 87 (about amino acid 203 to about amino acid 736 based on the residue positions in SEQ ID NO: 25 (AAV9)). The proteins further comprising deamidation in about 50% to about 100% of positions N57 (VP1 only), N329, N452, and/or N512, based on the parental capsid residue positions. In certain embodiments, the capsid comprises VP proteins which are highly deamidated in all of these positions. In certain embodiments, the percentage of deamidation in one or more of these highly deamidated positions is over 50%, over 55%, over 60%, over 65%, over 70%, over 75%, over 80%, over 85%, over 90%, over 95%, or about 70% to about 100%, or values therebetween.

In certain embodiments, the rAAV comprises a mutant AAV9-PYVRGDP capsid or a mutant AAVhu68-PYVRGDP capsid, which comprises mutant VP1, mutant VP2, and mutant VP3 proteins, each having a heterogenous population of proteins comprising the PYVRGDP (SEQ ID NO: 14) peptide insert. In certain embodiments, the proteins are further characterized by having three or four highly deamidated asparagines in positions: N57, N329, N452 and/or N512, and optional deamidation in other positions within the parental capsid sequence. In certain embodiments, a rAAV has a capsid comprising AAV9-PYVRGDP (or AAVhu68-PYVRGDP)-VP proteins in which each the VP1, VP2 VP3 proteins are a heterogenous population and comprise the VP3 region of SEQ ID NO: 89 (about amino acid 203 to about amino acid 736 based on the residue positions in SEQ ID NO: 25 (AAV9)). The proteins further comprising deamidation in about 50% to about 100% of positions N57 (VP1 only), N329, N452, and/or N512, based on the parental capsid residue positions. In certain embodiments, the capsid comprises VP proteins which are highly deamidated in all of these positions. In certain embodiments, the percentage of deamidation in one or more of these highly deamidated positions is over 50%, over 55%, over 60%, over 65%, over 70%, over 75%, over 80%, over 85%, over 90%, over 95%, or about 70% to about 100%, or values therebetween.

In certain embodiments, the rAAV comprises a mutant AAV9-VVRGDPQ capsid or a mutant AAVhu68-VVRGDPQ capsid, which comprises mutant VP1, mutant VP2, and mutant VP3 proteins, each having a heterogenous population of proteins comprising the VVRGDPQ (SEQ ID NO: 15) peptide insert. In certain embodiments, the proteins are further characterized by having three or four highly deamidated asparagines in positions: N57, N329, N452 and/or N512, and optional deamidation in other positions within the parental capsid sequence. In certain embodiments, a rAAV has a capsid comprising AAV9-VVRGDPQ (or AAVhu68-VVRGDPQ)-VP proteins in which each the VP1, VP2 VP3 proteins are a heterogenous population and comprise the VP3 region of SEQ ID NO: 91 (about amino acid 203 to about amino acid 736 based on the residue positions in SEQ ID NO: 25 (AAV9)). The proteins further comprising deamidation in about 50% to about 100% of positions N57 (VP1 only), N329, N452, and/or N512, based on the parental capsid residue positions. In certain embodiments, the capsid comprises VP proteins which are highly deamidated in all of these positions. In certain embodiments, the percentage of deamidation in one or more of these highly deamidated positions is over 50%, over 55%, over 60%, over 65%, over 70%, over 75%, over 80%, over 85%, over 90%, over 95%, or about 70% to about 100%, or values therebetween.

In certain embodiments, the rAAV comprises a mutant AAV9-VVQRGDV capsid or a mutant AAVhu68-VVQRGDV capsid, which comprises mutant VP1, mutant VP2, and mutant VP3 proteins, each having a heterogenous population of proteins comprising the VVQRGDV (SEQ ID NO: 17) peptide insert. In certain embodiments, the proteins are further characterized by having three or four highly deamidated asparagines in positions: N57, N329, N452 and/or N512, and optional deamidation in other positions within the parental capsid sequence. In certain embodiments, a rAAV has a capsid comprising AAV9-VVQRGDV (or AAVhu68-VVQRGDV)-VP proteins in which each the VP1, VP2 VP3 proteins are a heterogenous population and comprise the VP3 region of SEQ ID NO: 93 (about amino acid 203 to about amino acid 736 based on the residue positions in SEQ ID NO: 25 (AAV9)). The proteins further comprising deamidation in about 50% to about 100% of positions N57 (VP1 only), N329, N452, and/or N512, based on the parental capsid residue positions. In certain embodiments, the capsid comprises VP proteins which are highly deamidated in all of these positions. In certain embodiments, the percentage of deamidation in one or more of these highly deamidated positions is over 50%, over 55%, over 60%, over 65%, over 70%, over 75%, over 80%, over 85%, over 90%, over 95%, or about 70% to about 100%, or values therebetween.

In certain embodiments, the rAAV comprises a mutant AAV9-QHRGDTQ capsid or a mutant AAVhu68-QHRGDTQ capsid, which comprises mutant VP1, mutant VP2, and mutant VP3 proteins, each having a heterogenous population of proteins comprising the QHRGDTQ (SEQ ID NO: 18) peptide insert. In certain embodiments, the proteins are further characterized by having three or four highly deamidated asparagines in positions: N57, N329, N452 and/or N512, and optional deamidation in other positions within the parental capsid sequence. In certain embodiments, a rAAV has a capsid comprising AAV9-QHRGDTQ (or AAVhu68-QHRGDTQ)-VP proteins in which each the VP1, VP2 VP3 proteins are a heterogenous population and comprise the VP3 region of SEQ ID NO: 95 (about amino acid 203 to about amino acid 736 based on the residue positions in SEQ ID NO: 25 (AAV9)). The proteins further comprising deamidation in about 50% to about 100% of positions N57 (VP1 only), N329, N452, and/or N512, based on the parental capsid residue positions. In certain embodiments, the capsid comprises VP proteins which are highly deamidated in all of these positions. In certain embodiments, the percentage of deamidation in one or more of these highly deamidated positions is over 50%, over 55%, over 60%, over 65%, over 70%, over 75%, over 80%, over 85%, over 90%, over 95%, or about 70% to about 100%, or values therebetween.

In certain embodiments, the rAAV comprises a mutant AAV9-QIRGDLR capsid or a mutant AAVhu68-QIRGDLR capsid, which comprises mutant VP1, mutant VP2, and mutant VP3 proteins, each having a heterogenous population of proteins comprising the QIRGDLR (SEQ ID NO: 19) peptide insert. In certain embodiments, the proteins are further characterized by having three or four highly deamidated asparagines in positions: N57, N329, N452 and N512, and optional deamidation in other positions within the parental capsid sequence. In certain embodiments, a rAAV has a capsid comprising AAV9-QIRGDLR (or AAVhu68-QIRGDLR)-VP proteins in which each the VP1, VP2 VP3 proteins are a heterogenous population and comprise the VP3 region of SEQ ID NO: 97 (about amino acid 203 to about amino acid 736 based on the residue positions in SEQ ID NO: 25 (AAV9)). The proteins further comprising deamidation in about 50% to about 100% of positions N57 (VP1 only), N329, N452, and/or N512, based on the parental capsid residue positions. In certain embodiments, the capsid comprises VP proteins which are highly deamidated in all of these positions. In certain embodiments, the percentage of deamidation in one or more of these highly deamidated positions is over 50%, over 55%, over 60%, over 65%, over 70%, over 75%, over 80%, over 85%, over 90%, over 95%, or about 70% to about 100%, or values therebetween.

In certain embodiments, the rAAV comprises a mutant AAV9-RGDYAQV capsid or a mutant AAVhu68-RGDYAQV capsid, which comprises mutant VP1, mutant VP2, and mutant VP3 proteins, each having a heterogenous population of proteins comprising the RGDYAQV (SEQ ID NO: 20) peptide insert. In certain embodiments, the proteins are further characterized by having three or four highly deamidated asparagines in positions: N57, N329, N452 and N512, and optional deamidation in other positions within the parental capsid sequence. In certain embodiments, a rAAV has a capsid comprising AAV9-RGDYAQV (or AAVhu68-RGDYAQV)-VP proteins in which each the VP1, VP2 VP3 proteins are a heterogenous population and comprise the VP3 region of SEQ ID NO: 99 (about amino acid 203 to about amino acid 736 based on the residue positions in SEQ ID NO: 25 (AAV9)). The proteins further comprising deamidation in about 50% to about 100% of positions N57 (VP1 only), N329, N452, and/or N512, based on the parental capsid residue positions. In certain embodiments, the capsid comprises VP proteins which are highly deamidated in all of these positions. In certain embodiments, the percentage of deamidation in one or more of these highly deamidated positions is over 50%, over 55%, over 60%, over 65%, over 70%, over 75%, over 80%, over 85%, over 90%, over 95%, or about 70% to about 100%, or values therebetween.

In certain embodiments, the rAAV comprises a mutant AAV9-IGRGDPN capsid or a mutant AAVhu68-IGRGDPN capsid, which comprises mutant VP1, mutant VP2, and mutant VP3 proteins, each having a heterogenous population of proteins comprising the IGRGDPN (SEQ ID NO: 21) peptide insert. In certain embodiments, the proteins are further characterized by having three or four highly deamidated asparagines in positions: N57, N329, N452 and N512, and optional deamidation in other positions within the parental capsid sequence. In certain embodiments, a rAAV has a capsid comprising AAV9-IGRGDPN (or AAVhu68-IGRGDPN)-VP proteins in which each the VP1, VP2 VP3 proteins are a heterogenous population and comprise the VP3 region of SEQ ID NO: 101 (about amino acid 203 to about amino acid 736 based on the residue positions in SEQ ID NO: 25 (AAV9)). The proteins further comprising deamidation in about 50% to about 100% of positions N57 (VP1 only), N329, N452, and/or N512, based on the parental capsid residue positions. In certain embodiments, the capsid comprises VP proteins which are highly deamidated in all of these positions. In certain embodiments, the percentage of deamidation in one or more of these highly deamidated positions is over 50%, over 55%, over 60%, over 65%, over 70%, over 75%, over 80%, over 85%, over 90%, over 95%, or about 70% to about 100%, or values therebetween.

In certain embodiments, the rAAV comprises a mutant AAV9-RGDLHGY capsid or a mutant AAVhu68-RGDLHGY capsid, which comprises mutant VP1, mutant VP2, and mutant VP3 proteins, each having a heterogenous population of proteins comprising the RGDLHGY (SEQ ID NO: 22) peptide insert. In certain embodiments, the proteins are further characterized by having three or four highly deamidated asparagines in positions: N57, N329, N452 and N512, and optional deamidation in other positions within the parental capsid sequence. In certain embodiments, a rAAV has a capsid comprising AAV9-RGDLHGY (or AAVhu68-RGDLHGY)-VP proteins in which each the VP1, VP2 VP3 proteins are a heterogenous population and comprise the VP3 region of SEQ ID NO: 103 (about amino acid 203 to about amino acid 736 based on the residue positions in SEQ ID NO: 25 (AAV9)). The proteins further comprising deamidation in about 50% to about 100% of positions N57 (VP1 only), N329, N452, and/or N512, based on the parental capsid residue positions. In certain embodiments, the capsid comprises VP proteins which are highly deamidated in all of these positions. In certain embodiments, the percentage of deamidation in one or more of these highly deamidated positions is over 50%, over 55%, over 60%, over 65%, over 70%, over 75%, over 80%, over 85%, over 90%, over 95%, or about 70% to about 100%, or values therebetween.

In certain embodiments, the rAAV comprises a mutant AAV9-RGDYSTM capsid or a mutant AAVhu68-RGDYSTM capsid, which comprises mutant VP1, mutant VP2, and mutant VP3 proteins, each having a heterogenous population of proteins comprising the RGDYSTM (SEQ ID NO: 23) peptide insert. In certain embodiments, the proteins are further characterized by having three or four highly deamidated asparagines in positions: N57, N329, N452 and N512, and optional deamidation in other positions within the parental capsid sequence. In certain embodiments, a rAAV has a capsid comprising AAV9-RGDYSTM (or AAVhu68-RGDYSTM)-VP proteins in which each the VP1, VP2 VP3 proteins are a heterogenous population and comprise the VP3 region of SEQ ID NO: 105 (about amino acid 203 to about amino acid 736 based on the residue positions in SEQ ID NO: 25 (AAV9)). The proteins further comprising deamidation in about 50% to about 100% of positions N57 (VP1 only), N329, N452, and/or N512, based on the parental capsid residue positions. In certain embodiments, the capsid comprises VP proteins which are highly deamidated in all of these positions. In certain embodiments, the percentage of deamidation in one or more of these highly deamidated positions is over 50%, over 55%, over 60%, over 65%, over 70%, over 75%, over 80%, over 85%, over 90%, over 95%, or about 70% to about 100%, or values therebetween.

In certain embodiments, the rAAV comprises a mutant AAV9-PYQRGDH capsid or a mutant AAVhu68-PYQRGDH capsid, which comprises mutant VP1, mutant VP2, and mutant VP3 proteins, each having a heterogenous population of proteins comprising the PYQRGDH (SEQ ID NO: 24) peptide insert. In certain embodiments, the proteins are further characterized by having three or four highly deamidated asparagines in positions: N57, N329, N452 and N512, and optional deamidation in other positions within the parental capsid sequence. In certain embodiments, a rAAV has a capsid comprising AAV9-PYQRGDH (or AAVhu68-PYQRGDH)-VP proteins in which each the VP1, VP2 VP3 proteins are a heterogenous population and comprise the VP3 region of SEQ ID NO: 107 (about amino acid 203 to about amino acid 736 based on the residue positions in SEQ ID NO: 25 (AAV9)). The proteins further comprising deamidation in about 50% to about 100% of positions N57 (VP1 only), N329, N452, and/or N512, based on the parental capsid residue positions. In certain embodiments, the capsid comprises VP proteins which are highly deamidated in all of these positions. In certain embodiments, the percentage of deamidation in one or more of these highly deamidated positions is over 50%, over 55%, over 60%, over 65%, over 70%, over 75%, over 80%, over 85%, over 90%, over 95%, or about 70% to about 100%, or values therebetween.

In certain embodiments, the rAAV comprises a mutant AAV9-GQVRVGV capsid or a mutant AAVhu68-GQVRVGV capsid, which comprises mutant VP1, mutant VP2, and mutant VP3 proteins, each having a heterogenous population of proteins comprising the GQVRVGV (SEQ ID NO: 3) peptide insert. In certain embodiments, the proteins are further characterized by having three or four highly deamidated asparagines in positions: N57, N329, N452 and N512, and optional deamidation in other positions within the parental capsid sequence. In certain embodiments, a mutant rAAV9 or a mutant rAAVhu68 has a mutant capsid comprising AAV-GQVRVGV-VP proteins in which each the VP1, VP2 VP3 proteins are a heterogenous population having deamidation in about 50% to about 100% of positions N57 (VP1 only), N329, N452, and/or N512, based on the parental capsid residue positions. In certain embodiments, the capsid comprises VP proteins which are highly deamidated in all of these positions. In certain embodiments, the percentage of deamidation in one or more of these highly deamidated positions is over 50%, over 55%, over 60%, over 65%, over 70%, over 75%, over 80%, over 85%, over 90%, over 95%, or about 70% to about 100%, or values therebetween.

In certain embodiments, the rAAV comprises a mutant AAV9-MDAHYVR capsid or a mutant AAVhu68-MDAHYVR capsid, which comprises mutant VP1, mutant VP2, and mutant VP3 proteins, each having a heterogenous population of proteins comprising the MDAHYVR (SEQ ID NO: 4) peptide insert. In certain embodiments, the proteins are further characterized by having three or four highly deamidated asparagines in positions: N57, N329, N452 and N512, and optional deamidation in other positions within the parental capsid sequence. In certain embodiments, a mutant rAAV9 or a mutant rAAVhu68 has a mutant capsid comprising AAV-MDAHYVR-VP proteins in which each the VP1, VP2 VP3 proteins are a heterogenous population having deamidation in about 50% to about 100% of positions N57 (VP1 only), N329, N452, and/or N512, based on the parental capsid residue positions. In certain embodiments, the capsid comprises VP proteins which are highly deamidated in all of these positions. In certain embodiments, the percentage of deamidation in one or more of these highly deamidated positions is over 50%, over 55%, over 60%, over 65%, over 70%, over 75%, over 80%, over 85%, over 90%, over 95%, or about 70% to about 100%, or values therebetween.

In certain embodiments, the rAAV comprises a mutant AAV9-TQAVPLK capsid or a mutant AAVhu68-TQAVPLK capsid, which comprises mutant VP1, mutant VP2, and mutant VP3 proteins, each having a heterogenous population of proteins comprising the TQAVPLK (SEQ ID NO: 5) peptide insert. In certain embodiments, the proteins are further characterized by having three or four highly deamidated asparagines in positions: N57, N329, N452 and N512, and optional deamidation in other positions within the parental capsid sequence. In certain embodiments, a mutant rAAV9 or a mutant rAAVhu68 has a mutant capsid comprising AAV-TQAVPLK-VP proteins in which each the VP1, VP2 VP3 proteins are a heterogenous population having deamidation in about 50% to about 100% of positions N57 (VP1 only), N329, N452, and/or N512, based on the parental capsid residue positions. In certain embodiments, the capsid comprises VP proteins which are highly deamidated in all of these positions. In certain embodiments, the percentage of deamidation in one or more of these highly deamidated positions is over 50%, over 55%, over 60%, over 65%, over 70%, over 75%, over 80%, over 85%, over 90%, over 95%, or about 70% to about 100%, or values therebetween.

In certain embodiments, the rAAV comprises a mutant AAV9-VVHQTGL capsid or a mutant AAVhu68-VVHQTGL capsid, which comprises mutant VP1, mutant VP2, and mutant VP3 proteins, each having a heterogenous population of proteins comprising the VVHQTGL (SEQ ID NO: 6) peptide insert. In certain embodiments, the proteins are further characterized by having three or four highly deamidated asparagines in positions: N57, N329, N452 and N512, and optional deamidation in other positions within the parental capsid sequence. In certain embodiments, a mutant rAAV9 or a mutant rAAVhu68 has a mutant capsid comprising AAV-VVHQTGL-VP proteins in which each the VP1, VP2 VP3 proteins are a heterogenous population having deamidation in about 50% to about 100% of positions N57 (VP1 only), N329, N452, and/or N512, based on the parental capsid residue positions. In certain embodiments, the capsid comprises VP proteins which are highly deamidated in all of these positions. In certain embodiments, the percentage of deamidation in one or more of these highly deamidated positions is over 50%, over 55%, over 60%, over 65%, over 70%, over 75%, over 80%, over 85%, over 90%, over 95%, or about 70% to about 100%, or values therebetween.

In certain embodiments, the rAAV comprises a mutant AAV9-MAISRER capsid or a mutant AAVhu68-MAISRER capsid, which comprises mutant VP1, mutant VP2, and mutant VP3 proteins, each having a heterogenous population of proteins comprising the MAISRER (SEQ ID NO: 7) peptide insert. In certain embodiments, the proteins are further characterized by having three or four highly deamidated asparagines in positions: N57, N329, N452 and N512, and optional deamidation in other positions within the parental capsid sequence. In certain embodiments, a mutant rAAV9 or a mutant rAAVhu68 has a mutant capsid comprising AAV-MAISRER-VP proteins in which each the VP1, VP2 VP3 proteins are a heterogenous population having deamidation in about 50% to about 100% of positions N57 (VP1 only), N329, N452, and/or N512, based on the parental capsid residue positions. In certain embodiments, the capsid comprises VP proteins which are highly deamidated in all of these positions. In certain embodiments, the percentage of deamidation in one or more of these highly deamidated positions is over 50%, over 55%, over 60%, over 65%, over 70%, over 75%, over 80%, over 85%, over 90%, over 95%, or about 70% to about 100%, or values therebetween.

In certain embodiments, the rAAV comprises a mutant AAV9-IVRGDPA capsid or a mutant AAVhu68-IVRGDPA capsid, which comprises mutant VP1, mutant VP2, and mutant VP3 proteins, each having a heterogenous population of proteins comprising the IVRGDPA (SEQ ID NO: 8) peptide insert. In certain embodiments, the proteins are further characterized by having three or four highly deamidated asparagines in positions: N57, N329, N452 and N512, and optional deamidation in other positions within the parental capsid sequence. In certain embodiments, a mutant rAAV9 or a mutant rAAVhu68 has a mutant capsid comprising AAV-IVRGDPA-VP proteins in which each the VP1, VP2 VP3 proteins are a heterogenous population having deamidation in about 50% to about 100% of positions N57 (VP1 only), N329, N452, and/or N512, based on the parental capsid residue positions. In certain embodiments, the capsid comprises VP proteins which are highly deamidated in all of these positions. In certain embodiments, the percentage of deamidation in one or more of these highly deamidated positions is over 50%, over 55%, over 60%, over 65%, over 70%, over 75%, over 80%, over 85%, over 90%, over 95%, or about 70% to about 100%, or values therebetween.

In certain embodiments, the rAAV comprises a mutant AAV9-MIRGDVK capsid or a mutant AAVhu68-MIRGDVK capsid, which comprises mutant VP1, mutant VP2, and mutant VP3 proteins, each having a heterogenous population of proteins comprising the MIRGDVK (SEQ ID NO: 9) peptide insert. In certain embodiments, the proteins are further characterized by having three or four highly deamidated asparagines in positions: N57, N329, N452 and N512, and optional deamidation in other positions within the parental capsid sequence. In certain embodiments, a mutant rAAV9 or a mutant rAAVhu68 has a mutant capsid comprising AAV-MIRGDVK-VP proteins in which each the VP1, VP2 VP3 proteins are a heterogenous population having deamidation in about 50% to about 100% of positions N57 (VP1 only), N329, N452, and/or N512, based on the parental capsid residue positions. In certain embodiments, the capsid comprises VP proteins which are highly deamidated in all of these positions. In certain embodiments, the percentage of deamidation in one or more of these highly deamidated positions is over 50%, over 55%, over 60%, over 65%, over 70%, over 75%, over 80%, over 85%, over 90%, over 95%, or about 70% to about 100%, or values therebetween.

In certain embodiments, the rAAV comprises a mutant AAV9-PTRGDVK capsid or a mutant AAVhu68-PTRGDVK capsid, which comprises mutant VP1, mutant VP2, and mutant VP3 proteins, each having a heterogenous population of proteins comprising the PTRGDVK (SEQ ID NO: 16) peptide insert. In certain embodiments, the proteins are further characterized by having three or four highly deamidated asparagines in positions: N57, N329, N452 and N512, and optional deamidation in other positions within the parental capsid sequence. In certain embodiments, a mutant rAAV9 or a mutant rAAVhu68 has a mutant capsid comprising AAV-PTRGDVK-VP proteins in which each the VP1, VP2 VP3 proteins are a heterogenous population having deamidation in about 50% to about 100% of positions N57 (VP1 only), N329, N452, and/or N512, based on the parental capsid residue positions. In certain embodiments, the capsid comprises VP proteins which are highly deamidated in all of these positions. In certain embodiments, the percentage of deamidation in one or more of these highly deamidated positions is over 50%, over 55%, over 60%, over 65%, over 70%, over 75%, over 80%, over 85%, over 90%, over 95%, or about 70% to about 100%, or values therebetween.

In certain embodiments, the rAAV comprises a mutant AAV9-RGDYREL capsid or a mutant AAVhu68-RGDYREL capsid, which comprises mutant VP1, mutant VP2, and mutant VP3 proteins, each having a heterogenous population of proteins comprising the RGDYREL (SEQ ID NO: 128) peptide insert. In certain embodiments, the proteins are further characterized by having three or four highly deamidated asparagines in positions: N57, N329, N452 and N512, and optional deamidation in other positions within the parental capsid sequence. In certain embodiments, a mutant rAAV9 or a mutant rAAVhu68 has a mutant capsid comprising AAV-RGDYREL-VP proteins in which each the VP1, VP2 VP3 proteins are a heterogenous population having deamidation in about 50% to about 100% of positions N57 (VP1 only), N329, N452, and/or N512, based on the parental capsid residue positions. In certain embodiments, the capsid comprises VP proteins which are highly deamidated in all of these positions. In certain embodiments, the percentage of deamidation in one or more of these highly deamidated positions is over 50%, over 55%, over 60%, over 65%, over 70%, over 75%, over 80%, over 85%, over 90%, over 95%, or about 70% to about 100%, or values therebetween.

In certain embodiments, the rAAV comprises an AAV capsid wherein the AAV capsid proteins comprises an exogenous peptide which is immediately preceded by “AQ”. In certain embodiments, the rAAV comprises an AAV capsid wherein the AAV capsid proteins comprise exogenous peptide wherein the exogenous peptide is flanked by “AQ” (e.g., “AQ-IIRGDPA (SEQ ID NO: 1)”, “AQ-IIRGDPA (SEQ ID NO: 1)-AQ”, or “IIRGDPA (SEQ ID NO: 1)-AQ”). In certain embodiments, the rAAV comprises an AAV capsid wherein the AAV capsid proteins comprises exogenous peptide which is immediately preceded by the native residues of the parent AAV which may be unmodified at the amino (N—) terminus, and/or at the carboxy (COO—) terminus. In certain embodiments, the rAAV comprises an AAV capsid wherein the AAV capsid protein comprises exogenous peptide which is immediately preceded by the native residues of the parent AAV which may be mutated at the amino (N—) terminus, and/or at the carboxy (COO—) terminus. In certain embodiments, wherein the parental capsid is an AAV9 capsid or other Clade F capsid, the AAV9 parental capsid or other Clade F parental capsid is unmodified at the residues flanking the inserted exogenous targeting peptide. In certain embodiments, the rAAV comprises an AAV capsid wherein the AAV capsid proteins comprise an exogenous peptide wherein the exogenous peptide is flanked by “SAQ” at the amino (N—) terminus of the exogenous peptide. In certain embodiments, the rAAV comprises an AAV capsid wherein the AAV capsid protein comprises exogenous peptide wherein the exogenous peptide is flanked by “AQA” at the carboxy (COO—) terminus. In certain embodiments, wherein the parent capsid is an AAV9 capsid or other Clade F capsid, the AAV9 parent capsid or other Clade F parent capsid is modified (i.e., mutated) at the residues flanking the inserted exogenous targeting peptide. In certain embodiments, the rAAV comprises an AAV capsid wherein the AAV capsid protein comprises an exogenous peptide wherein the exogenous peptide is flanked by a mutated trimer “ENT” at amino (N—) terminus of the exogenous peptide. In certain embodiments, the rAAV comprises an AAV capsid wherein the AAV capsid proteins comprise an exogenous peptide wherein the exogenous peptide is flanked by mutated trimer “SHQ”, SWQ”, SAI”, “GAQ”, “FAQ”, “QAQ”, “AAQ”, “SGQ”, “SGM” at amino (N—) terminus of the exogenous peptide. In certain embodiments, the rAAV comprises an AAV capsid wherein the AAV capsid proteins comprise exogenous peptide wherein the exogenous peptide is flanked by mutated trimer “QQA”, “NQA”, “AMA”, “AQC”, “GQA”, “ARA”, “GRA” at carboxy (COO—) terminus. In other embodiments, the flanking residues may be modified, e.g., where a non-Clade F parental AAV is selected and/or to reduce the number of AAV residues inserted.

In certain embodiments, the rAAV comprises an AAV capsid having AAV capsid proteins comprising an exogenous peptide that is flanked by modified amino acid residues, wherein the capsid proteins comprise the amino acid sequence of “SHQIIRGDPAQQA” (SEQ ID NO: 39). In certain embodiments, the rAAV comprises an AAV capsid having AAV capsid proteins comprising an exogenous peptide that is flanked by modified amino acid residues, wherein the capsid proteins comprise an amino acid sequence at least 99% identical to “SHQIIRGDPAQQA” (SEQ ID NO: 39). In certain embodiments, the rAAV comprises an AAV capsid having AAV capsid proteins comprising an exogenous peptide that is flanked by modified amino acid residues, wherein the capsid proteins comprise the amino acid sequence of “SAQIIRGDMQAQA” (SEQ ID NO: 40). In certain embodiments, the rAAV comprises an AAV capsid having AAV capsid proteins comprising an exogenous peptide that is flanked by modified amino acid residues, wherein the capsid proteins comprise an amino acid sequence at least 99% identical to “SAQIIRGDMQAQA” (SEQ ID NO: 40. In certain embodiments, the rAAV comprises an AAV capsid having AAV capsid proteins comprising an exogenous peptide that is flanked by modified amino acid residues, wherein the capsid proteins comprise the amino acid sequence of “SWQITRGDPAAQA” (SEQ ID NO: 41). In certain embodiments, the rAAV comprises an AAV capsid having AAV capsid proteins comprising an exogenous peptide that is flanked by modified amino acid residues, wherein the capsid proteins comprise an amino acid sequence at least 99% identical to “SWQITRGDPAAQA” (SEQ ID NO: 41). In certain embodiments, the rAAV comprises an AAV capsid having AAV capsid proteins comprising an exogenous peptide that is flanked by modified amino acid residues, wherein the capsid proteins comprise the amino acid sequence of “SAIIIRGDPHAQA” (SEQ ID NO: 42). In certain embodiments, the rAAV comprises an AAV capsid having capsid proteins comprising an exogenous peptide that is flanked by modified amino acid residues, wherein the capsid proteins comprises an amino acid sequence at least 99% identical to “SAIIIRGDPHAQA” (SEQ ID NO: 42). In certain embodiments, the rAAV comprises an AAV capsid having AAV capsid proteins comprising an exogenous peptide that is flanked by modified amino acid residues, wherein the capsid proteins comprise the amino acid sequence of “GAQIIRGDPQAQA” (SEQ ID NO: 43). In certain embodiments, the rAAV comprises an AAV capsid having AAV capsid proteins comprising an exogenous peptide that is flanked by modified amino acid residues, wherein the capsid proteins comprise an amino acid sequence at least 99% identical to “GAQIIRGDPQAQA” (SEQ ID NO: 43). In certain embodiments, the rAAV comprises an AAV capsid having AAV capsid proteins comprising an exogenous peptide that is flanked by modified amino acid residues, wherein the capsid proteins comprise the amino acid sequence of “GAQIIRGDPAQQA” (SEQ ID NO: 44). In certain embodiments, the rAAV comprises an AAV capsid having capsid proteins comprising an exogenous peptide that is flanked by modified amino acid residues, wherein the capsid proteins comprise an amino acid sequence at least 99% identical to “GAQIIRGDPAQQA” (SEQ ID NO: 44). In certain embodiments, the rAAV comprises an AAV capsid having AAV capsid proteins comprising an exogenous peptide that is flanked by modified amino acid residues, wherein the capsid proteins comprise the amino acid sequence of “FAQIIRGDQAAQA” (SEQ ID NO: 45). In certain embodiments, the rAAV comprises an AAV capsid having AAV capsid proteins comprising an exogenous peptide that is flanked by modified amino acid residues, wherein the capsid proteins comprise an amino acid sequence at least 99% identical to “FAQIIRGDQAAQA” (SEQ ID NO: 45). In certain embodiments, the rAAV comprises an AAV capsid having AAV capsid proteins comprising an exogenous peptide that is flanked by modified amino acid residues, wherein the capsid proteins comprise the amino acid sequence of “SAQIIRGDNANQA” (SEQ ID NO: 46). In certain embodiments, the rAAV comprises an AAV capsid having AAV capsid proteins comprising an exogenous peptide that is flanked by modified amino acid residues, wherein the capsid proteins comprise an amino acid sequence at least 99% identical to “SAQIIRGDNANQA” (SEQ ID NO: 46). In certain embodiments, the rAAV comprises an AAV capsid having AAV capsid protein comprising an exogenous peptide that is flanked by modified amino acid residues, wherein the capsid proteins comprise the amino acid sequence of “QAQIIRGDPQAQA” (SEQ ID NO: 47). In certain embodiments, the rAAV comprises an AAV capsid having AAV capsid proteins comprising an exogenous peptide that is flanked by modified amino acid residues, wherein the capsid proteins comprise an amino acid sequence at least 99% identical to “QAQIIRGDPQAQA” (SEQ ID NO: 47). In certain embodiments, the rAAV comprises an AAV capsid having AAV capsid proteins comprising an exogenous peptide that is flanked by modified amino acid residues, wherein the capsid proteins comprise the amino acid sequence of “AAQYIRGDPAAQA” (SEQ ID NO: 48). In certain embodiments, the rAAV comprises an AAV capsid having AAV capsid proteins comprising an exogenous peptide that is flanked by modified amino acid residues, wherein the capsid proteins comprise an amino acid sequence at least 99% identical to “AAQYIRGDPAAQA” (SEQ ID NO: 48). In certain embodiments, the rAAV comprises an AAV capsid having AAV capsid proteins comprising an exogenous peptide that is flanked by modified amino acid residues, wherein the capsid proteins comprise the amino acid sequence of “SGQVIRGDPAAQA” (SEQ ID NO: 49). In certain embodiments, the rAAV comprises an AAV capsid having AAV capsid proteins comprising an exogenous peptide that is flanked by modified amino acid residues, wherein the capsid proteins comprise an amino acid sequence at least 99% identical to “SGQVIRGDPAAQA” (SEQ ID NO: 49). In certain embodiments, the rAAV comprises an AAV capsid having AAV capsid protein comprising an exogenous peptide that is flanked by modified amino acid residues, wherein the capsid proteins comprise the amino acid sequence of “SAQIGRGDPQAQA” (SEQ ID NO: 50). In certain embodiments, the rAAV comprises an AAV capsid having AAV capsid protein comprising an exogenous peptide that is flanked by modified amino acid residues, wherein the capsid proteins comprise an amino acid sequence at least 99% identical to “SAQIGRGDPQAQA” (SEQ ID NO: 50). In certain embodiments, the rAAV comprises an AAV capsid having AAV capsid proteins comprising an exogenous peptide that is flanked by modified amino acid residues, wherein the capsid proteins comprise the amino acid sequence of “SAQIIRGDGAAMA” (SEQ ID NO: 51). In certain embodiments, the rAAV comprises an AAV capsid having AAV capsid proteins comprising an exogenous peptide that is flanked by modified amino acid residues, wherein the capsid proteins comprise an amino acid sequence at least 99% identical to “SAQIIRGDGAAMA” (SEQ ID NO: 51). In certain embodiments, the rAAV comprises an AAV capsid having AAV capsid proteins comprising an exogenous peptide that is flanked by modified amino acid residues, wherein the capsid proteins comprise the amino acid sequence of “SGMIIRGDPAAQA” (SEQ ID NO: 52). In certain embodiments, the rAAV comprises an AAV capsid having AAV capsid protein comprising an exogenous peptide that is flanked by modified amino acid residues, wherein the capsid proteins comprise an amino acid sequence at least 99% identical to “SGMIIRGDPAAQA” (SEQ ID NO: 52). In certain embodiments, the rAAV comprises an AAV capsid having AAV capsid proteins comprising an exogenous peptide that is flanked by modified amino acid residues, wherein the capsid proteins comprise the amino acid sequence of “SAQIIRGDNAAQC” (SEQ ID NO: 53). In certain embodiments, the rAAV comprises an AAV capsid having capsid proteins comprising an exogenous peptide that is flanked by modified amino acid residues, wherein the capsid proteins comprise an amino acid sequence at least 99% identical to “SAQIIRGDNAAQC” (SEQ ID NO: 53). In certain embodiments, the rAAV comprises an AAV capsid having AAV capsid proteins comprising an exogenous peptide that is flanked by modified amino acid residues, wherein the capsid proteins comprise the amino acid sequence of “QAQIIRGDPAAQA” (SEQ ID NO: 54). In certain embodiments, the rAAV comprises an AAV capsid having AAV capsid proteins comprising an exogenous peptide that is flanked by modified amino acid residues, wherein the capsid proteins comprise an amino acid sequence at least 99% identical to “QAQIIRGDPAAQA” (SEQ ID NO: 54). In certain embodiments, the rAAV comprises an AAV capsid having AAV capsid proteins comprising an exogenous peptide that is flanked by modified amino acid residues, wherein the capsid proteins comprise the amino acid sequence of “SAQIIRGDPAAQA” (SEQ ID NO: 55). In certain embodiments, the rAAV comprises an AAV capsid having AAV capsid proteins comprising an exogenous peptide that is flanked by modified amino acid residues, wherein the capsid proteins comprise an amino acid sequence at least 99% identical to “SAQIIRGDPAAQA” (SEQ ID NO: 55). In certain embodiments, the rAAV comprises an AAV capsid having AAV capsid proteins comprising an exogenous peptide that is flanked by modified amino acid residues, wherein the capsid proteins comprise the amino acid sequence of “SAYAVARGDVAQA” (SEQ ID NO: 56). In certain embodiments, the rAAV comprises an AAV capsid having AAV capsid proteins comprising an exogenous peptide that is flanked by modified amino acid residues, wherein the capsid proteins comprise an amino acid sequence at least 99% identical to “SAYAVARGDVAQA” (SEQ ID NO: 56). In certain embodiments, the rAAV comprises an AAV capsid having AAV capsid proteins comprising an exogenous peptide that is flanked by modified amino acid residues, wherein the capsid proteins comprise the amino acid sequence of “SAQQVIRGDVGQA” (SEQ ID NO: 57). In certain embodiments, the rAAV comprises an AAV capsid having AAV capsid proteins comprising an exogenous peptide that is flanked by modified amino acid residues, wherein the capsid proteins comprise an amino acid sequence at least 99% identical to “SAQQVIRGDVGQA” (SEQ ID NO: 57). In certain embodiments, the rAAV comprises an AAV capsid having AAV capsid proteins comprising an exogenous peptide that is flanked by modified amino acid residues, wherein the capsid proteins comprise the amino acid sequence of “SAQAGIRGDVARA” (SEQ ID NO: 58). In certain embodiments, the rAAV comprises an AAV capsid having AAV capsid proteins comprising an exogenous peptide that is flanked by modified amino acid residues, wherein the capsid proteins comprise an amino acid sequence at least 99% identical to “SAQAGIRGDVARA” (SEQ ID NO: 58). In certain embodiments, the rAAV comprises an AAV capsidhaving AAV capsid proteins comprising an exogenous peptide that is flanked by modified amino acid residues, wherein the capsid proteins comprise the amino acid sequence of “GAQAAIRGDVAQA” (SEQ ID NO: 59). In certain embodiments, the rAAV comprises an AAV capsid having AAV capsid proteins comprising an exogenous peptide that is flanked by modified amino acid residues, wherein the capsid proteins comprises an amino acid sequence at least 99% identical to “GAQAAIRGDVAQA” (SEQ ID NO: 59). In certain embodiments, the rAAV comprises an AAV capsid having AAV capsid proteins comprising an exogenous peptide that is flanked by modified amino acid residues, wherein the capsid proteins comprise the amino acid sequence of “SWQAVVRGDVAQA” (SEQ ID NO: 60). In certain embodiments, the rAAV comprises an AAV capsid having AAV capsid proteins comprising an exogenous peptide that is flanked by modified amino acid residues, wherein the capsid proteins comprises an amino acid sequence at least 99% identical to “SWQAVVRGDVAQA” (SEQ ID NO: 60). In certain embodiments, the rAAV comprises an AAV capsid having AAV capsid protein comprises exogenous peptide that is flanked by modified amino acid residues, wherein the capsid comprises the amino acid sequence of “QAQA VIRGDVAQA” (SEQ ID NO: 61). In certain embodiments, the rAAV comprises an AAV capsid having AAV capsid proteins comprising an exogenous peptide that is flanked by modified amino acid residues, wherein the capsid proteins comprise an amino acid sequence at least 99% identical to “QAQAVIRGDVAQA” (SEQ ID NO: 61). In certain embodiments, the rAAV comprises an AAV capsid having AAV capsid proteins comprising an exogenous peptide that is flanked by modified amino acid residues, wherein the capsid proteins comprise the amino acid sequence of “SAQAVIRGDVGRA” (SEQ ID NO: 62). In certain embodiments, the rAAV comprises an AAV capsid having AAV capsid proteins comprising an exogenous peptide that is flanked by modified amino acid residues, wherein the capsid proteins comprise an amino acid sequence at least 99% identical to “SAQAVIRGDVGRA” (SEQ ID NO: 62). In certain embodiments, the rAAV comprises an AAV capsid having AAV capsid proteins comprising an exogenous peptide that is flanked by modified amino acid residues, wherein the capsid proteins comprise the amino acid sequence of “SGQAVIRGDVARA” (SEQ ID NO: 63). In certain embodiments, the rAAV comprises an AAV capsid having AAV capsid proteins comprising an exogenous peptide that is flanked by modified amino acid residues, wherein the capsid proteins comprise an amino acid sequence at least 99% identical to “SGQAVIRGDVARA” (SEQ ID NO: 63). In certain embodiments, the rAAV comprises an AAV capsid having AAV capsid protein comprising an exogenous peptide that is flanked by modified amino acid residues, wherein the capsid proteins comprise the amino acid sequence of “SAYAVIRGDVAQA” (SEQ ID NO: 64). In certain embodiments, the rAAV comprises an AAV capsid having AAV capsid proteins comprising an exogenous peptide that is flanked by modified amino acid residues, wherein the capsid proteins comprise an amino acid sequence at least 99% identical to “SAYAVIRGDVAQA” (SEQ ID NO: 64). In certain embodiments, the rAAV comprises an AAV capsid having AAV capsid proteins comprising an exogenous peptide that is flanked by modified amino acid residues, wherein the capsid proteins comprises the amino acid sequence of “SWQAAIRGDVAQA” (SEQ ID NO: 65). In certain embodiments, the rAAV comprises an AAV capsid having AAV capsid proteins comprising an exogenous peptide that is flanked by modified amino acid residues, wherein the capsid proteins comprise an amino acid sequence at least 99% identical to “SWQAAIRGDVAQA” (SEQ ID NO: 65). In certain embodiments, the rAAV comprises an AAV capsid having AAV capsid proteins comprising an exogenous peptide that is flanked by modified amino acid residues, wherein the capsid proteins comprise the amino acid sequence of “SAQAYIRGDVAQA” (SEQ ID NO: 66). In certain embodiments, the rAAV comprises an AAV capsid having AAV capsid proteins comprising an exogenous peptide that is flanked by modified amino acid residues, wherein the capsid comprises an amino acid sequence at least 99% identical to “SAQAYIRGDVAQA” (SEQ ID NO: 66). In certain embodiments, the rAAV comprises an AAV capsid having AAV capsid proteins comprising an exogenous peptide that is flanked by modified amino acid residues, wherein the capsid proteins comprise the amino acid sequence of “SAMAVIRGDVAQA” (SEQ ID NO: 67). In certain embodiments, the rAAV comprises an AAV capsid having AAV capsid proteins comprising an exogenous peptide that is flanked by modified amino acid residues, wherein the capsid proteins comprises an amino acid sequence at least 99% identical to “SAMAVIRGDVAQA” (SEQ ID NO: 67). In certain embodiments, the rAAV comprises an AAV capsid having AAV capsid proteins comprising an exogenous peptide that is flanked by modified amino acid residues, wherein the capsid proteins comprise the amino acid sequence of “SAHAVIRGDVAQA” (SEQ ID NO: 68). In certain embodiments, the rAAV comprises an AAV capsid having AAV capsid proteins comprising an exogenous peptide that is flanked by modified amino acid residues, wherein the capsid proteins comprise an amino acid sequence at least 99% identical to “SAHAVIRGDVAQA” (SEQ ID NO: 68). In certain embodiments, the rAAV comprises an AAV capsid having AAV capsid proteins comprising an exogenous peptide that is flanked by modified amino acid residues, wherein the capsid proteins comprise the amino acid sequence of “SAQAVVRGDVAQA” (SEQ ID NO: 69). In certain embodiments, the rAAV comprises an AAV capsid having AAV capsid proteins comprising an exogenous peptide that is flanked by modified amino acid residues, wherein the capsid proteins comprise an amino acid sequence at least 99% identical to “SAQAVVRGDVAQA” (SEQ ID NO: 69). In certain embodiments, the rAAV comprises an AAV capsid having AAV capsid proteins comprising an exogenous peptide that is flanked by modified amino acid residues, wherein the capsid proteins comprise the amino acid sequence of “SHQAVIRGDVAQA” (SEQ ID NO: 70). In certain embodiments, the rAAV comprises an AAV capsid having AAV capsid proteins comprising an exogenous peptide that is flanked by modified amino acid residues, wherein the capsid proteins comprises an amino acid sequence at least 99% identical to “SHQAVIRGDVAQA” (SEQ ID NO: 70). In certain embodiments, the rAAV comprises an AAV capsid having AAV capsid proteins comprising an exogenous peptide that is flanked by modified amino acid residues, wherein the capsid proteins comprising the amino acid sequence of “SAQAVIRGDVAQA” (SEQ ID NO: 81). In certain embodiments, the rAAV comprises an AAV capsid having AAV capsid proteins comprises an exogenous peptide that is flanked by modified amino acid residues, wherein the capsid proteins comprise an amino acid sequence at least 99% identical to “SAQAVIRGDVAQA” (SEQ ID NO: 81).

In certain embodiments, capsids from Clade F AAV such as AAVhu68 or AAV9 are selected for parental capsids. Methods of generating vectors having the AAV9 capsid or AAVhu68 capsid, and/or chimeric capsids derived from AAV9 have been described. See, e.g., U.S. Pat. No. 7,906,111, which is incorporated by reference herein. See also, U.S. Provisional Patent Application No. 63/093,275, filed Oct. 18, 2020, which is incorporated herein by reference. Other AAV serotypes which transduce nasal cells or another suitable target (e.g., muscle or lung) may be selected as sources for capsids of AAV viral vectors including, e.g., AAV1, AAV2, AAV3, AAV4, AAV5, AAV6, AAV6.2, AAV7, AAV8, AAV9, rh10, AAVrh64R1, AAVrh64R2, rh8, AAVrh32.33 (See, e.g., US Published Patent Application No. 2007-0036760-A1; US Published Patent Application No. 2009-0197338-A1; and EP 1310571). See also, WO 2003/042397 (AAV7 and other simian AAV), U.S. Pat. Nos. 7,790,449 and 7,282,199 (AAV8), WO 2005/033321 (AAV9), and WO 2006/110689, or yet to be discovered, or a recombinant AAV based thereon, may be used as a source for the AAV capsid. See, e.g., WO 2020/223232 A1 (AAV rh90), WO 2020/223231 A1 International Application No. PCT/US21/45945, filed Aug. 13, 2021 (AAV rh91), and WO 2020/223236 A1 (AAV rh92, AAV rh93, AAV rh91.93), which are incorporated herein by reference in its entirety. These documents also describe other AAV which may be selected for generating AAV and are incorporated by reference. In some embodiments, an AAV capsid (cap) for use in the viral vector can be generated by mutagenesis (i.e., by insertions, deletions, or substitutions) of one of the aforementioned AAV caps or its encoding nucleic acid. In some embodiments, the AAV capsid is chimeric, comprising domains from two or three or four or more of the aforementioned AAV capsid proteins. In some embodiments, the AAV capsid is a mosaic of Vp1, Vp2, and Vp3 (also referred to as vp1, vp2, vp3, or VP1, VP2, VP3) monomers from two or three different AAVs or recombinant AAVs. In some embodiments, an rAAV composition comprises more than one of the aforementioned caps.

In certain embodiments, the mutant AAV capsid is produced by engineering a nucleic acid sequence encoding an exogenous targeting peptide insert into the AAV VP1 coding sequence. In certain embodiments, the coding sequence for the exogenous targeting peptide insert is SEQ ID NO: 108, or a sequence 95% to 100%, or at least 99%, identical thereto encoding SEQ ID NO: 1 (IIRGDPA). In certain embodiments, the coding sequence for the exogenous targeting peptide insert is SEQ ID NO: 109, or a sequence 95% to 100%, or at least 99% identical thereto encoding SEQ ID NO: 2 (AVIRGDV). In certain embodiments, the coding sequence for the exogenous targeting peptide insert is SEQ ID NO: 110, or a sequence 95% to 100%, or at least 99%, identical thereto encoding SEQ ID NO: 8 (IVRGDPA). In certain embodiments, the coding sequence for the exogenous targeting peptide insert is SEQ ID NO: 111, or a sequence 95% to 100%, or at least 99%, identical thereto encoding SEQ ID NO: 9 (MIRGDVK). In certain embodiments, the coding sequence for the exogenous targeting peptide insert is SEQ ID NO: 112, or a sequence 95% to 100%, or at least 99%, identical thereto encoding SEQ ID NO: 10 (AQHRGDV). In certain embodiments, the coding sequence for the exogenous targeting peptide insert is SEQ ID NO: 113, or a sequence 95% to 100% identical, or at least 99% identical, thereto encoding SEQ ID NO: 11 (VSRGDPN). In certain embodiments, the coding sequence for the exogenous targeting peptide insert is SEQ ID NO: 114, or a sequence 95% to 100% identical, or at least 99% identical, thereto encoding SEQ ID NO: 12 (VSRGDPA). In certain embodiments, the coding sequence for the exogenous targeting peptide insert is SEQ ID NO: 115, or a sequence 95% to 100% identical, or at least 99% identical, thereto encoding SEQ ID NO: 13 (PLVRGDI). In certain embodiments, the coding sequence for the exogenous targeting peptide insert is SEQ ID NO: 116, or a sequence 95% to 100% identical, or at least 99% identical, thereto encoding SEQ ID NO: 14 (PYVRGDP). In certain embodiments, the coding sequence for the exogenous targeting peptide insert is SEQ ID NO: 117, or a sequence 95% to 100%, or at least 99% identical, thereto encoding SEQ ID NO: 15 (VVRGDPQ). In certain embodiments, the coding sequence for the exogenous targeting peptide insert is SEQ ID NO: 118, or a sequence 95% to 100%, or at least 99% identical, thereto encoding SEQ ID NO: 17 (VVQRGDV). In certain embodiments, the coding sequence for the exogenous targeting peptide insert is SEQ ID NO: 119, or a sequence 95% to 100% identical, or at least 99% identical, thereto encoding SEQ ID NO: 18 (QHRGDTQ). In certain embodiments, the coding sequence for the exogenous targeting peptide insert is SEQ ID NO: 120, or a sequence 95% to 100% identical, or at least 99% identical, thereto encoding SEQ ID NO: 19 (QIRGDLR). In certain embodiments, the coding sequence for the exogenous targeting peptide insert is SEQ ID NO: 121, or a sequence 95% to 100% identical, or at least 99%, identical thereto encoding SEQ ID NO: 20 (RGDYAQV). In certain embodiments, the coding sequence for the exogenous targeting peptide insert is SEQ ID NO: 122, or a sequence 95% to 100%, or at least 99% identical, thereto encoding SEQ ID NO: 21 (IGRGDPN). In certain embodiments, the coding sequence for the exogenous targeting peptide insert is SEQ ID NO: 123, or a sequence 95% to 100%, or at least 99% identical, thereto encoding SEQ ID NO: 22 (RGDLHGY). In certain embodiments, the coding sequence for the exogenous targeting peptide insert is SEQ ID NO: 124, or a sequence 95% to 100% identical, or at least 99% identical, thereto encoding SEQ ID NO: 23 (RGDYSTM). In certain embodiments, the coding sequence for the exogenous targeting peptide insert is SEQ ID NO: 125, or a sequence 95% to 100% identical, or at least 99% identical, thereto encoding SEQ ID NO: 24 (PYQRGDH).

In certain embodiments, the exogenous peptide is inserted between amino acids 588 and 589 in the AAVhu68 capsid (as based on amino acid sequence of SEQ ID NO: 26). In other embodiments, exogenous peptide is inserted between amino acids 588 and 589 in the AAV9 capsid (as based on amino acid sequence of SEQ ID NO: 25). Still other suitable locations for these inserts may be determined. In still other embodiments, these peptides may be used in other vectors or compositions for targeting.

In certain embodiments, the coding sequence of a mutant AAV9 capsid having the exogenous targeting peptide inserted in the hypervariable region between amino acids 588 and 589 in the AAV9 parental capsid is: SEQ ID NO: 72 (IIRGDPA), SEQ ID NO: 74 (AVIRGDV), SEQ ID NO: 76 (IVRGDPA), SEQ ID NO: 78 (MIRGDVK), SEQ ID NO: 80 (AQHRGDV), SEQ ID NO: 82 (VSRGDPN), SEQ ID NO: 84 (VSRGDPA), SEQ ID NO: 86 (PLVRGDI), SEQ ID NO: 88 (PYVRGDP), SEQ ID NO: 90 (VVRGDPQ), SEQ ID NO: 92 (VVQRGDV), SEQ ID NO: 94 (QHRGDTQ), SEQ ID NO: 96 (QIRGDLR), SEQ ID NO: 98 (RGDYAQV), SEQ ID NO: 100 (IGRGDPN), SEQ ID NO: 102 (RGDLHGY), SEQ ID NO: 104 (RGDYSTM), or SEQ ID NO: 106 (PYQRGDH).

In other embodiments, a mutant AAV9 is encoded by any nucleic acid sequence encoding the amino acid sequence of SEQ ID NO: 73 (IIRGDPA mutant VP1), any nucleic acid sequence encoding the amino acid sequence of SEQ ID NO: 75 (AVIRGDV mutant VP1), any nucleic acid sequence encoding the amino acid sequence of SEQ ID NO: 77 (IVRGDPA mutant VP1), any nucleic acid sequence encoding the amino acid sequence of SEQ ID NO: 79 (MIRGDVK mutant VP1), any nucleic acid sequence encoding the amino acid sequence of SEQ ID NO: 81 (AQHRGDV mutant VP1), any nucleic acid sequence encoding the amino acid sequence of SEQ ID NO: 83 (VSRGDPN mutant VP1), any nucleic acid sequence encoding the amino acid sequence of SEQ ID NO: 85 (VSRGDPA mutant VP1), any nucleic acid sequence encoding the amino acid sequence of SEQ ID NO: 87 (PLVRGDI mutant VP1), any nucleic acid sequence encoding the amino acid sequence of SEQ ID NO: 89 (PYVRGDP mutant VP1), any nucleic acid sequence encoding the amino acid sequence of SEQ ID NO: 91 (VVRGDPQ mutant VP1), any nucleic acid sequence encoding the amino acid sequence of SEQ ID NO: 93 (VVQRGDV mutant VP1), any nucleic acid sequence encoding the amino acid sequence of SEQ ID NO: 95 (QHRGDTQ mutant VP1), any nucleic acid sequence encoding the amino acid sequence of SEQ ID NO: 97 (QIRGDLR mutant VP1), any nucleic acid sequence encoding the amino acid sequence of SEQ ID NO: 99 (RGDYAQV mutant VP1), any nucleic acid sequence encoding the amino acid sequence of SEQ ID NO: 101 (IGRGDPN mutant VP1), any nucleic acid sequence encoding the amino acid sequence of SEQ ID NO: 103 (RGDLHGY mutant VP1), any nucleic acid sequence encoding the amino acid sequence of SEQ ID NO: 105 (RGDYSTM mutant VP1), or any nucleic acid sequence encoding the amino acid sequence of SEQ ID NO: 407 (PYQRGDH mutant VP1).

In certain embodiments, the coding sequence of a mutant AAV9 capsid is SEQ ID NO: 72, or a sequence 95% to 100% identical, or at least 99% identical, thereto encoding SEQ ID NO: 73 (IIRGDPA mutant VP1). In certain embodiments, the coding sequence of a mutant AAV9 capsid is SEQ ID NO: 74, or a sequence 95% to 100% identical, or at least 99% identical, thereto encoding SEQ ID NO: 75 (AVIRGDV mutant VP1). In certain embodiments, the coding sequence of a mutant AAV9 capsid is SEQ ID NO: 76, or a sequence 95% to 100% identical, or at least 99% identical, thereto encoding SEQ ID NO: 77 (IVRGDPA mutant VP1). In certain embodiments, the coding sequence of a mutant AAV9 capsid is SEQ ID NO: 78, or a sequence 95% to 100% identical, or at least 99% identical, thereto encoding SEQ ID NO: 79 (MIRGDVK mutant VP1). In certain embodiments, the coding sequence of a mutant AAV9 capsid is SEQ ID NO: 80, or a sequence 95% to 100% identical, or at least 99% identical thereto, encoding SEQ ID NO: 81 (AQHRGDV mutant VP1). In certain embodiments, the coding sequence of a mutant AAV9 capsid is SEQ ID NO: 82, or a sequence 95% to 100% identical, or at least 99% identical, thereto encoding SEQ ID NO: 83 (VSRGDPN mutant VP1). In certain embodiments, the coding sequence of a mutant AAV9 capsid is SEQ ID NO: 84, or a sequence 95% to 100% identical, or at least 99% identical, thereto encoding SEQ ID NO: 85 (VSRGDPA mutant VP1). In certain embodiments, the coding sequence of a mutant AAV9 capsid is SEQ ID NO: 86, or a sequence 95% to 100% identical, or at least 99% identical, thereto encoding SEQ ID NO: 87 (PLVRGDI mutant VP1). In certain embodiments, the coding sequence of a mutant AAV9 capsid is SEQ ID NO: 88, or a sequence 95% to 100% identical, or at least 99% identical, thereto encoding SEQ ID NO: 89 (PYVRGDP mutant VP1). In certain embodiments, the coding sequence of a mutant AAV9 capsid is SEQ ID NO: 90, or a sequence 95% to 100% identical, or at least 99% identical, thereto encoding SEQ ID NO: 91 (VVRGDPQ mutant VP1). In certain embodiments, the coding sequence of a mutant AAV9 capsid is SEQ ID NO: 92, or a sequence 95% to 100% identical, or at least 99% identical, thereto encoding SEQ ID NO: 93 (VVQRGDV mutant VP1). In certain embodiments, the coding sequence of a mutant AAV9 capsid is SEQ ID NO: 94, or a sequence 95% to 100% identical, or at least 99% identical, thereto encoding SEQ ID NO: 95 (QHRGDTQ mutant VP1). In certain embodiments, the coding sequence of a mutant AAV9 capsid is SEQ ID NO: 96, or a sequence 95% to 100% identical, or at least 99% identical, thereto encoding SEQ ID NO: 97 (QIRGDLR mutant VP1). In certain embodiments, the coding sequence of a mutant AAV9 capsid is SEQ ID NO: 98, or a sequence 95% to 100% identical, or at least 99% identical, thereto encoding SEQ ID NO: 99 (RGDYAQV mutant VP1). In certain embodiments, the coding sequence of a mutant AAV9 capsid is SEQ ID NO: 100, or a sequence 95% to 100% identical, or at least 99% identical, thereto encoding SEQ ID NO: 101 (IGRGDPN mutant VP1). In certain embodiments, the coding sequence of a mutant AAV9 capsid is SEQ ID NO: 102, or a sequence 95% to 100% identical, or at least 99% identical, thereto encoding SEQ ID NO: 103 (RGDLHGY mutant VP1). In certain embodiments, the coding sequence of a mutant AAV9 capsid is SEQ ID NO: 104, or a sequence 95% to 100% identical, or at least 99% identical, thereto encoding SEQ ID NO: 105 (RGDYSTM mutant VP1). In certain embodiments, the coding sequence of a mutant AAV9 capsid is SEQ ID NO: 106, or a sequence 95% to 100% identical, or at least 99% identical, thereto encoding SEQ ID NO: 107 (PYQRGDH mutant VP1).

As used herein, the term “clade” as it relates to groups of AAV refers to a group of AAV which are phylogenetically related to one another as determined using a Neighbor-Joining algorithm by a bootstrap value of at least 75% (of at least 1000 replicates) and a Poisson correction distance measurement of no more than 0.05, based on alignment of the AAV vp1 amino acid sequence. The Neighbor-Joining algorithm has been described in the literature. See, e.g., M. Nei and S. Kumar, Molecular Evolution and Phylogenetics (Oxford University Press, New York (2000). Computer programs are available that can be used to implement this algorithm. For example, the MEGA v2.1 program implements the modified Nei-Gojobori method. Using these techniques and computer programs, and the sequence of an AAV vp1 capsid protein, one of skill in the art can readily determine whether a selected AAV is contained in one of the clades identified herein, in another clade, or is outside these clades. See, e.g., G Gao, et al, J Virol, 2004 June; 78 (12): 6381-6388, which identifies Clades A, B, C, D, E and F, and provides nucleic acid sequences of novel AAV, GenBank Accession Numbers AY530553 to AY530629. See, also, WO 2005/033321.

As used herein, an “AAV9 capsid” is a self-assembled AAV capsid composed of multiple AAV9 vp proteins. The AAV9 vp proteins are typically expressed as alternative splice variants encoded by a nucleic acid sequence which encodes the vp1 amino acid sequence of GenBank accession: AAS99264. These splice variants result in proteins of different length. In certain embodiments, “AAV9 capsid” includes an AAV having an amino acid sequence which is 99% identical to AAS99264 or 99% identical thereto. See, also, WO 2019/168961, published Sep. 6, 2019, including Table G providing the deamidation pattern for AAV9. See, also U.S. Pat. No. 7,906,111 and WO 2005/033321. When specified, “AAV9 variants” may include those described in, e.g., WO2016/049230, U.S. Pat. No. 8,927,514, US 2015/0344911, and U.S. Pat. No. 8,734,809.

A rAAVhu68 is composed of an AAVhu68 capsid and a vector genome. An AAVhu68 capsid is an assembly of a heterogenous population of vp1, a heterogenous population of vp2, and a heterogenous population of vp3 proteins. As used herein when used to refer to vp capsid proteins, the term “heterogenous” or any grammatical variation thereof, refers to a population consisting of elements that are not the same, for example, having vp1, vp2 or vp3 monomers (proteins) with different modified amino acid sequences. See, also, PCT/US2018/019992, WO 2018/160582, entitled “Adeno-Associated Virus (AAV) Clade F Vector and Uses Therefor”, and which are incorporated herein by reference in its entirety.

For other recombinant viral vectors, suitable exposed portions of the viral capsid or envelope protein which is responsible for targeting specificity are selected for insertion of the targeting peptide. For example, in an adenovirus, it may be desirable to modify the hexon protein. In a lentivirus, an envelope fusion protein may modified comprise one or more copies of the targeting motif. For vaccinia virus, the major glycoprotein may be modified to comprise one or more copies of the targeting motif. Suitably, these recombinant viral vectors are replication-defective for safety purposes.

### Expression Cassette and Vectors

Vector genomic sequences which are packaged into an AAV capsid and delivered to a host cell are typically composed of, at a minimum, a transgene and its regulatory sequences, and AAV inverted terminal repeats (ITRs). The transgene is a nucleic acid coding sequence, heterologous to the vector sequences, which encodes a polypeptide, protein, functional RNA molecule (e.g., miRNA, miRNA inhibitor) or other gene product, of interest. The nucleic acid coding sequence is operatively linked to regulatory components in a manner which permits transgene transcription, translation, and/or expression in a cell of a target tissue.

The AAV sequences of the vector typically comprise the cis-acting AAV 5′ and AAV 3′ inverted terminal repeat (ITR) sequences (See, e.g., B. J. Carter, in “Handbook of Parvoviruses”, ed., P. Tijsser, CRC Press, pp. 155 168 (1990)). The ITR sequences are about 145 base pairs (bp) in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J. Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences (also referred to as “AAV 5′ ITR”, “5′ ITR”, “AAV 5′ITR”, or “5′ ITR”, “AAV 3′ ITR”, “3′ ITR”, “AAV 3′ ITR”, or “3′ ITR”). In one embodiment, the ITRs are from an AAV different than that supplying a capsid. In one embodiment, the ITR sequences are from AAV2. However, ITRs from other AAV sources may be selected. A shortened version of the 5′ ITR, termed AITR, has been described in which the D-sequence and terminal resolution site (trs) are deleted. In certain embodiments, the vector genome includes a shortened AAV2 ITR of 130 base pairs, wherein the external A elements is deleted. Without wishing to be bound by theory, it is believed that the shortened ITR reverts back to the wild-type (WT) length of 145 base pairs during vector DNA amplification using the internal (A′) element as a template. In other embodiments, full-length AAV 5′ and 3′ ITRs are used. Where the source of the ITRs is from AAV2 and the AAV capsid is from another AAV source, the resulting vector may be termed pseudotyped. However, other configurations of these elements may be suitable.

In certain embodiments, the provided herein is rAAV comprising a nucleic acid molecule comprising a vector genome comprising at least one AAV ITR at the extreme 5′ and/or extreme 3′ end of the nucleic acid molecule which is the vector genome and an expression cassette. In certain embodiments, the vector genome is a nucleic acid molecule which comprises a 5′-AAV ITR, the expression cassette and a 3′-AAV ITR.

In certain embodiments, the rAAV comprises vector genome comprising a nucleic acid molecule comprising, 5′ to 3′, AAV-5′ITR—an optional enhancer—a promoter—an optional intron-coding sequence (e.g., test transgene)—polyadenylation (polyA) signal sequence-AAV3′-ITR. In other embodiments, the orientation of the ITRs may change from the orientation presented in the vector genome of the nucleic acid used in production (e.g., a plasmid). Thus, in certain embodiments, the rAAV may comprise a vector genome flanked by 3′ and 5′ AAV ITRs, respectively. In certain embodiments, the rAAV may comprise a vector genome flanked by two 5′ AAV ITRs. In certain embodiments, the rAAV may comprise a vector genome flanked by two 3′ AAV ITRs. In other embodiments, an rAAV as provided herein may be partially truncated such that the 5′ AAV ITR and/or the 3′ AAV ITR is not detectable in the vector genome packaged in a final rAAV product.

In addition to the major elements identified above for the recombinant AAV vector, the vector also includes conventional control elements necessary which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest.

The regulatory control elements typically contain a promoter sequence as part of the expression control sequences, e.g., located between the selected 5′ ITR sequence and the coding sequence. Constitutive promoters, regulatable promoters [see, e.g., WO 2011/126808 and WO 2013/04943], tissue specific promoters, or a promoter responsive to physiologic cues may be used may be utilized in the vectors described herein.

Examples of constitutive promoters suitable for controlling expression of the therapeutic products include, but are not limited to chicken beta (β)-actin (CB) promoter, CB7 promoter (promoter comprising a cytomegalovirus immediate-early (CMV IE) enhancer and the chicken β-actin promoter, optionally with spacer sequence, optionally with a chimeric intron comprising chicken beta actin intron and further comprising a chicken beta-actin splicing donor (including the exon sequence, chicken beta actin intron) and rabbit beta-globin splicing acceptor), human cytomegalovirus (CMV) promoter, ubiquitin C promoter (UbC), the early and late promoters of simian virus 40 (SV40), U6 promoter, metallothionein promoters, EF1α promoter, ubiquitin promoter, hypoxanthine phosphoribosyl transferase (HPRT) promoter, dihydrofolate reductase (DHFR) promoter (Scharfmann et al., Proc. Natl. Acad. Sci. USA 88:4626-4630 (1991), adenosine deaminase promoter, phosphoglycerol kinase (PGK) promoter, pyruvate kinase promoter phosphoglycerol mutase promoter, the β-actin promoter (Lai et al., Proc. Natl. Acad. Sci. USA 86:10006-10010 (1989)), the long terminal repeats (LTR) of Moloney Leukemia Virus and other retroviruses, the thymidine kinase promoter of Herpes Simplex Virus and other constitutive promoters known to those of skill in the art. Examples of tissue- or cell-specific promoters suitable for use in the present invention include, but are not limited to, endothelin-I (ET-I) and Flt-I, which are specific for endothelial cells, FoxJ1 (that targets ciliated cells). In other embodiments, selection of cardiac-specific promoters may be desired. See, e.g., R. M. Deviatiirov, et al, “Human library of cardiac promoters and enhancers”, bioRxiv, pp. 1-27, bioRxiv preprint; posted Jun. 15, 2020, which is incorporated herein by reference in its entirety. Preferably, such promoters are of human origin. In other embodiments, selection of muscle-specific promoters may be desired, e.g., muscle creatine kinase promoter, human skeletal α-actin promoter, desmin gene promoter. See, e.g., Skopenkova, V. V., Muscle Specific Promoters for Gene Therapy, Acta Naturae, 2021 January-March; 13 (1): 47-58, which is incorporated herein by reference in its entirety.

In certain embodiments, the regulatory sequences comprise one or more of a promoter, an enhancer, an intron, a transcription factor, a transcription terminator, an efficient RNA processing signals such as splicing and polyadenylation signals (polyA), a sequences that stabilize cytoplasmic mRNA, for example Woodchuck Hepatitis Virus (WHP) Posttranscriptional Regulatory Element (WPRE), and sequences that enhance translation efficiency (i.e., Kozak consensus sequence). In certain embodiments the selected promoter is a constitutive promoter. In certain embodiments, the promoter is a ubiquitous promoter. For example such promoters may include chicken beta-actin (CB) promoter, hybrid of a cytomegalovirus immediate-early enhancer and the chicken β-actin promoter (a CB7 promoter), human cytomegalovirus (CMV) promoter, ubiquitin C promoter (UbC), the early and late promoters of simian virus 40 (SV40), U6 promoter, metallothionein promoters, EF1α promoter, ubiquitin promoter, hypoxanthine phosphoribosyl transferase (HPRT) promoter, dihydrofolate reductase (DHFR) promoter (Scharfmann et al., Proc. Natl. Acad. Sci. USA 88:4626-4630 (1991), adenosine deaminase promoter, phosphoglycerol kinase (PGK) promoter, pyruvate kinase promoter phosphoglycerol mutase promoter, the β-actin promoter (Lai et al., Proc. Natl. Acad. Sci. USA 86:10006-10010 (1989)), the long terminal repeats (LTR) of Moloney Leukemia Virus and other retroviruses, the thymidine kinase promoter of Herpes Simplex Virus and other constitutive promoters known to those of skill in the art.

In certain embodiments, the promoter is a tissue- or cell specific-promoter. In certain embodiments, the promoter is cardiac specific promoter, e.g., cardiac troponin T (cTNT), desmin (DES), alpha-myosin heavy chain (α-MHC), myosin light chain 2 (MLC-2) promoters. See also, Pacak, C. A., et al., Tissue specific promoters improve specificity of AAV9 mediated transgene expression following intra-vascular gene delivery in neonatal mice, Genetic Vaccines and Therapy 2008, 6:13. In certain embodiments, the expression cassette comprises a promoter which is a chicken cardiac Troponin T promoter (also referred to as chicken TnT or chTnT). In certain embodiments, the promoter is a hybrid cardiac promoter comprising a cytomegalovirus immediate early (CMV IE) enhancer and a chicken cardiac troponin T (chicken cTnT or chTnT) promoter. See also, International Patent Application No. PCT/US2022/082384, filed Dec. 24, 2022, now published WO 2023/122804, which is incorporated herein by reference in its entirety. In certain embodiments, and enhancer is a α-myosin heavy-chain enhancer.

Inducible promoters suitable for controlling expression of the therapeutic product include promoters responsive to exogenous agents (e.g., pharmacological agents) or to physiological cucs. These response elements include, but are not limited to, a hypoxia response element (HRE) that binds HIF-Iα and β, a metal-ion response element such as described by Mayo et al. (1982, Cell 29:99-108); Brinster et al. (1982, Nature 296:39-42) and Searle et al. (1985, Mol. Cell. Biol. 5:1480-1489); or a heat shock response element such as described by Nouer et al. (in: Heat Shock Response, ed. Nouer, L., CRC, Boca Raton, Fla., ppI67-220, 1991).

In certain embodiments, expression of the gene product is controlled by a regulatable promoter that provides tight control over the transcription of the sequence encoding the gene product, e.g., a pharmacological agent, or transcription factors activated by a pharmacological agent or in alternative embodiments, physiological cues. Promoter systems that are non-leaky and that can be tightly controlled are preferred. Examples of regulatable promoters which are ligand-dependent transcription factor complexes that may be used in the invention include, without limitation, members of the nuclear receptor superfamily activated by their respective ligands (e.g., glucocorticoid, estrogen, progestin, retinoid, ecdysone, and analogs and mimetics thereof) and rTTA activated by tetracycline. In one aspect of the invention, the gene switch is an EcR-based gene switch. Examples of such systems include, without limitation, the systems described in U.S. Pat. Nos. 6,258,603, 7,045,315, U.S. Published Patent Application Nos. 2006/0014711, 2007/0161086, and International Published Application No. WO 01/70816. Examples of chimeric ecdysone receptor systems are described in U.S. Pat. No. 7,091,038, U.S. Published Patent Application Nos. 2002/0110861, 2004/0033600, 2004/0096942, 2005/0266457, and 2006/0100416, and International Published Application Nos. WO 01/70816, WO 02/066612, WO 02/066613, WO 02/066614, WO 02/066615, WO 02/29075, and WO 2005/108617, each of which is incorporated by reference in its entirety. An example of a non-steroidal ecdysone agonist-regulated system is the RheoSwitch® Mammalian Inducible Expression System (New England Biolabs, Ipswich, MA).

Still other promoter systems may include response elements including but not limited to a tetracycline (tet) response element (such as described by Gossen & Bujard (1992, Proc. Natl. Acad. Sci. USA 89:5547-551); or a hormone response element such as described by Lee et al. (1981, Nature 294:228-232); Hynes et al. (1981, Proc. Natl. Acad. Sci. USA 78:2038-2042); Klock et al. (1987, Nature 329:734-736); and Israel & Kaufman (1989, Nucl. Acids Res. 17:2589-2604) and other inducible promoters known in the art. Using such promoters, expression of the soluble hACE2 construct can be controlled, for example, by the Tet-on/off system (Gossen et al., 1995, Science 268:1766-9; Gossen et al., 1992, Proc. Natl. Acad. Sci. USA., 89 (12): 5547-51); the TetR-KRAB system (Urrutia R., 2003, Genome Biol., 4 (10): 231; Deuschle U et al., 1995, Mol Cell Biol. (4): 1907-14); the mifepristone (RU486) regulatable system (Geneswitch; Wang Y et al., 1994, Proc. Natl. Acad. Sci. USA., 91 (17): 8180-4; Schillinger et al., 2005, Proc. Natl. Acad. Sci. U S A. 102 (39): 13789-94); and the humanized tamoxifen-dep regulatable system (Roscilli et al., 2002, Mol. Ther. 6 (5): 653-63).

In another aspect, the gene switch is based on heterodimerization of FK506 binding protein (FKBP) with FKBP rapamycin associated protein (FRAP) and is regulated through rapamycin or its non-immunosuppressive analogs. Examples of such systems, include, without limitation, the ARGENT™ Transcriptional Technology (ARIAD Pharmaceuticals, Cambridge, Mass.) and the systems described in U.S. Pat. Nos. 6,015,709, 6,117,680, 6,479,653, 6,187,757, and 6,649,595, U.S. Publication No. 2002/0173474, U.S. Publication No. 200910100535, U.S. Pat. Nos. 5,834,266, 7,109,317, 7,485,441, 5,830,462, 5,869,337, 5,871,753, 6,011,018, 6,043,082, 6,046,047, 6,063,625, 6,140,120, 6,165,787, 6,972,193, 6,326,166, 7,008,780, 6,133,456, 6,150,527, 6,506,379, 6,258,823, 6,693,189, 6,127,521, 6,150,137, 6,464,974, 6,509,152, 6,015,709, 6,117,680, 6,479,653, 6,187,757, 6,649,595, 6,984,635, 7,067,526, 7,196,192, 6,476,200, 6,492,106, WO 94/18347, WO 96/20951, WO 96/06097, WO 97/31898, WO 96/41865, WO 98/02441, WO 95/33052, WO 99110508, WO 99110510, WO 99/36553, WO 99/41258, WO 01114387, ARGENT™ Regulated Transcription Retrovirus Kit, Version 2.0 (9109102), and ARGENT™ Regulated Transcription Plasmid Kit, Version 2.0 (9109/02), each of which is incorporated herein by reference in its entirety. The Ariad system is designed to be induced by rapamycin and analogs thereof referred to as “rapalogs”. Examples of suitable rapamycins are provided in the documents listed above in connection with the description of the ARGENT™ system. In one embodiment, the molecule is rapamycin [e.g., marketed as Rapamunc™ by Pfizer]. In another embodiment, a rapalog known as AP21967 [ARIAD] is used. Examples of these dimerizer molecules that can be used in the present invention include, but are not limited to rapamycin, FK506, FK1012 (a homodimer of FK506), rapamycin analogs (“rapalogs”) which are readily prepared by chemical modifications of the natural product to add a “bump” that reduces or eliminates affinity for endogenous FKBP and/or FRAP. Examples of rapalogs include, but are not limited to such as AP26113 (Ariad), AP1510 (Amara, J. F., et al., 1997, Proc Natl Acad Sci USA, 94 (20): 10618-23) AP22660, AP22594, AP21370, AP22594, AP23054, AP1855, AP1856, AP1701, AP1861, AP1692 and AP1889, with designed ‘bumps’ that minimize interactions with endogenous FKBP. Still other rapalogs may be selected, e.g., AP23573 [Merck]. In certain embodiments, rapamycin or a suitable analog may be delivered locally to the AAV-transfected cells of the nasopharynx. This local delivery may be by intranasal injection, topically to the cells via bolus, cream, or gel. See, US Patent Application US 2019/0216841 A1, which is incorporated herein by reference.

Other suitable enhancers include those that are appropriate for a desired target tissue indication. In one embodiment, the expression cassette comprises one or more expression enhancers. In one embodiment, the expression cassette contains two or more expression enhancers. These enhancers may be the same or may differ from one another. For example, an enhancer may include a CMV immediate early enhancer. This enhancer may be present in two copies which are located adjacent to one another. Alternatively, the dual copies of the enhancer may be separated by one or more sequences. In still another embodiment, the expression cassette further contains an intron, e.g., the chicken beta-actin intron. Other suitable introns include those known in the art, e.g., such as are described in WO 2011/126808. Examples of suitable polyadenylation (polyA) sequences include, e.g., rabbit beta globin (rBG), SV40, SV50, bovine growth hormone (bGH), human growth hormone, and synthetic polyAs. Optionally, one or more sequences may be selected to stabilize mRNA. An example of such a sequence is a modified WPRE sequence, which may be engineered upstream of the poly A sequence and downstream of the coding sequence (see, e.g., MA Zanta-Boussif, et al, Gene Therapy (2009) 16:605-619).

In certain embodiments, the expression cassette may include one or more expression enhancers such as post-transcriptional regulatory element from hepatitis viruses of woodchuck (WPRE), human (HPRE), ground squirrel (GPRE) or arctic ground squirrel (AGSPRE); or a synthetic post-transcriptional regulatory element. These expression-enhancing elements are particularly advantageous when placed in a 3′ UTR and can significantly increase mRNA stability and/or protein yield. In certain embodiments, the expressions cassettes provided include a regulator sequence that is a woodchuck hepatitis virus posttranscriptional regulatory element (WPRE) or a variant thereof. Suitable WPRE sequences are provided in the vector genomes described herein and are known in the art (e.g., such as those are described in U.S. Pat. Nos. 6,136,597, 6,287,814, and 7,419,829, which are incorporated by reference). In certain embodiments, the WPRE is a variant that has been mutated to eliminate expression of the woodchuck hepatitis B virus X (WHX) protein, including, for example, mutations in the start codon of the WHX gene. In certain embodiments, modified WPRE element is engineered to eliminate expression of the WHX protein, wherein the modified WPRE is a mutated version that contains five-point mutations in the putative promoter region of the WHX gene, along with an additional mutation in the start codon of the WHX gene (ATG mutated to TTG). This mutant WPRE is considered sufficient to eliminate expression of truncated WHX protein based on sensitive flow cytometry analyses of various human cell lines transduced with lentivirus containing a WPRE-GFP fusion construct (Zanta-Boussif et al., 2009). See also, Kingsman S. M., Mitrophanous K., & Olsen J. C. (2005), Potential Oncogene Activity of the Woodchuck Hepatitis Post-Transcriptional Regulatory Element (WPRE). Gene Ther. 12 (1): 3-4; and Zanta-Boussif M. A., Charrier S., Brice-Ouzet A., Martin S., Opolon P., Thrasher A. J., Hope T. J., & Galy A. (2009), Validation of a Mutated Pre-Sequence Allowing High and Sustained Transgene Expression While Abrogating Why-X Protein Synthesis: Application to the Gene Therapy of Was, Gene Ther. 16 (5): 605-19, both of which are incorporated herein by reference in its entirety. In other embodiments, enhancers are selected from a non-viral source. In certain embodiments, no WPRE sequence is present.

An AAV vector genome may include a sequence encoding multiple gene products (e.g., encoding one or more a protein, peptide, miR, miR seed or target). In certain embodiments, the transgene may be used to correct or ameliorate gene deficiencies, which may include deficiencies in which normal genes are expressed at less than normal levels or deficiencies in which the functional gene product is not expressed. Alternatively, the transgene may provide a product to a cell which is not natively expressed in the cell type or in the host. A preferred type of transgene sequence encodes a therapeutic protein or polypeptide which is expressed in a host cell. The invention further includes using multiple transgenes. In certain situations, a different transgene may be used to encode each subunit of a protein, or to encode different peptides or proteins. This is desirable when the size of the DNA encoding the protein subunit is large, e.g., for an immunoglobulin, the platelet-derived growth factor, or a dystrophin protein. In certain situations, a different transgene may be used to encode each subunit of a protein (e.g., an immunoglobulin domain, an immunoglobulin heavy chain, an immunoglobulin light chain). In one embodiment, a cell produces the multi-subunit protein following infected/transfection with the virus containing each of the different subunits. In another embodiment, different subunits of a protein may be encoded by the same transgene. An IRES is desirable when the size of the DNA encoding each of the subunits is small, e.g., the total size of the DNA encoding the subunits and the IRES is less than five kilobases. As an alternative to an IRES, the DNA may be separated by sequences encoding a 2A peptide, which self-cleaves in a post-translational event. See, e.g., ML Donnelly, et al, (January 1997) J. Gen. Virol., 78 (Pt 1): 13-21; S. Furler, S et al, (June 2001) Gene Ther., 8 (11): 864-873; H. Klump, et al., (May 2001) Gene Ther., 8 (10): 811-817. This 2A peptide is significantly smaller than IRES, making it well suited for use when space is a limiting factor. More often, when the transgene is large, consists of multi-subunits, or two transgenes are co-delivered, rAAV carrying the desired transgene(s) or subunits are co-administered to allow them to concatamerize in vivo to form a single vector genome. In such an embodiment, a first AAV may carry an expression cassette which expresses a single transgene and a second AAV may carry an expression cassette which expresses a different transgene for co-expression in the host cell. However, the selected transgene may encode any biologically active product or other product, e.g., a product desirable for study.

In addition to the elements identified above for the expression cassette, the vector also includes conventional control elements which are operably linked to the coding sequence in a manner which permits transcription, translation and/or expression of the encoded product in a cell transfected with the plasmid vector or infected with the virus produced by the invention. Examples of other suitable transgenes are provided herein. As used herein, “operably linked” sequences include both expression control sequences that are contiguous with the gene of interest and expression control sequences that act in trans or at a distance to control the gene of interest.

Expression control sequences include appropriate enhancer; transcription factor; transcription terminator; promoter; efficient RNA processing signals such as splicing and polyadenylation (polyA) signals; sequences that stabilize cytoplasmic mRNA, for example Woodchuck Hepatitis Virus (WHP) Posttranscriptional Regulatory Element (WPRE); sequences that enhance translation efficiency (i.e., Kozak consensus sequence); sequences that enhance protein stability; and when desired, sequences that enhance secretion of the encoded product.

In one embodiment, the regulatory sequences are selected such that the total rAAV vector genome is about 2.0 to about 5.5 kilobases in size. In one embodiment, it is desirable that the rAAV vector genome approximate the size of the native AAV genome. Thus, in one embodiment, the regulatory sequences are selected such that the total rAAV vector genome is about 4.7 kb in size. In another embodiment, the total rAAV vector genome is less about 5.2 kb in size. The size of the vector genome may be manipulated based on the size of the regulatory sequences including the promoter, enhancer, intron, poly A, etc. See, Wu et al, Mol Ther, January 2010 18 (1): 80-6, which is incorporated herein by reference.

Thus, in one embodiment, an intron is included in the vector. Suitable introns include chicken beta-actin intron, the human beta globin IVS2 (Kelly et al, Nucleic Acids Research, 43 (9): 4721-32 (2015)); the Promega chimeric intron (Almond, B. and Schenborn, E. T. A Comparison of pCI-neo-Vector and pcDNA4/HisMax Vector); and the hFIX intron. Various introns suitable herein are known in the art and include, without limitation, those found at bpg.utoledo.edu/~afedorov/lab/eid.html, which is incorporated herein by reference. See also, Shepelev V., Fedorov A. Advances in the Exon-Intron Database. Briefings in Bioinformatics 2006, 7:178-185, which is incorporated herein by reference.

In certain embodiments, the mutant rAAV comprises an expression cassette which further comprising at least one miRNA target sequences operably linked to a selected transgene, optionally in its 3′ UTR and/or its 5′ UTR. In certain embodiments, the mutant rAAV comprises a vector genome (comprising an expression cassette) which further comprises at least one miRNA seed, binding site or full sequence. MicroRNAs (or miRNA or miR) are 19-25 nucleotide noncoding RNAs that bind to the sites of nucleic acid targets and down-regulate gene expression either by reducing nucleic acid molecule stability or by inhibiting translation. In some embodiments, a microRNA sequence comprises a seed region, e.g., a sequence in the region of positions 2-8 of the mature microRNA, which has Watson-Crick sequence fully or partially complementarity to the miRNA target sequence of the nucleic acid. Such at least one miRNA may be used in combinations, including in an expression cassette or a vector genome also comprising a coding sequence for therapeutic protein, enzyme, or other moiety, and which is operably linked to the coding sequence. In certain embodiments, the vector genome may contain one miRNA to eight miRNA sequences, which are the same or different. Optionally, the vector genome does not contain therapeutic transgenes other than miRNA sequences.

In some embodiments, the miRNA binding site is complementary to a miRNA expressed in a DRG (dorsal root ganglion) neuron, e.g., a miR183, and/or a miR182, binding site. In some embodiments, the miR binding site complementary to a miR expressed in expressed in a DRG neuron comprises a nucleotide sequence disclosed, e.g., in WO2020/132455, and in WO 2023/087019, the contents of which are incorporated by reference herein in its entirety.

As another non-limiting example, a vector genome (expression cassette) may comprise miR-122 miRNA to modulate, e.g., reduce, the expression of a gene product in the liver. In some embodiments, the vector genome (expression cassette) may comprise a miRNA, e.g., a miR-142-3p, to modulate, e.g., reduce, the expression, of the gene product in a cell or tissue of the hematopoietic lineage, including for example immune cells (e.g., antigen presenting cells or APC, including dendritic cells (DCs), macrophages, and B-lymphocytes). Other suitable miRNA may include, e.g., miR-206 (skeletal muscle), miR-018b, miR-431.See. e.g., Kim., H. K., Muscle-specific microRNA miR-206 promotes muscle differentiation, The Journal of Cell Biology, 2006, 174 (5): 677-687; WO 2019/035690A1; WO 2019/035690A1; WO 2022/147181A1, and Brazilian Patent Publication No. BR102018067702A2, which are all incorporate herein by reference.

Several different vector genomes were generated in the studies described herein. However, it will be understood by the skilled artisan that other genomic configurations, including other regulatory sequences may be substituted for the promoter, enhancer and other coding sequences may be selected.

rAAV Vector Production

For use in producing an AAV viral vector (e.g., a recombinant (r) AAV), the expression cassettes can be carried on any suitable vector, e.g., a plasmid, which is delivered to a production (packaging) host cell. The plasmids useful in this invention may be engineered such that they are suitable for replication and packaging in vitro in prokaryotic cells, insect cells, mammalian cells, among others. Suitable transfection techniques and packaging host cells are known and/or can be readily designed by one of skill in the art. In certain embodiments, the production host cell is a human cell or insect cell. In certain embodiments, the production host cell in is HEK293 cell, HuH-7 cell, BHK cell, or Vero cell. In certain embodiments, production host cell is in a suspension cell culture.

In certain embodiments, provided herein is a production host cell comprising a recombinant nucleic acid molecule as described herein, a nucleic acid sequence encoding an AAV capsid protein, and sufficient AAV rep functions and helper functions to permit packaging of the vector genome into the AAV capsid.

In certain embodiments, the insertion of the exogenous targeting peptide, wherein the exogenous targeting peptide comprises “Xn-n-mer-Xm”, wherein: (i) Xn is 0, 1, 2 or 3 amino acid residues independently selected from any amino acid; (ii) n-mer selected from IIRGDPA (SEQ ID NO: 1), AVIRGDV (SEQ ID NO: 2), IVRGDPA (SEQ ID NO: 8), MIRGDVK (SEQ ID NO: 9), AQHRGDV (SEQ ID NO: 10), VSRGDPN (SEQ ID NO: 11), VSRGDPA (SEQ ID NO: 12), PLVRGDI (SEQ ID NO: 13), PYVRGDP (SEQ ID NO: 14), VVRGDPQ (SEQ ID NO: 15), PTRGDVK (SEQ ID NO: 16), VVQRGDV (SEQ ID NO: 17), QHRGDTQ (SEQ ID NO: 18), QIRGDLR (SEQ ID NO: 19), RGDYAQV (SEQ ID NO: 20), IGRGDPN (SEQ ID NO: 21), RGDLHGY (SEQ ID NO: 22), RGDYSTM (SEQ ID NO: 23), or PYQRGDH (SEQ ID NO: 24), or an n-mer sequence of at least 6, at least 7 or full length consecutive amino acids of any one of the n-mers; and (iii) Xm is 0, 1, 2, or 3 amino acid residues independently selected from any amino acid, wherein the inclusion of exogenous targeting peptide into an AAV capsid provides advantages in production as compared to the method without inclusion of at least one copy of motif in AAV capsid, and wherein the production cells are 293 cells.

In certain embodiments, a host cell is stably or transiently transfected with a genetic element (e.g., a plasmid or other nucleic acid molecule) which expresses a mutant AAV capsid as provided herein. In certain embodiments, such a genetic element comprises a nucleic acid sequence encoding a mutant AAV VP1 coding sequence comprising the mutant peptide(s) inserted therein, operably linked to expression control sequences which enable expression of the AAV capsid proteins in the packaging host cell. In certain embodiments, the coding sequence for the peptide insert is a sequence encoding SEQ ID NO: 73 (IIRGDPA), which is optionally a nucleic acid sequence of SEQ ID NO: 72 or a sequence 95% to 100% identical, or at least 99% identical, thereto. In certain embodiments, the coding sequence for the peptide insert is a sequence encoding SEQ ID NO: 75 (AVIRGDV), which is optionally a nucleic acid sequence of SEQ ID NO: 74 or a sequence 95% to 100% identical, or at least 99% identical thereto. In certain embodiments, the coding sequence for the peptide insert is a sequence encoding SEQ ID NO: 77 (IVRGDPA), which is optionally a nucleic acid sequence of SEQ ID NO: 76 or a sequence 95% to 100% identical, or at least 99% identical thereto. In certain embodiments, the coding sequence for the peptide insert is a sequence encoding SEQ ID NO: 79 (MIRGDVK), which is optionally a nucleic acid sequence of SEQ ID NO: 78 or a sequence 95% to 100% identical, or at least 99% identical thereto. In certain embodiments, the coding sequence for the peptide insert is a sequence encoding SEQ ID NO: 81 (AQHRGDV), which is optionally a nucleic acid sequence of SEQ ID NO: 80 or a sequence 95% to 100% identical, or at least 99% identical thereto. In certain embodiments, the coding sequence for the peptide insert is a sequence encoding SEQ ID NO: 83 (VSRGDPN), which is optionally a nucleic acid sequence of SEQ ID NO: 82 or a sequence 95% to 100% identical, or at least 99% identical thereto. In certain embodiments, the coding sequence for the peptide insert is a sequence encoding SEQ ID NO: 85 (VSRGDPA), which is optionally a nucleic acid sequence of SEQ ID NO: 84 or a sequence 95% to 100% identical, or at least 99% identical thereto. In certain embodiments, the coding sequence for the peptide insert is a sequence encoding SEQ ID NO: 87 (PLVRGDI), which is optionally a nucleic acid sequence of SEQ ID NO: 86 or a sequence 95% to 100% identical, or at least 99% identical thereto. In certain embodiments, the coding sequence for the peptide insert is a sequence encoding SEQ ID NO: 89 (PYVRGDP), which is optionally a nucleic acid sequence of SEQ ID NO: 88 or a sequence 95% to 100% identical, or at least 99% identical thereto. In certain embodiments, the coding sequence for the peptide insert is a sequence encoding SEQ ID NO: 91 (VVRGDPQ), which is optionally a nucleic acid sequence of SEQ ID NO: 90 or a sequence 95% to 100% identical, or at least 99% identical thereto. In certain embodiments, the coding sequence for the peptide insert is a sequence encoding SEQ ID NO: 93 (VVQRGDV), which is optionally a nucleic acid sequence of SEQ ID NO: 92 or a sequence 95% to 100% identical, or at least 99% identical thereto. In certain embodiments, the coding sequence for the peptide insert is a sequence encoding SEQ ID NO: 95 (QHRGDTQ), which is optionally a nucleic acid sequence of SEQ ID NO: 94 or a sequence 95% to 100% identical, or at least 99% identical thereto. In certain embodiments, the coding sequence for the peptide insert is a sequence encoding SEQ ID NO: 97 (QIRGDLR), which is optionally a nucleic acid sequence of SEQ ID NO: 96 or a sequence 95% to 100% identical, or at least 99% identical thereto. In certain embodiments, the coding sequence for the peptide insert is a sequence encoding SEQ ID NO: 99 (RGDYAQV), which is optionally a nucleic acid sequence of SEQ ID NO: 98 or a sequence 95% to 100% identical, or at least 99% identical thereto. In certain embodiments, the coding sequence for the peptide insert is a sequence encoding SEQ ID NO: 101 (IGRGDPN), which is optionally a nucleic acid sequence of SEQ ID NO: 100 or a sequence 95% to 100% identical, or at least 99% identical thereto. In certain embodiments, the coding sequence for the peptide insert is a sequence encoding SEQ ID NO: 103 (RGDLHGY), which is optionally a nucleic acid sequence of SEQ ID NO: 102 or a sequence 95% to 100% identical, or at least 99% identical thereto. In certain embodiments, the coding sequence for the peptide insert is a sequence encoding SEQ ID NO: 105 (RGDYSTM), which is optionally a nucleic acid sequence of SEQ ID NO: 104 or a sequence 95% to 100% identical, or at least 99% identical thereto. In certain embodiments, the coding sequence for the peptide insert is a sequence encoding SEQ ID NO: 107 (PYQRGDH), which is optionally a nucleic acid sequence of SEQ ID NO: 106 or a sequence 95% to 100% identical, or at least 99% identical thereto.

In certain embodiments, a host cell is stably or transiently transfected with a genetic element (e.g., a plasmid or other nucleic acid molecule) which expresses a mutant AAV capsid as provided herein. In certain embodiments, such a genetic element comprises a nucleic acid sequence encoding a mutant AAV VP1 coding sequence comprising the mutant peptide(s) (i.e., the exogenous targeting peptide(s)) inserted therein, operably linked to expression control sequences which enable expression of the AAV capsid proteins in the packaging host cell. In certain embodiments, the mutant peptides are engineered between amino acids 588 and 589 in the AAVhu68 capsid. In other embodiments, these peptides are inserted between amino acids 588 and 589 in the AAV9 capsid. Still other suitable locations for these inserts may be determined. In still other embodiments, these peptides may be used in other vectors or compositions for targeting. In certain embodiments, the coding sequence of a mutant AAV9 capsid having the exogenous targeting peptide inserted in the hypervariable region between amino acids 588 and 589 in the AAV9 parental capsid is: SEQ ID NO: 72 (IIRGDPA), SEQ ID NO: 74 (AVIRGDV), SEQ ID NO: 76 (IVRGDPA), SEQ ID NO: 78 (MIRGDVK), SEQ ID NO: 80 (AQHRGDV), SEQ ID NO: 82 (VSRGDPN), SEQ ID NO: 84 (VSRGDPA), SEQ ID NO: 86 (PLVRGDI), SEQ ID NO: 88 (PYVRGDP), SEQ ID NO: 90 (VVRGDPQ), SEQ ID NO: 92 (VVQRGDV), SEQ ID NO: 94 (QHRGDTQ), SEQ ID NO: 96 (QIRGDLR), SEQ ID NO: 98 (RGDYAQV), SEQ ID NO: 100 (IGRGDPN), SEQ ID NO: 102 (RGDLHGY), SEQ ID NO: 104 (RGDYSTM), or SEQ ID NO: 106 (PYQRGDH). In other embodiments, a mutant AAV9 is encoded by any nucleic acid sequence encoding the amino acid sequence of SEQ ID NO: 73 (IIRGDPA mutant VP1), any nucleic acid sequence encoding the amino acid sequence of SEQ ID NO: 75 (AVIRGDV mutant VP1), any nucleic acid sequence encoding the amino acid sequence of SEQ ID NO: 77 (IVRGDPA mutant VP1), any nucleic acid sequence encoding the amino acid sequence of SEQ ID NO: 79 (MIRGDVK mutant VP1), any nucleic acid sequence encoding the amino acid sequence of SEQ ID NO: 81 (AQHRGDV mutant VP1), any nucleic acid sequence encoding the amino acid sequence of SEQ ID NO: 83 (VSRGDPN mutant VP1), any nucleic acid sequence encoding the amino acid sequence of SEQ ID NO: 85 (VSRGDPA mutant VP1), any nucleic acid sequence encoding the amino acid sequence of SEQ ID NO: 87 (PLVRGDI mutant VP1), any nucleic acid sequence encoding the amino acid sequence of SEQ ID NO: 89 (PYVRGDP mutant VP1), any nucleic acid sequence encoding the amino acid sequence of SEQ ID NO: 91 (VVRGDPQ mutant VP1), any nucleic acid sequence encoding the amino acid sequence of SEQ ID NO: 93 (VVQRGDV mutant VP1), any nucleic acid sequence encoding the amino acid sequence of SEQ ID NO: 95 (QHRGDTQ mutant VP1), any nucleic acid sequence encoding the amino acid sequence of SEQ ID NO: 97 (QIRGDLR mutant VP1), any nucleic acid sequence encoding the amino acid sequence of SEQ ID NO: 99 (RGDYAQV mutant VP1), any nucleic acid sequence encoding the amino acid sequence of SEQ ID NO: 101 (IGRGDPN mutant VP1), any nucleic acid sequence encoding the amino acid sequence of SEQ ID NO: 103 (RGDLHGY mutant VP1), any nucleic acid sequence encoding the amino acid sequence of SEQ ID NO: 105 (RGDYSTM mutant VP1), or any nucleic acid sequence encoding the amino acid sequence of SEQ ID NO: 407 (PYQRGDH mutant VP1).

Methods of preparing AAV-based vectors (e.g., having an AAV9 or another AAV capsid) are known. See, e.g., US Published Patent Application No. 2007/0036760 (Feb. 15, 2007), which is incorporated by reference herein. The invention is not limited to the use of AAV9 or other clade F AAV amino acid sequences, but encompasses peptides and/or proteins containing the terminal β-galactose binding generated by other methods known in the art, including, e.g., by chemical synthesis, by other synthetic techniques, or by other methods. The sequences of any of the AAV capsids provided herein can be readily generated using a variety of techniques. Suitable production techniques are well known to those of skill in the art. See, e.g., Sambrook et al, Molecular Cloning: A Laboratory Manual, Cold Spring Harbor Press (Cold Spring Harbor, NY). Alternatively, peptides can also be synthesized by the well-known solid phase peptide synthesis methods (Merrifield, (1962) J. Am. Chem. Soc., 85:2149; Stewart and Young, Solid Phase Peptide Synthesis (Freeman, San Francisco, 1969) pp. 27-62). These methods may involve, e.g., culturing a host cell which contains a nucleic acid sequence encoding an AAV capsid; a functional rep gene; a minigene composed of, at a minimum, AAV inverted terminal repeats (ITRs) and a transgene; and sufficient helper functions to permit packaging of the minigene into the AAV capsid protein. These and other suitable production methods are within the knowledge of those of skill in the art and are not a limitation of the present invention.

The components required to be cultured in the host cell to package an AAV minigene in an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contains the rep and/or cap proteins under the control of inducible promoters. Still other stable host cells may be generated by one of skill in the art.

These rAAVs are particularly well suited to gene delivery for therapeutic purposes and for preventing infection. Further, the compositions of the invention may also be used for production of a desired gene product in vitro. For in vitro production, a desired product (e.g., a protein) may be obtained from a desired culture following transfection of host cells with a rAAV containing the molecule encoding the desired product and culturing the cell culture under conditions which permit expression. The expressed product may then be purified and isolated, as desired. Suitable techniques for transfection, cell culturing, purification, and isolation are known to those of skill in the art. Methods for generating and isolating AAVs suitable for use as vectors are known in the art. See generally, e.g., Grieger & Samulski, 2005, “Adeno-associated virus as a gene therapy vector: Vector development, production and clinical applications,” Adv. Biochem. Engin/Biotechnol. 99:119-145; Buning et al., 2008, “Recent developments in adeno-associated virus vector technology,” J. Gene Med. 10:717-733; and the references cited below, each of which is incorporated herein by reference in its entirety. For packaging a transgene into virions, the ITRs are the only AAV components required in cis in the same construct as the nucleic acid molecule containing the expression cassettes. The cap and rep genes can be supplied in trans.

In one embodiment, the expression cassettes described herein are engineered into a genetic element (e.g., a shuttle plasmid) which transfers the immunoglobulin construct sequences carried thereon into a packaging host cell for production a viral vector. In one embodiment, the selected genetic element may be delivered to an AAV packaging cell by any suitable method, including transfection, electroporation, liposome delivery, membrane fusion techniques, high velocity DNA-coated pellets, viral infection and protoplast fusion. Stable AAV packaging cells can also be made. Alternatively, the expression cassettes may be used to generate a viral vector other than AAV, or for production of mixtures of antibodies in vitro. The methods used to make such constructs are known to those with skill in nucleic acid manipulation and include genetic engineering, recombinant engineering, and synthetic techniques. See, e.g., Molecular Cloning: A Laboratory Manual, ed. Green and Sambrook, Cold Spring Harbor Press, Cold Spring Harbor, NY (2012).

The term “AAV intermediate” or “AAV vector intermediate” refers to an assembled rAAV capsid which lacks the desired genomic sequences packaged therein. These may also be termed an “empty” capsid. Such a capsid may contain no detectable genomic sequences of an expression cassette, or only partially packaged genomic sequences which are insufficient to achieve expression of the gene product. These empty capsids are non-functional to transfer the gene of interest to a host cell.

The recombinant AAV described herein may be generated using techniques which are known. See, e.g., WO 2003/042397; WO 2005/033321, WO 2006/110689; U.S. Pat. No. 7,588,772 B2. Such a method involves culturing a host cell which contains a nucleic acid sequence encoding an AAV capsid; a functional rep gene; an expression cassette composed of, at a minimum, AAV inverted terminal repeats (ITRs) and a transgene; and sufficient helper functions to permit packaging of the expression cassette into the AAV capsid protein. Methods of generating the capsid, coding sequences therefore, and methods for production of rAAV vectors have been described. See, e.g., Gao, et al, Proc. Natl. Acad. Sci. U.S.A. 100 (10), 6081-6086 (2003) and US 2013/0045186A1.

In certain embodiment, the rAAV are generated (manufactured) using triple transfection techniques. In certain embodiments the rAAV are generated using a stable mammalian cell line. In certain embodiments, the stable cell line comprises one or more of: (a) a first plurality of polynucleotide molecules which comprise a coding sequence for at least one adeno-associated virus (AAV) replicase (Rep) protein necessary for production of a replication-defective rAAV vector (Rep52 and Rep78), wherein said rep proteins coding sequences are operably linked to a doxycycline-inducible promoter which directs expression of the rep proteins in the cell line; (b) at least a second plurality of polynucleotide molecules each encoding adenovirus (Ad) helper proteins necessary for production of a replication-defective rAAV vector comprising at least an Ad E2A DNA Binding Protein (DBP) coding sequence, and Ad E4ORF6 coding sequence, wherein the Ad E2A DBP coding sequences and the Ad E4ORF6 coding sequences are operably linked to a doxycycline-inducible promoter which direct expression of the Ad helper proteins in the cell line; (c) a nucleic acid molecule comprising an Ad E1 coding sequence operably linked to a constitutive promoter which directs expression of the Ad E1 in the cell line; (d) at least a third plurality of nucleic acid molecules each of which comprises an AAV VP1 coding sequence which encodes AAV VP1 proteins, AAV VP2 proteins and AAV VP3 proteins which self-assemble to form an AAV capsid following expression in the cell, said AAV VP1 coding sequence being operably linked to a promoter which directs expression of the VP1 coding sequences in the cell line. See also, U.S. Provisional Patent Application No. 63/490,222, filed Mar. 14, 2023, which is incorporated herein by reference.

In one embodiment, cells are manufactured in a suitable cell culture (e.g., HEK 293 cells). Methods for manufacturing the gene therapy vectors described herein include methods well known in the art such as generation of plasmid DNA used for production of the gene therapy vectors, generation of the vectors, and purification of the vectors. In some embodiments, the gene therapy vector is an AAV vector and the plasmids generated are an AAV cis-plasmid encoding the AAV genome and the gene of interest for packaging into the capsid, an AAV trans-plasmid containing AAV rep and cap genes, and an adenovirus helper plasmid. The vector generation process can include method steps such as initiation of cell culture, passage of cells, seeding of cells, transfection of cells with the plasmid DNA, post-transfection medium exchange to serum free medium, and the harvest of vector-containing cells and culture media. The harvested vector-containing cells and culture media are referred to herein as crude cell harvest. In yet another system, the gene therapy vectors are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

The crude cell harvest may thereafter be subject method steps such as concentration of the vector harvest, diafiltration of the vector harvest, microfluidization of the vector harvest, nuclease digestion of the vector harvest, filtration of microfluidized intermediate, crude purification by chromatography, crude purification by ultracentrifugation, buffer exchange by tangential flow filtration, and/or formulation and filtration to prepare bulk vector.

A two-step affinity chromatography purification at high salt concentration followed anion exchange resin chromatography are used to purify the vector drug product and to remove empty capsids. These methods are described in more detail in International Patent Application No. PCT/US2016/065970, filed Dec. 9, 2016, and U.S. Pat. No. 11,098,286 B2, entitled “Scalable Purification Method for AAV9”, which are incorporated by reference. Purification methods for AAV8, International Patent Application No. PCT/US2016/065976, filed Dec. 9, 2016, and U.S. Pat. No. 11,015,174 B2, entitled “Scalable Purification Method for AAV8”, which are incorporated herein by reference. Purification methods for rh 10, International Patent Application No. PCT/US16/066013, filed Dec. 9, 2016, and U.S. Pat. No. 11,028,372 B2, entitled “Scalable Purification Method for AAVrh10”, which are incorporated herein by reference. Purification methods for AAV1, International Patent Application No. PCT/US2016/065974, filed Dec. 9, 2016, and U.S. Pat. No. 11,015,173 B2, entitled “Scalable Purification Method for AAV1”, which are incorporated herein by reference.

To calculate empty and full particle content, vp3 band volumes for a selected sample (e.g., in examples herein an iodixanol gradient-purified preparation where number of GC=number of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt)/mL. Pt/mL divided by GC/mL gives the ratio of particles to genome copies (pt/GC). Pt/mL-GC/mL gives empty pt/mL. Empty pt/mL divided by pt/mL and ×100 gives the percentage of empty particles.

Generally, methods for assaying for empty capsids and AAV vector particles with packaged genomes have been known in the art. See, e.g., Grimm et al., Gene Therapy (1999) 6:1322-1330; and Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Virol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e., SYPRO ruby or coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

Additionally, another example of measuring empty to full particle ratio is also known in the art. Sedimentation velocity, as measured in an analytical ultracentrifuge (AUC) can detect aggregates, other minor components as well as providing good quantitation of relative amounts of different particle species based upon their different sedimentation coefficients. This is an absolute method based on fundamental units of length and time, requiring no standard molecules as references. Vector samples are loaded into cells with 2-channel charcoal-epon centerpieces with 12 mm optical path length. The supplied dilution buffer is loaded into the reference channel of each cell. The loaded cells are then placed into an AN-60Ti analytical rotor and loaded into a Beckman-Coulter ProteomeLab XL-I analytical ultracentrifuge equipped with both absorbance and RI detectors. After full temperature equilibration at 20° C. the rotor is brought to the final run speed of 12,000 rpm. A280 scans are recorded approximately every 3 minutes for ~5.5 hours (110 total scans for each sample). The raw data is analyzed using the c(s) method and implemented in the analysis program SEDFIT. The resultant size distributions are graphed and the peaks are integrated. The percentage values associated with each peak represent the peak area fraction of the total area under all peaks and are based upon the raw data generated at 280 nm; many labs use these values to calculate empty: full particle ratios. However, because empty and full particles have different extinction coefficients at this wavelength, the raw data can be adjusted accordingly. The ratio of the empty particle and full monomer peak values both before and after extinction coefficient-adjustment is used to determine the empty-full particle ratio.

In one aspect, an optimized q-PCR method is used which utilizes a broad-spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2-fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000-fold) and subjected to TaqMan analysis as described in the standard assay. Quantification also can be done using ViroCyt or flow cytometry.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April; 25 (2): 115-25. doi: 10.1089/hgtb.2013.131. Epub 2014 Feb. 14.

In certain embodiments, the manufacturing process for rAAV as described herein (e.g., comprising engineered rAAV) involves method as described in U.S. Provisional Patent Application No. 63/371,597, filed Aug. 16, 2022, and U.S. Provisional Patent Application No. 63/371,592, filed Aug. 16, 2022, which are incorporated herein by reference in its entirety.

### Therapeutic Proteins and Delivery Systems

In certain embodiments, provided here are fusion partners, conjugate partners and recombinant vectors containing the muscle targeting peptide provided herein, comprising “Xn-n-mer-Xm”, wherein: (i) Xn is 0, 1, 2 or 3 amino acid residues independently selected from any amino acid; (ii) the n-mer is IIRGDPA (SEQ ID NO: 1), AVIRGDV (SEQ ID NO: 2), IVRGDPA (SEQ ID NO: 8), MIRGDVK (SEQ ID NO: 9), AQHRGDV (SEQ ID NO: 10), VSRGDPN (SEQ ID NO: 11), VSRGDPA (SEQ ID NO: 12), PLVRGDI (SEQ ID NO:13), PYVRGDP (SEQ ID NO: 14), VVRGDPQ (SEQ ID NO: 15), PTRGDVK (SEQ ID NO: 16), VVQRGDV (SEQ ID NO: 17), QHRGDTQ (SEQ ID NO: 18), QIRGDLR (SEQ ID NO: 19), RGDYAQV (SEQ ID NO: 20), IGRGDPN (SEQ ID NO: 21), RGDLHGY (SEQ ID NO: 22), RGDYSTM (SEQ ID NO: 23), or PYQRGDH (SEQ ID NO: 24), or an n-mer sequence of at least 6, at least 7, or the full-length consecutive amino acids of any one of the n-mers; and (iii) Xm is 0, 1, 2, or 3 amino acid residues independently selected from any amino acid, which are useful with a variety of different therapeutic proteins, polypeptides, nanoparticles, and delivery systems. Examples of proteins and compounds useful in compositions provided herein and targeted delivery are described below. It will be understood that the viral vectors, nanoparticles and other delivery systems contain sequences encoding the selected proteins (or conjugates) for expression in vivo.

In some embodiments, provided herein is an rAAV having a modified capsid with one or more exogenous targeting peptides, wherein the exogenous targeting peptides comprise “Xn-n-mer-Xm”, wherein: (i) Xn is 0, 1, 2 or 3 amino acid residues independently selected from any amino acid; (ii) the n-mer is IIRGDPA (SEQ ID NO: 1), AVIRGDV (SEQ ID NO: 2), IVRGDPA (SEQ ID NO: 8), MIRGDVK (SEQ ID NO: 9), AQHRGDV (SEQ ID NO: 10), VSRGDPN (SEQ ID NO: 11), VSRGDPA (SEQ ID NO: 12), PLVRGDI (SEQ ID NO:13), PYVRGDP (SEQ ID NO: 14), VVRGDPQ (SEQ ID NO: 15), PTRGDVK (SEQ ID NO: 16), VVQRGDV (SEQ ID NO: 17), QHRGDTQ (SEQ ID NO: 18), QIRGDLR (SEQ ID NO: 19), RGDYAQV (SEQ ID NO: 20), IGRGDPN (SEQ ID NO: 21), RGDLHGY (SEQ ID NO: 22), RGDYSTM (SEQ ID NO: 23), or PYQRGDH (SEQ ID NO: 24), or an n-mer sequence of at least 6, at least 7, or the full-length consecutive amino acids of any one of the n-mers; and (iii) Xm is 0, 1, 2, or 3 amino acid residues independently selected from any amino acid, and the rAAV comprises a vector genome comprising the desired transgene and promoter for use in the target cells as detailed above is optionally assessed for contamination by conventional methods and then formulated into a pharmaceutical composition intended for administration to a subject in need thereof. Such formulation involves the use of a pharmaceutically and/or physiologically acceptable vehicle or carrier, such as buffered saline or other buffers, e.g., HEPES, to maintain pH at appropriate physiological levels, and, optionally, other medicinal agents, pharmaceutical agents, stabilizing agents, buffers, carriers, adjuvants, diluents, etc. For injection, the carrier will typically be a liquid.

In certain embodiments, proteins, polypeptides, nanoparticles, and/or delivery systems including viral vectors (rAAV) and nanoparticles, comprising the exogenous targeting peptide provided herein, are useful in treatment of one or more of cardiac and/or skeletal (e.g., gastrocnemius) muscle-based disorders. Such disease and/or disorders may include, without limitation, auto immune disease, cancer, muscular dystrophy, a neuro-muscular disease, a sugar or glycogen storage disease, cardiomyopathy, infectious disease affecting the muscle cell. More specifically, such disease and/or disorders may include, without limitation, Huntington's disease, a Myotonic Dystrophy (Type 1 or Type 2), Facioscapulohumeral muscular dystrophy (FSHD), Duchene muscular dystrophy, Becker Muscular dystrophy, Limb-Girdle muscular dystrophy, Emery Dreifuss muscular dystrophy, Oculopharyngeal muscular dystrophy, Barth syndrome, MPS type III disease, Pompe disease, Fabry Disease, Charcot-Marie-Tooth disease, Friedreich's Ataxia, dilated cardiomyopathy, hypertrophic cardiomyopathy, DMD-associated cardiomyopathy, Myotubular myopathy, Primary merosin deficiency, Dannon disease, idiopathic dilated cardiomyopathy (DCM) or a disease associated with a mutation in the LMNA gene. Examples of genes and proteins those associated with disease and/or disorders, e.g., spinal muscular atrophy (SMA, SMN1), Duchenne Type Muscular dystrophy, Friedrichs Ataxia (e.g., frataxin), cardiomyopathy (LMNA), Charcot-Marie-Tooth disease (MFN2). See also, U.S. Provisional Patent Application No. 63/293,680, filed Dec. 24, 2021, International Patent Application No. PCT/US2021/041406, filed Jul. 13, 2021, now Publication No. WO 2022/015715A1, International Patent Application No. PCT/US2022/076939, filed Sep. 23, 2022, International Patent Application No. PCT/US2020/066167, filed Dec. 18, 2020, now Publication No. WO 2021/127533A1, International Patent Application No. PCT/US2022/025879, filed Apr. 22, 2022, now Publication No. WO 2022/226263A1, and International Patent Application No. PCT/US2021/054145, filed Oct. 8, 2021, now Publication No. WO2022/076803, which are all incorporated herein by reference in their entireties.

In certain embodiments, the protein useful in compositions provided herein is encoded by a transgene sequence including hormones and growth and differentiation factors including, without limitation, insulin, glucagon, glucagon-like peptide 1 (GLP-1), growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoictins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor α (TGFα), platelet-derived growth factor (PDGF), insulin growth factors I and II (IGF-I and IGF-II), any one of the transforming growth factor β superfamily, including TGF β, activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), lysosomal acid lipase (LIPA or LAL), neurturin, agrin, any one of the family of semaphorins/collapsins, netrin-1 and netrin-2, hepatocyte growth factor (HGF), ephrins, noggin, sonic hedgehog and tyrosine hydroxylase. Other useful transgene encode lysosomal enzymes that cause mucopolysaccharidoses (MPS), including α-L-iduronidase (MPSI), iduronate sulfatase (MPSII), heparan N-sulfatase (sulfaminidase) (MPS IIIA, Sanfilippo A), α-N-acetyl-glucosaminidase (MPS IIIB, Sanfilippo B), acetyl-CoA: α-glucosaminide acetyltransferase (MPS IIIC, Sanfilippo C), N-acetylglucosamine 6-sulfatase (MPS IIID, Sanfilippo D), galactose 6-sulfatase (MPS IVA, Morquio A), β-Galactosidase (MPS IVB, Morquio B), N-acetyl-galactosamine 4-sulfatase (MPS VI, Maroteaux-Lamy), β-Glucuronidase (MPS VII, Sly), and hyaluronidase (MPS IX).

In certain embodiments, the protein useful in compositions provided herein is encoded by a transgene sequence including a reporter sequence, which upon expression produces a detectable signal. Such reporter sequences include, without limitation, DNA sequences encoding β-lactamase, β-galactosidase (LacZ), alkaline phosphatase, thymidine kinase, green fluorescent protein (GFP), enhanced GFP (EGFP), chloramphenicol acetyltransferase (CAT), luciferase, membrane bound proteins including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc.

An rAAV having a mutant rAAV capsid as provided herein has a vector genome which comprises nucleic acid sequence encoding proteins, for example that act as transcriptional repressors, antisense molecules, ribozymes, small inhibitory nucleic acid sequences, for example but are not limited to RNAi, shRNAi, siRNA, micro RNAi (mRNAi or miRNA), antisense oligonucleotides etc. These may be in additional to or in alternative to a protein to be delivered.

### Compositions and Uses

Provided herein are compositions containing at least one rAAV stock (e.g., an rAAV9 engineered stock or rAAVhu68 engineered stock, wherein engineered capsid comprises an exogenous targeting motif as described herein) and an optional carrier, excipient and/or preservative.

In one aspect, provided is a pharmaceutical composition comprising a rAAV as described herein in a formulation buffer. In one embodiment, the rAAV is formulated at about 1×10⁹ genome copies (GC)/mL to about 1×10¹⁴ GC/mL. In a further embodiment, the rAAV is formulated at about 3×10⁹ GC/mL to about 3×10¹³ GC/mL. In yet a further embodiment, the rAAV is formulated at about 1×10⁹ GC/mL to about 1×10¹³ GC/mL. In one embodiment, the rAAV is formulated at least about 1×10¹¹ GC/mL.

Provided herein, also, are compositions containing at least one therapeutic protein, polypeptide, nanoparticles and/or delivery system comprising the targeting motif as provided herein, and an optional carrier, excipient and/or preservative.

Provided herein, also, are methods of use of compositions as described herein. In certain embodiments, a method for targeted therapy to muscle cells comprising administering to a patient in need thereof a stock of the rAAV as described herein, wherein a therapeutic is targeted for delivery to muscle cells (e.g., cardiac cells (heart), skeletal muscle cells (e.g., gastrocnemius)), and is de-targeted for cells in liver.

Additionally, provided herein is a method of delivering of a transgene to one or more muscle cells of a subject comprising administering to the subject a recombinant adeno-associated virus (rAAV) vector comprising engineered capsid proteins comprising exogenous targeting peptides, wherein the exogenous targeting peptides comprise “Xn-n-mer-Xm”, wherein: (i) Xn is 0, 1, 2 or 3 amino acid residues independently selected from any amino acid; (ii) the n-mer is IIRGDPA (SEQ ID NO: 1), AVIRGDV (SEQ ID NO: 2), IVRGDPA (SEQ ID NO: 8), MIRGDVK (SEQ ID NO: 9), AQHRGDV (SEQ ID NO: 10), VSRGDPN (SEQ ID NO: 11), VSRGDPA (SEQ ID NO: 12), PLVRGDI (SEQ ID NO:13), PYVRGDP (SEQ ID NO: 14), VVRGDPQ (SEQ ID NO: 15), PTRGDVK (SEQ ID NO: 16), VVQRGDV (SEQ ID NO: 17), QHRGDTQ (SEQ ID NO: 18), QIRGDLR (SEQ ID NO: 19), RGDYAQV (SEQ ID NO: 20), IGRGDPN (SEQ ID NO: 21), RGDLHGY (SEQ ID NO: 22), RGDYSTM (SEQ ID NO: 23), or PYQRGDH (SEQ ID NO: 24), or an n-mer sequence of at least 6, at least 7, or full-length consecutive amino acids of any one of the n-mers; and (iii) Xm is 0, 1, 2, or 3 amino acid residues independently selected from any amino acid, and rAAV further comprising a vector genome comprising the transgene operably linked to regulatory sequences that direct expression of the transgene in muscle cell.

In certain embodiments, the target muscle cell is a cardiac, smooth, and/or skeletal muscle cell. In certain embodiments, the transgene encodes a secreted gene product. In certain embodiments, the AAV vector is delivered intravenously.

Provided herein are also uses of an rAAV having a engineered capsid with one or more exogenous targeting peptides, to target muscle cells at higher levels of transduction than achieved using an AAV9 vector, wherein the exogenous targeting peptide comprises “Xn-n-mer-Xm”, wherein: (i) Xn is 0, 1, 2 or 3 amino acid residues independently selected from any amino acid; (ii) the n-mer is IIRGDPA (SEQ ID NO: 1), AVIRGDV (SEQ ID NO: 2), IVRGDPA (SEQ ID NO: 8), MIRGDVK (SEQ ID NO: 9), AQHRGDV (SEQ ID NO: 10), VSRGDPN (SEQ ID NO: 11), VSRGDPA (SEQ ID NO: 12), PLVRGDI (SEQ ID NO:13), PYVRGDP (SEQ ID NO: 14), VVRGDPQ (SEQ ID NO: 15), PTRGDVK (SEQ ID NO: 16), VVQRGDV (SEQ ID NO: 17), QHRGDTQ (SEQ ID NO: 18), QIRGDLR (SEQ ID NO: 19), RGDYAQV (SEQ ID NO: 20), IGRGDPN (SEQ ID NO: 21), RGDLHGY (SEQ ID NO: 22), RGDYSTM (SEQ ID NO: 23), or PYQRGDH (SEQ ID NO: 24), or an n-mer sequence of at least 6, at least 7, or the full-length consecutive amino acids of any one of the n-mers; and (iii) Xm is 0, 1, 2, or 3 amino acid residues independently selected from any amino acid.

In certain embodiments, a composition may contain at least a second, different rAAV stock. This second vector stock may vary from the first by having a different AAV capsid and/or a different vector genome. In certain embodiments, a composition as described herein may contain a different vector expressing an expression cassette as described herein, or another active component (e.g., an antibody construct, another biologic, and/or a small molecule drug).

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host. Delivery vehicles such as liposomes, nanocapsules, microparticles, microspheres, lipid particles, vesicles, and the like, may be used for the introduction of the compositions of the present invention into suitable host cells. In particular, the rAAV vector delivered transgenes may be formulated for delivery either encapsulated in a lipid particle, a liposome, a vesicle, a nanosphere, or a nanoparticle or the like.

In one embodiment, a composition includes a final formulation suitable for delivery to a subject, e.g., is an aqueous liquid suspension buffered to a physiologically compatible pH and salt concentration. Suitably, the final formulation is adjusted to a physiologically acceptable pH, e.g., the pH may be in the range of pH 6 to 9, or pH 6.5 to 7.5, pH 7.0 to 7.7, or pH 7.2 to 7.8. For intravenous delivery, a pH of 6.8 to about 7.2 may be desired. However, other pHs within the broadest ranges and these subranges may be selected for other routes of delivery. Optionally, one or more surfactants are present in the formulation. In another embodiment, the composition may be transported as a concentrate which is diluted for administration to a subject. In other embodiments, the composition may be lyophilized and reconstituted at the time of administration.

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol (polyethylene glycol)-15 Hydroxystearate), LABRASOL® (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

In another embodiment, the composition includes a carrier, diluent, excipient and/or adjuvant. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The buffer/carrier should include a component that prevents the rAAV, from sticking to the infusion tubing but does not interfere with the rAAV binding activity in vivo. A suitable surfactant, or combination of surfactants, may be selected from among non-ionic surfactants that are nontoxic. In one embodiment, a difunctional block copolymer surfactant terminating in primary hydroxyl groups is selected, e.g., such as Poloxamer 188 (also known under the commercial names Pluronic® F68 [BASF], Lutrol® F68, Synperonic® F68, Kolliphor® P188) which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy-oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

In certain embodiments, the formulation may contain a buffered saline aqueous solution not comprising sodium bicarbonate. Such a formulation may contain a buffered saline aqueous solution comprising one or more of sodium phosphate, sodium chloride, potassium chloride, calcium chloride, magnesium chloride and mixtures thereof, in water, such as a Harvard's buffer. In one embodiment, the buffer is phosphate-buffered saline (PBS). In one embodiment, the formulation buffer PBS with total salt concentration of 200 mM, 0.001% (w/v) pluronic F68 (Final Formulation Buffer, FFB).

Optionally, the compositions of the invention may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The compositions according to the present invention may comprise a pharmaceutically acceptable carrier, such as defined above. Suitably, the compositions described herein comprise an effective amount of one or more AAV suspended in a pharmaceutically suitable carrier and/or admixed with suitable excipients designed for delivery to the subject via injection, or for delivery by another route and/or device.

In certain embodiments, the composition comprises a viral vector (i.e., rAAV vector). The vectors are administered in sufficient amounts to transfect the cells and to provide sufficient levels of gene transfer and expression to provide a therapeutic benefit without undue adverse effects, or with medically acceptable physiological effects, which can be determined by those skilled in the medical arts. In certain embodiments, the vectors are formulated for delivery via systemic or direct delivery to a desired organ (e.g., lung), oral inhalation, intratracheal, intraarterial, intraocular, intravenous, intramuscular, subcutaneous, intradermal, and other parenteral routes of administration.

As used herein, the term “dosage” or “amount” can refer to the total dosage or amount delivered to the subject in the course of treatment, or the dosage or amount delivered in a single unit (or multiple unit or split dosage) administration. Dosages of the viral vector will depend primarily on factors such as the condition being treated, the age, weight and health of the patient, and may thus vary among patients. For example, a therapeutically effective human dosage of the viral vector is generally in the range of from about 25 to about 1000 microliters to about 5 mL of aqueous suspending liquid containing doses of from about 10⁹ to 4×10¹⁴ GC of AAV vector. The dosage will be adjusted to balance the therapeutic benefit against any side effects and such dosages may vary depending upon the therapeutic application for which the recombinant vector is employed. The levels of expression of the transgene can be monitored to determine the frequency of dosage resulting in viral vectors, preferably AAV vectors containing the minigene. Optionally, dosage regimens similar to those described for therapeutic purposes may be utilized for immunization using the compositions of the invention.

The replication-defective virus compositions can be formulated in dosage units to contain an amount of replication-defective virus that is in the range of about 1.0×10⁹ GC to about 1.0×10¹⁶ GC (to treat an average subject of 70 kg in body weight) including all integers or fractional amounts within the range, and preferably 1.0×10¹² GC to 1.0×10¹⁴ GC for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 1×10¹⁰ to about 1×10¹² GC per dose including all integers or fractional amounts within the range. In certain embodiments, the rAAV compositions is formulated in dosage units to contain about 1×10¹³ GC/kg. In certain embodiments, the rAAV compositions is formulated in dosage units to contain about 2.5×10¹³ GC/kg. In certain embodiments, the rAAV compositions is formulated in dosage units to contain about 5×10¹³ GC/kg.

In one embodiment, for human application the dose can range from 109 GC to about 7×10¹³ GC per dose including all integers or fractional amounts within the range.

These above doses may be administered in a variety of volumes of carrier, excipient or buffer formulation, ranging from about 25 to about 1000 microliters, or higher volumes, including all numbers within the range, depending on the size of the area to be treated, the viral titer used, the route of administration, and the desired effect of the method. In one embodiment, the volume of carrier, excipient or buffer is at least about 25 μL. In one embodiment, the volume is about 50 μL. In another embodiment, the volume is about 75 μL. In another embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 225 μL. In yet another embodiment, the volume is about 250 μL. In yet another embodiment, the volume is about 275 μL. In yet another embodiment, the volume is about 300 μL. In yet another embodiment, the volume is about 325 μL. In another embodiment, the volume is about 350 μL. In another embodiment, the volume is about 375 μL. In another embodiment, the volume is about 400 μL. In another embodiment, the volume is about 450 μL. In another embodiment, the volume is about 500 μL. In another embodiment, the volume is about 550 μL. In another embodiment, the volume is about 600 μL. In another embodiment, the volume is about 650 μL. In another embodiment, the volume is about 700 μL. In another embodiment, the volume is between about 700 and 1000 μL.

In one embodiment, the rAAV constructs may be delivered in doses of about 1×10⁹ GCs to about 1×10¹⁵, or about 1×10¹¹ to 5×10¹³ GC. Suitable volumes for delivery of these doses and concentrations may be determined by one of skill in the art. For example, volumes of about 1 μL to 150 mL may be selected, with the higher volumes being selected for adults. Typically, for newborn infants a suitable volume is about 0.5 mL to about 10 mL, for older infants, about 0.5 mL to about 15 mL may be selected. For toddlers, a volume of about 0.5 mL to about 20 mL may be selected. For children, volumes of up to about 30 mL may be selected. For pre-teens and teens, volumes up to about 50 mL may be selected. Other suitable volumes and dosages may be determined. The dosage will be adjusted to balance the therapeutic benefit against any side effects and such dosages may vary depending upon the therapeutic application for which the recombinant vector is employed.

The compositions according to the present invention may comprise a pharmaceutically acceptable carrier, such as defined above. Suitably, the compositions described herein comprise an effective amount of one or more AAV suspended in a pharmaceutically suitable carrier and/or admixed with suitable excipients designed for delivery to the subject via injection.

The composition, the suspension or the pharmaceutical compositions described herein are designed for delivery to subjects in need thereof by any suitable route or a combination of different routes. In certain embodiments, the rAAV or the pharmaceutical composition comprises a formulation buffer suitable for intravenous administration to a patient in the need thereof.

In certain embodiments, provided herein is a composition comprising one or more exogenous muscle cell-targeting peptide(s) comprising “Xn-n-mer-Xm”, wherein: (i) Xn is 0, 1, 2 or 3 amino acid residues independently selected from any amino acid; (ii) the n-mer selected is IIRGDPA (SEQ ID NO: 1), AVIRGDV (SEQ ID NO: 2), IVRGDPA (SEQ ID NO: 8), MIRGDVK (SEQ ID NO: 9), AQHRGDV (SEQ ID NO: 10), VSRGDPN (SEQ ID NO: 11), VSRGDPA (SEQ ID NO: 12), PLVRGDI (SEQ ID NO:13), PYVRGDP (SEQ ID NO: 14), VVRGDPQ (SEQ ID NO: 15), PTRGDVK (SEQ ID NO: 16), VVQRGDV (SEQ ID NO: 17), QHRGDTQ (SEQ ID NO: 18), QIRGDLR (SEQ ID NO: 19), RGDYAQV (SEQ ID NO: 20), IGRGDPN (SEQ ID NO: 21), RGDLHGY (SEQ ID NO: 22), RGDYSTM (SEQ ID NO: 23), or PYQRGDH (SEQ ID NO: 24), or an n-mer sequence of at least 6, at least 7, or the full-length consecutive amino acids of any one of the n-mers; and (iii) Xm is 0, 1, 2, or 3 amino acid residues independently selected from any amino acid, together with one or more of a physiologically compatible carrier, excipient, and/or aqueous suspension base. Further provided are compositions comprising nucleic acid sequences encoding same.

In certain embodiments, a composition comprising a fusion polypeptide or protein, or a nucleic acid sequence encoding the fusion polypeptide or protein, or a nanoparticle containing same are provided. The composition may further comprise one or more of a physiologically compatible carrier, excipient, and/or aqueous suspension base.

In certain embodiments, a nucleic acid sequence encoding the fusion polypeptide protein is encapsulated in a lipid nanoparticle (LNP). As used herein, the phrase “lipid nanoparticle” or “nanoparticle” refers to a transfer vehicle comprising one or more lipids (e.g., cationic lipids, non-cationic lipids, and PEG-modified lipids). Preferably, the lipid nanoparticles are formulated to deliver one or more nucleic acid sequences to one or more target cells (e.g., muscle cell (cardiac, skeletal, smooth)). Examples of suitable lipids include, for example, the phosphatidyl compounds (e.g., phosphatidylglycerol, phosphatidylcholine, phosphatidylserine, phosphatidylethanolamine, sphingolipids, cerebrosides, and gangliosides). Also contemplated is the use of polymers as transfer vehicles, whether alone or in combination with other transfer vehicles. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, dendrimers and polyethylenimine. In one embodiment, the transfer vehicle is selected based upon its ability to facilitate the transfection of a nucleic acid sequence encapsulated therein to a target cell. Useful lipid nanoparticles for nucleic acid sequence comprise a cationic lipid to encapsulate and/or enhance the delivery of such nucleic acid sequence into the target cell that will act as a depot for protein production. As used herein, the phrase “cationic lipid” refers to any of a number of lipid species that carry a net positive charge at a selected pH, such as physiological pH. The contemplated lipid nanoparticles may be prepared by including multi-component lipid mixtures of varying ratios employing one or more cationic lipids, non-cationic lipids and PEG-modified lipids. Several cationic lipids have been described in the literature, many of which are commercially available. See, e.g., WO2014/089486, US 2018/0353616A1, and U.S. Pat. No. 8,853,377B2, which are incorporated by reference. In certain embodiments, LNP formulation is performed using routine procedures comprising cholesterol, ionizable lipid, helper lipid, PEG-lipid and polymer forming a lipid bilayer around encapsulated nucleic acid sequence (Kowalski et al., 2019, Mol. Ther. 27 (4): 710-728). In some embodiments, LNP comprises a cationic lipids (i.e. N-[1-(2,3-dioleoyloxy) propyl]-N,N,N-trimethylammonium chloride (DOTMA), or 1,2-dioleoyl-3-trimethylammonium-propane (DOTAP)) with helper lipid DOPE. In some embodiments, LNP comprises an ionizable lipid Dlin-MC3-DMA ionizable lipids, or diketopiperazine-based ionizable lipids (cKK-E12). In some embodiments, polymer comprises a polyethyleneimine (PEI), or a poly(β-amino) esters (PBAEs). See, e.g., WO2014/089486, US 2018/0353616A1, US2013/0037977A1, WO2015/074085A1, U.S. Pat. No. 9,670,152B2, and U.S. Pat. No. 8,853,377B2, which are incorporated by reference. In certain embodiments, a lipid nanoparticle (LNP) comprises at least one exogenous targeting peptide comprising “Xn-n-mer-Xm”, wherein: (i) Xn is 0, 1, 2 or 3 amino acid residues independently selected from any amino acid; (ii) the n-mer is IIRGDPA (SEQ ID NO: 1), AVIRGDV (SEQ ID NO: 2), IVRGDPA (SEQ ID NO: 8), MIRGDVK (SEQ ID NO: 9), AQHRGDV (SEQ ID NO: 10), VSRGDPN (SEQ ID NO: 11), VSRGDPA (SEQ ID NO: 12), PLVRGDI (SEQ ID NO: 13), PYVRGDP (SEQ ID NO: 14), VVRGDPQ (SEQ ID NO: 15), PTRGDVK (SEQ ID NO: 16), VVQRGDV (SEQ ID NO: 17), QHRGDTQ (SEQ ID NO: 18), QIRGDLR (SEQ ID NO: 19), RGDYAQV (SEQ ID NO: 20), IGRGDPN (SEQ ID NO: 21), RGDLHGY (SEQ ID NO: 22), RGDYSTM (SEQ ID NO: 23), or PYQRGDH (SEQ ID NO: 24), or an n-mer sequence of at least 6, at least 7, or the full-length consecutive amino acids of any one of the n-mers; and (iii) Xm is 0, 1, 2, or 3 amino acid residues independently selected from any amino acid, i.e., decorated surface with targeting peptide. In certain embodiments, a lipid nanoparticle (LNP) comprises at least one IIRGDPA (SEQ ID NO: 1) peptide. In certain embodiments, a lipid nanoparticle (LNP) comprises at least one AVIRGDV (SEQ ID NO: 2) peptide.

In certain embodiments, provided herein is a composition comprising, e.g., an rAAV having an engineered capsid with at least one exogenous targeting peptide comprising “Xn-n-mer-Xm”, wherein: (i) Xn is 0, 1, 2 or 3 amino acid residues independently selected from any amino acid; (ii) the n-mer is IIRGDPA (SEQ ID NO: 1), AVIRGDV (SEQ ID NO: 2), IVRGDPA (SEQ ID NO: 8), MIRGDVK (SEQ ID NO: 9), AQHRGDV (SEQ ID NO: 10), VSRGDPN (SEQ ID NO: 11), VSRGDPA (SEQ ID NO: 12), PLVRGDI (SEQ ID NO:13), PYVRGDP (SEQ ID NO: 14), VVRGDPQ (SEQ ID NO: 15), PTRGDVK (SEQ ID NO: 16), VVQRGDV (SEQ ID NO: 17), QHRGDTQ (SEQ ID NO: 18), QIRGDLR (SEQ ID NO: 19), RGDYAQV (SEQ ID NO: 20), IGRGDPN (SEQ ID NO: 21), RGDLHGY (SEQ ID NO: 22), RGDYSTM (SEQ ID NO: 23), or PYQRGDH (SEQ ID NO: 24), or a an-mer sequence of at least 6, at least 7, or the full-length consecutive amino acids of any one of the n-mers; and (iii) Xm is 0, 1, 2, or 3 amino acid residues independently selected from any amino acid is useful for delivering a therapeutic to a patient in need thereof. In certain embodiments, a composition comprising an rAAV having a modified capsid with at least one IIRGDPA (SEQ ID NO: 1) peptide is useful for delivering a therapeutic to a patient in need thereof, wherein the therapeutic is targeted for delivery to muscle cell. In certain embodiments, a composition comprising an rAAV having a modified capsid with at least one core AVIRGDV (SEQ ID NO: 2 is useful for delivering a therapeutic to a patient in need thereof, wherein the therapeutic is targeted for delivery to in muscle cell. In certain embodiments, a composition comprising an rAAV having a modified capsid with at least one IIRGDPA (SEQ ID NO: 1) peptide is useful for delivering a therapeutic to a patient in need thereof, wherein the therapeutic is targeted for delivery to muscle cell, and de-targeted for liver. In certain embodiments, a composition comprising an rAAV having a modified capsid with at least one core AVIRGDV (SEQ ID NO: 2 is useful for delivering a therapeutic to a patient in need thereof, wherein the therapeutic is targeted for delivery to in muscle cell, and de-targeted for liver.

In certain embodiments, the methods and compositions are useful for treatment of mitochondrial cardiomyopathy associated with Barth Syndrome. Barth Syndrome is a rare, X-linked recessive disorder characterized by a loss of function mutation in TAZ gene (i.e., amenable gene therapy). Bart Syndrome is associated with pediatric onset cardiomyopathy (i.e., by age 5) with neutropenia, mild mitochondrial myopathy (skeletal muscle weakness), and mild intellectual impairment. See also, Sabbah, H. N., Barth syndrome cardiomyopathy: targeting the mitochondria with elamipretide, Heart Failure Reviews (2021) 26:237-253, which is incorporated herein by reference in its entirety.

In certain embodiments, the methods and compositions are useful for treatment of autosomal dominant form of long-QT syndrome caused by a loss-of-function and partial dominant negative mutations in KCNQ1 gene (i.e., amenable to gene replacement or knockdown/replace approach). The autosomal dominant form of long-QT syndrome is associated with syncope and sudden cardiac death usually occurring during exercise or emotional stress, and many patients remain at-risk despite standard of care (beta blockers, cardiac sympathetic denervation) and require Implantable Cardioverter Defibrillator (ICD). See also, Huang H., et al., Mechanisms of KCNQ1 channel dysfunction in long QT syndrome involving voltage sensor domain mutations, Sci. Adv. 2018, 4:1-12, epub Mar. 7, 2018, which is incorporated herein by reference in its entirety.

In certain embodiments, the methods and compositions are useful for treatment of hypertrophic cardiomyopathy. In certain embodiments, the methods and compositions may be used in treatment of hypertrophic cardiomyopathy caused by loss-of-function mutations in the MYBPC3 gene (i.e., amenable to gene therapy). See also, Mearini G., et al., Mybpc3 gene therapy for neonatal cardiomyopathy enables long-term disease prevention in mice, Nature Communication, 2014, 5:5515, epub Dec. 2, 2014, which is incorporated herein by reference in its entirety.

In certain embodiments, the methods and compositions are useful for treatment of transthyretin amyloid cardiomyopathy (ATTR-CM). In certain embodiments, the methods and compositions may be used in treatment of ATTR-CM caused by mutations in the transthyretin (TTR) gene (i.e., amenable to gene therapy). See also, Yamamoto H, Yokochi T. Transthyretin cardiac amyloidosis: an update on diagnosis and treatment. ESC Heart Fail. 2019 December; 6 (6): 1128-1139; and Jain A, Zahra F. Transthyretin Amyloid Cardiomyopathy (ATTR-CM) [Updated 2023 Apr. 27]. In: StatPearls [Internet]. Treasure Island (FL): StatPearls Publishing; 2023 January, ncbi.nlm.nih.gov/books/NBK574531/, which are incorporated herein by reference in their entirety.

In certain embodiments, the methods and compositions are useful for treatment of long WT syndrome type 2 caused by a loss-of-function mutation in hERG (Kv11.1; also, Kv11.1 voltage-gated potassium channel) gene. See also, Curran M E., et al., A Molecular Basis for Cardiac Arrhythmia: HERG Mutations Cause Long QT Syndrome, Cell, Voi. 80, 795-803, Mar. 10, 1995, and Hylten-Cavallius, L., et al., Patients With Long-QT Syndrome Caused by Impaired hERG-Encoded Kv11.1 Potassium Channel Have Exaggerated Endocrine Pancreatic and Incretin Function Associated With Reactive Hypoglycemia, Circulation, 2017; 135:1705-1719, which are incorporated herein by reference in their entirety.

In certain embodiments, the methods and compositions are useful for treatment of LMNA cardiomyopathy or a disease caused by loss-of-function mutation in the LMNA gene. See also, Kang, S., et al., Laminopathies; Mutations on single gene and various human genetic diseases, BMB Rep. 2018; 51 (7): 327-337, and U.S. Provisional Patent application No. 63/293,680, filed Dec. 24, 2021, International Patent Application No. PCT/US2022/082383, filed Dec. 24, 2022, now Publication No. WO 2023/122803 A1, published Jun. 29, 2023, which are incorporated herein by reference in their entirety.

In certain embodiments, the methods and compositions are useful for treatment of heart failure, ischemia-reperfusion injury, myocardial infarction, ventricular remodeling, or a disease associated with extracellular superoxide dismutase 3 (SOD3 or EcSOD). See also, US Patent Application Publication No. US20130136729A1, which is incorporated herein by reference in its entirety.

In certain embodiments, the methods and compositions are useful for treatment of myocardial infarction, reduced ejection fraction of the heart or a disease associated with myc transcription factor, cyclin T 1 and cyclin-dependent kinase 9 (CDK9). See also, International Patent Application Publication No. WO2020/165603A1, which is incorporated herein by reference in its entirety.

In certain embodiments, the methods and compositions are useful for treatment of heart failure, or heart tissue damage, or degeneration, or a disease associated with cyclin A2 protein. See also, International Patent Application Publication No. WO2020/051296A1, which is incorporated herein by reference in its entirety.

In certain embodiments, the methods and compositions are useful for treatment of dilated cardiomyopathy (DCM), a heart failure, a cardiac fibrosis, a heart inflammation, an ischemic heart disease, a myocardial infarction, an ischemic/reperfusion (I/R) related injuries, a transverse aortic constriction, or a disease associated with YY1 or BMP7 protein. See also, International Patent Application Publication No. WO2021/021021A1, which is incorporated herein by reference in its entirety.

In certain embodiments, the methods and compositions are useful for treatment of dilated cardiomyopathy, or a disease associated with cardiac Apoptosis Repressor with Caspase Recruitment Domain (cARC). See also, International Patent Application Publication No. WO2021/016126A1, which is incorporated herein by reference in its entirety.

Symptoms of cardiomyopathy or a disease associated with a mutation in a LMNA gene, KCNQ1 gene, MYBPC3 gene, TAZ gene, or hERG gene include atrioventricular (AV) conduction block, atrial fibrillation, arrhythmia including atrial arrhythmia such as atrial flutter and atrial tachycardia, and ventricular arrhythmias including sustained ventricular tachycardias, and ventricular fibrillation (VF) and/or heart failure.

In certain embodiments, the methods and compositions described herein are useful to ameliorate one or more symptoms of cardiomyopathy including increased average life span, and/or reduction in progression towards heart failure.

In certain embodiments, the methods and compositions are useful for treatment, or to ameliorate one or more symptoms of muscular dystrophy.

In certain embodiments, the methods and compositions are useful for treatment, or to ameliorate one or more symptoms of Duchenne muscular dystrophy (Dystrophin, DMD), Becker muscular dystrophy (Dystrophin, DMD), Danon disease (LAMP2), Myotubular myopathy (Myotubularin, MRM1), Primary merosin deficiency (Merosin, LAMA2), Pompe disease (α-1,4-Glucosidase, GAA), Limb-girdle muscular dystrophy (Calpain 3, CAPN3), Oculopharyngeal muscular dystrophy (PABPN1), or muscular dystrophies associated with Dysferlin (DYSF), α-Sarcoglycan (LGMD 2D), β-Sarcoglycan (SGCB) or Fukutinrelated protein (FKRP) proteins.

In certain embodiments, a rAAV having a modified capsid as described herein may be delivered in a co-therapeutic regimen which further comprises one or more other active components. In certain embodiments, the regimen may involve co-administration of an immunomodulatory component. Such an immunomodulatory regimen may include, e.g., but are not limited to immunosuppressants such as, a glucocorticoid, steroids, antimetabolites, T-cell inhibitors, a macrolide (e.g., a rapamycin or rapalog), and cytostatic agents including an alkylating agent, an anti-metabolite, a cytotoxic antibiotic, an antibody, or an agent active on immunophilin. The immune suppressant may include a nitrogen mustard, nitrosourea, platinum compound, methotrexate, azathioprine, mercaptopurine, fluorouracil, dactinomycin, an anthracycline, mitomycin C, bleomycin, mithramycin, IL-2 receptor-(CD25−) or CD3-directed antibodies, anti-IL-2 antibodies, cyclosporin, tacrolimus, sirolimus, IFN-β, IFN-γ, an opioid, or TNF-α (tumor necrosis factor-alpha) binding agent. In certain embodiments, the immunosuppressive therapy may be started prior to the gene therapy administration. Such therapy may involve co-administration of two or more drugs, the (e.g., prednelisone, micophenolate mofetil (MMF) and/or sirolimus (i.e., rapamycin)) on the same day. One or more of these drugs may be continued after gene therapy administration, at the same dose or an adjusted dose. Such therapy may be for about 1 week, about 15 days, about 30 days, about 45 days, 60 days, or longer, as needed. Still other co-therapeutics may include, e.g., anti-IgG enzymes, which have been described as being useful for depleting anti-AAV antibodies (and thus may permit administration to patients testing above a threshold level of antibody for the selected AAV capsid), and/or delivery of anti-FcRN antibodies which is described, e.g., in WO 2021/257668, filed 23 Dec. 2021, (claiming priority to U.S. Provisional Patent Application No. 63/040,381, filed Jun. 17, 2020, US Provisional Patent Application No. 62/135,998, filed Jan. 11, 2021, and U.S. Provisional Patent Application No. 63/152,085, filed Feb. 22, 2021) entitled “Compositions and Methods for Treatment of Gene Therapy Patients”, and/or one or more of a) a steroid or combination of steroids and/or (b) an IgG-cleaving enzyme, (c) an inhibitor of Fc-IgE binding; (d) an inhibitor of Fc-IgM binding; (e) an inhibitor of Fc-IgA binding; and/or (f) gamma interferon.

### Kit

In certain embodiments, a kit is provided which includes a concentrated vector suspended in a formulation (optionally frozen), optional dilution buffer, and devices and components required for intravenous administration. In another embodiment, the kit may additional or alternatively include components for intravenous delivery. In one embodiment, the kit provides sufficient buffer to allow for injection. Such buffer may allow for about a 1:1 to a 1:5 dilution of the concentrated vector, or more. In other embodiments, higher or lower amounts of buffer or sterile water are included to allow for dose titration and other adjustments by the treating clinician. In still other embodiments, one or more components of the device are included in the kit. Suitable dilution buffer is available, such as, a saline, a phosphate buffered saline (PBS) or a glycerol/PBS.

It should be understood that the compositions in kit described herein are intended to be applied to other compositions, regiments, aspects, embodiments and methods described across the Specification.

An “immunoglobulin molecule” is a protein containing the immunologically-active portions of an immunoglobulin heavy chain and immunoglobulin light chain covalently coupled together and capable of specifically combining with antigen. Immunoglobulin molecules are of any type (e.g., IgG, IgE, IgM, IgD, IgA and IgY), class (e.g., IgG1, IgG2, IgG3, IgG4, IgA1 and IgA2) or subclass. The terms “antibody” and “immunoglobulin” may be used interchangeably herein.

“Neutralizing antibody titer” (NAb titer) a measurement of how much neutralizing antibody (e.g., anti-AAV NAb) is produced which neutralizes the physiologic effect of its targeted epitope (e.g., an AAV). Anti-AAV NAb titers may be measured as described in, e.g., Calcedo, R., et al., Worldwide Epidemiology of Neutralizing Antibodies to Adeno-Associated Viruses. Journal of Infectious Diseases, 2009, 199 (3): p. 381-390, which is incorporated by reference herein.

As used herein when used to refer to vp capsid proteins, the term “heterogenous” or any grammatical variation thereof, refers to a population consisting of elements that are not the same, for example, having vp1, vp2 or vp3 monomers (proteins) with different modified amino acid sequences. SEQ ID NO: 26 provides the encoded amino acid sequence of the AAVhu68 vp1 protein. The term “heterogenous” as used in connection with vp1, vp2 and vp3 proteins (alternatively termed isoforms), refers to differences in the amino acid sequence of the vp1, vp2 and vp3 proteins within a capsid. The AAV capsid contains subpopulations within the vp1 proteins, within the vp2 proteins and within the vp3 proteins which have modifications from the predicted amino acid residues. These subpopulations include, at a minimum, certain deamidated asparagine (N or Asn) residues. For example, certain subpopulations comprise at least one, two, three or four highly deamidated asparagines (N) positions in asparagine-glycine (N-G) pairs and optionally further comprising other deamidated amino acids, wherein the deamidation results in an amino acid change and other optional modifications.

As used herein, a “subpopulation” of vp proteins refers to a group of vp proteins which has at least one defined characteristic in common and which consists of at least one group member to less than all members of the reference group, unless otherwise specified. For example, a “subpopulation” of vp1 proteins is at least one (1) vp1 protein and less than all vp1 proteins in an assembled AAV capsid, unless otherwise specified. A “subpopulation” of vp3 proteins may be one (1) vp3 protein to less than all vp3 proteins in an assembled AAV capsid, unless otherwise specified. For example, vp1 proteins may be a subpopulation of vp proteins; vp2 proteins may be a separate subpopulation of vp proteins, and vp3 are yet a further subpopulation of vp proteins in an assembled AAV capsid. In another example, vp1, vp2 and vp3 proteins may contain subpopulations having different modifications, e.g., at least one, two, three or four highly deamidated asparagines, e.g., at asparagine-glycine pairs. Unless otherwise specified, highly deamidated refers to at least 45% deamidated, at least 50% deamidated, at least 60% deamidated, at least 65% deamidated, at least 70%, at least 75%, at least 80%, at least 85%, at least 90%, at least 95%, 97%, 99%, up to about 100% deamidated, 50% to 100% deamidated, 70% to 100% deamidated, 75% to 100% deamidated, or 70% to 90% deamidated at a referenced amino acid position, as compared to the predicted amino acid sequence at the reference amino acid position. Such percentages may be determined using 2D-gel, mass spectrometry techniques, or other suitable techniques.

As used herein, a “stock” of rAAV refers to a population of rAAV. Despite heterogeneity in their capsid proteins due to deamidation, rAAV in a stock are expected to share an identical vector genome. A stock can include rAAV having capsids with, for example, heterogeneous deamidation patterns characteristic of the selected AAV capsid proteins and a selected production system. The stock may be produced from a single production system or pooled from multiple runs of the production system. A variety of production systems, including but not limited to those described herein, may be selected. See, e.g., WO 2019/168961, published Sep. 6, 2019, including Table G providing the deamidation pattern for AAV9 and WO 2020/160582, filed Sep. 7, 2018. See, also, e.g., WO 2020/223231, published Nov. 5, 2020 (rh91, including table with deamidation pattern), U.S. Provisional Patent Application No. 63/065,616, filed Aug. 14, 2020, and U.S. Provisional Patent Application No. 63/109,734, filed Nov. 4, 2020, and International Patent Application No. PCT/US21/45945, filed Aug. 13, 2021, which are all incorporated herein by reference in its entirety.

The compositions described herein may be used in a regimen involving co-administration of other active agents. Any suitable method or route can be used to administer such other agents. Routes of administration include, for example, systemic, oral, intravenous, intraperitoneal, subcutaneous, or intramuscular administration. Optionally, the AAV compositions described herein may also be administered by one of these routes.

The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

The term “heterologous” when used with reference to a protein or a nucleic acid indicates that the protein or the nucleic acid comprises two or more sequences or subsequences which are not found in the same relationship to each other in nature. For instance, the nucleic acid is typically recombinantly produced, having two or more sequences from unrelated genes arranged to make a new functional nucleic acid. For example, in one embodiment, the nucleic acid has a promoter from one gene arranged to direct the expression of a coding sequence from a different gene. Thus, with reference to the coding sequence, the promoter is heterologous.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”-containing only the transgene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

A “recombinant AAV” or “rAAV” is a DNAse-resistant viral particle containing two elements, an AAV capsid and a vector genome containing at least non-AAV coding sequences packaged within the AAV capsid. In certain embodiments, the capsid contains about 60 proteins composed of vp1 proteins, vp2 proteins, and vp3 proteins, which self-assemble to form the capsid. Unless otherwise specified, “recombinant AAV” or “rAAV” may be used interchangeably with the phrase “rAAV vector”. The rAAV is a “replication-defective virus” or “viral vector”, as it lacks any functional AAV rep gene or functional AAV cap gene and cannot generate progeny. In certain embodiments, the only AAV sequences are the AAV inverted terminal repeat sequences (ITRs), typically located at the extreme 5′ and 3′ ends of the vector genome in order to allow the gene and regulatory sequences located between the ITRs to be packaged within the AAV capsid.

The term “nuclease-resistant” indicates that the AAV capsid has assembled around the expression cassette which is designed to deliver a transgene to a host cell and protects these packaged genomic sequences from degradation (digestion) during nuclease incubation steps designed to remove contaminating nucleic acids which may be present from the production process.

As used herein, the term “host cell” may refer to the packaging cell line in which the rAAV is produced from the plasmid. In the alternative, the term “host cell” may refer to the target cell in which expression of the transgene is desired.

As used herein, a “vector genome” refers to the nucleic acid sequence packaged inside the rAAV capsid which forms a viral particle. Such a nucleic acid sequence contains AAV inverted terminal repeat sequences (ITRs). In the examples herein, a vector genome contains, at a minimum, from 5′ to 3′, an AAV 5′ ITR, expression cassette comprising coding sequence(s) (i.e., transgene(s)), and an AAV 3′ ITR. In certain embodiments, the ITRs are from AAV2, a different source AAV than the capsid, or other than full-length ITRs may be selected. In certain embodiments, the ITRs are from the same AAV source as the AAV which provides the rep function during production or a trans-complementing AAV. Further, other ITRs, e.g., self-complementary (scAAV) ITRs, may be used. Both single-stranded AAV and self-complementary (sc) AAV are encompassed with the rAAV. The transgene is a nucleic acid coding sequence, heterologous to the vector sequences, which encodes a polypeptide, protein, functional RNA molecule (e.g., miRNA, miRNA inhibitor) or other gene product, of interest. The nucleic acid coding sequence is operatively linked to regulatory components in a manner which permits transgene transcription, translation, and/or expression in a cell of a target tissue. Suitable components of a vector genome are discussed in more detail herein. In one example, a “vector genome” contains, at a minimum, from 5′ to 3′, a vector-specific sequence, a nucleic acid sequence encoding protein of interest operably linked to regulatory control sequences (which direct their expression in a target cell), where the vector-specific sequence may be a terminal repeat sequence which specifically packages the vector genome into a viral vector capsid or envelope protein. For example, AAV inverted terminal repeats are utilized for packaging into AAV and certain other parvovirus capsids.

As used herein, “operably linked” sequences include both expression control sequences that are contiguous with the gene of interest and expression control sequences that act in trans or at a distance to control the gene of interest.

In certain embodiments, non-viral genetic elements used in manufacture of a rAAV, will be referred to as vectors (e.g., production vectors). In certain embodiments, these vectors are plasmids, but the use of other suitable genetic elements is contemplated. Such production plasmids may encode sequences expressed during rAAV production, e.g., AAV capsid or rep proteins required for production of a rAAV, which are not packaged into the rAAV. Alternatively, such a production plasmid may carry the vector genome which is packaged into the rAAV.

As used herein, a “parental capsid” refers to a non-mutated, non-engineered or a non-modified capsid selected from parvovirus or other viruses (e.g., AAV, adenovirus, HSV, RSV, etc.). In certain embodiments, the parental capsid includes any naturally occurring AAV capsids comprising a wild-type genome encoding for capsid proteins (i.e., vp proteins), wherein the capsid proteins direct the AAV transduction and/or tissue-specific tropism. In some embodiments, the parent capsid is selected from AAV which natively targets muscle cell. In other embodiments, the parental capsid is selected from AAV which do not natively target muscle cell.

As used herein, the terms “target cell” and “target tissue” can refer to any cell or tissue which is intended to be transduced by the subject AAV vector. The term may refer to any one or more of muscle, liver, lung, airway epithelium, central nervous system, neurons, eye (ocular cells), or heart. In one embodiment, the target tissue is muscle tissue. In certain embodiments, the target cell is one or more muscle cell type (e.g., cardio muscle cell, gastrocnemius muscle cell, deltoid muscle cell, a soleus muscle cell, a biceps brachii muscle cell or a diaphragm muscle cell).

As used herein, a “cardiac cell” refers to general cardiac tissue cells including but not limited to heart cells, cardiac muscle cells (cardiomyocyte), conduction cells, fibroblasts, endothelial cells, smooth muscle cells and peri-vascular cells.

As used herein, a “variant capsid” or a “variant AAV” or “variant AAV capsid” refers to a modified capsid, engineered capsid or a mutated capsid, wherein the capsid protein comprises an insertion of a tissue-specific targeting peptide, wherein modified insert is not a naturally occurring mutant.

As used herein, an “expression cassette” refers to a nucleic acid molecule which comprises a biologically useful nucleic acid sequence (e.g., a gene cDNA encoding a protein, enzyme or other useful gene product, mRNA, etc.) and regulatory sequences operably linked thereto which direct or modulate transcription, translation, and/or expression of the nucleic acid sequence and its gene product. As used herein, “operably linked” sequences include both regulatory sequences that are contiguous or non-contiguous with the nucleic acid sequence and regulatory sequences that act in trans or cis nucleic acid sequence. Such regulatory sequences typically include, e.g., one or more of a promoter, an enhancer, an intron, a Kozak sequence, a polyadenylation sequence, and a TATA signal. The expression cassette may contain regulatory sequences upstream (5′ (5′) to) of the gene sequence, e.g., one or more of a promoter, an enhancer, an intron, etc., and one or more of an enhancer, or regulatory sequences downstream (3′ (3′) to) a gene sequence, e.g., 3′ untranslated region (3′ UTR) comprising a polyadenylation site, among other elements. In certain embodiments, the regulatory sequences are operably linked to the nucleic acid sequence of a gene product, wherein the regulatory sequences are separated from nucleic acid sequence of a gene product by an intervening nucleic acid sequences, i.e., 5′-untranslated regions (5′UTR). In certain embodiments, the expression cassette comprises nucleic acid sequence of one or more of gene products. In some embodiments, the expression cassette can be a monocistronic or a bicistronic expression cassette. In other embodiments, the term “transgene” refers to one or more DNA sequences from an exogenous source which are inserted into a target cell. Typically, such an expression cassette can be used for generating a viral vector and contains the coding sequence for the gene product described herein flanked by packaging signals of the vector genome and other expression control sequences such as those described herein. In certain embodiments, a vector genome may contain two or more expression cassettes.

The term “translation” in the context of the present invention relates to a process at the ribosome, wherein an mRNA strand controls the assembly of an amino acid sequence to generate a protein or a peptide.

The term “expression” is used herein in its broadest meaning and comprises the production of RNA or of RNA and protein. Expression may be transient or may be stable.

The term “substantial homology” or “substantial similarity,” when referring to a nucleic acid, or fragment thereof, indicates that, when optimally aligned with appropriate nucleotide insertions or deletions with another nucleic acid (or its complementary strand), there is nucleotide sequence identity in at least about 95 to 99% of the aligned sequences. Preferably, the homology is over full-length sequence, or an open reading frame thereof, or another suitable fragment which is at least 15 nucleotides in length. Examples of suitable fragments are described herein.

The term “percent (%) identity”, “sequence identity”, “percent sequence identity”, or “percent identical” in the context of nucleic acid sequences refers to the residues in the two sequences which are the same when aligned for correspondence. The length of sequence identity comparison may be over the full-length of the genome, the full-length of a gene coding sequence, or a fragment of at least about 500 to 5000 nucleotides, is desired. However, identity among smaller fragments, e.g., of at least about nine nucleotides, usually at least about 20 to 24 nucleotides, at least about 28 to 32 nucleotides, at least about 36 or more nucleotides, may also be desired.

Percent identity may be readily determined for amino acid sequences over the full-length of a protein, polypeptide, about 32 amino acids, about 330 amino acids, or a peptide fragment thereof or the corresponding nucleic acid sequence coding sequences. A suitable amino acid fragment may be at least about 7 amino acids in length, and may be up to about 700 amino acids.

Examples of suitable fragments are described herein. By the term “highly conserved” is meant at least 80% identity, preferably at least 90% identity, and more preferably, over 97% identity. Identity is readily determined by one of skill in the art by resort to algorithms and computer programs known by those of skill in the art.

Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence.

Identity may be determined by preparing an alignment of the sequences and through the use of a variety of algorithms and/or computer programs known in the art or commercially available (e.g., BLAST, ExPASy; Clustal Omega; FASTA; using, e.g., Needleman-Wunsch algorithm, Smith-Waterman algorithm). Alignments are performed using any of a variety of publicly or commercially available Multiple Sequence Alignment Programs. Multiple sequence alignment programs are available for nucleic acid sequences. Examples of such programs include, “Clustal Omega”, “Clustal W”, “MUSCLE”, “CAP Sequence Assembly”, “BLAST”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 10.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 10.1, herein incorporated by reference. Sequence alignment programs are also available for amino acid sequences, e.g., the “Clustal Omega”, “Clustal X”, “MUSCLE”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27 (13): 2682-2690 (1999).

In certain embodiments, an effective amount may be determined based on an animal model, rather than a human patient.

As described above, the term “about” when used to modify a numerical value means a variation of ±10%, (±10%, e.g., ±1, ±2, ±3, ±4, ±5, ±6, +7, ±8, ±9, ±10, or values therebetween) from the reference given, unless otherwise specified.

In certain instances, the term “E+#” or the term “e+#” is used to reference an exponent. For example, “5E10” or “5e10” is 5×10¹⁰. These terms may be used interchangeably.

As used throughout this specification and the claims, the terms “comprise” and “contain” and its variants including, “comprises”, “comprising”, “contains” and “containing”, among other variants, is inclusive of other components, elements, integers, steps and the like. The term “consists of” or “consisting of” are exclusive of other components, elements, integers, steps and the like.

It is to be noted that the term “a” or “an”, refers to one or more, for example, “an enhancer”, is understood to represent one or more enhancer(s). As such, the terms “a” (or “an”), “one or more,” and “at least one” is used interchangeably herein.

With regard to the description of these inventions, it is intended that each of the compositions herein described, is useful, in another embodiment, in the methods of the invention. In addition, it is also intended that each of the compositions herein described as useful in the methods, is, in another embodiment, itself an embodiment of the invention.

Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

### EXAMPLES

The following examples are illustrative only and are not a limitation on the invention described herein.

There are currently no approved AAV gene therapies for cardiac disorders. One reason for this is the inherent difficulty of transducing cardiac myocytes via systemic AAV injection. Though appreciable cardiac transduction is achievable via the naturally occurring AAV variant AAV9, the specificity of this transduction is lacking. Indeed, the majority of injected vector ends up transducing the liver, and thus the higher doses of vector required for therapeutically relevant cardiac transduction risks liver damage, adverse immune responses, and potentially death. One potential solution to this problem is localized injection of gene therapy agents; however this technology is invasive and difficult to control, thus posing another set of safety risks that will require further innovation to overcome. A primary goal in the gene therapy field then is to create gene transfer agents that can specifically and non-invasively transduce the cells of the myocardium. Thus, there is an unmet clinical need for AAV vectors that can more efficiently transduce the myocardium than the current standards in the field.

Similarly, AAV9 is historically poor at transducing skeletal muscle, a great hindrance to the current efforts aimed at treating numerous genetic disorders such as Duchenne's Muscular Dystrophy. AAV vectors with enhanced skeletal muscle tropism are thus another strong target for viral vector engineering.

It has been shown that small peptide insertions into flexible loops on the surface of the AAV capsid can mediate interactions with new cellular receptors. In one case discovered at CalTech (AAV9-PHP.B), a seven amino acid peptide inserted into the HVR8 loop on AAV9 mediates interaction with Ly6a, a GPI-anchored receptor on the brain vasculature of some mouse strains. This interaction drives transport of AAV9-PHP.B across the blood brain barrier, resulting in about 50-fold higher transduction of brain cells than AAV9.

Studies were conducted to identify peptide inserts that can improve AAV9 capsid transduction of both skeletal and cardiac muscle. To accomplish this, we leveraged previous work in AAV capsid engineering that indicated the well-studied RGD peptide motif (which is a targeting motif for numerous integrins) was able to target AAV vectors to skeletal muscle (and to a lesser extent cardiac muscle) in the context of the AAV9 HVR8 loop.

We generated a library of AAV9 insertion mutants containing hundreds of thousands of RGD containing peptides, all inserted individually at the HVR8 locus (between position 588 and 589). Each variant was barcoded such that the transgene it carried was the capsid protein it was encased in, allowing for analysis of relative abundance for each variant after RNA extraction from transduced tissue. This library was injected at a high does intravenously into Rhesus macaques, and at two weeks animals were sacrificed for RNA extraction and analysis.

We then collected total RNA, enriching mRNA from it and converting that mRNA into cDNA, followed by next generation sequencing of the resultant cDNA library. Thus, by sequencing all expressed genomes in both cardiac and skeletal muscle, we were able to pick hits for validation in a second, less noisy and more targeted library experiment. Variants were selected for high levels of enrichment (tissue rpm/injected vector library rpm) across a range of vector library representations. Additionally, some vectors from a separate screen in NHPs (driven by the synapsin promoter but still expressing in heart) were added to the second round screen as well. The second round library also included a number of important controls. First were AAV9-PHP.B (insert TLAVPFK (SEQ ID NO: 37)) and a related insert (TLAGPFK (SEQ ID NO: 38)), both of which perform similarly to AAV9 in the heart despite having an insert at site 588. We also included a number of previously reported vectors with known skeletal muscle tropism as positive controls. Each variant was coded for with three different synonymous codons, to give better confidence in performance metrics based on clustering of synonymous variants. From this secondary screen we were able to identify a large number of variants with 2-22× increase in both cardiac and skeletal muscle transduction.

### Example 1. Production of rAAV Comprising a Gene (Protein) of Interest

In the studies herein, engineered rAAVs comprising engineered AAV capsids comprising exogenous targeting peptides were generated and comparative studies were performed. In some cases, a rAAV comprising aGFP gene were generated and used to evaluate rAAV transduction and transgene expression. In some cases, a rAAV comprising a test gene X (TGX, wherein X is 1, 2, 3, etc.) were generated and used to evaluate rAAV transduction and gene expression.

The rAAV were generated using triple transfection techniques, utilizing (1) a cis plasmid encoding AAV2 rep proteins and the AAV9 VP 1 cap gene, (2) a cis plasmid comprising adenovirus helper genes not provided by the packaging cell line which expresses adenovirus Ela, and (3) a trans plasmid containing the vector genome for packaging in the AAV capsid. See, e.g., US 2020/0056159. The trans plasmid is designed to contain the vector genome comprising transgene of interest (e.g., GFP). The vector genome contains an AAV 5′ inverted terminal repeat (ITR) and an AAV 3′ ITR at the extreme 5′ and 3′ end, respectively. The ITRs flank the sequences of the expression cassette packaged into the AAV capsid which have sequences encoding protein of interest. The expression cassette further comprises regulatory sequences operably linked to the protein coding sequences, the regulatory control sequence of which include at least one or more of promoter, enhancer, polyA sequence.

The nucleic acid molecule comprising the vector genome packaged in the capsid contains an AAV2-5′ inverted terminal repeat (ITR) and an AAV2-3′ ITR at the extreme 5′ and 3′ end of the vector genome, respectively. The vector genome further comprises between the ITRs, the expression cassette packaged into the AAV capsid which have sequences encoding a Test Transgene 2 (TT2). The expression cassette further comprises regulatory sequences operably linked to the engineered coding sequences, the regulatory control sequence of which includes an optional enhancer, a promoter, an optional intron (e.g., a hybrid CB7 promoter comprising CMV IE enhancer, a chicken beta actin promoter, a chicken beta actin intron and optional spacer sequences), wherein the expression cassette further includes a rabbit beta-globin (RBG) polyA.

Productivity and recovery summary of the production (e.g., using iCellis 200m2 with downstream chromatography purification) of rAAV comprising TT2 is summarized in Table A (recovery summary), Table B (productivity), and Table C (enrichment summary) below.

TABLE A

| | AAVhu68 | | AAV9-AVIRGDV | | AAV9-IIRGDPA | |
|---|---|---|---|---|---|---|
| | Total GC | Recovery | Total GC | Recovery | Total GC | Recovery |
| Clarified | 3.68 × 10¹⁵ | | 4.59 × 10¹⁵ | | 4.47 × 10¹⁵ | |
| Bulk | | | | | | |
| Harvest | | | | | | |
| Downstream | 3.14 × 10¹⁵ | 85% | 4.6 × 10¹⁵ | >99% | 4.57 × 10¹⁵ | >99% |
| (DS) Step A | | | | | | |
| DS Step B | 1.90 × 10¹⁵ | 52% | 2.45 × 10¹⁵ | 53% | 2.07 × 10¹⁵ | 46% |
| DS Step C | 1.27 × 10¹⁵ | 35% | 1.63 × 10¹⁵ | 36% | 1.4 × 10¹⁵ | 31% |
| BDS | 1.30 × 10¹⁵ | 35% | 1.30 × 10¹⁵ | 28% | 1.25 × 10¹⁵ | 28% |

TABLE B

| | AAVhu68 | AAV9-VIRGDV | AAV9-IRGDPA |
|---|---|---|---|
| BDS Pro- | 6.50 × 10¹² | 6.50 × 10¹² | 6.25 × 10¹² |
| ductivity | | | |

TABLE C

| | AAVhu68 | AAV9-AVIRGDV | AAV9-IIRGDPA |
|---|---|---|---|
| DS Step B | 19%:8%:73% | 24%:5%:71 | 27%:0%:73% |
| Full: | | | |
| Partial: | | | |
| Empty ratio | | | |
| DS Step C | 77%:0%:23% | 77%:11%:12% | 79%: 10%:11% |

These results show that productivity and enrichment values were comparable among the AAVhu68, AAV9-IIRGDPA, and AAV9-AVIRGDV capsids in the yield and manufacturability assessment.

Additionally, we performed mass spectrometry analysis (N=1) of deamidation of the rAAV capsid comprising the AAV9 mutant AVIRGDV. These results are shown in Table D.

TABLE D

| | |
|---|---|
| % Coverage | 93.6 |
| N57 + Deamidation | 82.5 |
| N66 + Deamidation | 4.4 |
| N94 + Deamidation | 4.6 |
| ~N304 + Deamidation | 8.6 |
| N329 + Deamidation | 90.6 |
| N452 + Deamidation | 96.1 |
| N512 + Deamidation | 90.1 |
| ~N562 + Deamidation | 2.6 |
| N717 + Deamidation | 2.9 |
| S149 + Phosphorylation | 14.5 |
| M436 + Oxidation | 3.6 |
| ~M518 + Oxidation | 6.5 |
| ~M524 + Oxidation | 4.4 |
| M559 + Oxidation | 6.1 |
| ~M605 + Oxidation | 12.2 |
| M642 + Oxidation | 0.7 |
| ~M647 + Oxidation | 0.8 |

As can be seen, the AAV9-AVIRGDV mutant retains the deamidation pattern of AAV9 [see, US 2020/0407750 A1, incorporated herein by reference], in that it is highly deamidated (at least 50% deamidation) at positions N57, N329, N452 and N512.

### Example 2: Improving AAV9 for Heart Transduction

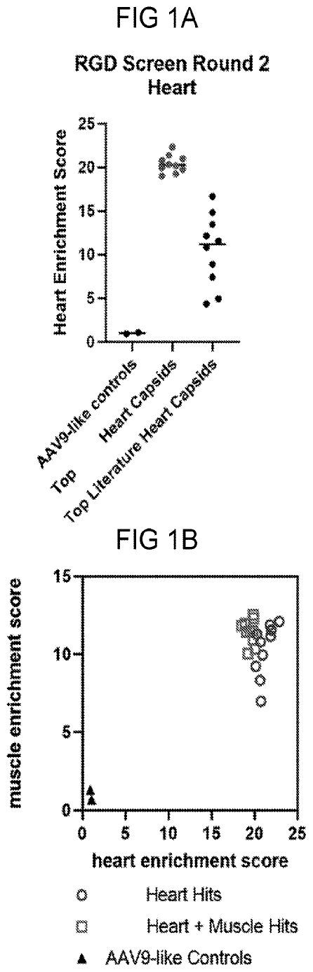

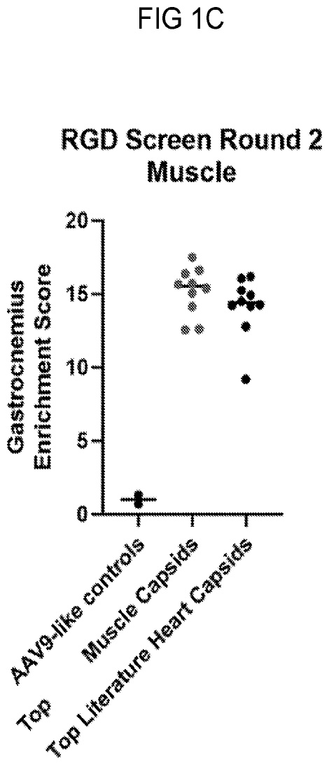

In this study, we designed and generated an AAV9 library to include a comprehensive collection of all possible RGD 7-mer peptides (inserted in HVRVIII (HVR8) region of AAV9 capsid), which was used for selection by 2-rounds of selection in NHP (dose 5×10¹³ GC/kg, with heart and gastrocnemius (muscle) tissues collected on day 14) with focusing on the heart and muscle (i.e., distinct integrin makeup heart vs. muscle). In Round 2 of the NHP heart selection, a mini library design with embedded control sequences, AAV9-like negative control vectors, and best reported RGD variants from literature as positive controls were used. FIG. 1A shows plotted heart enrichment scores from the RGD screen round 2 for top performing heart candidates in comparison to the top literature capsids. Additionally, we observed that many RGD hits had excellent heart and skeletal muscle transduction profiles. FIG. 1B shows muscle enrichment scores from the RGD screen round 2 for top performing heart and muscle candidates in comparison to the top heart candidates. In round 2 of the NHP muscle selection, unique RGD insert variants with muscle-targeting activity were observed. FIG. 1C shows gastrocnemius enrichment scores from the RGD screen round 2 for top performing muscle candidates in comparison to the top literature heart candidates.

Next, we used vector genome barcoding to evaluate over 30 engineered capsids, in comparison to controls, in a single animal. First, we looked at individual vector production at pre-clinical scale, then we evaluate top heart and muscle capsid variants individually at full dose (5E12 (5×10¹²) GC/kg).

Overall, a comprehensive screen of AAV9-RGD variants in NHP yielded hits with enhanced heart and skeletal muscle transduction versus AAV9 capsids. Top RGD variants are summarized in Table 1 below.

TABLE 1

| | | | | | | | SEQ ID |
|---|---|---|---|---|---|---|---|
| AA₁ | AA₂ | AA₃ | AA₄ | AA₅ | AA₆ | AA₇ | NO: |
| I | I | R | G | D | P | A | 1 |
| A | V | I | R | G | D | V | 2 |
| I | V | R | G | D | P | A | 8 |
| M | I | R | G | D | V | K | 9 |
| A | Q | H | R | G | D | V | 10 |
| V | S | R | G | D | P | N | 11 |
| V | S | R | G | D | P | A | 12 |
| P | L | V | R | G | D | I | 13 |
| P | Y | V | R | G | D | P | 14 |
| V | V | R | G | D | P | Q | 15 |
| P | T | R | G | D | V | K | 16 |
| V | V | Q | R | G | D | V | 17 |
| Q | H | R | G | D | T | Q | 18 |
| Q | I | R | G | D | L | R | 19 |
| R | G | D | Y | A | Q | V | 20 |
| I | G | R | G | D | P | N | 21 |
| R | G | D | L | H | G | Y | 22 |
| R | G | D | Y | S | T | M | 23 |
| P | Y | Q | R | G | D | H | 24 |

Next, we performed a barcode cardiac vector study that included the top 9 heart vectors, top 10 gastrocnemius vectors, MyoAAV (literature control), AAV9 (negative control), Gal vectors (modified amino acids in the Gal-binding pocket of AAV capsid), and 5 W503A. Vectors were administered to NHP (n=1) at a dose of 1×10¹³ GC/kg, with time points observed at D14. Table 2 shows results of the barcode study with normalized RNA values. Table 3 shows results of the barcode study with normalized DNA values. All values were normalized to AAV9. MYOAAV4E is a previously published vector with good performance in heart and muscle.

TABLE 2

| | | Normalized RNA | | | |
|---|---|---|---|---|---|
| SEQ ID NO | Vector | L ventricle | R Ventricle | Liver | Gastroc. |
| 25 | AAV9 | 1.00 | 1.00 | 1.00 | 1.00 |
| 1 | IIRGDPA | 1.21 | 1.27 | 0.54 | 1.08 |
| 2 | AVIRGDV | 1.94 | 1.89 | 1.07 | 2.03 |
| 8 | IVRGDPA | 0.36 | 0.36 | 0.30 | 0.25 |
| 9 | MIRGDVK | 1.80 | 2.04 | 1.23 | 1.70 |
| 10 | AQHRGDV | 1.04 | 1.14 | 0.99 | 1.41 |
| 11 | VSRGDPN | 1.82 | 1.83 | 1.84 | 1.89 |
| 12 | VSRGDPA | 1.92 | 2.02 | 1.32 | 1.91 |
| 13 | PLVRGDI | 1.43 | 1.41 | 1.61 | 1.57 |
| 14 | PYVRGDP | 2.13 | 2.28 | 1.59 | 2.29 |
| 15 | VVRGDPQ | 1.62 | 1.70 | 1.85 | 1.65 |
| 16 | PTRGDLK | 1.75 | 1.96 | 1.17 | 1.71 |
| 17 | VVQRGDV | 1.58 | 1.61 | 2.15 | 1.57 |
| 18 | QHRGDTQ | 1.74 | 1.97 | 1.81 | 1.72 |
| 19 | QIRGDLR | 2.50 | 3.02 | 1.85 | 2.44 |
| 20 | RGDYAQV | 2.29 | 2.52 | 0.96 | 5.98 |
| 21 | IGRGDPN | 1.16 | 1.18 | 0.88 | 1.19 |
| 22 | RGDLHGY | 2.50 | 3.68 | 1.05 | 5.88 |
| 23 | RGDYSTM | 2.60 | 3.36 | 0.84 | 6.43 |
| 128 | RGDYREL | 2.16 | 2.91 | 0.63 | 4.46 |
| | MyoAAV_4E | 5.04 | 6.47 | 1.61 | 12.63 |
| | W503A | 0.12 | 0.12 | 0.00 | 0.27 |
| | IIRGDPA-HRH | 0.21 | 0.21 | 0.16 | 0.31 |
| | IIRGDPA-VRH | 0.64 | 0.68 | 0.54 | 0.70 |
| | RGDYSQI-HRH | 0.77 | 0.94 | 0.00 | 1.80 |
| | RGDYSQI-VRH | 2.07 | 2.68 | 0.17 | 4.46 |
| | AAV9 SRH | 0.45 | 0.39 | 1.33 | 0.48 |
| | AAV9 VRH | 0.43 | 0.45 | 0.93 | 0.52 |
| | AAV9 HRH | 0.40 | 0.42 | 0.57 | 0.57 |

TABLE 3

| | | Normalized DNA | | | |
|---|---|---|---|---|---|
| SEQ ID NO | Vector | L ventricle | R Ventricle | Liver | Gastroc. |
| 25 | AAV9 | 1.00 | 1.00 | 1.00 | 1.00 |
| 1 | IIRGDPA | 0.67 | 0.63 | 0.49 | 0.43 |
| 2 | AVIRGDV | 1.17 | 1.21 | 1.04 | 0.16 |
| 8 | IVRGDPA | 0.22 | 0.27 | 0.14 | 0.16 |
| 9 | MIRGDVK | 0.61 | 0.79 | 1.08 | 0.42 |
| 10 | AQHRGDV | 0.70 | 0.81 | 0.73 | 0.55 |
| 11 | VSRGDPN | 1.23 | 1.09 | 1.11 | 0.85 |
| 12 | VSRGDPA | 1.16 | 1.26 | 1.13 | 0.94 |
| 13 | PLVRGDI | 0.98 | 1.05 | 1.00 | 0.84 |
| 14 | PYVRGDP | 1.32 | 1.46 | 1.02 | 1.06 |
| 15 | VVRGDPQ | 1.06 | 1.06 | 1.02 | 0.88 |
| 16 | PTRGDLK | 0.61 | 0.82 | 1.31 | 0.57 |
| 17 | VVQRGDV | 1.00 | 1.17 | 0.95 | 0.82 |
| 18 | QHRGDTQ | 0.98 | 1.01 | 1.04 | 0.82 |
| 19 | QIRGDLR | 0.80 | 1.10 | 1.31 | 0.55 |
| 20 | RGDYAQV | 1.27 | 1.29 | 0.64 | 1.36 |
| 21 | IGRGDPN | 0.70 | 0.71 | 0.65 | 0.50 |
| 22 | RGDLHGY | 1.24 | 1.46 | 0.66 | 1.30 |
| 23 | RGDYSTM | 1.58 | 1.65 | 0.85 | 1.76 |
| 128 | RGDYREL | 1.19 | 1.25 | 0.65 | 1.08 |
| | MyoAAV_4E | 1.74 | 1.94 | 1.03 | 1.86 |
| | W503A | 0.05 | 0.08 | 0.01 | 0.05 |
| | IIRGDPA-HRH | 0.11 | 0.16 | 0.16 | 0.13 |
| | IIRGDPA-VRH | 0.29 | 0.39 | 0.60 | 0.20 |
| | RGDYSQI-HRH | 0.32 | 0.30 | 0.06 | 0.28 |
| | RGDYSQI-VRH | 0.76 | 0.89 | 0.30 | 0.71 |
| | AAV9 SRH | 0.30 | 0.42 | 0.62 | 0.47 |
| | AAV9 VRH | 0.40 | 0.51 | 0.69 | 0.38 |
| | AAV9 HRH | 0.29 | 0.38 | 0.47 | 0.27 |

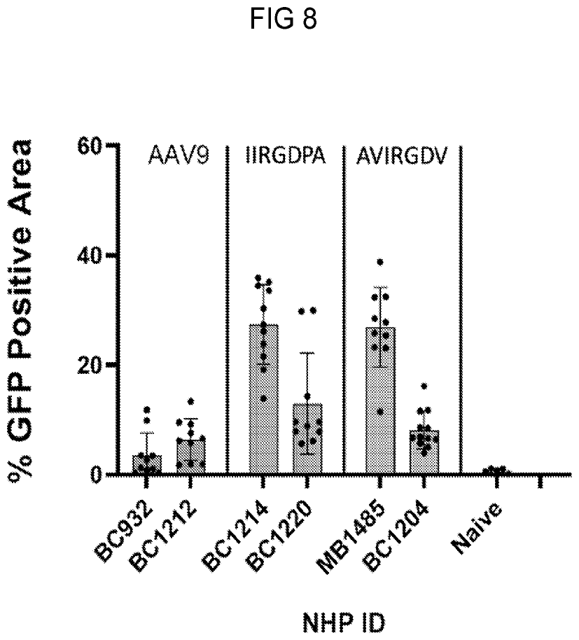

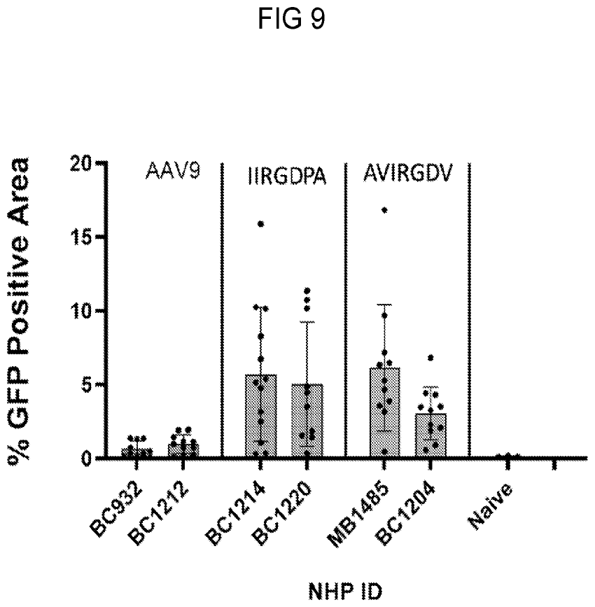

Next, we performed testing of individual heart capsids for cardiac transduction, in which AAV9, AAV9-IIRGDPA, and AAV9-AVIRGDV vectors were administered to NHPs (n=8) at a dose of 1×10¹³ GC/kg. On D13-15 tissues samples were collected and histological analyses were performed, including staining, imaging, and quantification (i.e., computer analyzed). Representative IHC microscopy images of heart left longitudinal tissue samples (dark brown in IHC, positive for GFP) were taken (data not shown). FIG. 8 shows percent GFP-positive area as quantified from IHC analysis of heart (longitudinal) tissue samples. Representative IHC microscopy images of heart left transverse tissue samples (dark brown in IHC, positive for GFP) were taken (data not shown). FIG. 9 shows percent GFP-positive area as quantified from IHC analysis of heart (transverse) tissue samples. Table 4 shows a summary of the values calculated from IHC analysis (% positive cells for each slide per NHP).

TABLE 4

| | Left Ventricle | | | Right Ventricle | | |
|---|---|---|---|---|---|---|
| | Longitudinal | | Transverse | Longitudinal | Transverse | |
| AAV9 | 1.00 | | 1.00 | 1.00 | 1.00 | |
| IIRGDPA | 4.14 | 4.1 | 8.38 | 3.95 | 6.59 | 6.7 |
| AVIRGDV | 3.33 | 3.5 | 6.56 | 3.76 | 5.20 | 5.8 |

Next, we examined RNA (expression PCR), DNA (biodistribution PCR), and protein expression (ELISA) biodistribution in the collected tissue samples following administration of the AAV9, AAV9-IIRGDPA, and AAV9-AVIRGDV vectors. DNA and RNA analyses were performed on 3-4 pieces of tissue per NHP. There were 2 NHPs per vector. Left and right ventricles of the heart were examined separately.

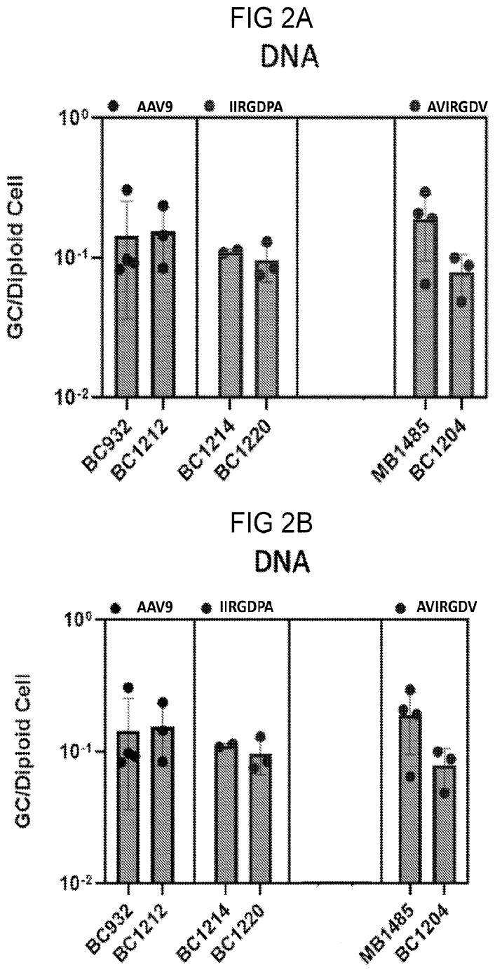

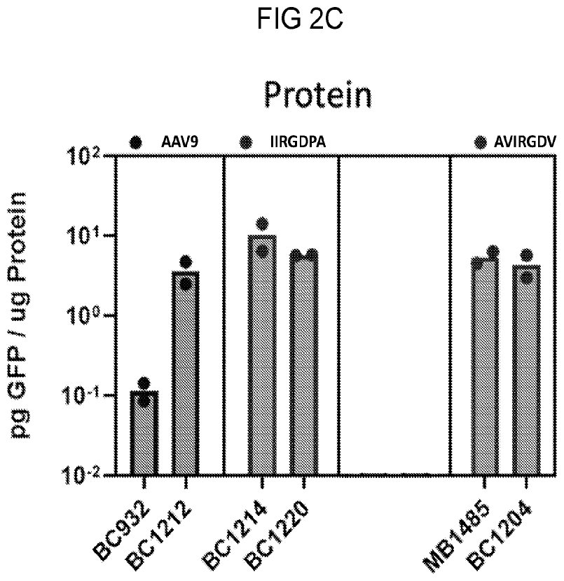

FIGS. 2A to 2C show RNA, DNA, and protein expression levels in the collected tissue samples of heart left ventricle following administration of the AAV9, AAV9-IIRGDPA, and AAV9-AVIRGDV vectors. FIG. 2A shows DNA levels, plotted as GC/diploid cell. FIG. 2B shows RNA levels, plotted as copy number (#)/100 ng RNA. FIG. 2C shows protein expression levels, plotted as pg GFP/μg protein. Table 5 shows a summary of quantified values.

TABLE 5
Left Ventricle
DNA

| AAV9 | IIRGDPA | AVIRGDV |
|---|---|---|
| 0.96 | 0.74 | 1.27 |
| 1.04 | 0.65 | 0.53 |
| 1.00 | 0.69 | 0.90 |

RNA

| AAV9 | IIRGDPA | AVIRGDV |
|---|---|---|
| 0.15 | 5.90 | 2.55 |
| 1.85 | 2.63 | 0.79 |
| 1.00 | 4.26 | 1.67 |

Protein

| AAV9 | IIRGDPA | AVIRGDV |
|---|---|---|
| 0.06 | 5.54 | 2.90 |
| 1.94 | 3.07 | 2.33 |
| 1.00 | 4.30 | 2.62 |

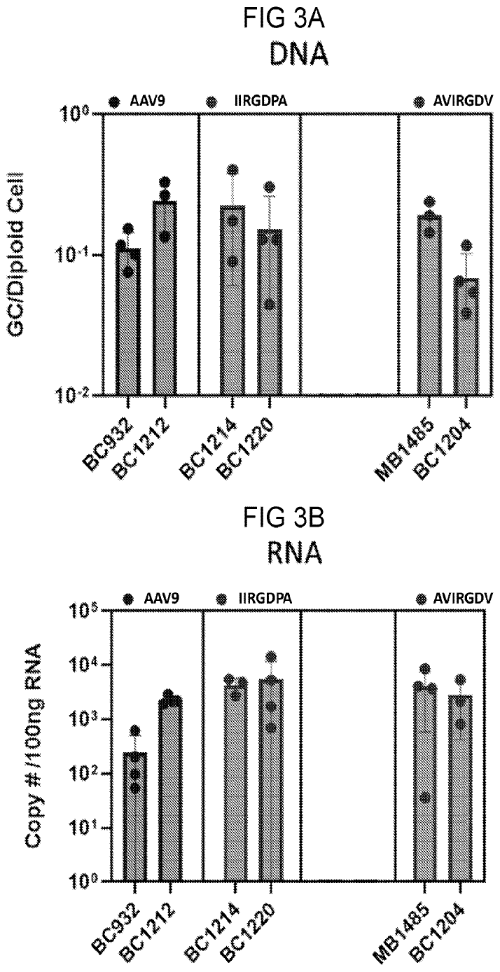

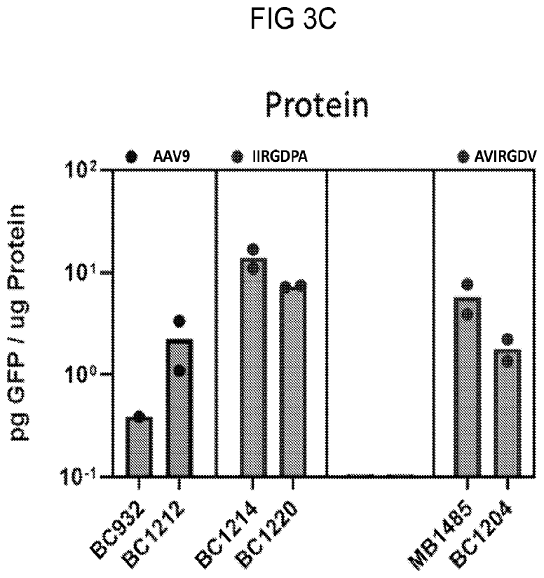

FIGS. 3A to 3C show RNA, DNA, and protein expression levels in the collected tissue samples of heart right ventricle following administration of the AAV9, AAV9-IIRGDPA, and AAV9-AVIRGDV vectors. FIG. 3A shows DNA levels, plotted as GC/diploid cell. FIG. 3B shows RNA levels, plotted as copy number (#)/100 ng RNA. FIG. 3C shows protein expression levels, plotted as pg GFP/μg protein. Table 6 shows a summary of quantified values.

TABLE 6
Right Ventricle
DNA

| AAV9 | IIRGDPA | AVIRGDV |
|---|---|---|
| 0.63 | 1.25 | 1.08 |
| 1.37 | 0.85 | 0.31 |
| 1.00 | 1.05 | 0.69 |

RNA

| AAV9 | IIRGDPA | AVIRGDV |
|---|---|---|
| 0.19 | 3.42 | 3.25 |
| 1.81 | 4.35 | 2.21 |
| 1.00 | 3.88 | 2.73 |

Protein

| AAV9 | IIRGDPA | AVIRGDV |
|---|---|---|
| 0.30 | 10.61 | 4.42 |
| 1.70 | 5.59 | 1.37 |
| 1.00 | 8.10 | 2.89 |

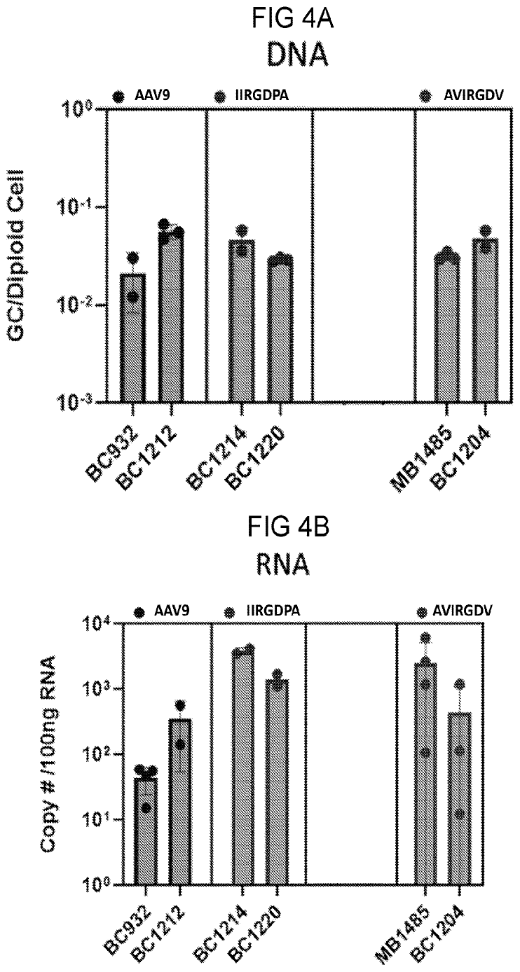

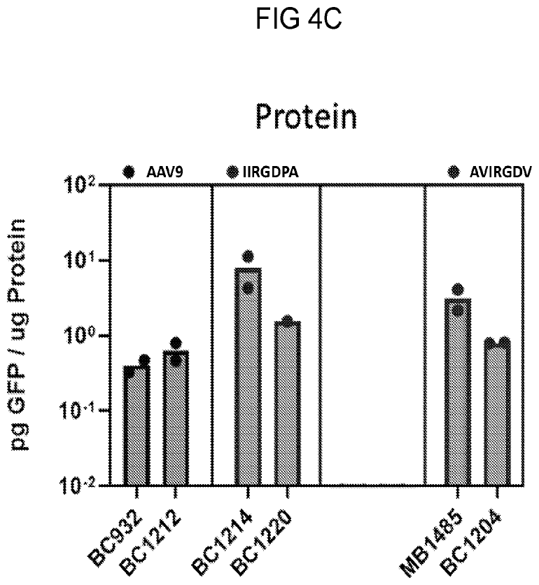

FIGS. 4A to 4C show RNA, DNA, and protein expression levels in the collected tissue samples of gastrocnemius following administration of the AAV9, AAV9-IIRGDPA, and AAV9-AVIRGDV vectors. FIG. 4A shows DNA levels, plotted as GC/diploid cell.

FIG. 4B shows RNA levels, plotted as copy number (#)/100 ng RNA. FIG. 4C shows protein expression levels, plotted as pg GFP/μg protein. Table 7 shows a summary of quantified values.

TABLE 7
Gastrocnemius
DNA

| AAV9 | IIRGDPA | AVIRGDV |
|---|---|---|
| 0.54 | 1.20 | 0.81 |
| 1.48 | 0.75 | 1.24 |
| 1.00 | 0.98 | 1.02 |

RNA

| AAV9 | IIRGDPA | AVIRGDV |
|---|---|---|
| 0.22 | 19.35 | 12.65 |
| 1.78 | 7.04 | 2.20 |
| 1.00 | 13.19 | 7.42 |

Protein

| AAV9 | IIRGDPA | AVIRGDV |
|---|---|---|
| 0.77 | 15.08 | 6.05 |
| 1.23 | 3.02 | 1.55 |
| 1.00 | 9.05 | 3.80 |

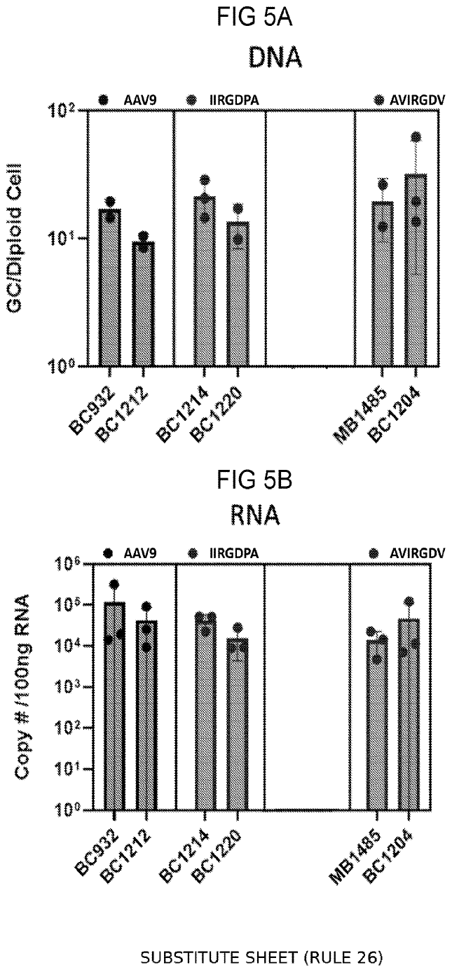

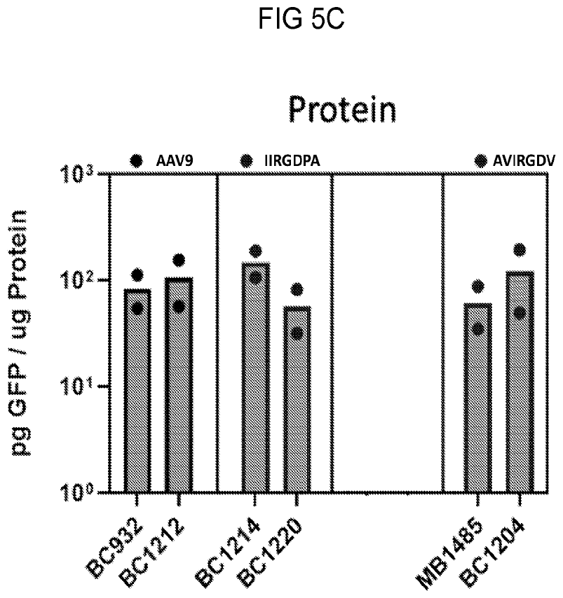

FIGS. 5A to 5C show RNA, DNA, and protein expression levels in the collected tissue samples of liver following administration of the AAV9, AAV9-IIRGDPA, and AAV9-AVIRGDV vectors. FIG. 5A shows DNA levels, plotted as GC/diploid cell. FIG. 5B shows RNA levels, plotted as copy number (#)/100 ng RNA. FIG. 5C shows protein expression levels, plotted as pg GFP/μg protein. Table 8 shows a summary of quantified values

TABLE 8
Liver
DNA

| AAV9 | IIRGDPA | AVIRGDV |
|---|---|---|
| 1.28 | 1.61 | 0.98 |
| 0.72 | 1.02 | 2.40 |
| 1.00 | 1.32 | 1.69 |

RNA

| AAV9 | IIRGDPA | AVIRGDV |
|---|---|---|
| 1.47 | 0.52 | 0.17 |
| 0.53 | 0.19 | 0.58 |
| 1.00 | 0.35 | 0.37 |

Protein

| AAV9 | IIRGDPA | AVIRGDV |
|---|---|---|
| 0.88 | 1.56 | 0.65 |
| 1.12 | 0.60 | 1.28 |
| 1.00 | 1.08 | 0.96 |

The results indicated that the AAV9-IIRGDPA capsid showed the best transduction and protein expression in heart and muscle (more specifically in left ventricle), while AAV9-AVIRGDV still performed better than AAV9. AAV9-AVIRGDV had similar or less DNA in the heart and muscle compared to AAV9.

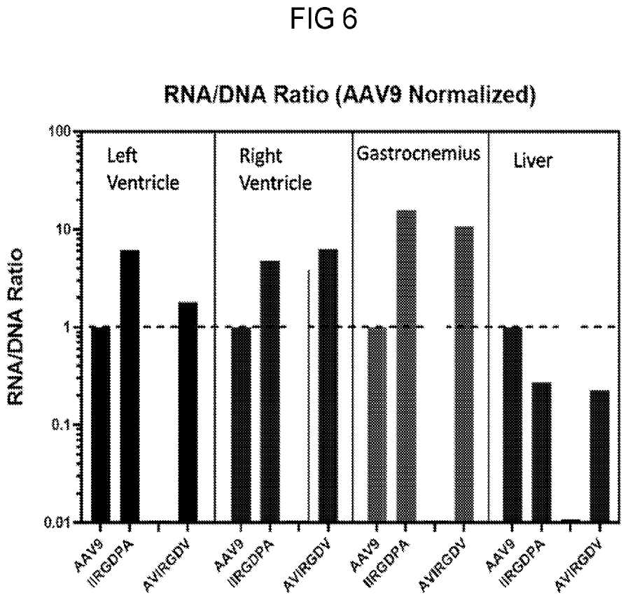

FIG. 6 shows RNA/DNA ratios (normalized to AAV9) for AAV9, AAV9-IIRGDPA, and AAV9-AVIRGDV vector biodistribution. Table 9 shows a summary of quantified values.

TABLE 9
Left Ventricle

| AAV9 | IIRGDPA | AVIRGDV |
|---|---|---|
| 0.16 | 8.18 | 2.07 |
| 1.84 | 4.19 | 1.54 |
| 1.00 | 6.19 | 1.80 |

Right Ventricle

| AAV9 | IIRGDPA | AVIRGDV |
|---|---|---|
| 0.38 | 3.35 | 3.71 |
| 1.62 | 6.27 | 8.79 |
| 1.00 | 4.81 | 6.25 |

Gastrocnemius

| AAV9 | IIRGDPA | AVIRGDV |
|---|---|---|
| 0.50 | 19.78 | 19.18 |
| 1.50 | 11.52 | 2.19 |
| 1.00 | 15.65 | 10.68 |

Liver

| AAV9 | IIRGDPA | AVIRGDV |
|---|---|---|
| 1.22 | 0.34 | 0.19 |
| 0.78 | 0.20 | 0.26 |
| 1.00 | 0.27 | 0.22 |

Tables 10A to 10D show molecular summaries for individual hits.

TABLE 10A
Left Ventricle

| | Individual | | Barcode | |
|---|---|---|---|---|
| | RNA | DNA | RNA | DNA |
| AAV9 | 1.00 | 1.00 | 1.00 | 1.00 |
| IIRGDPA | 4.26 | 0.69 | 1.21 | 0.67 |
| AVIRGDV | 1.67 | 0.90 | 1.94 | 1.17 |

TABLE 10B
Right Ventricle

| | Individual | | Barcode | |
|---|---|---|---|---|
| | RNA | DNA | RNA | DNA |
| AAV9 | 10.00 | 1.00 | 1.00 | 1.00 |
| IIRGDPA | 3.88 | 1.05 | 1.27 | 0.63 |
| AVIRGDV | 2.73 | 0.69 | 1.89 | 1.21 |

TABLE 10C
Gastrocnemius

| | Individual | | Barcode | |
|---|---|---|---|---|
| | RNA | DNA | RNA | DNA |
| AAV9 | 1.00 | 1.00 | 1.00 | 1.00 |
| IIRGDPA | 13.19 | 0.98 | 1.08 | 0.43 |
| AVIRGDV | 7.42 | 1.02 | 2.03 | 0.77 |

TABLE 10D
Liver

| | Individual | | Barcode | |
|---|---|---|---|---|
| | RNA | DNA | RNA | DNA |
| AAV9 | 1.00 | 1.00 | 1.00 | 1.00 |
| IIRGDPA | 0.35 | 1.32 | 0.54 | 0.49 |
| AVIRGDV | 0.37 | 1.69 | 1.07 | 1.04 |

Additionally, Table 11 below shows a summary of yield scores for AAV9-exogenous peptides, normalized to AAV9.

TABLE 11

| | | | | | Heart E | Gastro E | Yield |
|---|---|---|---|---|---|---|---|
| | Inclusion | Heart E | Gastro E | Yield | AAV9 | AAV9 | AAV9 |
| peptide | Criteria | Rank | Rank | score | norm | norm | norm |
| IIRGDPA | Gastro hit | 1 | 21 | 1.57 | 22.90 | 12.11 | 1.32 |
| AVIRGDV | Heart hit | 2 | 26 | 1.47 | 21.96 | 11.56 | 1.24 |
| MIRGDVK | Heart hit | 3 | 33 | 1.52 | 21.88 | 11.16 | 1.28 |
| IVRGDPA | Heart hit | 4 | 23 | 1.66 | 21.80 | 11.87 | 1.40 |
| PYVRGDP | Heart hit | 5 | 56 | 1.44 | 20.95 | 9.96 | 1.21 |
| PLVRGDI | Heart hit | 7 | 38 | 1.46 | 20.70 | 10.79 | 1.23 |
| IGRGDPN | Inf vector | 8 | 144 | 0.76 | 20.65 | 8.33 | 0.64 |
| PYQRGDH | Heart hit | 9 | 30 | 1.40 | 20.30 | 11.28 | 1.18 |
| QIRGDLR | Heart hit | 10 | 87 | 1.41 | 20.16 | 9.24 | 1.19 |

Table 12 below shoes a summary of molecular results (RNA levels, DNA levels, and GFP ELISA) of rAAV9-exogeneous peptide vector performance, as normalized to AAV9. These data represent the average of 2 NHPs and at least 5 extractions from each. AAV9-IIRGDPA had the best RNA and protein expression in both the heart and gastrocnemius. AAV9-AVIRGDV was the second best of the tested individual vectors.

TABLE 12

| | | | |
|---|---|---|---|
| Tissue | DNA | RNA | ELISA |

IIRGDPA

| | | | |
|---|---|---|---|
| Heart | 0.8 | 12.7 | 9.4 |
| Gastroc. | 2.7 | 26.7 | 27.1 |
| Liver | 1.9 | 1.0 | 1.4 |

AVIRGDV

| | | | |
|---|---|---|---|
| Heart | 1.1 | 7.0 | 3.4 |
| Gastroc. | 1.6 | 8.0 | 8.7 |
| Liver | 1.3 | 0.3 | 1.3 |

### Example 3: Evaluation of Engineered rAAV9 for Transduction

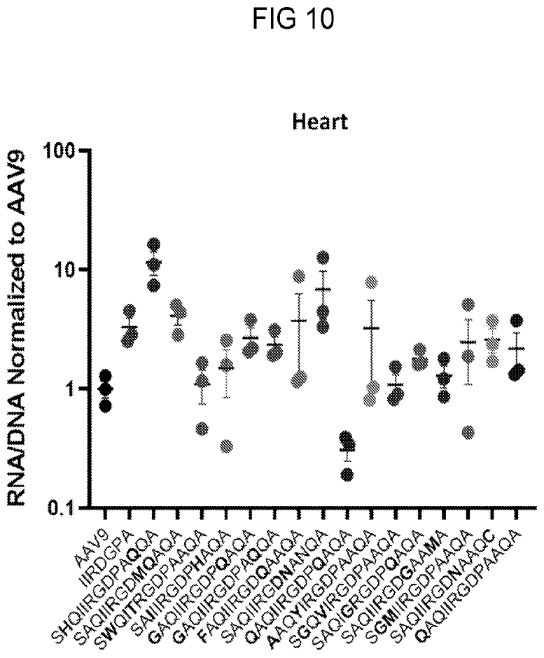

In this study, we performed testing of individual heart capsids (i.e., engineered rAAV comprising exogenous targeting peptide) for cardiac, gastrocnemius, liver cell transduction, in which AAV9, AAV9-IIRGDPA, AAV9-AVIRGDV, and AAV9-X vectors (including modified flanking regions comprising IIRGDPA or AVORGDV) were administered to mice (n=111) at a dose of 5×10¹² GC/kg. Mice were observed for 14 days, after which mice were necropsied. Heart, liver, and gastrocnemius tissues were collected for biodistribution, and histological analysis was performed. Table 13 below shows the AAV9-X variants evaluated (FIGS. 10-13).

TABLE 13

| SEQ ID NO | Peptide | FC |
|---|---|---|
| 39 | SHQIIRGDPAQQA | 2.62 |
| 40 | SAQIIRGDMQAQA | 2.51 |
| 41 | SWQITRGDPAAQA | 2.25 |
| 42 | SAIIIRGDPHAQA | 2.31 |
| 43 | GAQIIRGDPQAQA | 2.10 |
| 44 | GAQIIRGDPAQQA | 2.03 |
| 45 | FAQIIRGDQAAQA | 2.16 |
| 46 | SAQIIRGDNANQA | 2.33 |
| 47 | QAQIIRGDPQAQA | 2.14 |
| 48 | AAQYIRGDPAAQA | 2.03 |
| 49 | SGQVIRGDPAAQA | 1.95 |
| 50 | SAQIGRGDPQAQA | 1.93 |
| 51 | SAQIIRGDGAAMA | 1.92 |
| 52 | SGMIIRGDPAAQA | 1.89 |
| 53 | SAQIIRGDNAAQC | 2.28 |
| 54 | QAQIIRGDPAAQA | 1.84 |
| 55 | SAQIIRGDPAAQA | 1.59 |
| 56 | SAYAVARGDVAQA | 3.97 |
| 57 | SAQQVIRGDVGQA | 3.07 |
| 58 | SAQAGIRGDVARA | 2.08 |
| 59 | GAQAAIRGDVAQA | 2.03 |
| 60 | SWQAVVRGDVAQA | 1.99 |
| 61 | QAQAVIRGDVAQA | 1.74 |
| 62 | SAQAVIRGDVGRA | 1.89 |
| 63 | SGQAVIRGDVARA | 1.88 |
| 64 | SAYAVIRGDVAQA | 1.69 |
| 65 | SWQAAIRGDVAQA | 1.91 |
| 66 | SAQAYIRGDVAQA | 1.64 |
| 67 | SAMAVIRGDVAQA | 1.65 |
| 68 | SAHAVIRGDVAQA | 1.65 |
| 69 | SAQAVVRGDVAQA | 1.60 |
| 70 | SHQAVIRGDVAQA | 1.65 |
| 71 | SAQAVIRGDVAQA | 1.44 |

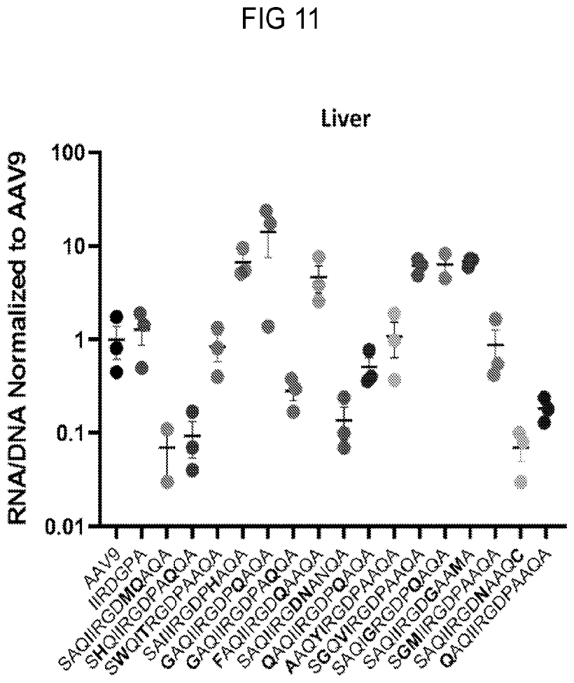

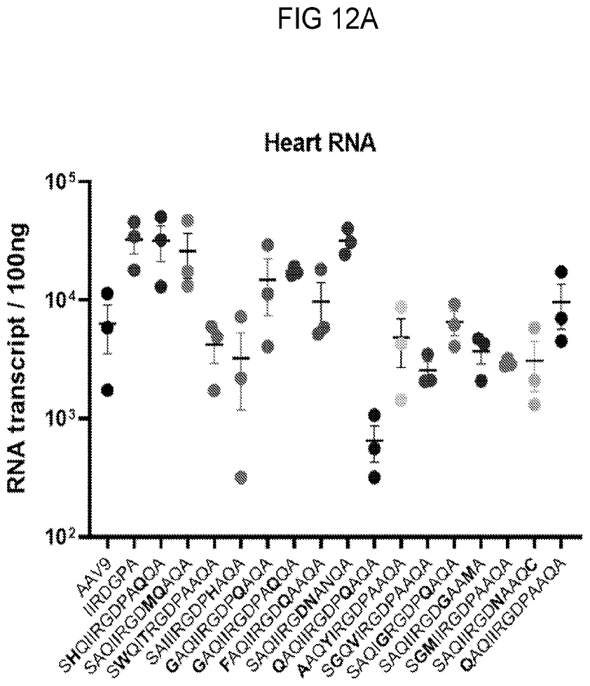

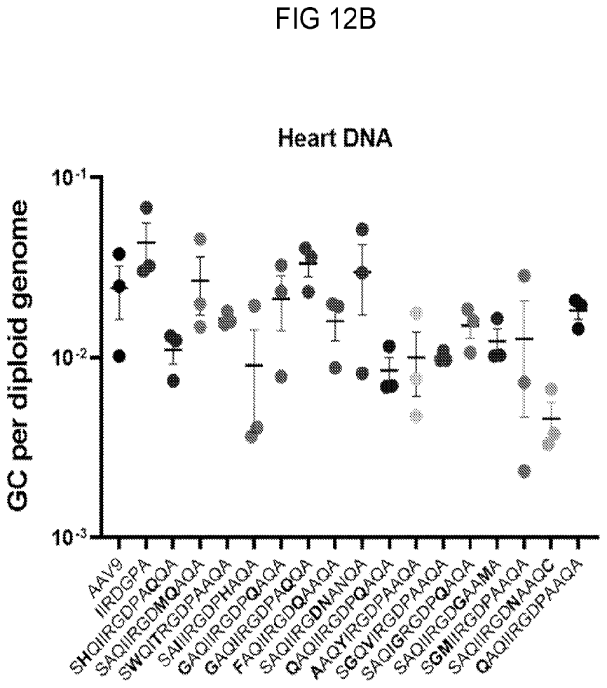

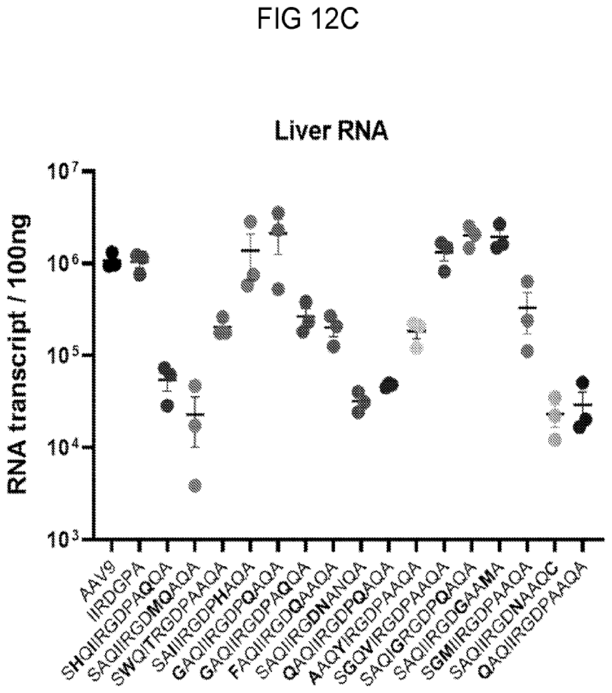

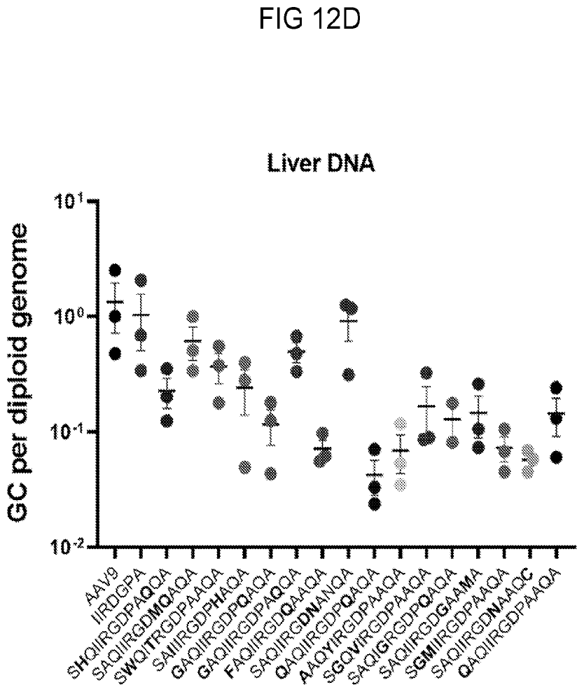

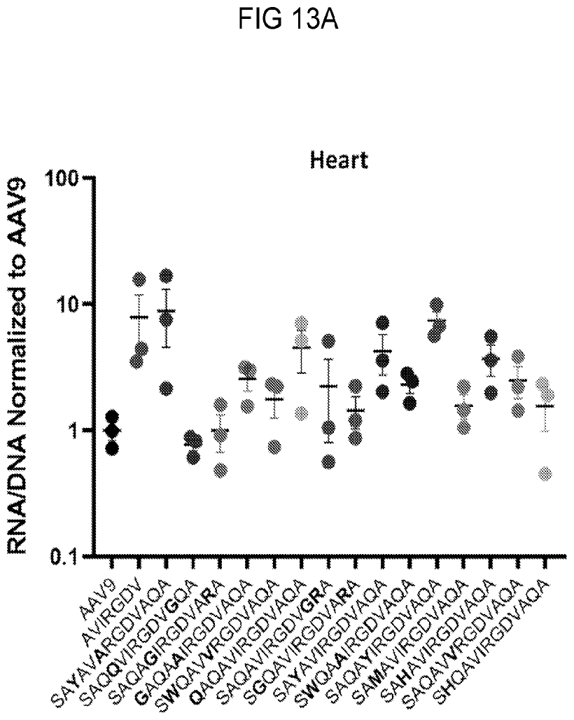

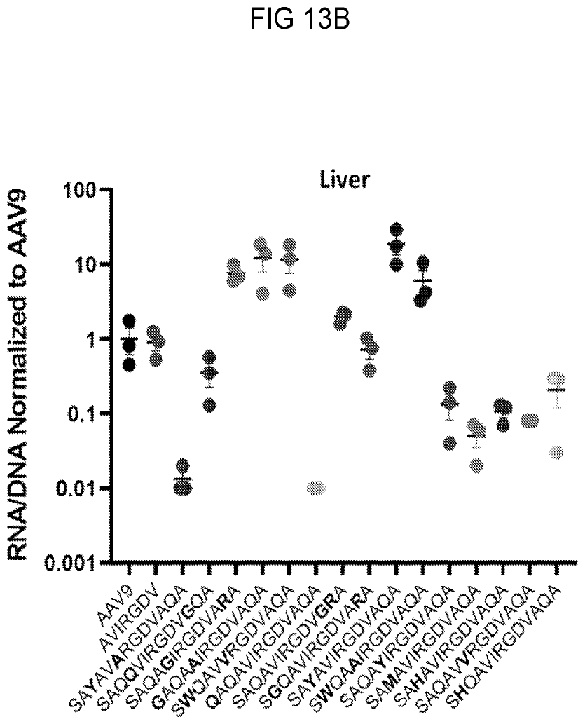

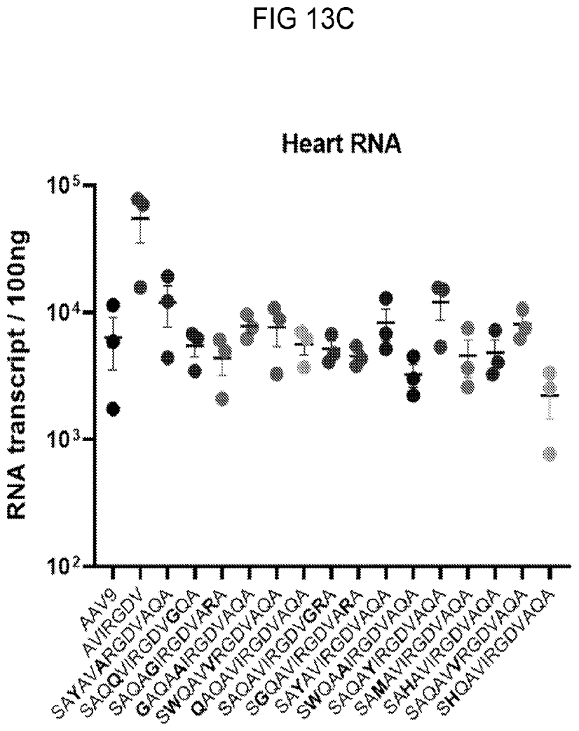

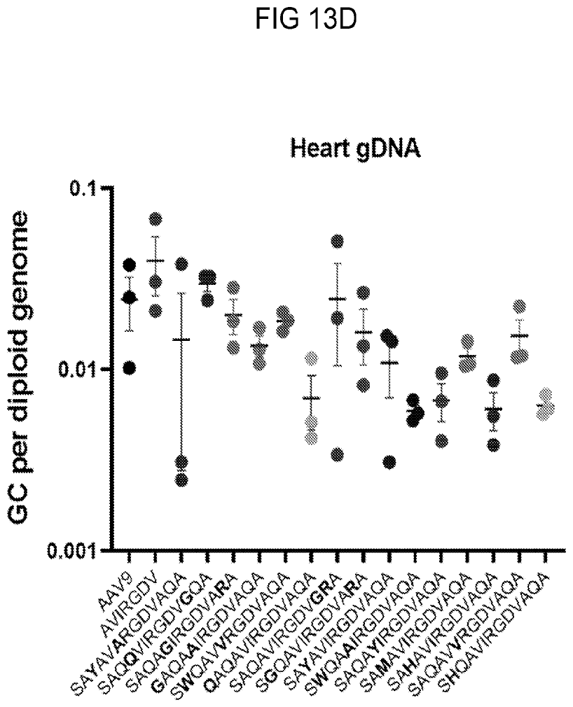

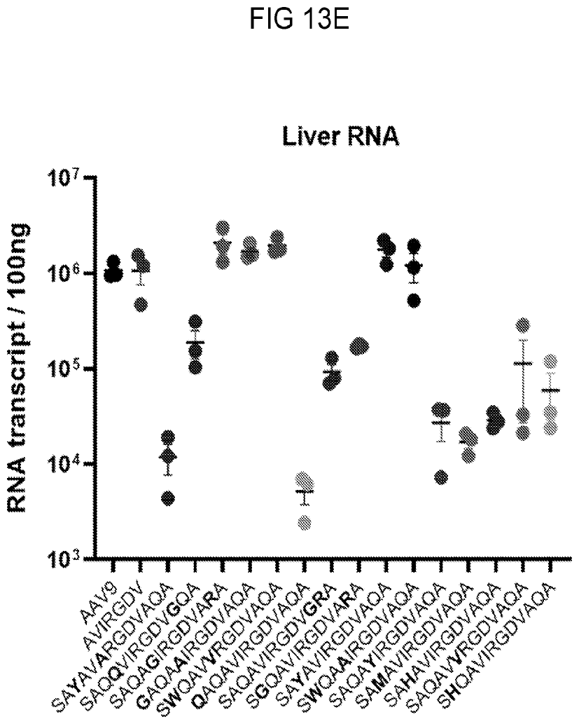

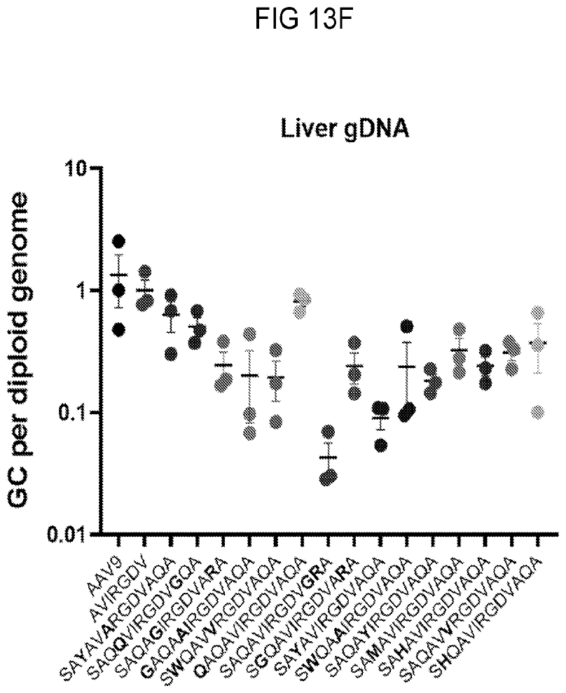

FIG. 10 shows RNA/DNA ratios (normalized to AAV9) for AAV9, AAV9-IIRGDPA, and AAV9-X vectors as analyzed in heart tissue. FIG. 11 shows RNA/DNA ratios (normalized to AAV9) for AAV9, AAV9-IIRGDPA, and AAV9-X vectors as analyzed in liver tissues. FIG. 12A shows RNA levels in heart tissue post-rAAV transduction, plotted as RNA transcript/100 ng. FIG. 12B shows DNA levels in heart tissue post-rAAV transduction, plotted as GC per diploid genome. FIG. 12C shows RNA levels in liver tissue post-rAAV transduction, plotted as RNA transcript/100 ng. FIG. 12D shows DNA levels in liver tissue post-rAAV transduction, plotted as GC per diploid genome. FIG. 13A shows RNA/DNA ratios (normalized to AAV9) for AAV9, AAV9-AVIRGDV, and AAV9-X vectors as analyzed in heart tissue. FIG. 13B shows RNA/DNA ratios (normalized to AAV9) for AAV9, AAV9-AVIRGDV, and AAV9-X vectors as analyzed in liver tissues. FIG. 13C shows RNA levels in heart tissue post-rAAV transduction, plotted as RNA transcript/100 ng. FIG. 13D shows DNA levels in heart tissue post-rAAV transduction, plotted as GC per diploid genome. FIG. 13E shows RNA levels in liver tissue post-rAAV transduction, plotted as RNA transcript/100 ng. FIG. 13F shows DNA levels in liver tissue post-rAAV transduction, plotted as GC per diploid genome.

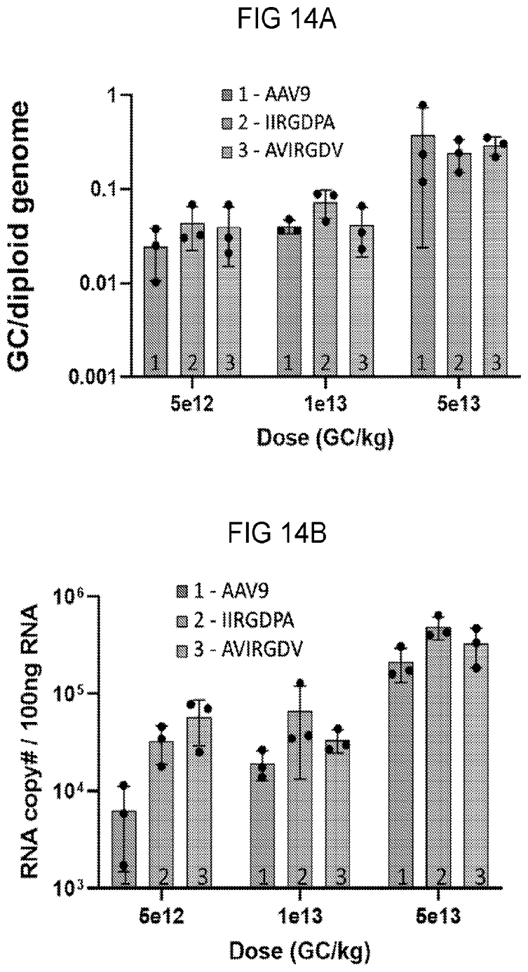

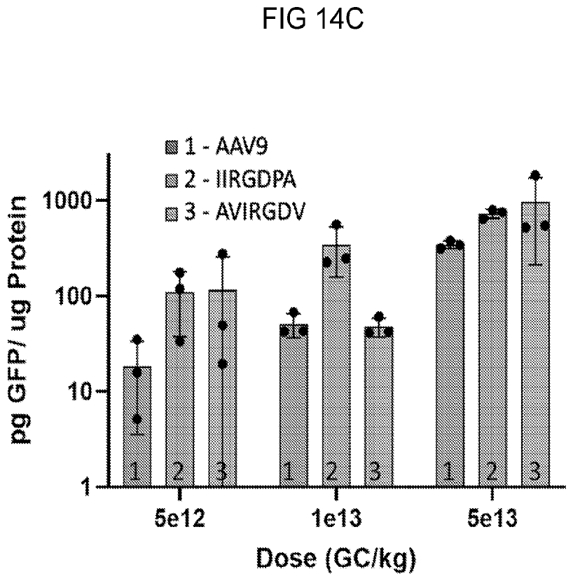

Additionally, we performed a dose-response study to evaluate transduction of the engineered rAAVs (comprising CB7.CI.eGFP.WPRE.rBG expression cassette, including barcoded GFP). In this study, mice (n=3/group) were administered rAAV9, rAAV9-IIRGDPA, and rAAV9-AVIRGDV at 5×10¹² (5e12) GC/kg, 1×10¹³ (1e13) GC/kg, or 5×10¹³ (5e13) GC/kg. Mice were then necropsied at day 14. Heart, liver, and gastrocnemius tissues were collected and analyzed for DNA, RNA and protein expression levels (FIGS. 14-16).

FIG. 14A shows DNA levels in heart tissue post-rAAV transduction, plotted as GC/diploid cell. FIG. 14B shows RNA levels in heart tissue post-rAAV transduction, plotted as copy number (#)/100 ng RNA. FIG. 14C shows protein expression levels in heart tissue post-rAAV transduction, plotted as pg GFP/μg protein. Table 14 below shows summary of the quantified values for DNA, RNA, and protein expression levels as plotted in FIGS. 14A-14C.

TABLE 14

| | | | |
|---|---|---|---|
| Average FC | AAV9 | AAV9-IIRGDPA | AAV9-AVIRGDV |

DNA

| | | | |
|---|---|---|---|
| 5 × 10¹² | 1.0 | 1.8 | 1.6 |
| 1 × 10¹³ | 1.0 | 1.8 | 1.0 |
| 5 × 10¹³ | 1.0 | 0.6 | 0.8 |

RNA

| | | | |
|---|---|---|---|
| 5 × 10¹² | 1.0 | 5.2 | 9.1 |
| 1 × 10¹³ | 1.0 | 3.5 | 1.7 |
| 5 × 10¹³ | 1.0 | 2.3 | 1.5 |

Protein

| | | | |
|---|---|---|---|
| 5 × 10¹² | 1.0 | 5.9 | 6.2 |
| 1 × 10¹³ | 1.0 | 6.8 | 0.9 |
| 5 × 10¹³ | 1.0 | 2.1 | 2.8 |

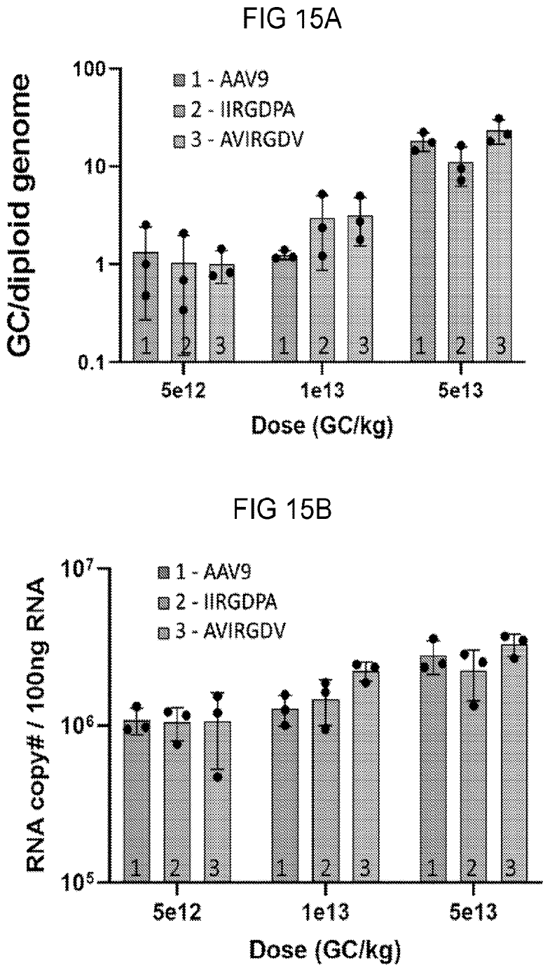

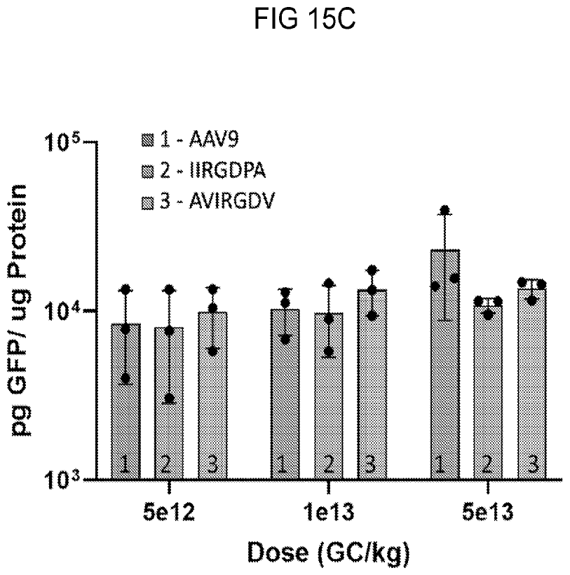

FIG. 15A shows DNA levels in liver tissue post-rAAV transduction, plotted as GC/diploid cell. FIG. 15B shows RNA levels in liver tissue post-rAAV transduction, plotted as copy number (#)/100 ng RNA. FIG. 15C shows protein expression levels in liver tissue post-rAAV transduction, plotted as pg GFP/μg protein. Table 15 below shows summary of the quantified values for DNA, RNA, and protein expression levels as plotted in FIGS. 15A-15C.

TABLE 15

| | | | |
|---|---|---|---|
| Average FC | AAV9 | AAV9-IIRGDPA | AAV9-AVIRGDV |

DNA

| | | | |
|---|---|---|---|
| 5 × 10¹² | 1.0 | 0.8 | 0.8 |
| 1 × 10¹³ | 1.0 | 2.4 | 2.5 |
| 5 × 10 13 | 1.0 | 0.6 | 1.3 |

RNA

| | | | |
|---|---|---|---|
| 5 × 10¹² | 1.0 | 1.0 | 1.0 |
| 1 × 10¹³ | 1.0 | 1.2 | 1.7 |
| 5 × 10¹³ | 1.0 | 0.8 | 1.2 |

Protein

| | | | |
|---|---|---|---|
| 5 × 10¹² | 1.0 | 1.0 | 1.2 |
| 1 × 10¹³ | 1.0 | 0.9 | 1.3 |
| 5 × 10¹³ | 1.0 | 0.5 | 0.6 |

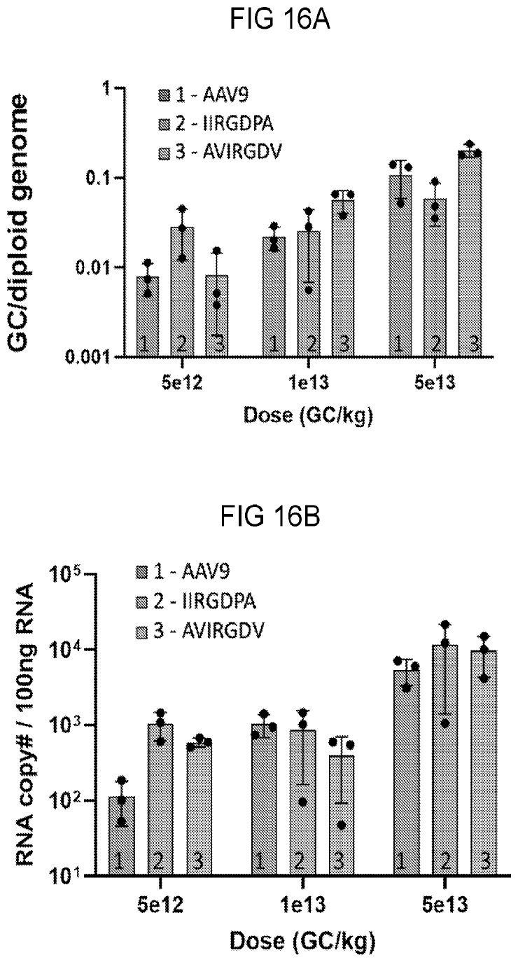

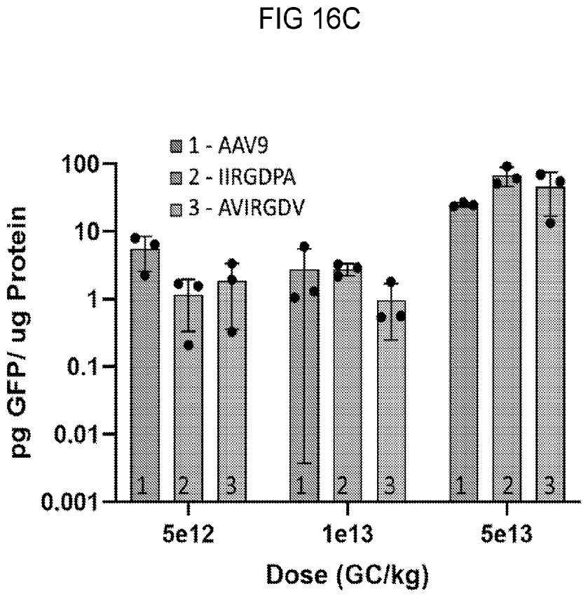

FIG. 16A shows DNA levels in gastrocnemius tissue post-rAAV transduction, plotted as GC/diploid cell. FIG. 16B shows RNA levels in gastrocnemius tissue post-rAAV transduction, plotted as copy number (#)/100 ng RNA. FIG. 16C shows protein expression levels in gastrocnemius tissue post-rAAV transduction, plotted as pg GFP/μg protein. Table 16 below shows summary of the quantified values for DNA, RNA, and protein expression levels as plotted in FIGS. 16A-16C.

TABLE 16

| | | | |
|---|---|---|---|
| Average FC | AAV9 | AAV9-IIRGDPA | AAV9-AVIRGDV |

DNA

| | | | |
|---|---|---|---|
| 5 × 10¹² | 1.0 | 3.6 | 1.0 |
| 1 × 10¹³ | 1.0 | 1.2 | 2.6 |
| 5 × 10¹³ | 1.0 | 0.5 | 1.9 |

RNA

| | | | |
|---|---|---|---|
| 5 × 10¹² | 1.0 | 9.2 | 5.2 |
| 1 × 10¹³ | 1.0 | 0.8 | 0.4 |
| 5 × 10¹³ | 1.0 | 2.2 | 1.8 |

Protein

| | | | |
|---|---|---|---|
| 5 × 10¹² | 1.0 | 0.2 | 0.3 |
| 1 × 10¹³ | 1.0 | 1.0 | 0.3 |
| 5 × 10¹³ | 1.0 | 2.7 | 1.8 |

The results showed similar DNA, RNA, and protein expression levels post-rAAV9 and engineered rAAV transduction when in liver tissue, and confirmed improved DNA, RNA, protein expression levels in heart and gastrocnemius tissues.

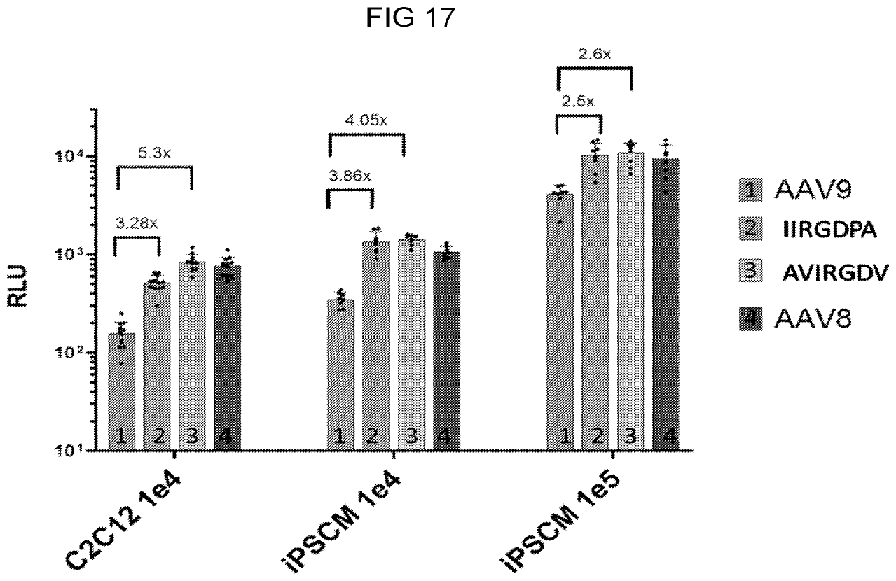

Next, we performed an assay to evaluate in vitro transduction of induced pluripotent stem cell (iPSC)-derived cardiomyocytes (iPSCM) and C2C12 cells (myoblast cell line) with rAAV9 and engineered rAAV9. Briefly, in this study, iPSCM cells were plated at 2.5×10⁴ (2.5e4) per well on day before transduction (D-1), cells were then transduced (DO), media was changes in the next 24 hours, and plates were evaluated for fluorescent signal (i.e., using plate reader) on D3. FIG. 17 shows GFP-expression levels in iPSCM cells and C2C12 cells post rAAV transduction, plotted as relative light units (RLU, from microplate reading). These data showed improved transduction by the engineered rAAV over AAV9.

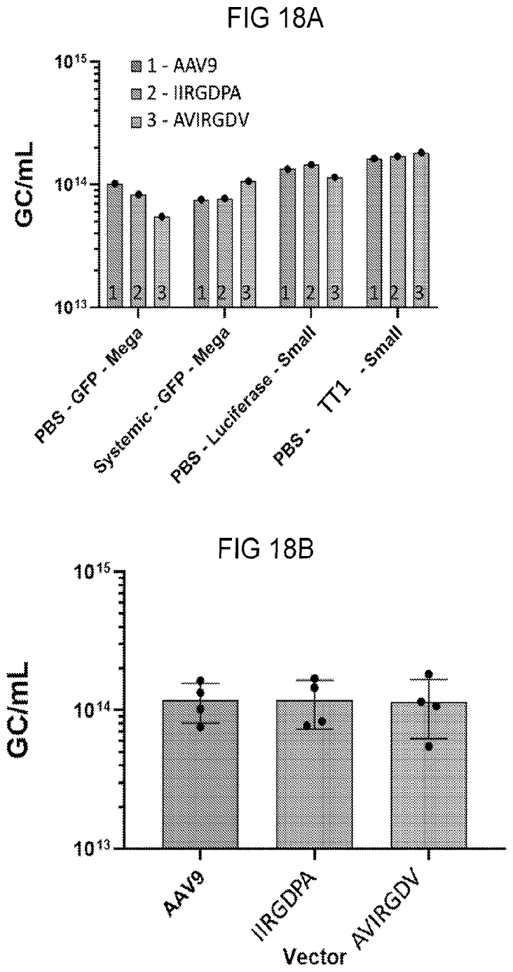

Additionally, we examined manufacturability of engineered rAAV9 vectors as compared with AAV9. FIG. 18A shows titers of rAAV (rAAV9, AAV9-IIRGDPA, and AAV9-AVIRGDV) plotted as GC/mL as measured in PBS or PBS with 0.001% Pluronic formulations, and as obtained from Mega and Small scale of rAAV preparations. FIG. 18B shows pooled titer of (rAAV9, AAV9-IIRGDPA, AAV9-AVIRGDV) plotted as GC/mL as measured in PBS or PBS with 0.001% Pluronic formulations, and as obtained from Mega and Small scale of rAAV preparations. Table 17 below shows a summary of the manufacturability results (FIG. 18).

TABLE 17

| Manufac- | | | |
|---|---|---|---|
| turability | | | |
| Formulation - | Titer | | |
| Transgene - | | AAV9- | AAV9- |
| Scale | AAV9 | IIRGDPA | AVIRGDV |
| PBS - GFP - | 1.02 × 10¹⁴ | 8.32 × 10¹³ | 5.48 × 10¹³ |
| Mega | | | |
| Systemic - | 7.58 × 10¹³ | 7.73 × 10¹³ | 1.07 × 10¹⁴ |
| GFP - | | | |
| Mega | | | |
| PBS - | 1.34 × 10¹⁴ | 1.46 × 10¹⁴ | 1.15 × 10¹⁴ |
| Luciferase - | | | |
| Small | | | |
| PBS - Test | 1.63 × 10¹⁴ | 1.7 × 10¹⁴ | 1.82 × 10¹⁴ |
| Transgene1 | (AAVhu68) | | |
| (TT1) - | | | |
| Small | | | |

### Example 4: Further Evaluation of Engineered rAAV9 Comprising Test Transgenes (TTx)

In this study, we evaluated AAV9-IIRGDPA, AAV9-AVIRGDV, and AAVhu68 vectors comprising Test Transgene 1 (TT1) to evaluate transduction and potency of the vectors. We performed capsid comparison in mice evaluating survival and body weight in wild-type (WT) and knock out mice (transgene gene knock out, KO). Mice were administered engineered rAAV9 intravenously at a dose of dose was 3E13 (3×10¹³) GC/kg (time points per group: 7, 14, 28, 42, 56 and 90). Mice are monitored and examined with imaging (echocardiography with ECG) and collecting body weight measurement. Necropsy is performed at day 7, 14, 28, 42, 56 and 90.

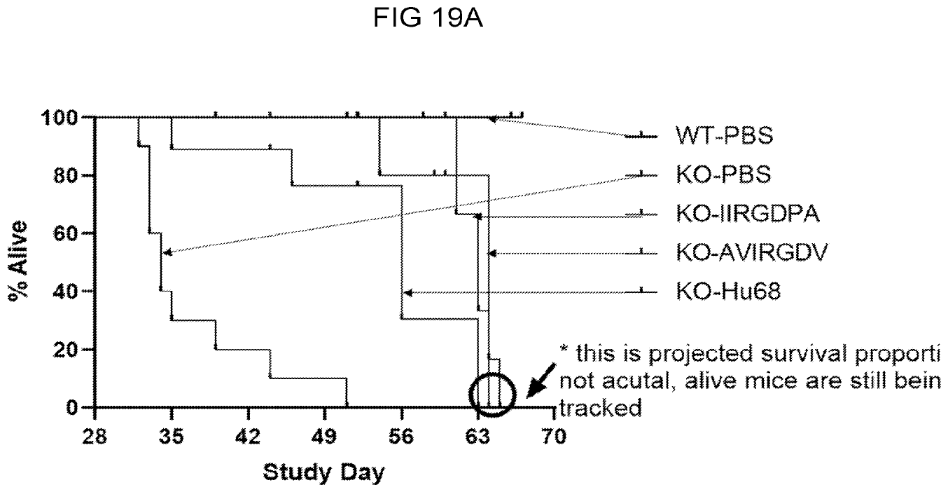

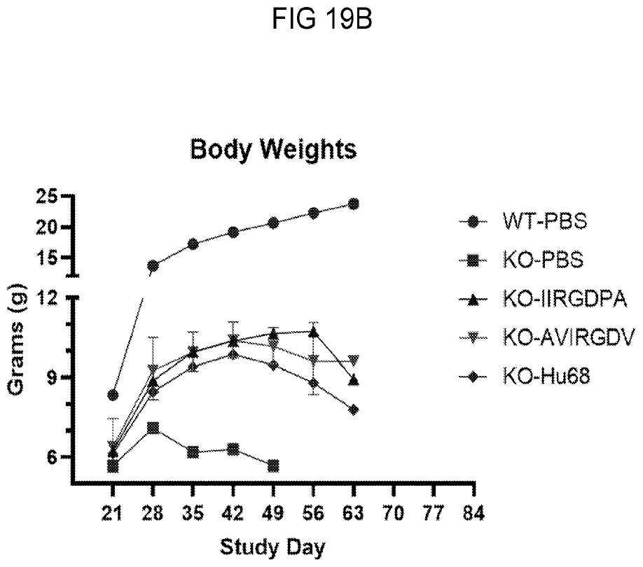

FIG. 19A shows results of an ongoing survival study, plotted as probability of survival. The results show similar and/or better survival observed in mice when administered the AAV9-IIRGDPA or AAV9-AVIRGDV vectors in comparison to AAVhu68 vectors, all comprising TT1. FIG. 19B shows results of measured body weight, plotted as grams (g). The results show similar and/or better weight as measured in mice, as compared to WT, when administered the AAV9-IIRGDPA or AAV9-AVIRGDV vectors in comparison to AAVhu68 vectors, all comprising TT1.

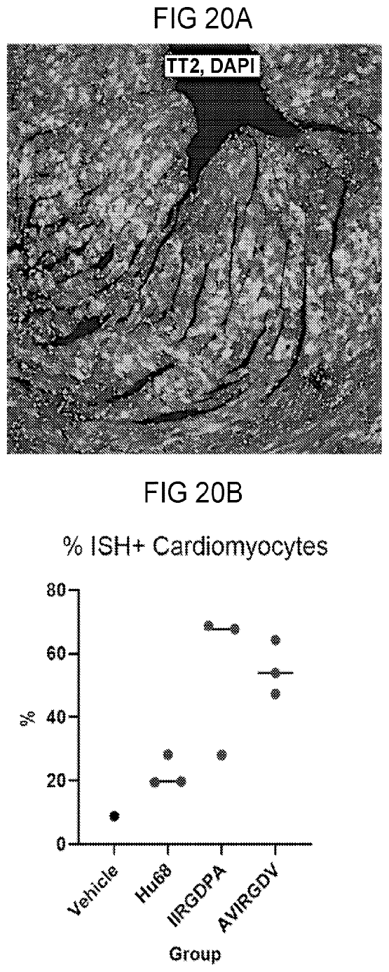

Next, we examined the potency of AAV vectors comprising Test Transgene 2 (TT2) in mice. Briefly, in this study, mice (n=3 per treatment group, at 4-6 weeks of age at the time of enrolment) were administered AAV vector (AAVhu68.CB7.TT2.rBG, AAV9-AVIRGDV.CB7.TT2.rBG, and AAV9-IIRGDPA.CB7.TT2.rBG) at a dose of 2×10¹³ (2e13) GC/kg or PBS via tail IV injection, following 7 days mice were necropsied and tissues were collected for biodistribution and in situ hybridization (ISH) analysis. FIG. 20A shows representative ISH microscopy images of heart and liver tissue. FIG. 20B show quantified percent ISH-positive cardiomyocytes in various treatment groups. The result show improved expression levels of TT2 in heart tissue when mice were administered AAV9-AVIRGDV or AAV9-IIRGDPA.

Overall, transduction was observed in all AAV-treated mice. Greatest heart transduction was observed in mice administered with AAV9-IIRGDPA and the lowest level of transduction was observed in mice administered the AAVhu68 vector. Liver transduction was comparable in mice administered the AAVhu68 and AAV9-AVIRGDV vectors, and lowest in mice administered the AAV9-IIRGDPA vector. These results confirm AAV9-IIRGDPA's heart-targeting and liver-de-targeting properties.

Additionally, in this study, we evaluated pharmacology and safety of rAAV.TT2 comprising a cardio tropic AAV capsid. AAVhu68, AAV9-IIRGDPA, and AAV9-AVIRGDV vectors were administered intravenously to Cynomolgus Macaques at low dose (2×10¹³ or 2e13 GC/kg; n=2). During days DO to D28, monitoring of potential acute toxicity was performed (e.g., liver injury, thrombotic microangiopathy (TMA)). Additionally, EKG and nerve conduction studies were performed on DO, D28, D60, and D90. On D90, necropsy was performed, and tissues were collected for analysis, including DNA and RNA biodistribution, ISH, whole slide morphometry, H&E pathology.

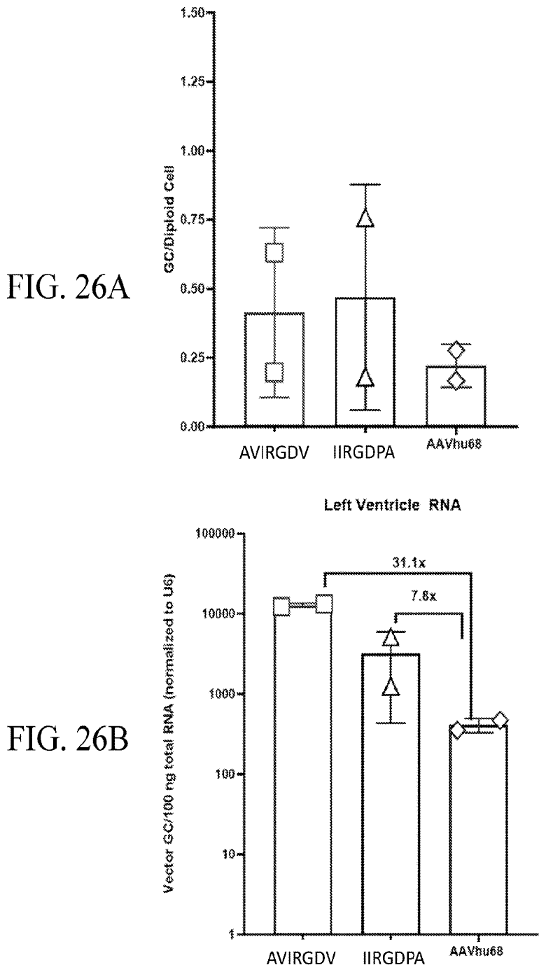

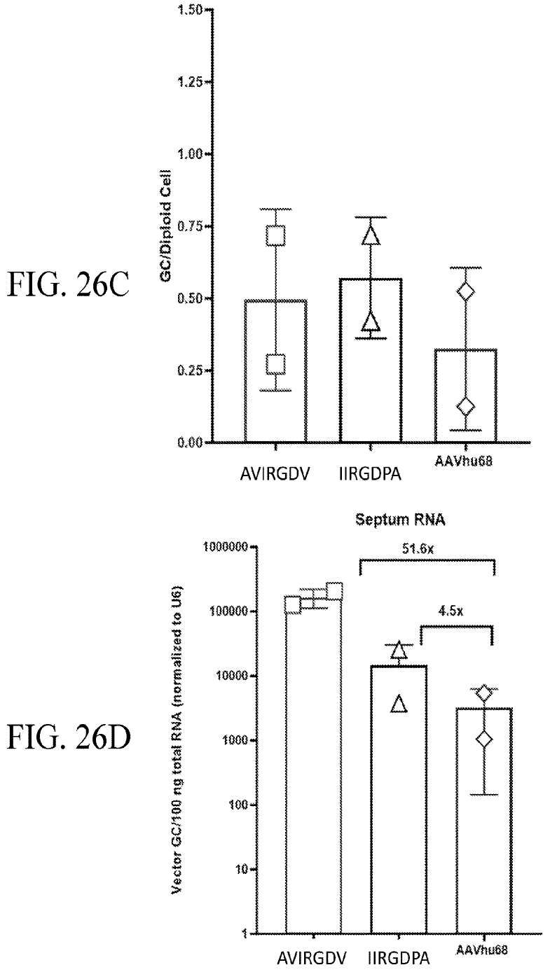

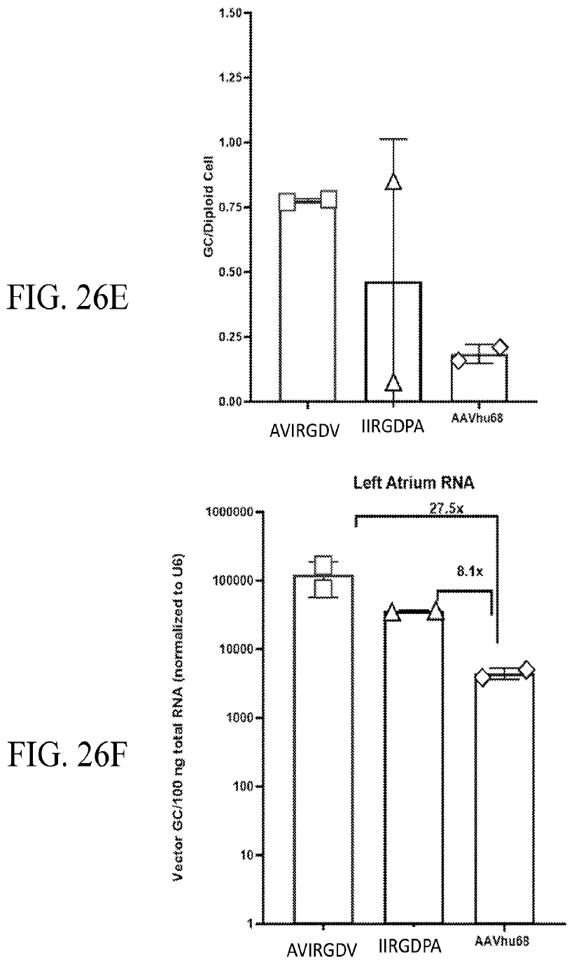

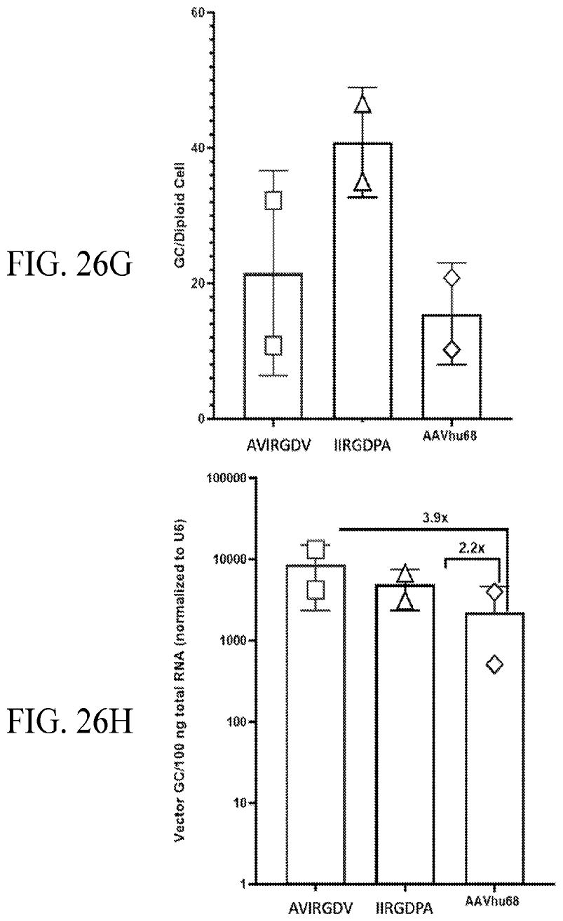

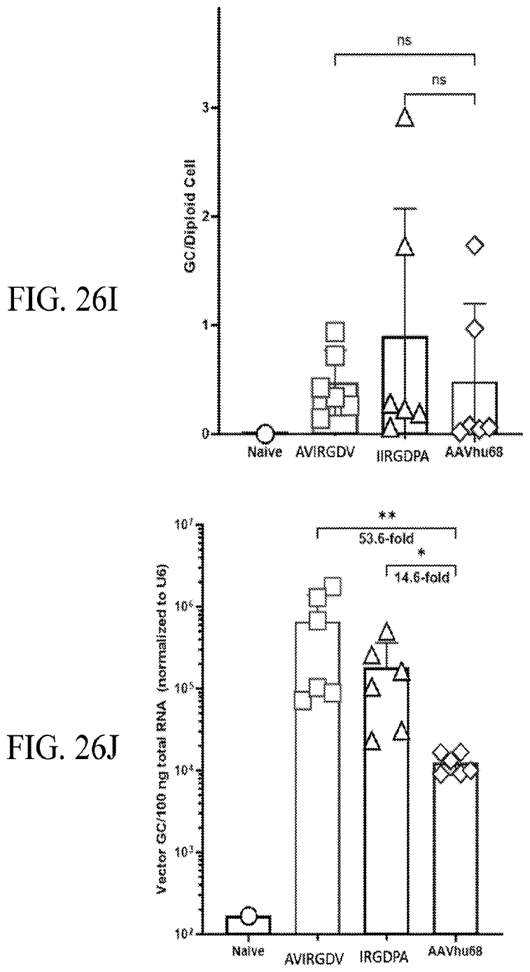

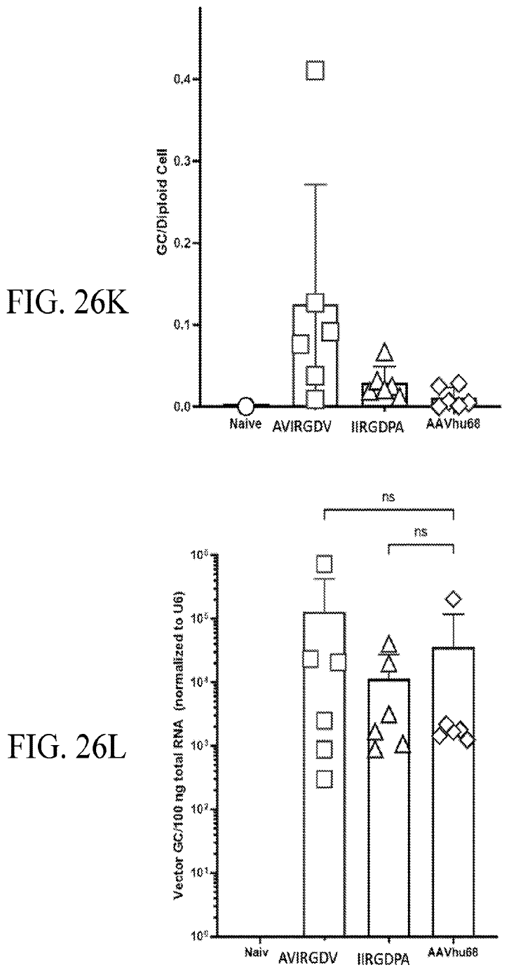

FIG. 26A shows TT2 DNA levels in left ventricle tissue, plotted as GC/diploid cell, following AAVhu68. TT2, AAV9-IIRGDPA. TT2, or AAV9-AVIRGDV.TT2 administration. FIG. 26B shows TT2 RNA levels in left ventricle tissue, plotted as vector GC/100 ng total RNA (as normalized to U6), following AAVhu68.TT2, AAV9-IIRGDPA.TT2, or AAV9-AVIRGDV.TT2 administration. FIG. 26C shows TT2 DNA levels in septum tissue, plotted as GC/diploid cell, following AAVhu68.TT2, AAV9-IIRGDPA.TT2, or AAV9-AVIRGDV.hTT2 administration. FIG. 26D shows TT2 RNA levels in septum tissue, plotted as vector GC/100 ng total RNA (as normalized to U6), following AAVhu68. TT2, AAV9-IIRGDPA.TT2, or AAV9-AVIRGDV.TT2 administration. FIG. 26E shows TT2 DNA levels in left atrium tissue, plotted as GC/diploid cell, following AAVhu68.TT2, AAV9-IIRGDPA. TT2, or AAV9-AVIRGDV.TT2 administration. FIG. 26F shows TT2 RNA levels in left atrium tissue, plotted as vector GC/100 ng total RNA (as normalized to U6), following AAVhu68.TT2, AAV9-IIRGDPA.TT2, or AAV9-AVIRGDV.TT2 administration. FIG. 26G shows TT2 DNA levels in liver, plotted as GC/diploid cell, following AAVhu68.TT2, AAV9-IIRGDPA.TT2, or AAV9-AVIRGDV.TT2 administration. FIG. 26H shows TT2 RNA levels in liver, plotted as vector GC/100 ng total RNA (as normalized to U6), following AAVhu68.TT2, AAV9-IIRGDPA.TT2, or AAV9-AVIRGDV.TT2 administration. FIG. 26I shows TT2 DNA levels in diaphragm, plotted as GC/diploid cell, following AAVhu68. TT2, AAV9-IIRGDPA.TT2, or AAV9-AVIRGDV.TT2 administration. FIG. 26J shows TT2 RNA levels in diaphragm, plotted as vector GC/100 ng total RNA (as normalized to U6), following AAVhu68.TT2, AAV9-IIRGDPA.TT2, or AAV9-AVIRGDV.TT2 administration. FIG. 26K shows TT2 DNA levels in quadriceps tissue, plotted as GC/diploid cell, following AAVhu68.TT2, AAV9-IIRGDPA. TT2, or AAV9-AVIRGDV.TT2 administration. FIG. 26L shows TT2 RNA levels in quadriceps tissue, plotted as vector GC/100 ng total RNA (as normalized to U6), following AAVhu68.TT2, AAV9-IIRGDPA.TT2, or AAV9-AVIRGDV.TT2 administration. These results show that there was a 30-50-fold increase in cardiac expression of TT2 in heart tissue, and a 50-fold increase in diaphragm transduction following administration of the rAAV9-AVIRGDV.TT2 vector.

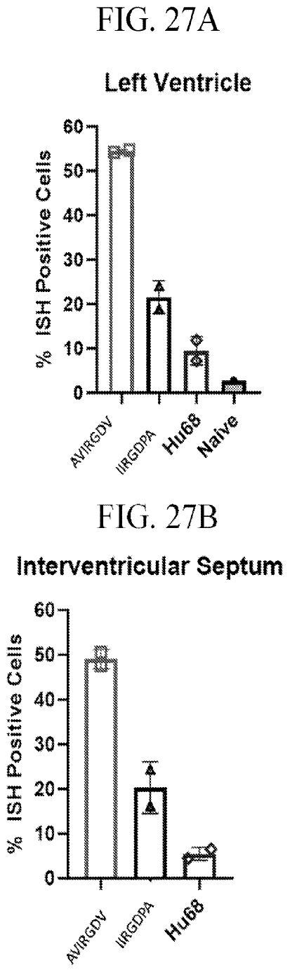

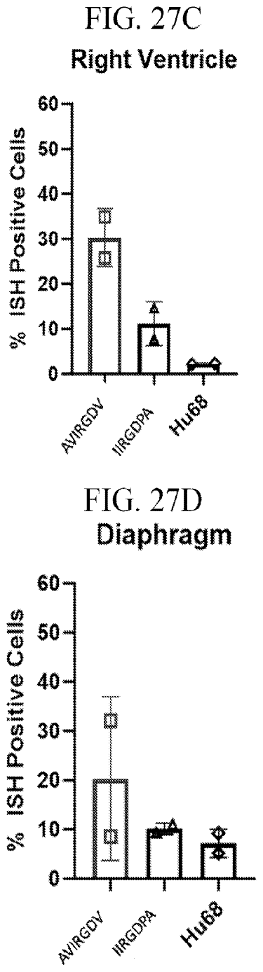

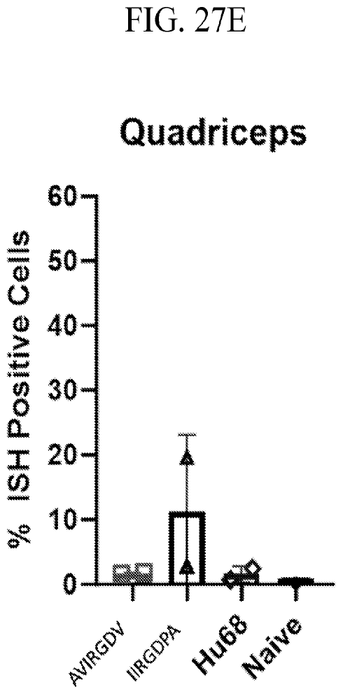

FIG. 27A shows results of ISH analysis, plotted as percent ISH-positive cells in tissue from the left ventricle of the heart. FIG. 27B shows results of ISH analysis, plotted as percent ISH-positive cells in tissue from the intraventricular septum of the heart. FIG. 27C shows results of ISH analysis, plotted as percent ISH-positive cells in tissue form the right ventricle of the heart. FIG. 27D shows results of ISH analysis, plotted as percent ISH-positive cells in diaphragm tissue. FIG. 27E shows results of ISH analysis, plotted as percent ISH-positive cells in quadricep tissue. These results confirm widespread and robust cardiac transduction in cardiac and diaphragm tissue, and mild transduction in quadricep tissue following administration of a rAAV9-AVIRGDV.TT2 vector.

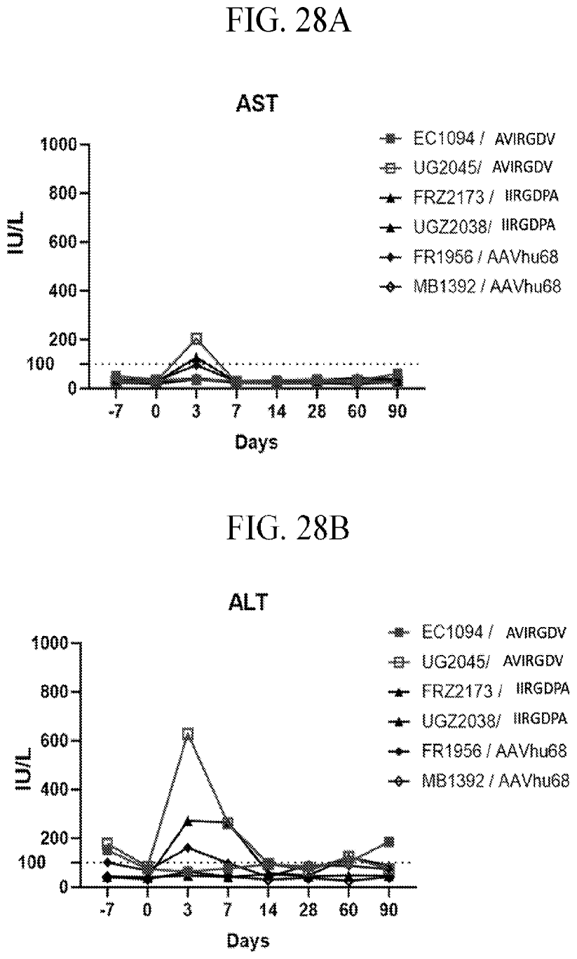

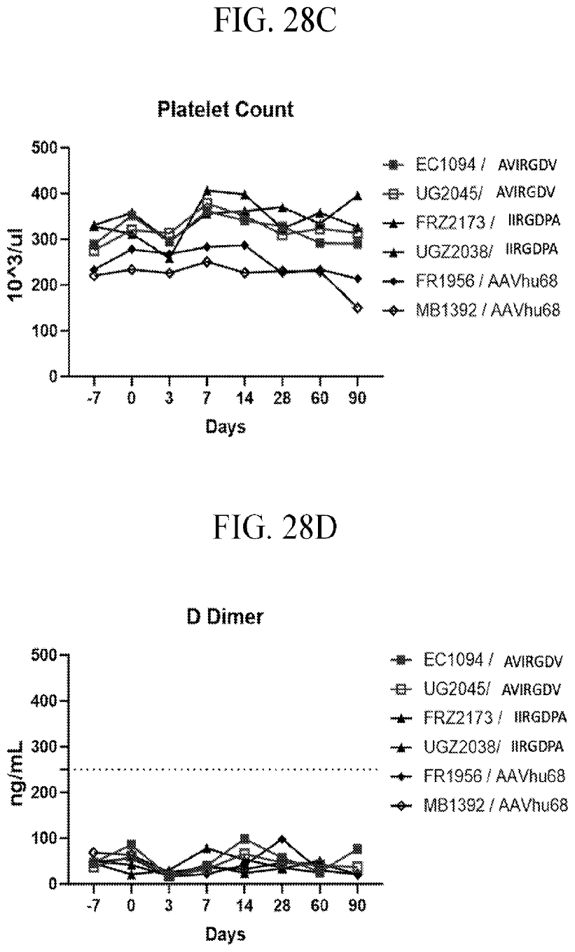

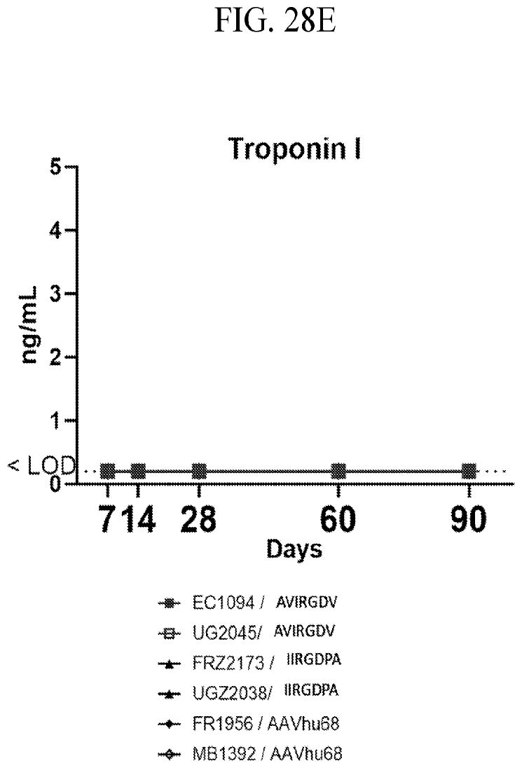

FIG. 28A shows levels of aspartate aminotransferase (AST) in blood samples on DO to D90 following AAVhu68. TT2, AAV9-IIRGDPA. TT2, or AAV9-AVIRGDV.TT2 administration. FIG. 28B shows levels of alanine aminotransferase (ALT) in blood samples on DO to D90 following AAVhu68.TT2, AAV9-IIRGDPA.TT2, or AAV9-AVIRGDV.TT2 administration. FIG. 28C shows levels of platelet counts in blood samples on DO to D90 following AAVhu68.TT2, AAV9-IIRGDPA.TT2, or AAV9-AVIRGDV.TT2 administration. FIG. 28D shows levels of d dimer in blood samples on DO to D90 following AAVhu68.TT2, AAV9-IIRGDPA.TT2, or AAV9-AVIRGDV.TT2 administration. FIG. 28E shows measured levels of troponin I in blood samples on DO to D90 following AAVhu68.TT2, AAV9-IIRGDPA. TT2, or AAV9-AVIRGDV.TT2 administration.

### Example 5: Evaluation of Engineered rAAV9 in NHP

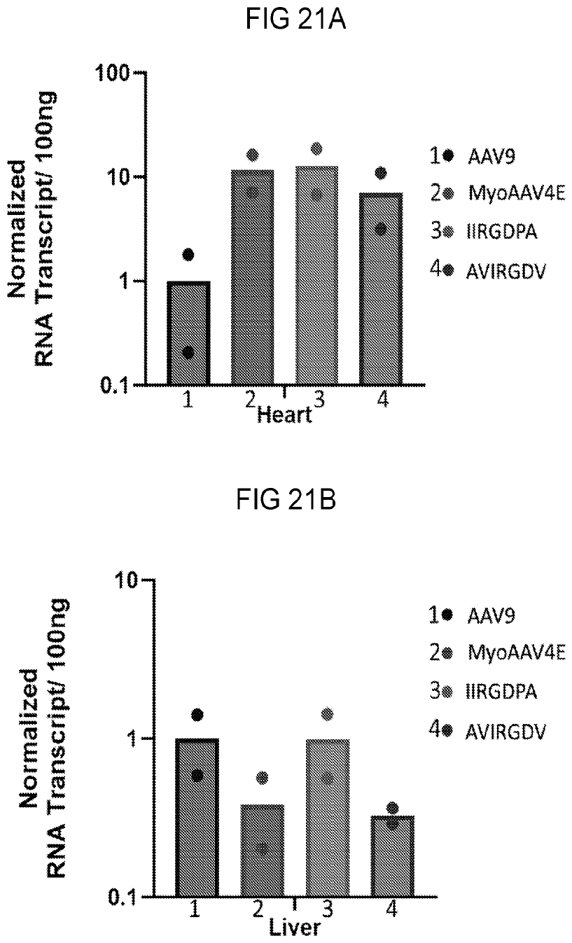

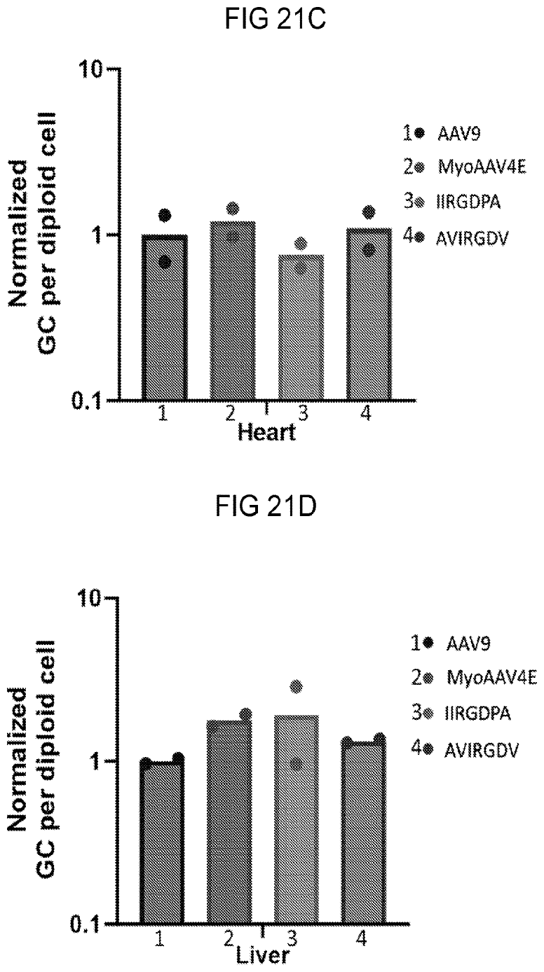

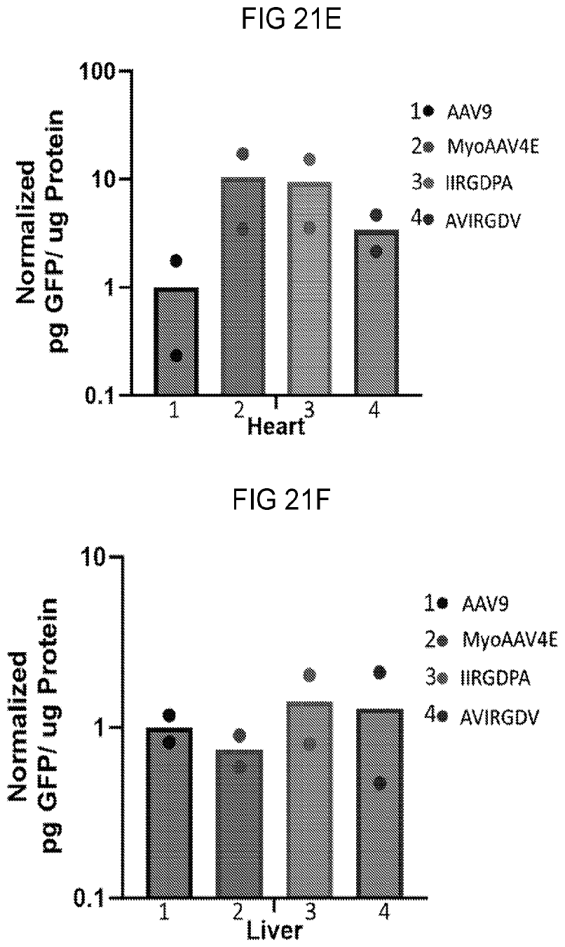

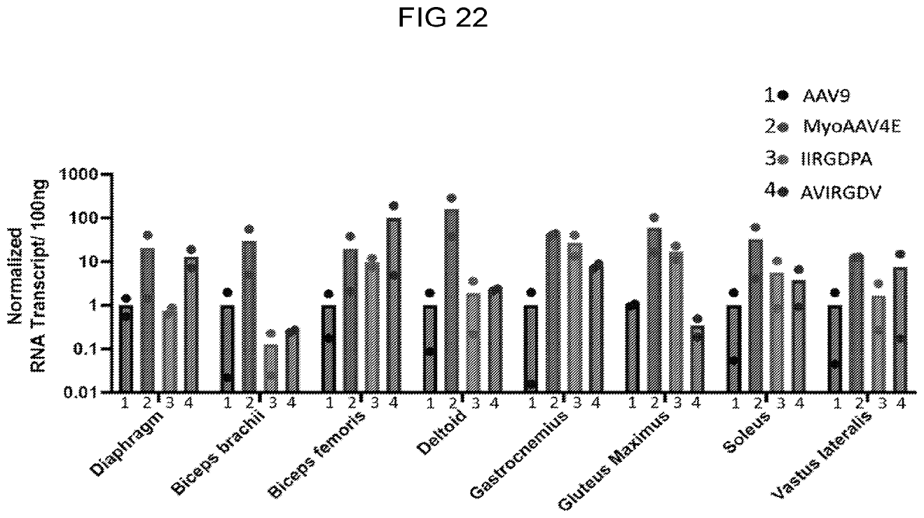

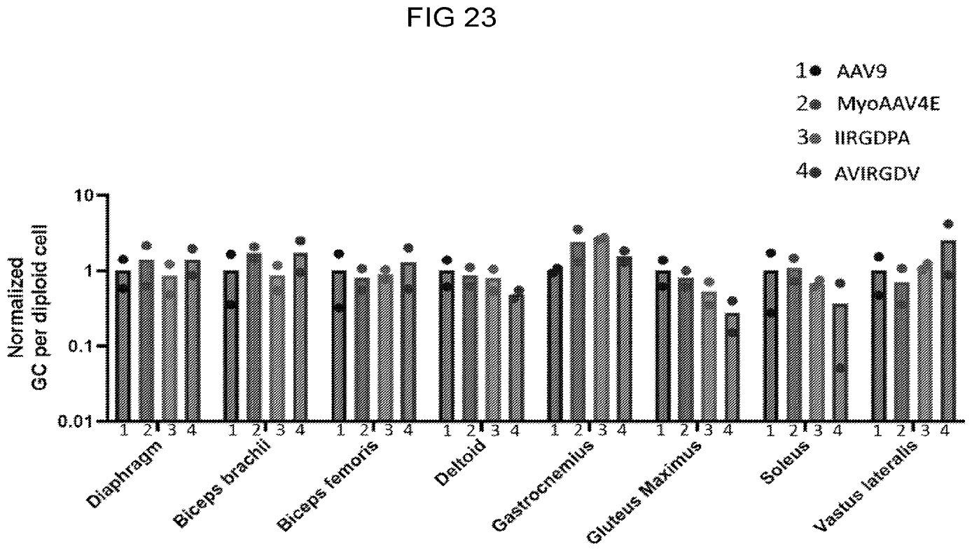

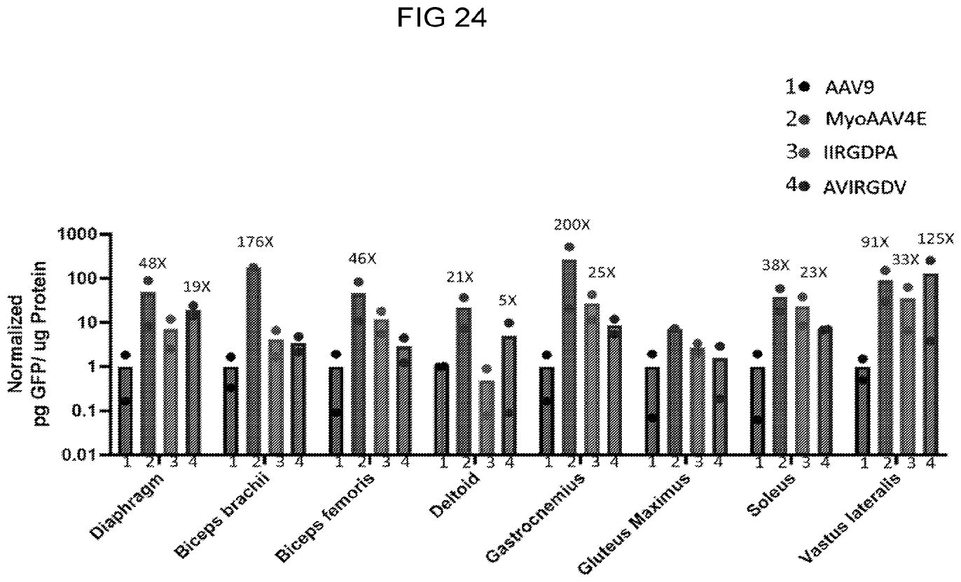

In this study, we performed testing of individual heart capsids (i.e., engineered rAAV comprising exogenous targeting peptides) for cardiac, gastrocnemius, liver cell transduction, in which AAV9, AAV9-IIRGDPA, AAV9-AVIRGDV, and AAV9-MyoAAV4E (literature control, Manini, A., Adeno-Associated Virus (AAV)-Mediated Gene Therapy for Duchenne Muscular Dystrophy: The Issue of Transgene Persistence, Front. Neurol., 2021, 12:814174) were administered to 2 NHPs at a dose of 1×10¹³ GC/kg intravenously. NHPs were observed for 14 days, after which they were necropsied. Heart, liver, and gastrocnemius tissues were collected and analyzed for biodistribution, and histological analysis was performed. FIG. 21A shows RNA levels, plotted as normalized RNA transcript copy number (#)/100 nanogram (ng) RNA in heart tissue. FIG. 21A shows RNA levels, plotted as normalized RNA transcript copy number (#)/100 ng RNA in heart tissue. FIG. 21B shows RNA levels, plotted as normalized RNA transcript copy number (#)/100 ng RNA in liver tissue. FIG. 21C shows DNA levels, plotted as normalized GC per diploid cell in heart tissue. FIG. 21D shows DNA levels, plotted as normalized GC per diploid cell in liver tissue. FIG. 21E shows protein (GFP) expression levels, plotted as normalized picogram (PG) GFP per microgram (μg) total protein from heart tissue. FIG. 21F shows protein (GFP) expression levels, plotted as normalized pg GFP per μg total protein from liver tissue. FIG. 22 shows RNA biodistribution results in various muscle tissue subtypes. FIG. 23 shows DNA biodistribution results in various muscle tissue subtypes. FIG. 24 shows protein (GFP) biodistribution results in various muscle tissue subtype, values for which are also summarized in Table 18 below.

TABLE 18

| | | | |
|---|---|---|---|
| Tissue | DNA | RNA | Protein |

MyoAAV4E

| | | | |
|---|---|---|---|
| Heart | 1.2 | 11 | 10.2 |
| Liver | 1.8 | 0.3 | 0.7 |
| Gastroc. | 2.4 | 40.0 | 266.1 |
| Diaphragm | 1.38 | 20.7 | 48.3 |
| Biceps Brachii | 1.7 | 30.0 | 176.2 |
| Soleus | 1.05 | 32.5 | 38.6 |
| Deltoid | 0.9 | 62.3 | 21.9 |
| Gluteus Maximus | 0.8 | 59.6 | 7.3 |
| Vastus Lateralis | 1.7 | 12.6 | 90.2 |

IIRGDPA

| | | | |
|---|---|---|---|
| Heart | 0.8 | 13 | 9.4 |
| Liver | 1.9 | 0.8 | 1.4 |
| Gastroc. | 2.67 | 26.0 | 27.1 |
| Diaphragm | 0.8 | 13.5 | 7.2 |
| Biceps Brachii | 0.8 | 0.2 | 4.1 |
| Soleus | 0.7 | 5.6 | 23.3 |
| Deltoid | 0.75 | 1.9 | 0.5 |
| Gluteus Maximus | 0.5 | 16.9 | 2.7 |
| Vastus Lateralis | 0.9 | 1.7 | 34.7 |

AVIRGDV

| | | | |
|---|---|---|---|
| Heart | 1.0 | 6 | 3.4 |
| Liver | 1.3 | 0.3 | 1.3 |
| Gastroc. | 1.5 | 8.0 | 8.7 |
| Diaphragm | 1.40 | 12.5 | 19.3 |
| Biceps Brachii | 1.75 | 0.2 | 3.5 |
| Soleus | 0.36 | 3.8 | 6.8 |
| Deltoid | 0.4 | 2.25 | 4.9 |
| Gluteus Maximus | 0.3 | 0.3 | 1.5 |
| Vastus Lateralis | 1.4 | 7.5 | 128.5 |

These results confirm AAV9-IIRGDPA's and AAV9-AVIRGDV's muscle cell-targeting and liver-de-targeting properties.

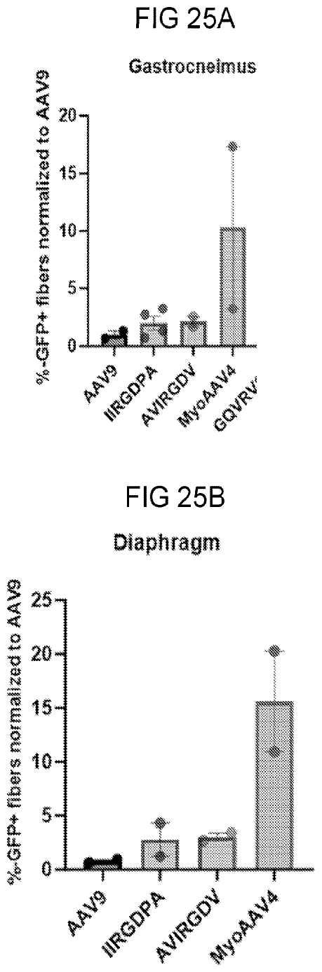

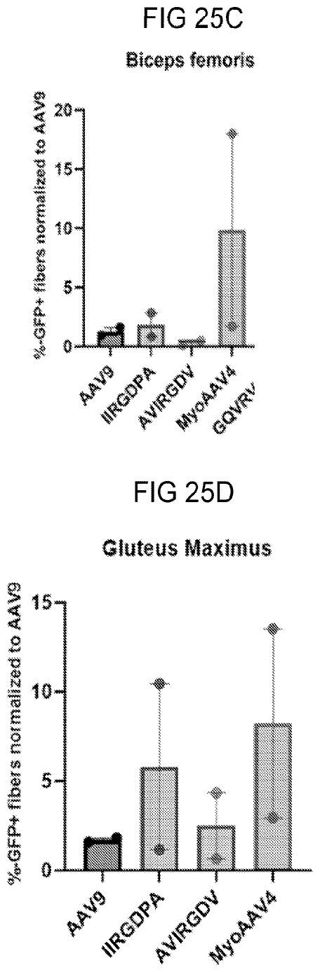

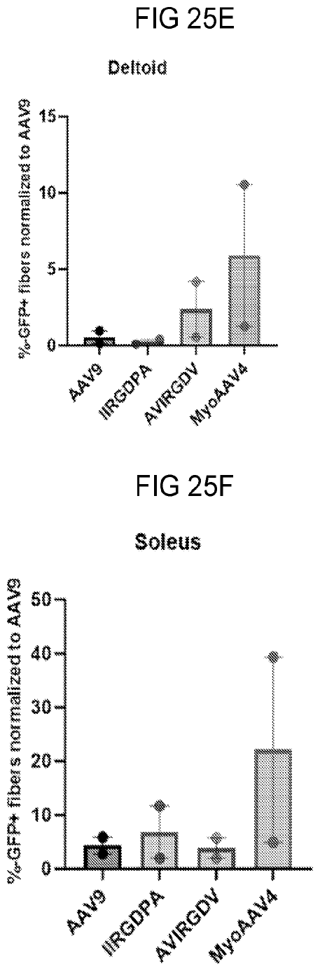

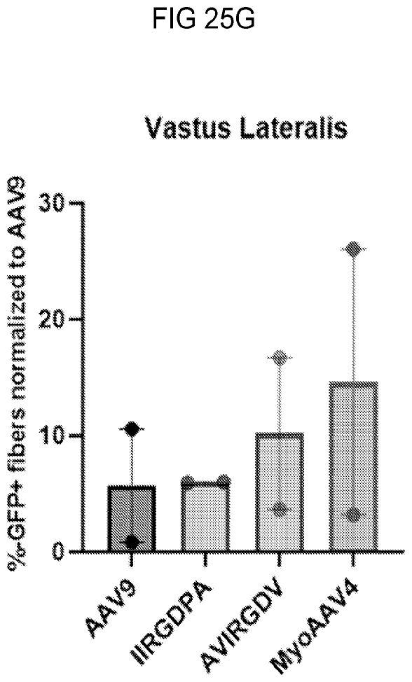

FIG. 25A shows results of the In situ hybridization (ISH) analysis in gastrocnemius tissue, plotted as percent GFP positive (normalized to an AAV9 control). FIG. 25B shows results of the ISH analysis in diaphragm tissue, plotted as percent GFP positive (normalized to an AAV9 control). FIG. 25C shows results of the ISH analysis in biceps femoris tissue, plotted as percent GFP positive (normalized to an AAV9 control). FIG. 25D shows results of the ISH analysis in gluteus maximus tissue, plotted as percent GFP positive (normalized to an AAV9 control). FIG. 25E shows results of the ISH analysis in deltoid tissue, plotted as percent GFP positive (normalized to an AAV9 control). FIG. 25F shows results of the ISH analysis in soleus tissue, plotted as percent GFP positive as (normalized to an AAV9 control). FIG. 25G shows results of the ISH analysis in vastus lateralis tissue, plotted as percent GFP positive (normalized to an AAV9 control).

Additionally, we performed a further barcode cardiac vector study using vectors including AAV9-RGDLHGY, AAV9-PYQRGDH, AAV9-RGDYAQV, MyoAAV (literature control), and AAV9 (negative control). Vectors were administered to cynomolgus macaques (n=2) at a dose of 5×10¹³ GC/kg. Barcode study results are summarized in Table 19 and Table 20 below.

TABLE 19

| | | | | Biceps | Biceps |
|---|---|---|---|---|---|
| Vector | Titer | Heart | Diaphragm | Brachii | Femoris |
| AAV9 | 8.29E+13 | 1 | 1 | 1 | 1 |
| MyoAAV4E | 3.52E+13 | 7.55 | 15.32 | 23.64 | 10.94 |
| RGDLHGY | 3.22E+13 | 1.59 | 2.77 | 7.51 | 8.49 |
| PYQRGDH | 3.49E+13 | 2.51 | 4.43 | 3.16 | 3.55 |
| RGDYAQV | 1.69E+13 | 2.71 | 4.07 | 9.99 | 7.57 |

TABLE 20

| | | | Glut | | Vastrus | |
|---|---|---|---|---|---|---|
| Vector | Deltoid | Gastroc. | Max | Soleus | Lateralis | Liver |
| AAV9 | 1 | 1 | 1 | 1 | 1 | 1 |
| MyoAAV4E | 20.4 | 14.33 | 10.4 | 19.96 | 16.08 | 0.98 |
| RGDLHGY | 6.18 | 7.33 | 6.46 | 6.95 | 9.08 | 0.56 |
| PYQRGDH | 2.88 | 2.95 | 2.11 | 2.94 | 3.88 | 1.09 |
| RGDYAQV | 9.14 | 8.24 | 8.02 | 8.68 | 8.85 | 0.76 |

All documents cited in this specification are incorporated herein by reference. US Provisional Patent Application No. 63/387,941, filed Dec. 17, 2022, and U.S. Provisional Patent Application No. 63/514,404, filed Jul. 19, 2023 are incorporated herein by reference in their entireties. While the invention has been described with reference to particular embodiments, it will be appreciated that modifications can be made without departing from the spirit of the invention. Such modifications are intended to fall within the scope of the appended claims.

1. A recombinant adeno-associated virus particle (rAAV) comprising:
(a) an adeno-associated virus (AAV) capsid comprising VP1 proteins, VP2 proteins and VP3 proteins, wherein the capsid proteins have an amino acid sequence comprising a hypervariable region comprising an exogenous targeting peptide, wherein the exogenous targeting peptide comprises “Xn-n-mer-Xm”, wherein:
(i) Xn is 0, 1, 2 or 3 amino acid residues independently selected from any amino acid;
(ii) the n-mer is AVIRGDV (SEQ ID NO: 2), IIRGDPA (SEQ ID NO: 1), RGDYAQV (SEQ ID NO: 20), RGDLHGY (SEQ ID NO: 22), PYQRGDH (SEQ ID NO: 24), IVRGDPA (SEQ ID NO: 8), MIRGDVK (SEQ ID NO: 9), AQHRGDV (SEQ ID NO: 10), VSRGDPN (SEQ ID NO: 11), VSRGDPA (SEQ ID NO: 12), PLVRGDI (SEQ ID NO: 13), PYVRGDP (SEQ ID NO: 14), VVRGDPQ (SEQ ID NO: 15), VVQRGDV (SEQ ID NO: 17), QHRGDTQ (SEQ ID NO: 18), QIRGDLR (SEQ ID NO: 19), IGRGDPN (SEQ ID NO: 21), or RGDYSTM (SEQ ID NO: 23), or an n-mer sequence of at least 6, at least 7, or the full-length consecutive amino acids of any one of the n-mers; and
(iii) Xm is 0, 1, 2, or 3 amino acid residues independently selected from any amino acid; and
(b) a vector genome packaged in the AAV capsid, wherein the vector genome comprises a nucleic acid sequence encoding a gene product under control of sequences which direct expression thereof.

2. The rAAV of claim 1, wherein the exogenous targeting peptide comprises:
(a)
(SEQ ID NO: 2)
AVIRGDV;
or
(b)
(SEQ ID NO: 1)
IIRGDPA.

3. The rAAV of claim 1, wherein the exogenous targeting peptide is inserted between any two contiguous amino acids in the hypervariable region VIII (HVRVIII) or the hypervariable region IV (HVRIV) at a suitable location of a parental AAV capsid.

4. The rAAV of claim 3, wherein the parental AAV capsid is AAV9, AAV8, AAV7, AAV6, AAV5, AAV4, AAV3, AAV1, AAVhu68, AAVhu95, AAVhu96, or AAVrh91.

5. The rAAV of claim 1, wherein the exogenous targeting peptide is inserted in the hypervariable region between amino acids 588 and 589 in an AAV9 parental capsid as determined based on the numbering of VP1 amino acid sequence of SEQ ID NO: 25, or is inserted in an analogous position in an AAV8, AAV7, AAV6, AAV5, AAV4, AAV3, AAV1, AAVhu68, AAVhu95, AAVhu96, or AAVrh91 parental capsid.

6. The rAAV of claim 1, wherein the exogenous targeting peptide is immediately preceded by “AQ”.

7. The rAAV of claim 1, wherein the rAAV capsid comprises AAV VP3 proteins having a mutant VP3 region of: amino acids 204 to 743 of SEQ ID NO: 73, 75, 77, 79, 81, 83, 85, 87, 89, 91, 93, 95, 97, 99, 101, 103, 105, or 107, further comprising highly deamidated residues at positions N57, N329, N452, and N512, wherein the deamidated position numbers are based on the residue positions of SEQ ID NO: 25 or SEQ ID NO: 26.

8. The rAAV of claim 1, wherein the rAAV capsid comprises AAV VP1 proteins, AAV VP2 proteins and AAV VP3 proteins having a mutant VP1 of: amino acids 1 to 743 of SEQ ID NO: 73, 75, 77, 79, 81, 83, 85, 87, 89, 91, 93, 95, 97, 99, 101, 103, 105, or 107, further comprising highly deamidated residues at positions N57, N329, N452 and N512, wherein the deamidated position numbers are based on the residue positions of SEQ ID NO: 25 or SEQ ID NO: 26.

9. The rAAV of claim 1, wherein the n-mer is encoded by the nucleic acid sequence of any one of SEQ ID NOs: 108-125, or a sequence at least 95% identical to any one of SEQ ID NOs: 108-125.

10. A composition comprising a stock of the rAAV of claim 1, and one or more of a physiologically compatible carrier, excipient, and/or aqueous suspension base.

11. A recombinant muscle cell-targeting peptide comprising “Xn-n-mer-Xm”, wherein:
(a) Xn is 0, 1, 2, or 3 amino acid residues independently selected from any amino acid;
(b) the n-mer is: AVIRGDV (SEQ ID NO: 2), IIRGDPA (SEQ ID NO: 1), IVRGDPA (SEQ ID NO: 8), MIRGDVK (SEQ ID NO: 9), AQHRGDV (SEQ ID NO: 10), VSRGDPN (SEQ ID NO: 11), VSRGDPA (SEQ ID NO: 12), PLVRGDI (SEQ ID NO:13), PYVRGDP (SEQ ID NO: 14), VVRGDPQ (SEQ ID NO: 15), VVQRGDV (SEQ ID NO: 17), QHRGDTQ (SEQ ID NO: 18), QIRGDLR (SEQ ID NO: 19), RGDYAQV (SEQ ID NO: 20), IGRGDPN (SEQ ID NO: 21), RGDLHGY (SEQ ID NO: 22), RGDYSTM (SEQ ID NO: 23), or PYQRGDH (SEQ ID NO: 24), or an n-mer sequence of at least 6, at least 7, or the full-length consecutive amino acids of any one of the n-mers; and
(c) Xm is 0, 1, 2, or 3 amino acid/s independently selected from any amino acid;
Optionally wherein the muscle cell-targeting peptide is conjugated to a nanoparticle, a second molecule, or a viral capsid protein.

12. The recombinant muscle cell-targeting peptide of claim 11, wherein the recombinant muscle cell-targeting peptide comprises:
(a)
(SEQ ID NO: 2)
AVIRGDV;
or
(b)
(SEQ ID NO: 1)
IIRGDPA.

13. The recombinant muscle targeting peptide of claim 11, wherein the recombinant muscle cell-targeting peptide targets a cardiac muscle cell.

14. The recombinant muscle cell-targeting peptide of claim 11, wherein the recombinant muscle cell-targeting peptide targets a skeletal muscle cell, optionally a gastrocnemius muscle cell.

15. A composition comprising the recombinant muscle cell-targeting peptide of claim 11, and one or more of a physiologically compatible carrier, excipient, and/or aqueous suspension base.

16. A nucleic acid molecule encoding the recombinant muscle cell-targeting peptide of claim 15.

17. A nucleic acid molecule comprising a mutant AAV capsid VP1 gene comprising a nucleic acid sequence encoding an n-mer of: AVIRGDV (SEQ ID NO: 2), IIRGDPA (SEQ ID NO: 1), RGDYAQV (SEQ ID NO: 20), RGDLHGY (SEQ ID NO: 22), PYQRGDH (SEQ ID NO: 24), IVRGDPA (SEQ ID NO: 8), MIRGDVK (SEQ ID NO: 9), AQHRGDV (SEQ ID NO: 10), VSRGDPN (SEQ ID NO: 11), VSRGDPA (SEQ ID NO: 12), PLVRGDI (SEQ ID NO:13), PYVRGDP (SEQ ID NO: 14), VVRGDPQ (SEQ ID NO: 15), VVQRGDV (SEQ ID NO: 17), QHRGDTQ (SEQ ID NO: 18), QIRGDLR (SEQ ID NO: 19), IGRGDPN (SEQ ID NO: 21), or RGDYSTM (SEQ ID NO: 23).

18. The nucleic acid molecule of claim 17, wherein the sequence encoding the n-mer is:
(a) SEQ ID NO: 109 or a sequence at least 99% identical thereto (encoding AVIRGDV);
(b) SEQ ID NO: 108 or a sequence at least 99% identical thereto (encoding IIRGDPA);
(c) SEQ ID NO: 110 or a sequence at least 99% identical thereto (encoding IVRGDPA);
(d) SEQ ID NO: 111 or a sequence at least 99% identical thereto (encoding MIRGDVK);
(e) SEQ ID NO: 112 or a sequence at least 99% identical thereto (encoding AQHRGDV);
(f) SEQ ID NO: 113 or a sequence at least 99% identical thereto (encoding VSRGDPN);
(g) SEQ ID NO: 114 or a sequence at least 99% identical thereto (encoding VSRGDPA);
(h) SEQ ID NO: 115 or a sequence at least 99% identical thereto (encoding PLVRGDI);
(i) SEQ ID NO: 116 or a sequence at least 99% identical thereto (encoding PYVRGDP);
(j) SEQ ID NO: 117 or a sequence at least 99% identical thereto (encoding VVRGDPQ);
(k) SEQ ID NO: 118 or a sequence at least 99% identical thereto (encoding VVQRGDV);
(l) SEQ ID NO: 119 or a sequence at least 99% identical thereto (encoding QHRGDTQ);
(m) SEQ ID NO: 120 or a sequence at least 99% identical thereto (encoding QIRGDLR);
(n) SEQ ID NO: 121 or a sequence at least 99% identical thereto (encoding RGDYAQV);
(o) SEQ ID NO: 122 or a sequence at least 99% identical thereto (encoding IGRGDPN);
(p) SEQ ID NO: 123 or a sequence at least 99% identical thereto (encoding RGDLHGY);
(q) SEQ ID NO: 124 or a sequence at least 99% identical thereto (encoding RGDYSTM); or
(r) SEQ ID NO: 125 or a sequence at least 99% identical thereto (encoding PYQRGDH).

19. The nucleic acid molecule of claim 17, wherein
the nucleic acid sequence encoding the AAV VP1 gene is SEQ ID NO: SEQ ID NO 74, 72, 76, 78, 80, 82, 84, 86, 88, 90, 92, 94, 96, 98, 100, 102, 104, or 106.

20. A fusion polypeptide or protein comprising the recombinant muscle cell-targeting peptide of claim 19 and a fusion partner that comprises at least one polypeptide or protein.

21. A composition comprising a fusion polypeptide or protein of claim 20 and one or more of a physiologically compatible carrier, excipient, and/or aqueous suspension base.

22-23. (canceled)

24. A method for targeted delivery of a gene product to muscle cells in a subject in need thereof, the method comprising administering to the subject a stock of the rAAV of claim 1, optionally wherein the muscle cells are cardiac muscle cells and/or skeletal muscle cells, optionally gastrocnemius muscle cells, deltoid muscle cells, soleus muscle cells, bicep brachii muscle cells or diaphragm muscle cells.

25. A method for treating a muscle cell disorder and/or a disease in a subject in need thereof, the method comprising delivering to the subject a stock of the rAAV of claim 1, wherein the encoded gene product is a protein, optionally an antibody.


SEQUENCE LISTING (128 sequences)

SEQ ID NO: 1 - protein, 7 aa, synthetic construct
IIRGDPA

SEQ ID NO: 2 - protein, 7 aa, synthetic construct
AVIRGDV

SEQ ID NO: 3 - protein, 7 aa, synthetic construct
GQVRVGV

SEQ ID NO: 4 - protein, 7 aa, synthetic construct
MDAHYVR

SEQ ID NO: 5 - protein, 7 aa, synthetic construct
TQAVPLK

SEQ ID NO: 6 - protein, 7 aa, synthetic construct
VVHQTGL

SEQ ID NO: 7 - protein, 7 aa, synthetic construct
MAISRER

SEQ ID NO: 8 - protein, 7 aa, synthetic construct
IVRGDPA

SEQ ID NO: 9 - protein, 7 aa, synthetic construct
MIRGDVK

SEQ ID NO: 10 - protein, 7 aa, synthetic construct
AQHRGDV

SEQ ID NO: 11 - protein, 7 aa, synthetic construct
VSRGDPN

SEQ ID NO: 12 - protein, 7 aa, synthetic construct
VSRGDPA

SEQ ID NO: 13 - protein, 7 aa, synthetic construct
PLVRGDI

SEQ ID NO: 14 - protein, 7 aa, synthetic construct
PYVRGDP

SEQ ID NO: 15 - protein, 7 aa, synthetic construct
VVRGDPQ

SEQ ID NO: 16 - protein, 7 aa, synthetic construct
PTRGDVK

SEQ ID NO: 17 - protein, 7 aa, synthetic construct
VVQRGDV

SEQ ID NO: 18 - protein, 7 aa, synthetic construct
QHRGDTQ

SEQ ID NO: 19 - protein, 7 aa, synthetic construct
QIRGDLR

SEQ ID NO: 20 - protein, 7 aa, synthetic construct
RGDYAQV

SEQ ID NO: 21 - protein, 7 aa, synthetic construct
IGRGDPN

SEQ ID NO: 22 - protein, 7 aa, synthetic construct
RGDLHGY

SEQ ID NO: 23 - protein, 7 aa, synthetic construct
RGDYSTM

SEQ ID NO: 24 - protein, 7 aa, synthetic construct
PYQRGDH

SEQ ID NO: 25 - protein, 736 aa, Adeno-associated virus
  source 1..736: note = serotype 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 26 - protein, 736 aa, Adeno-associated virus
  source 1..736: note = serotype hu68
MAADGYLPDWLEDNLSEGIREWWALKPGAPQPKANQQHQDNARGLVLPGYKYLGPGNGLDKGEPVNEADAAALEHDKAYDQQLKAGDNPYLKYNHADAEFQERLKEDTSFGGNLGRAVFQAKKRLLEPLGLVEEAAKTAPGKKRPVEQSPQEPDSSVGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 27 - protein, 50 aa, synthetic construct
IKTTNPVATESYGQVATNHQSAQAQAQTGWVQNQGILPGMVWQDRDVYLQ

SEQ ID NO: 28 - protein, 50 aa, synthetic construct
EEEIKTTNPVATEEYGIVADNLQQQNTAPQIGTVNSQGALPGMVWQNRDV

SEQ ID NO: 29 - protein, 50 aa, synthetic construct
IRPTNPVATEEYGIVSSNLQAANTAAQTQVVNNQGALPGMVWQNRDVYLQ

SEQ ID NO: 30 - protein, 50 aa, synthetic construct
ASNTALDNVMITDEEEIKATNPVATERFGTVAVNLQSSSTDPATGDVHVM

SEQ ID NO: 31 - protein, 50 aa, synthetic construct
QPVNRVAYNVGGQMATNNQSSTTAPATGTYNLQEIVPGSVWMERDVYLQG

SEQ ID NO: 32 - protein, 50 aa, synthetic construct
FTSEEELAATNATDTDMWGNLPGGDQSNSNLPTVDRLTALGAVPGMVWQN

SEQ ID NO: 33 - protein, 50 aa, synthetic construct
EEIRTTNPVATEQYGTVANNLQSSNTAPTTRTVNDQGALPGMVWQDRDVY

SEQ ID NO: 34 - protein, 50 aa, synthetic construct
RTTNPVATEQYGSVSTNLQRGNRQAATADVNTQGVLPGMVWGDRDVYLQG

SEQ ID NO: 35 - protein, 50 aa, synthetic construct
IKATNPVATERFGTVAVNFQSSSTDPATGDVHAMGALPGMVWQDRDVYLQ

SEQ ID NO: 36 - protein, 7 aa, synthetic construct
NDVRAVS

SEQ ID NO: 37 - protein, 7 aa, synthetic construct
TLAVPFK

SEQ ID NO: 38 - protein, 7 aa, synthetic construct
TLAGPFK

SEQ ID NO: 39 - protein, 13 aa, synthetic construct
SHQIIRGDPAQQA

SEQ ID NO: 40 - protein, 13 aa, synthetic construct
SAQIIRGDMQAQA

SEQ ID NO: 41 - protein, 13 aa, synthetic construct
SWQITRGDPAAQA

SEQ ID NO: 42 - protein, 13 aa, synthetic construct
SAIIIRGDPHAQA

SEQ ID NO: 43 - protein, 13 aa, synthetic construct
GAQIIRGDPQAQA

SEQ ID NO: 44 - protein, 13 aa, synthetic construct
GAQIIRGDPAQQA

SEQ ID NO: 45 - protein, 13 aa, synthetic construct
FAQIIRGDQAAQA

SEQ ID NO: 46 - protein, 13 aa, synthetic construct
SAQIIRGDNANQA

SEQ ID NO: 47 - protein, 13 aa, synthetic construct
QAQIIRGDPQAQA

SEQ ID NO: 48 - protein, 13 aa, synthetic construct
AAQYIRGDPAAQA

SEQ ID NO: 49 - protein, 13 aa, synthetic construct
SGQVIRGDPAAQA

SEQ ID NO: 50 - skipped

SEQ ID NO: 51 - protein, 13 aa, synthetic construct
SAQIIRGDGAAMA

SEQ ID NO: 52 - protein, 13 aa, synthetic construct
SGMIIRGDPAAQA

SEQ ID NO: 53 - protein, 13 aa, synthetic construct
SAQIIRGDNAAQC

SEQ ID NO: 54 - protein, 13 aa, synthetic construct
QAQIIRGDPAAQA

SEQ ID NO: 55 - protein, 13 aa, synthetic construct
SAQIIRGDPAAQA

SEQ ID NO: 56 - protein, 13 aa, synthetic construct
SAYAVARGDVAQA

SEQ ID NO: 57 - protein, 13 aa, synthetic construct
SAQQVIRGDVGQA

SEQ ID NO: 58 - protein, 13 aa, synthetic construct
SAQAGIRGDVARA

SEQ ID NO: 59 - protein, 13 aa, synthetic construct
GAQAAIRGDVAQA

SEQ ID NO: 60 - protein, 13 aa, synthetic construct
SWQAVVRGDVAQA

SEQ ID NO: 61 - protein, 13 aa, synthetic construct
QAQAVIRGDVAQA

SEQ ID NO: 62 - protein, 13 aa, synthetic construct
SAQAVIRGDVGRA

SEQ ID NO: 63 - protein, 13 aa, synthetic construct
SGQAVIRGDVARA

SEQ ID NO: 64 - protein, 13 aa, synthetic construct
SAYAVIRGDVAQA

SEQ ID NO: 65 - protein, 13 aa, synthetic construct
SWQAAIRGDVAQA

SEQ ID NO: 66 - protein, 13 aa, synthetic construct
SAQAYIRGDVAQA

SEQ ID NO: 67 - protein, 13 aa, synthetic construct
SAMAVIRGDVAQA

SEQ ID NO: 68 - protein, 13 aa, synthetic construct
SAHAVIRGDVAQA

SEQ ID NO: 69 - protein, 13 aa, synthetic construct
SAQAVVRGDVAQA

SEQ ID NO: 70 - protein, 13 aa, synthetic construct
SHQAVIRGDVAQA

SEQ ID NO: 71 - protein, 13 aa, synthetic construct
SAQAVIRGDVAQA

SEQ ID NO: 72 - DNA, 2233 bp, synthetic construct
  source 1..2233: mol_type = other DNA
  misc_feature 1765..1785: note = IIRGDPA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaaatcatccgcggcgacccagcagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaat

SEQ ID NO: 73 - protein, 743 aa, synthetic construct
  MOTIF 589..595: note = IIRGDPA
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQIIRGDPAAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 74 - DNA, 2232 bp, synthetic construct
  source 1..2232: mol_type = other DNA
  misc_feature 1765..1785: note = AVIRGDV
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagctgttattcgtggtgatgttgcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 75 - protein, 743 aa, synthetic construct
  MOTIF 589..595: note = AVIRGDV
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAVIRGDVAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 76 - DNA, 2232 bp, synthetic construct
  source 1..2232: mol_type = other DNA
  misc_feature 1765..1785: note = IVRGDPA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaaattgtccgtggcgatccagctgcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 77 - protein, 743 aa, synthetic construct
  MOTIF 589..595: note = IVRGDPA
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQIVRGDPAAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 78 - DNA, 2232 bp, synthetic construct
  source 1..2232: mol_type = other DNA
  misc_feature 1765..1785: note = MIRGDVK
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaaatgatccgtggcgatgtcaaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 79 - protein, 743 aa, synthetic construct
  MOTIF 589..595: note = MIRGDVK
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQMIRGDVKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 80 - DNA, 2232 bp, synthetic construct
  source 1..2232: mol_type = other DNA
  misc_feature 1765..1785: note = AQHRGDV
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagctcagcatcgcggtgacgttgcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 81 - protein, 743 aa, synthetic construct
  MOTIF 589..595: note = AQHRGDV
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQHRGDVAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 82 - DNA, 2232 bp, synthetic construct
  source 1..2232: mol_type = other DNA
  misc_feature 1765..1785: note = VSRGDPN
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagtttctcgtggtgatcctaatgcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 83 - protein, 743 aa, synthetic construct
  MOTIF 589..595: note = VSRGDPN
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQVSRGDPNAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 84 - DNA, 2232 bp, synthetic construct
  source 1..2232: mol_type = other DNA
  misc_feature 1765..1785: note = VSRGDPA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagtttctcgtggtgatcctgctgcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 85 - protein, 743 aa, synthetic construct
  MOTIF 589..595: note = VSRGDPA
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQVSRGDPAAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 86 - DNA, 2232 bp, synthetic construct
  source 1..2232: mol_type = other DNA
  misc_feature 1765..1785: note = PLVRGDI
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaacctcttgttcgcggtgacattgcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 87 - protein, 743 aa, synthetic construct
  MOTIF 589..595: note = PLVRGDI
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQPLVRGDIAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 88 - DNA, 2232 bp, synthetic construct
  source 1..2232: mol_type = other DNA
  misc_feature 1765..1785: note = PYVRGDP
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaaccttacgttcgcggtgaccctgcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 89 - protein, 743 aa, synthetic construct
  MOTIF 589..595: note = PYVRGDP
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQPYVRGDPAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 90 - DNA, 2232 bp, synthetic construct
  source 1..2232: mol_type = other DNA
  misc_feature 1765..1785: note = VVRGDPQ
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagttgttcgtggtgatcctcaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 91 - protein, 743 aa, synthetic construct
  MOTIF 589..595: note = VVRGDPQ
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQVVRGDPQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 92 - DNA, 2232 bp, synthetic construct
  source 1..2232: mol_type = other DNA
  misc_feature 1765..1785: note = VVQRGDV
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagttgtccaacgcggtgacgttgcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 93 - protein, 743 aa, synthetic construct
  MOTIF 589..595: note = VVQRGDV
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQVVQRGDVAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 94 - DNA, 2232 bp, synthetic construct
  source 1..2232: mol_type = other DNA
  misc_feature 1765..1785: note = QHRGDTQ
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaacaacatcgtggtgatactcaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 95 - protein, 743 aa, synthetic construct
  MOTIF 589..595: note = QHRGDTQ
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQQHRGDTQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 96 - DNA, 2232 bp, synthetic construct
  source 1..2232: mol_type = other DNA
  misc_feature 1765..1785: note = QIRGDLR
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaacaaattcgtggtgatttacgtgcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 97 - protein, 743 aa, synthetic construct
  MOTIF 589..595: note = QIRGDLR
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQQIRGDLRAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 98 - DNA, 2232 bp, synthetic construct
  source 1..2232: mol_type = other DNA
  misc_feature 1765..1785: note = RGDYAQV
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaacgtggtgattatgctcaagttgcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 99 - protein, 743 aa, synthetic construct
  MOTIF 589..595: note = RGDYAQV
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDYAQVAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 100 - DNA, 2232 bp, synthetic construct
  source 1..2232: mol_type = other DNA
  misc_feature 1765..1785: note = IGRGDPN
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaaattggccgtggcgatccaaatgcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 101 - protein, 743 aa, synthetic construct
  MOTIF 589..595: note = IGRGDPN
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQIGRGDPNAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 102 - DNA, 2232 bp, synthetic construct
  source 1..2232: mol_type = other DNA
  misc_feature 1765..1785: note = RGDLHGY
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaacgtggtgatttacatggttatgcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 103 - protein, 743 aa, synthetic construct
  MOTIF 589..595: note = RGDLHGY
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDLHGYAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 104 - DNA, 2232 bp, synthetic construct
  source 1..2232: mol_type = other DNA
  misc_feature 1765..1785: note = RGDYSTM
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaacgtggtgattattctactatggcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 105 - protein, 743 aa, synthetic construct
  MOTIF 589..595: note = RGDYSTM
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDYSTMAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 106 - DNA, 2232 bp, synthetic construct
  source 1..2232: mol_type = other DNA
  misc_feature 1765..1785: note = PYQRGDH
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaaccttaccaacgcggtgaccatgcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 107 - protein, 743 aa, synthetic construct
  MOTIF 589..595: note = PYQRGDH
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQPYQRGDHAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 108 - DNA, 21 bp, synthetic construct
  source 1..21: mol_type = other DNA
atcatccgcggcgacccagca

SEQ ID NO: 109 - DNA, 21 bp, synthetic construct
  source 1..21: mol_type = other DNA
gctgttattcgtggtgatgtt

SEQ ID NO: 110 - DNA, 21 bp, synthetic construct
  source 1..21: mol_type = other DNA
attgtccgtggcgatccagct

SEQ ID NO: 111 - DNA, 21 bp, synthetic construct
  source 1..21: mol_type = other DNA
atgatccgtggcgatgtcaaa

SEQ ID NO: 112 - DNA, 21 bp, synthetic construct
  source 1..21: mol_type = other DNA
gctcagcatcgcggtgacgtt

SEQ ID NO: 113 - DNA, 21 bp, synthetic construct
  source 1..21: mol_type = other DNA
gtttctcgtggtgatcctaat

SEQ ID NO: 114 - DNA, 21 bp, synthetic construct
  source 1..21: mol_type = other DNA
gtttctcgtggtgatcctgct

SEQ ID NO: 115 - DNA, 21 bp, synthetic construct
  source 1..21: mol_type = other DNA
cctcttgttcgcggtgacatt

SEQ ID NO: 116 - DNA, 21 bp, synthetic construct
  source 1..21: mol_type = other DNA
ccttacgttcgcggtgaccct

SEQ ID NO: 117 - DNA, 21 bp, synthetic construct
  source 1..21: mol_type = other DNA
gttgttcgtggtgatcctcaa

SEQ ID NO: 118 - DNA, 21 bp, synthetic construct
  source 1..21: mol_type = other DNA
gttgtccaacgcggtgacgtt

SEQ ID NO: 119 - DNA, 21 bp, synthetic construct
  source 1..21: mol_type = other DNA
caacatcgtggtgatactcaa

SEQ ID NO: 120 - DNA, 21 bp, synthetic construct
  source 1..21: mol_type = other DNA
caaattcgtggtgatttacgt

SEQ ID NO: 121 - DNA, 21 bp, synthetic construct
  source 1..21: mol_type = other DNA
cgtggtgattatgctcaagtt

SEQ ID NO: 122 - DNA, 21 bp, synthetic construct
  source 1..21: mol_type = other DNA
attggccgtggcgatccaaat

SEQ ID NO: 123 - DNA, 21 bp, synthetic construct
  source 1..21: mol_type = other DNA
cgtggtgatttacatggttat

SEQ ID NO: 124 - DNA, 21 bp, synthetic construct
  source 1..21: mol_type = other DNA
cgtggtgattattctactatg

SEQ ID NO: 125 - DNA, 21 bp, synthetic construct
  source 1..21: mol_type = other DNA
ccttaccaacgcggtgaccat

SEQ ID NO: 126 - DNA, 1863 bp, Adeno-associated virus
  source 1..1863: mol_type = other DNA; note = serotype 2
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaa

SEQ ID NO: 127 - protein, 621 aa, Adeno-associated virus
  source 1..621: note = serotype 2
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 128 - protein, 7 aa, synthetic construct
RGDYREL